(19) United States
(12) Patent Application Publication
(10) Pub. No.: US 2023/0257449 A1
(43) Pub. Date: Aug. 17, 2023

(54) COMPOSITIONS AND METHODS FOR TREATING AND PREVENTING INFLUENZA

(71) Applicant: VISTERRA, INC., Waltham, MA (US)

(72) Inventors: Karthik Viswanathan, Acton, MA (US); Brian Booth, West Roxbury, MA (US); Boopathy Ramakrishnan, Braintree, MA (US); Andrew M. Wollacott, Milton, MA (US); Gregory Babcock, Marlborough, MA (US); Zachary Shriver, Winchester, MA (US)

(21) Appl. No.: 17/783,966

(22) PCT Filed: Dec. 11, 2020
(86) PCT No.: PCT/US2020/064573

§ 371 Date: Jun. 9, 2022

Related U.S. Application Data
(60) Provisional application No. 62/946,772, filed on Dec. 11, 2019.
(60) Provisional application No. 62/985,623, filed on Mar. 5, 2020.
(60) Provisional application No. 63/028,938, filed on May 22, 2020.

(51) Int. Cl. C07K 16/10; C12N 15/63; A61P 31/16

(52) U.S. Cl. CPC C07K 16/1018; A61P 31/16; C12N 15/63; A61K 2039/505; C07K 2317/52; C07K 2317/565

(57) ABSTRACT

This disclosure relates to binding agents, e.g., antibody molecules, that bind hemagglutinin protein of influenza viruses, and methods of their use.

### CROSS REFERENCE TO RELATED APPLICATIONS

This application claims the benefit of U.S. Provisional Application No. 62/946,772, filed on Dec. 11, 2019, U.S. Provisional Application No. 62/985,623, filed on Mar. 5, 2020, and U.S. Provisional Application No. 63/028,938, filed on May 22, 2020. The contents of the aforementioned applications are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Dec. 10, 2020, is named P2029-7033WO_SL.txt and is 434,364 bytes in size.

### BACKGROUND

Influenza is a common seasonal virus which can be deadly. It is a highly mutable virus resulting in continually emerging new strains. Influenza results in about 35 million infections, about 400,000 hospitalizations, and about 49,000 deaths each season/year in the United States. Globally, there are about 5 million severe diseases and about 500,000 deaths relating to influenza each season/year. Influenza, particularly influenza A, caused global pandemics in 1918, 1957, 1968, and 2009, including about 50 million deaths in 1918 (Lambert and Fauci, N Engl J Med. 2010; 363(21): 2036-2044). Emerging pandemic threats are observed today, and according to the World Health Organization (WHO) new pandemics are expected. For example, the recent H7N9 outbreak in China were associated with high mortality (Kile et al., MMWR Morb Mortal Wkly Rep. 2017; 66(35): 928-932).

There is a need for developing new approaches for preventing and treating influenza, as well as disorders and conditions associated with influenza.

### SUMMARY

The disclosure is based, at least in part, on the discovery of human anti-hemagglutinin (HA) antibody molecules comprising functional and structural properties disclosed herein, e.g., anti-HA antibody molecules that bind a conserved and constrained region or epitope on influenza virus and uses thereof.

Accordingly, the disclosure features binding agents, e.g., antibody molecules, or preparations, or isolated preparations thereof, that bind hemagglutinin (HA) from influenza viruses. In an embodiment, a binding agent, e.g., an antibody molecule, is broad spectrum, and binds more than one HA, e.g., an HA from one or both of Group 1 or Group 2 strains of influenza A viruses. Therefore, in some embodiments, a binding agent, e.g., an antibody molecule, featured in the disclosure can treat or prevent infections by a Group 1 influenza virus and a Group 2 influenza virus. In other embodiments, a binding agent, e.g., an antibody molecule, featured in the disclosure can treat or prevent an infection by an influenza A virus. The binding agents, e.g., antibody molecules, share sufficient structural similarity with antibodies or variable regions disclosed herein such that they possess functional attributes of the antibodies disclosed herein. In some embodiments, the structural similarity can be in terms of three-dimensional structure, or linear amino acid sequence, or both. Without wishing to be bound by theory, it is believed that in an embodiment, the antibody molecules described herein can be used, as a single agent or combination therapy, to prevent an influenza infection a subject at risk of having an influenza, or to treat influenza in subjects exhibiting severe symptoms and/or infected with drug resistant strains.

In an aspect, the disclosure features an anti-HA antibody molecule (e.g., an anti-HA broadly neutralizing antibody (bNAb)), e.g., for seasonal prophylaxis of influenza, having one or more (e.g., 2, 3, 4, 5, 6, or all) of the following properties:
(i) targets an epitope that is conserved, e.g., across a plurality of influenza subtypes and/or strains (e.g., H1N1 and H3N2), and/or is constrained in its ability to mutate;
(ii) binds to a broad panel of HAs, e.g., seasonal coverage across H1 and H3;
(iii) has a Kd value less than or equal to about 1 nM, e.g., less than or equal to about 0.5, 0.6, 0.7, 0.8, 0.9, 1.0, 1.1, 1.2, 1.3, 1.4, 1.5, 2, 3, 4, 5, 6, 7, 8, 9, or 10 nM;
(iv) has an IC₅₀ value less than or equal to about 10 µg/mL (e.g., less than or equal to about 2 or 1 µg/mL) for neutralizing influenza virus (e.g., influenza A virus), e.g., one or more influenza strains, (e.g., an IC50 value less than or equal to about 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 4, 5, 6, 7, 8, 9, or 10 µg/mL);
(v) has a half-life (e.g., circulating half-life) that confers season long protection or is at least 5, 10, 15, 20, 25, 30, 40, 45, 50, 60, 70, 80, 90, 100, 150, 200 days, or more (e.g., 45 days or more), e.g., such that at a subcutaneous or intramuscular administered dose, the antibody molecule is present at the site of action at a concentration that is greater than the IC₅₀ value for over about the entire season;
(vi) has a solubility that is greater than 100 mg/mL (e.g., greater than 120 mg/mL, 150 mg/mL, 200 mg/mL, or 250 mg/mL), e.g., to support subcutaneous or intramuscular administration; or
(vii) has mucosal transport and availability in upper respiratory tract (URT).

In some embodiments, the antibody molecule targets an epitope that is conserved, e.g., across a plurality of influenza subtypes and/or strains (e.g., H1N1 and H3N2), and/or is constrained in its ability to mutate.

In some embodiments, the antibody molecule binds to a broad panel of HAs, e.g., seasonal coverage across H1 and H3.

In some embodiments, the antibody molecule has a Kd value less than or equal to about 1 nM, e.g., less than or equal to about 0.5, 0.6, 0.7, 0.8, 0.9, 1.0, 1.1, 1.2, 1.3, 1.4, 1.5, 2, 3, 4, 5, 6, 7, 8, 9, or 10 nM.

In some embodiments, the antibody molecule has an IC₅₀ value less than or equal to about 10 µg/mL (e.g., less than or equal to about 2 or 1 µg/mL) for neutralizing influenza virus (e.g., influenza A virus), e.g., one or more influenza strains, (e.g., an IC50 value less than or equal to about 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 4, 5, 6, 7, 8, 9, or 10 µg/mL).

In some embodiments, the antibody molecule has a half-life (e.g., circulating half-life) that confers season long protection or is at least 5, 10, 15, 20, 25, 30, 40, 45, 50, 60, 70, 80, 90, 100, 150, 200 days, or more (e.g., 45 days or more), e.g., such that at a subcutaneous or intramuscular administered dose, the antibody molecule is present at the site of action at a concentration that is greater than the IC₅₀ value for over about the entire season.

In some embodiments, the antibody molecule has a solubility that is greater than 100 mg/mL (e.g., greater than 120 mg/mL, 150 mg/mL, 200 mg/mL, or 250 mg/mL), e.g., to support subcutaneous or intramuscular administration.

In some embodiments, the antibody molecule has mucosal transport and availability in upper respiratory tract (URT).

In some embodiments, the antibody molecule is an anti-HA antibody molecule described herein.

In an aspect, the disclosure features an anti-HA antibody molecule comprising (a) one, two, or all of CDR1, CDR2, or CDR3 of a heavy chain variable region segment described herein (e.g., any of SEQ ID NOs: 1-39, 41-43, or 45-187); (b) one, two, or all of CDR1, CDR2, or CDR3 of a light chain variable region segment described herein (e.g., any of SEQ ID NOs: 188-298); or (c) both (a) and (b).

In some embodiments, the antibody molecule comprises CDR1, CDR2, and CDR3 of the heavy chain variable region segment and CDR1, CDR2, and CDR3 of the light chain variable region segment. In some embodiments, the antibody molecule comprises the heavy chain variable region segment, the light chain variable region segment, or both. In some embodiments, the antibody molecule comprises the heavy chain variable region segment and the light chain variable region segment.

In some embodiments, the antibody molecule comprises CDR1, CDR2, and CDR3 of a heavy chain variable region (VH) comprising the amino acid sequences of the corresponding VH CDRs of any one of VH1-VH184 (e.g., as described in Table 15, e.g., any of SEQ ID NOs: 299-318 for HCDR1, any of SEQ ID NOs: 319-348 for HCDR2, and/or any of SEQ ID NOs: 349-423 for HCDR3). In some embodiments, the antibody molecule comprises CDR1, CDR2, and CDR3 of a light chain variable region (VL) comprising the amino acid sequences of the corresponding VL CDRs of any one of VK-1 through VK-111 (e.g., as described in Table 16, e.g., any of SEQ ID NOs: 424-492 for LCDR1, any of SEQ ID NOs: 493-514 for HCDR2, and/or any of SEQ ID NOs: 515-525 for HCDR3). In some embodiments, the antibody molecule comprises (i) CDR1, CDR2, and CDR3 of a heavy chain variable region comprising the amino acid sequences of the corresponding VH CDRs of any one of VH1-VH184, and (ii) CDR1, CDR2, and CDR3 of a light chain variable region comprising the amino acid sequences of the corresponding VL CDRs of any one of VK-1 through VK-111.

In some embodiments, the antibody molecule comprises CDR1, CDR2, and CDR3 of a VH comprising the amino acid sequences of the corresponding VH CDRs of VH1. In some embodiments, the antibody molecule comprises CDR1, CDR2, and CDR3 of a VH comprising the amino acid sequences of the corresponding VH CDRs of VH2. In some embodiments, the antibody molecule comprises CDR1, CDR2, and CDR3 of a VH comprising the amino acid sequences of the corresponding VH CDRs of VH3. In some embodiments, the antibody molecule comprises CDR1, CDR2, and CDR3 of a VH comprising the amino acid sequences of the corresponding VH CDRs of VH4. In some embodiments, the antibody molecule comprises CDR1, CDR2, and CDR3 of a VH comprising the amino acid sequences of the corresponding VH CDRs of VH5. In some embodiments, the antibody molecule comprises CDR1, CDR2, and CDR3 of a VH comprising the amino acid sequences of the corresponding VH CDRs of VH6. In some embodiments, the antibody molecule comprises CDR1, CDR2, and CDR3 of a VH comprising the amino acid sequences of the corresponding VH CDRs of VH7. In some embodiments, the antibody molecule comprises CDR1, CDR2, and CDR3 of a VH comprising the amino acid sequences of the corresponding VH CDRs of VH8. In some embodiments, the antibody molecule comprises CDR1, CDR2, and CDR3 of a VH comprising the amino acid sequences of the corresponding VH CDRs of VH9. In some embodiments, the antibody molecule comprises CDR1, CDR2, and CDR3 of a VH comprising the amino acid sequences of the corresponding VH CDRs of VH10. In some embodiments, the antibody molecule comprises CDR1, CDR2, and CDR3 of a VH comprising the amino acid sequences of the corresponding VH CDRs of VH11. In some embodiments, the antibody molecule comprises CDR1, CDR2, and CDR3 of a VH comprising the amino acid sequences of the corresponding VH CDRs of VH12. In some embodiments, the antibody molecule comprises CDR1, CDR2, and CDR3 of a VH comprising the amino acid sequences of the corresponding VH CDRs of VH13. In some embodiments, the antibody molecule comprises CDR1, CDR2, and CDR3 of a VH comprising the amino acid sequences of the corresponding VH CDRs of VH14. In some embodiments, the antibody molecule comprises CDR1, CDR2, and CDR3 of a VH comprising the amino acid sequences of the corresponding VH CDRs of VH15. In some embodiments, the antibody molecule comprises CDR1, CDR2, and CDR3 of a VH comprising the amino acid sequences of the corresponding VH CDRs of VH16. In some embodiments, the antibody molecule comprises CDR1, CDR2, and CDR3 of a VH comprising the amino acid sequences of the corresponding VH CDRs of VH17. In some embodiments, the antibody molecule comprises CDR1, CDR2, and CDR3 of a VH comprising the amino acid sequences of the corresponding VH CDRs of VH18. In some embodiments, the antibody molecule comprises CDR1, CDR2, and CDR3 of a VH comprising the amino acid sequences of the corresponding VH CDRs of VH19. In some embodiments, the antibody molecule comprises CDR1, CDR2, and CDR3 of a VH comprising the amino acid sequences of the corresponding VH CDRs of VH20. In some embodiments, the antibody molecule comprises CDR1, CDR2, and CDR3 of a VH comprising the amino acid sequences of the corresponding VH CDRs of VH21. In some embodiments, the antibody molecule comprises CDR1, CDR2, and CDR3 of a VH comprising the amino acid sequences of the corresponding VH CDRs of VH22. In some embodiments, the antibody molecule comprises CDR1, CDR2, and CDR3 of a VH comprising the amino acid sequences of the corresponding VH CDRs of VH23. In some embodiments, the antibody molecule comprises CDR1, CDR2, and CDR3 of a VH comprising the amino acid sequences of the corresponding VH CDRs of VH24. In some embodiments, the antibody molecule comprises CDR1, CDR2, and CDR3 of a VH comprising the amino acid sequences of the corresponding VH CDRs of VH25. In some embodiments, the antibody molecule comprises CDR1, CDR2, and CDR3 of a VH comprising the amino acid sequences of the corresponding VH CDRs of VH26. In some embodiments, the antibody molecule comprises CDR1, CDR2, and CDR3 of a VH comprising the amino acid sequences of the corresponding VH CDRs of VH27. In some embodiments, the antibody molecule comprises CDR1, CDR2, and CDR3 of a VH comprising the amino acid sequences of the corresponding VH CDRs of VH28. In some embodiments, the antibody molecule comprises CDR1, CDR2, and CDR3 of a VH comprising the amino acid sequences of the corresponding VH CDRs of VH29. In some embodiments, the antibody molecule comprises CDR1, CDR2, and CDR3 of a VH comprising the amino acid sequences of the corresponding VH CDRs of VH30. In some embodiments, the antibody molecule comprises CDR1, CDR2, and CDR3 of a VH comprising the amino acid sequences of the corresponding VH CDRs of VH31. In some embodiments, the antibody molecule comprises CDR1, CDR2, and CDR3 of a VH comprising the amino acid sequences of the corresponding VH CDRs of VH32. In some embodiments, the antibody molecule comprises CDR1, CDR2, and CDR3 of a VH comprising the amino acid sequences of the corresponding VH CDRs of VH33. In some embodiments, the antibody molecule comprises CDR1, CDR2, and CDR3 of a VH comprising the amino acid sequences of the corresponding VH CDRs of VH34. In some embodiments, the antibody molecule comprises CDR1, CDR2, and CDR3 of a VH comprising the amino acid sequences of the corresponding VH CDRs of VH35. In some embodiments, the antibody molecule comprises CDR1, CDR2, and CDR3 of a VH comprising the amino acid sequences of the corresponding VH CDRs of VH36. In some embodiments, the antibody molecule comprises CDR1, CDR2, and CDR3 of a VH comprising the amino acid sequences of the corresponding VH CDRs of VH37. In some embodiments, the antibody molecule comprises CDR1, CDR2, and CDR3 of a VH comprising the amino acid sequences of the corresponding VH CDRs of VH38. In some embodiments, the antibody molecule comprises CDR1, CDR2, and CDR3 of a VH comprising the amino acid sequences of the corresponding VH CDRs of VH39. In some embodiments, the antibody molecule comprises CDR1, CDR2, and CDR3 of a VH comprising the amino acid sequences of the corresponding VH CDRs of VH40. In some embodiments, the antibody molecule comprises CDR1, CDR2, and CDR3 of a VH comprising the amino acid sequences of the corresponding VH CDRs of VH41. In some embodiments, the antibody molecule comprises CDR1, CDR2, and CDR3 of a VH comprising the amino acid sequences of the corresponding VH CDRs of VH42. In some embodiments, the antibody molecule comprises CDR1, CDR2, and CDR3 of a VH comprising the amino acid sequences of the corresponding VH CDRs of VH43. In some embodiments, the antibody molecule comprises CDR1, CDR2, and CDR3 of a VH comprising the amino acid sequences of the corresponding VH CDRs of VH44. In some embodiments, the antibody molecule comprises CDR1, CDR2, and CDR3 of a VH comprising the amino acid sequences of the corresponding VH CDRs of VH45. In some embodiments, the antibody molecule comprises CDR1, CDR2, and CDR3 of a VH comprising the amino acid sequences of the corresponding VH CDRs of VH46. In some embodiments, the antibody molecule comprises CDR1, CDR2, and CDR3 of a VH comprising the amino acid sequences of the corresponding VH CDRs of VH47. In some embodiments, the antibody molecule comprises CDR1, CDR2, and CDR3 of a VH comprising the amino acid sequences of the corresponding VH CDRs of VH48. In some embodiments, the antibody molecule comprises CDR1, CDR2, and CDR3 of a VH comprising the amino acid sequences of the corresponding VH CDRs of VH49. In some embodiments, the antibody molecule comprises CDR1, CDR2, and CDR3 of a VH comprising the amino acid sequences of the corresponding VH CDRs of VH50. In some embodiments, the antibody molecule comprises CDR1, CDR2, and CDR3 of a VH comprising the amino acid sequences of the corresponding VH CDRs of VH51. In some embodiments, the antibody molecule comprises CDR1, CDR2, and CDR3 of a VH comprising the amino acid sequences of the corresponding VH CDRs of VH52. In some embodiments, the antibody molecule comprises CDR1, CDR2, and CDR3 of a VH comprising the amino acid sequences of the corresponding VH CDRs of VH53. In some embodiments, the antibody molecule comprises CDR1, CDR2, and CDR3 of a VH comprising the amino acid sequences of the corresponding VH CDRs of VH54. In some embodiments, the antibody molecule comprises CDR1, CDR2, and CDR3 of a VH comprising the amino acid sequences of the corresponding VH CDRs of VH55. In some embodiments, the antibody molecule comprises CDR1, CDR2, and CDR3 of a VH comprising the amino acid sequences of the corresponding VH CDRs of VH56. In some embodiments, the antibody molecule comprises CDR1, CDR2, and CDR3 of a VH comprising the amino acid sequences of the corresponding VH CDRs of VH57. In some embodiments, the antibody molecule comprises CDR1, CDR2, and CDR3 of a VH comprising the amino acid sequences of the corresponding VH CDRs of VH58. In some embodiments, the antibody molecule comprises CDR1, CDR2, and CDR3 of a VH comprising the amino acid sequences of the corresponding VH CDRs of VH59. In some embodiments, the antibody molecule comprises CDR1, CDR2, and CDR3 of a VH comprising the amino acid sequences of the corresponding VH CDRs of VH60. In some embodiments, the antibody molecule comprises CDR1, CDR2, and CDR3 of a VH comprising the amino acid sequences of the corresponding VH CDRs of VH61. In some embodiments, the antibody molecule comprises CDR1, CDR2, and CDR3 of a VH comprising the amino acid sequences of the corresponding VH CDRs of VH62. In some embodiments, the antibody molecule comprises CDR1, CDR2, and CDR3 of a VH comprising the amino acid sequences of the corresponding VH CDRs of VH63. In some embodiments, the antibody molecule comprises CDR1, CDR2, and CDR3 of a VH comprising the amino acid sequences of the corresponding VH CDRs of VH64. In some embodiments, the antibody molecule comprises CDR1, CDR2, and CDR3 of a VH comprising the amino acid sequences of the corresponding VH CDRs of VH65. In some embodiments, the antibody molecule comprises CDR1, CDR2, and CDR3 of a VH comprising the amino acid sequences of the corresponding VH CDRs of VH66. In some embodiments, the antibody molecule comprises CDR1, CDR2, and CDR3 of a VH comprising the amino acid sequences of the corresponding VH CDRs of VH67. In some embodiments, the antibody molecule comprises CDR1, CDR2, and CDR3 of a VH comprising the amino acid sequences of the corresponding VH CDRs of VH68. In some embodiments, the antibody molecule comprises CDR1, CDR2, and CDR3 of a VH comprising the amino acid sequences of the corresponding VH CDRs of VH69. In some embodiments, the antibody molecule comprises CDR1, CDR2, and CDR3 of a VH comprising the amino acid sequences of the corresponding VH CDRs of VH70. In some embodiments, the antibody molecule comprises CDR1, CDR2, and CDR3 of a VH comprising the amino acid sequences of the corresponding VH CDRs of VH71. In some embodiments, the antibody molecule comprises CDR1, CDR2, and CDR3 of a VH comprising the amino acid sequences of the corresponding VH CDRs of VH72. In some embodiments, the antibody molecule comprises CDR1, CDR2, and CDR3 of a VH comprising the amino acid sequences of the corresponding VH CDRs of VH73. In some embodiments, the antibody molecule comprises CDR1, CDR2, and CDR3 of a VH comprising the amino acid sequences of the corresponding VH CDRs of VH74. In some embodiments, the antibody molecule comprises CDR1, CDR2, and CDR3 of a VH comprising the amino acid sequences of the corresponding VH CDRs of VH75. In some embodiments, the antibody molecule comprises CDR1, CDR2, and CDR3 of a VH comprising the amino acid sequences of the corresponding VH CDRs of VH76. In some embodiments, the antibody molecule comprises CDR1, CDR2, and CDR3 of a VH comprising the amino acid sequences of the corresponding VH CDRs of VH77. In some embodiments, the antibody molecule comprises CDR1, CDR2, and CDR3 of a VH comprising the amino acid sequences of the corresponding VH CDRs of VH78. In some embodiments, the antibody molecule comprises CDR1, CDR2, and CDR3 of a VH comprising the amino acid sequences of the corresponding VH CDRs of VH79. In some embodiments, the antibody molecule comprises CDR1, CDR2, and CDR3 of a VH comprising the amino acid sequences of the corresponding VH CDRs of VH80. In some embodiments, the antibody molecule comprises CDR1, CDR2, and CDR3 of a VH comprising the amino acid sequences of the corresponding VH CDRs of VH81. In some embodiments, the antibody molecule comprises CDR1, CDR2, and CDR3 of a VH comprising the amino acid sequences of the corresponding VH CDRs of VH82. In some embodiments, the antibody molecule comprises CDR1, CDR2, and CDR3 of a VH comprising the amino acid sequences of the corresponding VH CDRs of VH83. In some embodiments, the antibody molecule comprises CDR1, CDR2, and CDR3 of a VH comprising the amino acid sequences of the corresponding VH CDRs of VH84. In some embodiments, the antibody molecule comprises CDR1, CDR2, and CDR3 of a VH comprising the amino acid sequences of the corresponding VH CDRs of VH85. In some embodiments, the antibody molecule comprises CDR1, CDR2, and CDR3 of a VH comprising the amino acid sequences of the corresponding VH CDRs of VH86. In some embodiments, the antibody molecule comprises CDR1, CDR2, and CDR3 of a VH comprising the amino acid sequences of the corresponding VH CDRs of VH87. In some embodiments, the antibody molecule comprises CDR1, CDR2, and CDR3 of a VH comprising the amino acid sequences of the corresponding VH CDRs of VH88. In some embodiments, the antibody molecule comprises CDR1, CDR2, and CDR3 of a VH comprising the amino acid sequences of the corresponding VH CDRs of VH89. In some embodiments, the antibody molecule comprises CDR1, CDR2, and CDR3 of a VH comprising the amino acid sequences of the corresponding VH CDRs of VH90. In some embodiments, the antibody molecule comprises CDR1, CDR2, and CDR3 of a VH comprising the amino acid sequences of the corresponding VH CDRs of VH91. In some embodiments, the antibody molecule comprises CDR1, CDR2, and CDR3 of a VH comprising the amino acid sequences of the corresponding VH CDRs of VH92. In some embodiments, the antibody molecule comprises CDR1, CDR2, and CDR3 of a VH comprising the amino acid sequences of the corresponding VH CDRs of VH93. In some embodiments, the antibody molecule comprises CDR1, CDR2, and CDR3 of a VH comprising the amino acid sequences of the corresponding VH CDRs of VH94. In some embodiments, the antibody molecule comprises CDR1, CDR2, and CDR3 of a VH comprising the amino acid sequences of the corresponding VH CDRs of VH95. In some embodiments, the antibody molecule comprises CDR1, CDR2, and CDR3 of a VH comprising the amino acid sequences of the corresponding VH CDRs of VH96. In some embodiments, the antibody molecule comprises CDR1, CDR2, and CDR3 of a VH comprising the amino acid sequences of the corresponding VH CDRs of VH97. In some embodiments, the antibody molecule comprises CDR1, CDR2, and CDR3 of a VH comprising the amino acid sequences of the corresponding VH CDRs of VH98. In some embodiments, the antibody molecule comprises CDR1, CDR2, and CDR3 of a VH comprising the amino acid sequences of the corresponding VH CDRs of VH99. In some embodiments, the antibody molecule comprises CDR1, CDR2, and CDR3 of a VH comprising the amino acid sequences of the corresponding VH CDRs of VH100. In some embodiments, the antibody molecule comprises CDR1, CDR2, and CDR3 of a VH comprising the amino acid sequences of the corresponding VH CDRs of VH101. In some embodiments, the antibody molecule comprises CDR1, CDR2, and CDR3 of a VH comprising the amino acid sequences of the corresponding VH CDRs of VH102. In some embodiments, the antibody molecule comprises CDR1, CDR2, and CDR3 of a VH comprising the amino acid sequences of the corresponding VH CDRs of VH103. In some embodiments, the antibody molecule comprises CDR1, CDR2, and CDR3 of a VH comprising the amino acid sequences of the corresponding VH CDRs of VH104. In some embodiments, the antibody molecule comprises CDR1, CDR2, and CDR3 of a VH comprising the amino acid sequences of the corresponding VH CDRs of VH105. In some embodiments, the antibody molecule comprises CDR1, CDR2, and CDR3 of a VH comprising the amino acid sequences of the corresponding VH CDRs of VH106. In some embodiments, the antibody molecule comprises CDR1, CDR2, and CDR3 of a VH comprising the amino acid sequences of the corresponding VH CDRs of VH107. In some embodiments, the antibody molecule comprises CDR1, CDR2, and CDR3 of a VH comprising the amino acid sequences of the corresponding VH CDRs of VH108. In some embodiments, the antibody molecule comprises CDR1, CDR2, and CDR3 of a VH comprising the amino acid sequences of the corresponding VH CDRs of VH109. In some embodiments, the antibody molecule comprises CDR1, CDR2, and CDR3 of a VH comprising the amino acid sequences of the corresponding VH CDRs of VH110. In some embodiments, the antibody molecule comprises CDR1, CDR2, and CDR3 of a VH comprising the amino acid sequences of the corresponding VH CDRs of VH111. In some embodiments, the antibody molecule comprises CDR1, CDR2, and CDR3 of a VH comprising the amino acid sequences of the corresponding VH CDRs of VH112. In some embodiments, the antibody molecule comprises CDR1, CDR2, and CDR3 of a VH comprising the amino acid sequences of the corresponding VH CDRs of VH113. In some embodiments, the antibody molecule comprises CDR1, CDR2, and CDR3 of a VH comprising the amino acid sequences of the corresponding VH CDRs of VH114. In some embodiments, the antibody molecule comprises CDR1, CDR2, and CDR3 of a VH comprising the amino acid sequences of the corresponding VH CDRs of VH115. In some embodiments, the antibody molecule comprises CDR1, CDR2, and CDR3 of a VH comprising the amino acid sequences of the corresponding VH CDRs of VH116. In some embodiments, the antibody molecule comprises CDR1, CDR2, and CDR3 of a VH comprising the amino acid sequences of the corresponding VH CDRs of VH117. In some embodiments, the antibody molecule comprises CDR1, CDR2, and CDR3 of a VH comprising the amino acid sequences of the corresponding VH CDRs of VH118. In some embodiments, the antibody molecule comprises CDR1, CDR2, and CDR3 of a VH comprising the amino acid sequences of the corresponding VH CDRs of VH119. In some embodiments, the antibody molecule comprises CDR1, CDR2, and CDR3 of a VH comprising the amino acid sequences of the corresponding VH CDRs of VH1120. In some embodiments, the antibody molecule comprises CDR1, CDR2, and CDR3 of a VH comprising the amino acid sequences of the corresponding VH CDRs of VH121. In some embodiments, the antibody molecule comprises CDR1, CDR2, and CDR3 of a VH comprising the amino acid sequences of the corresponding VH CDRs of VH122. In some embodiments, the antibody molecule comprises CDR1, CDR2, and CDR3 of a VH comprising the amino acid sequences of the corresponding VH CDRs of VH123. In some embodiments, the antibody molecule comprises CDR1, CDR2, and CDR3 of a VH comprising the amino acid sequences of the corresponding VH CDRs of VH124. In some embodiments, the antibody molecule comprises CDR1, CDR2, and CDR3 of a VH comprising the amino acid sequences of the corresponding VH CDRs of VH125. In some embodiments, the antibody molecule comprises CDR1, CDR2, and CDR3 of a VH comprising the amino acid sequences of the corresponding VH CDRs of VH126. In some embodiments, the antibody molecule comprises CDR1, CDR2, and CDR3 of a VH comprising the amino acid sequences of the corresponding VH CDRs of VH127. In some embodiments, the antibody molecule comprises CDR1, CDR2, and CDR3 of a VH comprising the amino acid sequences of the corresponding VH CDRs of VH128. In some embodiments, the antibody molecule comprises CDR1, CDR2, and CDR3 of a VH comprising the amino acid sequences of the corresponding VH CDRs of VH129. In some embodiments, the antibody molecule comprises CDR1, CDR2, and CDR3 of a VH comprising the amino acid sequences of the corresponding VH CDRs of VH130. In some embodiments, the antibody molecule comprises CDR1, CDR2, and CDR3 of a VH comprising the amino acid sequences of the corresponding VH CDRs of VH131. In some embodiments, the antibody molecule comprises CDR1, CDR2, and CDR3 of a VH comprising the amino acid sequences of the corresponding VH CDRs of VH132. In some embodiments, the antibody molecule comprises CDR1, CDR2, and CDR3 of a VH comprising the amino acid sequences of the corresponding VH CDRs of VH133. In some embodiments, the antibody molecule comprises CDR1, CDR2, and CDR3 of a VH comprising the amino acid sequences of the corresponding VH CDRs of VH134. In some embodiments, the antibody molecule comprises CDR1, CDR2, and CDR3 of a VH comprising the amino acid sequences of the corresponding VH CDRs of VH135. In some embodiments, the antibody molecule comprises CDR1, CDR2, and CDR3 of a VH comprising the amino acid sequences of the corresponding VH CDRs of VH136. In some embodiments, the antibody molecule comprises CDR1, CDR2, and CDR3 of a VH comprising the amino acid sequences of the corresponding VH CDRs of VH137. In some embodiments, the antibody molecule comprises CDR1, CDR2, and CDR3 of a VH comprising the amino acid sequences of the corresponding VH CDRs of VH138. In some embodiments, the antibody molecule comprises CDR1, CDR2, and CDR3 of a VH comprising the amino acid sequences of the corresponding VH CDRs of VH139. In some embodiments, the antibody molecule comprises CDR1, CDR2, and CDR3 of a VH comprising the amino acid sequences of the corresponding VH CDRs of VH140. In some embodiments, the antibody molecule comprises CDR1, CDR2, and CDR3 of a VH comprising the amino acid sequences of the corresponding VH CDRs of VH141. In some embodiments, the antibody molecule comprises CDR1, CDR2, and CDR3 of a VH comprising the amino acid sequences of the corresponding VH CDRs of VH142. In some embodiments, the antibody molecule comprises CDR1, CDR2, and CDR3 of a VH comprising the amino acid sequences of the corresponding VH CDRs of VH143. In some embodiments, the antibody molecule comprises CDR1, CDR2, and CDR3 of a VH comprising the amino acid sequences of the corresponding VH CDRs of VH144. In some embodiments, the antibody molecule comprises CDR1, CDR2, and CDR3 of a VH comprising the amino acid sequences of the corresponding VH CDRs of VH145. In some embodiments, the antibody molecule comprises CDR1, CDR2, and CDR3 of a VH comprising the amino acid sequences of the corresponding VH CDRs of VH146. In some embodiments, the antibody molecule comprises CDR1, CDR2, and CDR3 of a VH comprising the amino acid sequences of the corresponding VH CDRs of VH147. In some embodiments, the antibody molecule comprises CDR1, CDR2, and CDR3 of a VH comprising the amino acid sequences of the corresponding VH CDRs of VH148. In some embodiments, the antibody molecule comprises CDR1, CDR2, and CDR3 of a VH comprising the amino acid sequences of the corresponding VH CDRs of VH149. In some embodiments, the antibody molecule comprises CDR1, CDR2, and CDR3 of a VH comprising the amino acid sequences of the corresponding VH CDRs of VH150. In some embodiments, the antibody molecule comprises CDR1, CDR2, and CDR3 of a VH comprising the amino acid sequences of the corresponding VH CDRs of VH151. In some embodiments, the antibody molecule comprises CDR1, CDR2, and CDR3 of a VH comprising the amino acid sequences of the corresponding VH CDRs of VH152. In some embodiments, the antibody molecule comprises CDR1, CDR2, and CDR3 of a VH comprising the amino acid sequences of the corresponding VH CDRs of VH153. In some embodiments, the antibody molecule comprises CDR1, CDR2, and CDR3 of a VH comprising the amino acid sequences of the corresponding VH CDRs of VH154. In some embodiments, the antibody molecule comprises CDR1, CDR2, and CDR3 of a VH comprising the amino acid sequences of the corresponding VH CDRs of VH155. In some embodiments, the antibody molecule comprises CDR1, CDR2, and CDR3 of a VH comprising the amino acid sequences of the corresponding VH CDRs of VH156. In some embodiments, the antibody molecule comprises CDR1, CDR2, and CDR3 of a VH comprising the amino acid sequences of the corresponding VH CDRs of VH157. In some embodiments, the antibody molecule comprises CDR1, CDR2, and CDR3 of a VH comprising the amino acid sequences of the corresponding VH CDRs of VH158. In some embodiments, the antibody molecule comprises CDR1, CDR2, and CDR3 of a VH comprising the amino acid sequences of the corresponding VH CDRs of VH159. In some embodiments, the antibody molecule comprises CDR1, CDR2, and CDR3 of a VH comprising the amino acid sequences of the corresponding VH CDRs of VH160. In some embodiments, the antibody molecule comprises CDR1, CDR2, and CDR3 of a VH comprising the amino acid sequences of the corresponding VH CDRs of VH161. In some embodiments, the antibody molecule comprises CDR1, CDR2, and CDR3 of a VH comprising the amino acid sequences of the corresponding VH CDRs of VH162. In some embodiments, the antibody molecule comprises CDR1, CDR2, and CDR3 of a VH comprising the amino acid sequences of the corresponding VH CDRs of VH163. In some embodiments, the antibody molecule comprises CDR1, CDR2, and CDR3 of a VH comprising the amino acid sequences of the corresponding VH CDRs of VH164. In some embodiments, the antibody molecule comprises CDR1, CDR2, and CDR3 of a VH comprising the amino acid sequences of the corresponding VH CDRs of VH165. In some embodiments, the antibody molecule comprises CDR1, CDR2, and CDR3 of a VH comprising the amino acid sequences of the corresponding VH CDRs of VH166. In some embodiments, the antibody molecule comprises CDR1, CDR2, and CDR3 of a VH comprising the amino acid sequences of the corresponding VH CDRs of VH167. In some embodiments, the antibody molecule comprises CDR1, CDR2, and CDR3 of a VH comprising the amino acid sequences of the corresponding VH CDRs of VH168. In some embodiments, the antibody molecule comprises CDR1, CDR2, and CDR3 of a VH comprising the amino acid sequences of the corresponding VH CDRs of VH169. In some embodiments, the antibody molecule comprises CDR1, CDR2, and CDR3 of a VH comprising the amino acid sequences of the corresponding VH CDRs of VH170. In some embodiments, the antibody molecule comprises CDR1, CDR2, and CDR3 of a VH comprising the amino acid sequences of the corresponding VH CDRs of VH171. In some embodiments, the antibody molecule comprises CDR1, CDR2, and CDR3 of a VH comprising the amino acid sequences of the corresponding VH CDRs of VH172. In some embodiments, the antibody molecule comprises CDR1, CDR2, and CDR3 of a VH comprising the amino acid sequences of the corresponding VH CDRs of VH173. In some embodiments, the antibody molecule comprises CDR1, CDR2, and CDR3 of a VH comprising the amino acid sequences of the corresponding VH CDRs of VH174. In some embodiments, the antibody molecule comprises CDR1, CDR2, and CDR3 of a VH comprising the amino acid sequences of the corresponding VH CDRs of VH175. In some embodiments, the antibody molecule comprises CDR1, CDR2, and CDR3 of a VH comprising the amino acid sequences of the corresponding VH CDRs of VH176. In some embodiments, the antibody molecule comprises CDR1, CDR2, and CDR3 of a VH comprising the amino acid sequences of the corresponding VH CDRs of VH177. In some embodiments, the antibody molecule comprises CDR1, CDR2, and CDR3 of a VH comprising the amino acid sequences of the corresponding VH CDRs of VH178. In some embodiments, the antibody molecule comprises CDR1, CDR2, and CDR3 of a VH comprising the amino acid sequences of the corresponding VH CDRs of VH179. In some embodiments, the antibody molecule comprises CDR1, CDR2, and CDR3 of a VH comprising the amino acid sequences of the corresponding VH CDRs of VH180. In some embodiments, the antibody molecule comprises CDR1, CDR2, and CDR3 of a VH comprising the amino acid sequences of the corresponding VH CDRs of VH181. In some embodiments, the antibody molecule comprises CDR1, CDR2, and CDR3 of a VH comprising the amino acid sequences of the corresponding VH CDRs of VH182. In some embodiments, the antibody molecule comprises CDR1, CDR2, and CDR3 of a VH comprising the amino acid sequences of the corresponding VH CDRs of VH183. In some embodiments, the antibody molecule comprises CDR1, CDR2, and CDR3 of a VH comprising the amino acid sequences of the corresponding VH CDRs of VH184.

In some embodiments, the antibody molecule comprises CDR1, CDR2, and CDR3 of a VL comprising the amino acid sequences of the corresponding VL CDRs of VK-1. In some embodiments, the antibody molecule comprises CDR1, CDR2, and CDR3 of a VL comprising the amino acid sequences of the corresponding VL CDRs of VK-2. In some embodiments, the antibody molecule comprises CDR1, CDR2, and CDR3 of a VL comprising the amino acid sequences of the corresponding VL CDRs of VK-3. In some embodiments, the antibody molecule comprises CDR1, CDR2, and CDR3 of a VL comprising the amino acid sequences of the corresponding VL CDRs of VK-4. In some embodiments, the antibody molecule comprises CDR1, CDR2, and CDR3 of a VL comprising the amino acid sequences of the corresponding VL CDRs of VK-5. In some embodiments, the antibody molecule comprises CDR1, CDR2, and CDR3 of a VL comprising the amino acid sequences of the corresponding VL CDRs of VK-6. In some embodiments, the antibody molecule comprises CDR1, CDR2, and CDR3 of a VL comprising the amino acid sequences of the corresponding VL CDRs of VK-7. In some embodiments, the antibody molecule comprises CDR1, CDR2, and CDR3 of a VL comprising the amino acid sequences of the corresponding VL CDRs of VK-8. In some embodiments, the antibody molecule comprises CDR1, CDR2, and CDR3 of a VL comprising the amino acid sequences of the corresponding VL CDRs of VK-9. In some embodiments, the antibody molecule comprises CDR1, CDR2, and CDR3 of a VL comprising the amino acid sequences of the corresponding VL CDRs of VK-10. In some embodiments, the antibody molecule comprises CDR1, CDR2, and CDR3 of a VL comprising the amino acid sequences of the corresponding VL CDRs of VK-11. In some embodiments, the antibody molecule comprises CDR1, CDR2, and CDR3 of a VL comprising the amino acid sequences of the corresponding VL CDRs of VK-12. In some embodiments, the antibody molecule comprises CDR1, CDR2, and CDR3 of a VL comprising the amino acid sequences of the corresponding VL CDRs of VK-13. In some embodiments, the antibody molecule comprises CDR1, CDR2, and CDR3 of a VL comprising the amino acid sequences of the corresponding VL CDRs of VK-14. In some embodiments, the antibody molecule comprises CDR1, CDR2, and CDR3 of a VL comprising the amino acid sequences of the corresponding VL CDRs of VK-15. In some embodiments, the antibody molecule comprises CDR1, CDR2, and CDR3 of a VL comprising the amino acid sequences of the corresponding VL CDRs of VK-16. In some embodiments, the antibody molecule comprises CDR1, CDR2, and CDR3 of a VL comprising the amino acid sequences of the corresponding VL CDRs of VK-17. In some embodiments, the antibody molecule comprises CDR1, CDR2, and CDR3 of a VL comprising the amino acid sequences of the corresponding VL CDRs of VK-18. In some embodiments, the antibody molecule comprises CDR1, CDR2, and CDR3 of a VL comprising the amino acid sequences of the corresponding VL CDRs of VK-19. In some embodiments, the antibody molecule comprises CDR1, CDR2, and CDR3 of a VL comprising the amino acid sequences of the corresponding VL CDRs of VK-20. In some embodiments, the antibody molecule comprises CDR1, CDR2, and CDR3 of a VL comprising the amino acid sequences of the corresponding VL CDRs of VK-21. In some embodiments, the antibody molecule comprises CDR1, CDR2, and CDR3 of a VL comprising the amino acid sequences of the corresponding VL CDRs of VK-22. In some embodiments, the antibody molecule comprises CDR1, CDR2, and CDR3 of a VL comprising the amino acid sequences of the corresponding VL CDRs of VK-23. In some embodiments, the antibody molecule comprises CDR1, CDR2, and CDR3 of a VL comprising the amino acid sequences of the corresponding VL CDRs of VK-24. In some embodiments, the antibody molecule comprises CDR1, CDR2, and CDR3 of a VL comprising the amino acid sequences of the corresponding VL CDRs of VK-25. In some embodiments, the antibody molecule comprises CDR1, CDR2, and CDR3 of a VL comprising the amino acid sequences of the corresponding VL CDRs of VK-26. In some embodiments, the antibody molecule comprises CDR1, CDR2, and CDR3 of a VL comprising the amino acid sequences of the corresponding VL CDRs of VK-27. In some embodiments, the antibody molecule comprises CDR1, CDR2, and CDR3 of a VL comprising the amino acid sequences of the corresponding VL CDRs of VK-28. In some embodiments, the antibody molecule comprises CDR1, CDR2, and CDR3 of a VL comprising the amino acid sequences of the corresponding VL CDRs of VK-29. In some embodiments, the antibody molecule comprises CDR1, CDR2, and CDR3 of a VL comprising the amino acid sequences of the corresponding VL CDRs of VK-30. In some embodiments, the antibody molecule comprises CDR1, CDR2, and CDR3 of a VL comprising the amino acid sequences of the corresponding VL CDRs of VK-31. In some embodiments, the antibody molecule comprises CDR1, CDR2, and CDR3 of a VL comprising the amino acid sequences of the corresponding VL CDRs of VK-32. In some embodiments, the antibody molecule comprises CDR1, CDR2, and CDR3 of a VL comprising the amino acid sequences of the corresponding VL CDRs of VK-33. In some embodiments, the antibody molecule comprises CDR1, CDR2, and CDR3 of a VL comprising the amino acid sequences of the corresponding VL CDRs of VK-34. In some embodiments, the antibody molecule comprises CDR1, CDR2, and CDR3 of a VL comprising the amino acid sequences of the corresponding VL CDRs of VK-35. In some embodiments, the antibody molecule comprises CDR1, CDR2, and CDR3 of a VL comprising the amino acid sequences of the corresponding VL CDRs of VK-36. In some embodiments, the antibody molecule comprises CDR1, CDR2, and CDR3 of a VL comprising the amino acid sequences of the corresponding VL CDRs of VK-37. In some embodiments, the antibody molecule comprises CDR1, CDR2, and CDR3 of a VL comprising the amino acid sequences of the corresponding VL CDRs of VK-38. In some embodiments, the antibody molecule comprises CDR1, CDR2, and CDR3 of a VL comprising the amino acid sequences of the corresponding VL CDRs of VK-39. In some embodiments, the antibody molecule comprises CDR1, CDR2, and CDR3 of a VL comprising the amino acid sequences of the corresponding VL CDRs of VK-40. In some embodiments, the antibody molecule comprises CDR1, CDR2, and CDR3 of a VL comprising the amino acid sequences of the corresponding VL CDRs of VK-41. In some embodiments, the antibody molecule comprises CDR1, CDR2, and CDR3 of a VL comprising the amino acid sequences of the corresponding VL CDRs of VK-42. In some embodiments, the antibody molecule comprises CDR1, CDR2, and CDR3 of a VL comprising the amino acid sequences of the corresponding VL CDRs of VK-43. In some embodiments, the antibody molecule comprises CDR1, CDR2, and CDR3 of a VL comprising the amino acid sequences of the corresponding VL CDRs of VK-44. In some embodiments, the antibody molecule comprises CDR1, CDR2, and CDR3 of a VL comprising the amino acid sequences of the corresponding VL CDRs of VK-45. In some embodiments, the antibody molecule comprises CDR1, CDR2, and CDR3 of a VL comprising the amino acid sequences of the corresponding VL CDRs of VK-46. In some embodiments, the antibody molecule comprises CDR1, CDR2, and CDR3 of a VL comprising the amino acid sequences of the corresponding VL CDRs of VK-47. In some embodiments, the antibody molecule comprises CDR1, CDR2, and CDR3 of a VL comprising the amino acid sequences of the corresponding VL CDRs of VK-48. In some embodiments, the antibody molecule comprises CDR1, CDR2, and CDR3 of a VL comprising the amino acid sequences of the corresponding VL CDRs of VK-49. In some embodiments, the antibody molecule comprises CDR1, CDR2, and CDR3 of a VL comprising the amino acid sequences of the corresponding VL CDRs of VK-50. In some embodiments, the antibody molecule comprises CDR1, CDR2, and CDR3 of a VL comprising the amino acid sequences of the corresponding VL CDRs of VK-51. In some embodiments, the antibody molecule comprises CDR1, CDR2, and CDR3 of a VL comprising the amino acid sequences of the corresponding VL CDRs of VK-52. In some embodiments, the antibody molecule comprises CDR1, CDR2, and CDR3 of a VL comprising the amino acid sequences of the corresponding VL CDRs of VK-53. In some embodiments, the antibody molecule comprises CDR1, CDR2, and CDR3 of a VL comprising the amino acid sequences of the corresponding VL CDRs of VK-54. In some embodiments, the antibody molecule comprises CDR1, CDR2, and CDR3 of a VL comprising the amino acid sequences of the corresponding VL CDRs of VK-55. In some embodiments, the antibody molecule comprises CDR1, CDR2, and CDR3 of a VL comprising the amino acid sequences of the corresponding VL CDRs of VK-56. In some embodiments, the antibody molecule comprises CDR1, CDR2, and CDR3 of a VL comprising the amino acid sequences of the corresponding VL CDRs of VK-57. In some embodiments, the antibody molecule comprises CDR1, CDR2, and CDR3 of a VL comprising the amino acid sequences of the corresponding VL CDRs of VK-58. In some embodiments, the antibody molecule comprises CDR1, CDR2, and CDR3 of a VL comprising the amino acid sequences of the corresponding VL CDRs of VK-59. In some embodiments, the antibody molecule comprises CDR1, CDR2, and CDR3 of a VL comprising the amino acid sequences of the corresponding VL CDRs of VK-60. In some embodiments, the antibody molecule comprises CDR1, CDR2, and CDR3 of a VL comprising the amino acid sequences of the corresponding VL CDRs of VK-61. In some embodiments, the antibody molecule comprises CDR1, CDR2, and CDR3 of a VL comprising the amino acid sequences of the corresponding VL CDRs of VK-62. In some embodiments, the antibody molecule comprises CDR1, CDR2, and CDR3 of a VL comprising the amino acid sequences of the corresponding VL CDRs of VK-63. In some embodiments, the antibody molecule comprises CDR1, CDR2, and CDR3 of a VL comprising the amino acid sequences of the corresponding VL CDRs of VK-64. In some embodiments, the antibody molecule comprises CDR1, CDR2, and CDR3 of a VL comprising the amino acid sequences of the corresponding VL CDRs of VK-65. In some embodiments, the antibody molecule comprises CDR1, CDR2, and CDR3 of a VL comprising the amino acid sequences of the corresponding VL CDRs of VK-66. In some embodiments, the antibody molecule comprises CDR1, CDR2, and CDR3 of a VL comprising the amino acid sequences of the corresponding VL CDRs of VK-67. In some embodiments, the antibody molecule comprises CDR1, CDR2, and CDR3 of a VL comprising the amino acid sequences of the corresponding VL CDRs of VK-68. In some embodiments, the antibody molecule comprises CDR1, CDR2, and CDR3 of a VL comprising the amino acid sequences of the corresponding VL CDRs of VK-69. In some embodiments, the antibody molecule comprises CDR1, CDR2, and CDR3 of a VL comprising the amino acid sequences of the corresponding VL CDRs of VK-70. In some embodiments, the antibody molecule comprises CDR1, CDR2, and CDR3 of a VL comprising the amino acid sequences of the corresponding VL CDRs of VK-71. In some embodiments, the antibody molecule comprises CDR1, CDR2, and CDR3 of a VL comprising the amino acid sequences of the corresponding VL CDRs of VK-72. In some embodiments, the antibody molecule comprises CDR1, CDR2, and CDR3 of a VL comprising the amino acid sequences of the corresponding VL CDRs of VK-73. In some embodiments, the antibody molecule comprises CDR1, CDR2, and CDR3 of a VL comprising the amino acid sequences of the corresponding VL CDRs of VK-74. In some embodiments, the antibody molecule comprises CDR1, CDR2, and CDR3 of a VL comprising the amino acid sequences of the corresponding VL CDRs of VK-75. In some embodiments, the antibody molecule comprises CDR1, CDR2, and CDR3 of a VL comprising the amino acid sequences of the corresponding VL CDRs of VK-76. In some embodiments, the antibody molecule comprises CDR1, CDR2, and CDR3 of a VL comprising the amino acid sequences of the corresponding VL CDRs of VK-77. In some embodiments, the antibody molecule comprises CDR1, CDR2, and CDR3 of a VL comprising the amino acid sequences of the corresponding VL CDRs of VK-78. In some embodiments, the antibody molecule comprises CDR1, CDR2, and CDR3 of a VL comprising the amino acid sequences of the corresponding VL CDRs of VK-79. In some embodiments, the antibody molecule comprises CDR1, CDR2, and CDR3 of a VL comprising the amino acid sequences of the corresponding VL CDRs of VK-80. In some embodiments, the antibody molecule comprises CDR1, CDR2, and CDR3 of a VL comprising the amino acid sequences of the corresponding VL CDRs of VK-81. In some embodiments, the antibody molecule comprises CDR1, CDR2, and CDR3 of a VL comprising the amino acid sequences of the corresponding VL CDRs of VK-82. In some embodiments, the antibody molecule comprises CDR1, CDR2, and CDR3 of a VL comprising the amino acid sequences of the corresponding VL CDRs of VK-83. In some embodiments, the antibody molecule comprises CDR1, CDR2, and CDR3 of a VL comprising the amino acid sequences of the corresponding VL CDRs of VK-84. In some embodiments, the antibody molecule comprises CDR1, CDR2, and CDR3 of a VL comprising the amino acid sequences of the corresponding VL CDRs of VK-85. In some embodiments, the antibody molecule comprises CDR1, CDR2, and CDR3 of a VL comprising the amino acid sequences of the corresponding VL CDRs of VK-86. In some embodiments, the antibody molecule comprises CDR1, CDR2, and CDR3 of a VL comprising the amino acid sequences of the corresponding VL CDRs of VK-87. In some embodiments, the antibody molecule comprises CDR1, CDR2, and CDR3 of a VL comprising the amino acid sequences of the corresponding VL CDRs of VK-88. In some embodiments, the antibody molecule comprises CDR1, CDR2, and CDR3 of a VL comprising the amino acid sequences of the corresponding VL CDRs of VK-89. In some embodiments, the antibody molecule comprises CDR1, CDR2, and CDR3 of a VL comprising the amino acid sequences of the corresponding VL CDRs of VK-90. In some embodiments, the antibody molecule comprises CDR1, CDR2, and CDR3 of a VL comprising the amino acid sequences of the corresponding VL CDRs of VK-91. In some embodiments, the antibody molecule comprises CDR1, CDR2, and CDR3 of a VL comprising the amino acid sequences of the corresponding VL CDRs of VK-92. In some embodiments, the antibody molecule comprises CDR1, CDR2, and CDR3 of a VL comprising the amino acid sequences of the corresponding VL CDRs of VK-93. In some embodiments, the antibody molecule comprises CDR1, CDR2, and CDR3 of a VL comprising the amino acid sequences of the corresponding VL CDRs of VK-94. In some embodiments, the antibody molecule comprises CDR1, CDR2, and CDR3 of a VL comprising the amino acid sequences of the corresponding VL CDRs of VK-95. In some embodiments, the antibody molecule comprises CDR1, CDR2, and CDR3 of a VL comprising the amino acid sequences of the corresponding VL CDRs of VK-96. In some embodiments, the antibody molecule comprises CDR1, CDR2, and CDR3 of a VL comprising the amino acid sequences of the corresponding VL CDRs of VK-97. In some embodiments, the antibody molecule comprises CDR1, CDR2, and CDR3 of a VL comprising the amino acid sequences of the corresponding VL CDRs of VK-98. In some embodiments, the antibody molecule comprises CDR1, CDR2, and CDR3 of a VL comprising the amino acid sequences of the corresponding VL CDRs of VK-99. In some embodiments, the antibody molecule comprises CDR1, CDR2, and CDR3 of a VL comprising the amino acid sequences of the corresponding VL CDRs of VK-100. In some embodiments, the antibody molecule comprises CDR1, CDR2, and CDR3 of a VL comprising the amino acid sequences of the corresponding VL CDRs of VK-101. In some embodiments, the antibody molecule comprises CDR1, CDR2, and CDR3 of a VL comprising the amino acid sequences of the corresponding VL CDRs of VK-102. In some embodiments, the antibody molecule comprises CDR1, CDR2, and CDR3 of a VL comprising the amino acid sequences of the corresponding VL CDRs of VK-103. In some embodiments, the antibody molecule comprises CDR1, CDR2, and CDR3 of a VL comprising the amino acid sequences of the corresponding VL CDRs of VK-104. In some embodiments, the antibody molecule comprises CDR1, CDR2, and CDR3 of a VL comprising the amino acid sequences of the corresponding VL CDRs of VK-105. In some embodiments, the antibody molecule comprises CDR1, CDR2, and CDR3 of a VL comprising the amino acid sequences of the corresponding VL CDRs of VK-106. In some embodiments, the antibody molecule comprises CDR1, CDR2, and CDR3 of a VL comprising the amino acid sequences of the corresponding VL CDRs of VK-107. In some embodiments, the antibody molecule comprises CDR1, CDR2, and CDR3 of a VL comprising the amino acid sequences of the corresponding VL CDRs of VK-108. In some embodiments, the antibody molecule comprises CDR1, CDR2, and CDR3 of a VL comprising the amino acid sequences of the corresponding VL CDRs of VK-109. In some embodiments, the antibody molecule comprises CDR1, CDR2, and CDR3 of a VL comprising the amino acid sequences of the corresponding VL CDRs of VK-110. In some embodiments, the antibody molecule comprises CDR1, CDR2, and CDR3 of a VL comprising the amino acid sequences of the corresponding VL CDRs of VK-111.

In some embodiments, the antibody molecule comprises CDR1, CDR2, and CDR3 of a VH comprising the amino acid sequences of the corresponding VH CDRs of VH107. In some embodiments, the antibody molecule comprises CDR1, CDR2, and CDR3 of a VH comprising the amino acid sequences of the corresponding VH CDRs of VH123. In some embodiments, the antibody molecule comprises CDR1, CDR2, and CDR3 of a VH comprising the amino acid sequences of the corresponding VH CDRs of VH148. In some embodiments, the antibody molecule comprises CDR1, CDR2, and CDR3 of a VH comprising the amino acid sequences of the corresponding VH CDRs of VH175. In some embodiments, the antibody molecule comprises CDR1, CDR2, and CDR3 of a VH comprising the amino acid sequences of the corresponding VH CDRs of VH176.

In some embodiments, the antibody molecule comprises CDR1, CDR2, and CDR3 of a VL comprising the amino acid sequences of the corresponding VL CDRs of VK-24. In some embodiments, the antibody molecule comprises CDR1, CDR2, and CDR3 of a VL comprising the amino acid sequences of the corresponding VL CDRs of VK-65. In some embodiments, the antibody molecule comprises CDR1, CDR2, and CDR3 of a VL comprising the amino acid sequences of the corresponding VL CDRs of VK-83. In some embodiments, the antibody molecule comprises CDR1, CDR2, and CDR3 of a VL comprising the amino acid sequences of the corresponding VL CDRs of VK-107. In some embodiments, the antibody molecule comprises CDR1, CDR2, and CDR3 of a VL comprising the amino acid sequences of the corresponding VL CDRs of VK-110. In some embodiments, the antibody molecule comprises CDR1, CDR2, and CDR3 of a VL comprising the amino acid sequences of the corresponding VL CDRs of VK-111.

In some embodiments, the antibody molecule comprises: (i) the CDR1, CDR2, and CDR3 of a VH comprising the amino acid sequences of the corresponding VH CDRs of VH123; and (ii) the CDR1, CDR2, and CDR3 of a VL comprising the amino acid sequences of the corresponding VL CDRs of VK-65. In some embodiments, the antibody molecule further comprises one or more Fc mutations, e.g., one, two, or all three mutations of FcMut215, as described herein (e.g., T307Q, Q311V, and/or A378V).

In some embodiments, the antibody molecule comprises: (i) the CDR1, CDR2, and CDR3 of a VH comprising the amino acid sequences of the corresponding VH CDRs of VH148; and (ii) the CDR1, CDR2, and CDR3 of a VL comprising the amino acid sequences of the corresponding VL CDRs of VK-65. In some embodiments, the antibody molecule further comprises one or more Fc mutations, e.g., one, two, or all three mutations of FcMut215, as described herein (e.g., T307Q, Q311V, and/or A378V).

In some embodiments, the antibody molecule comprises: (i) the CDR1, CDR2, and CDR3 of a VH comprising the amino acid sequences of the corresponding VH CDRs of VH175; and (ii) the CDR1, CDR2, and CDR3 of a VL comprising the amino acid sequences of the corresponding VL CDRs of VK-65. In some embodiments, the antibody molecule further comprises one or more Fc mutations, e.g., one, two, or all three mutations of FcMut215, as described herein (e.g., T307Q, Q311V, and/or A378V).

In some embodiments, the VH further comprises one or more (e.g., 2, 3, or 4) framework regions from a VH germline selected from VH1-69*04, VH3-30*01, VH3-30*02, VH3-30-03*03, and VH1-8*01, or as listed in Table 6. In some embodiments, the VL further comprises one or more (e.g., 2, 3, or 4) framework regions from a VL germline selected from Vκ1-39*01, Vκ4-1*01, and Vκ3-15*01, or as listed in Table 6. In some embodiments, the VH further comprises one or more (e.g., 2, 3, or 4) framework regions from a VH germline comprising one or more mutations at Vernier residues. In some embodiments, the VL further comprises one or more (e.g., 2, 3, or 4) framework regions from a VL germline comprising one or more mutations at Vernier residues.

In some embodiments, the antibody molecule comprises the VH CDRs, VL CDRs, and/or framework regions of any one antibody listed in Table 7, 13, or 14. In some embodiments, the antibody molecule comprises the VH CDRs and VL CDRs of any one antibody listed in Table 7, 13, or 14. In some embodiments, the antibody molecule comprises the VH CDRs and framework regions of any one antibody listed in Table 7, 13, or 14. In some embodiments, the antibody molecule comprises the VL CDRs and framework regions of any one antibody listed in Table 7, 13, or 14. In some embodiments, the antibody molecule comprises the VH CDRs, VL CDRs, and framework regions of any one antibody listed in Table 7, 13, or 14.

In some embodiments, the antibody molecule comprises one or more VH CDR mutations listed in Table 9.

In some embodiments, the antibody molecule comprises one or more VK CDR mutations listed in Table 10 and/or Table 11. In some embodiments, the antibody molecule comprises one or more LCDR (also referred to herein as VL CDR) mutations as listed in Table 12.

In some embodiments, the anti-HA antibody molecule comprises (a) a heavy chain variable region (VH) comprising an amino acid sequence having at least 80%, 85%, 90%, 95%, 96%, 97%, 98%, 99%, or 100% sequence identity to the amino acid sequence of a heavy chain variable region described herein (e.g., any of SEQ ID NOs: 1-39, 41-43, or 45-187); (b) a light chain variable region (VL) comprising an amino acid sequence having at least 80%, 85%, 90%, 95%, 96%, 97%, 98%, 99%, or 100% sequence identity to the amino acid sequence of a light chain variable region described herein (e.g., any of SEQ ID NOs: 188-298); or (c) both (a) and (b).

In some embodiments, the antibody molecule comprises a heavy chain variable region (VH) comprising an amino acid sequence having at least 80%, 85%, 90%, 95%, 96%, 97%, 98%, 99%, or 100% sequence identity to the amino acid sequence of any one of VH1-VH184. In some embodiments, the antibody molecule comprises a light chain variable region (VL) comprising an amino acid sequence having at least 80%, 85%, 90%, 95%, 96%, 97%, 98%, 99%, or 100% sequence identity to the amino acid sequence of any one of VK-1 through VK-111. In some embodiments, the antibody molecule comprises (i) a heavy chain variable region comprising an amino acid sequence having at least 80%, 85%, 90%, 95%, 96%, 97%, 98%, 99%, or 100% sequence identity to the amino acid sequence of any one of VH1-VH184, and (ii) a light chain variable region comprising an amino acid sequence having at least 80%, 85%, 90%, 95%, 96%, 97%, 98%, 99%, or 100% sequence identity to the amino acid sequence of any one of VK-1 through VK-111.

In some embodiments, the antibody molecule comprises a VH comprising an amino acid sequence having at least 80%, 85%, 90%, 95%, 96%, 97%, 98%, 99%, or 100% sequence identity to the amino acid sequence of VH1. In some embodiments, the antibody molecule comprises a VH comprising an amino acid sequence having at least 80%, 85%, 90%, 95%, 96%, 97%, 98%, 99%, or 100% sequence identity to the amino acid sequence of VH2. In some embodiments, the antibody molecule comprises a VH comprising an amino acid sequence having at least 80%, 85%, 90%, 95%, 96%, 97%, 98%, 99%, or 100% sequence identity to the amino acid sequence of VH3. In some embodiments, the antibody molecule comprises a VH comprising an amino acid sequence having at least 80%, 85%, 90%, 95%, 96%, 97%, 98%, 99%, or 100% sequence identity to the amino acid sequence of VH4. In some embodiments, the antibody molecule comprises a VH comprising an amino acid sequence having at least 80%, 85%, 90%, 95%, 96%, 97%, 98%, 99%, or 100% sequence identity to the amino acid sequence of VH5. In some embodiments, the antibody molecule comprises a VH comprising an amino acid sequence having at least 80%, 85%, 90%, 95%, 96%, 97%, 98%, 99%, or 100% sequence identity to the amino acid sequence of VH6. In some embodiments, the antibody molecule comprises a VH comprising an amino acid sequence having at least 80%, 85%, 90%, 95%, 96%, 97%, 98%, 99%, or 100% sequence identity to the amino acid sequence of VH7. In some embodiments, the antibody molecule comprises a VH comprising an amino acid sequence having at least 80%, 85%, 90%, 95%, 96%, 97%, 98%, 99%, or 100% sequence identity to the amino acid sequence of VH8. In some embodiments, the antibody molecule comprises a VH comprising an amino acid sequence having at least 80%, 85%, 90%, 95%, 96%, 97%, 98%, 99%, or 100% sequence identity to the amino acid sequence of VH9. In some embodiments, the antibody molecule comprises a VH comprising an amino acid sequence having at least 80%, 85%, 90%, 95%, 96%, 97%, 98%, 99%, or 100% sequence identity to the amino acid sequence of VH10. In some embodiments, the antibody molecule comprises a VH comprising an amino acid sequence having at least 80%, 85%, 90%, 95%, 96%, 97%, 98%, 99%, or 100% sequence identity to the amino acid sequence of VH11. In some embodiments, the antibody molecule comprises a VH comprising an amino acid sequence having at least 80%, 85%, 90%, 95%, 96%, 97%, 98%, 99%, or 100% sequence identity to the amino acid sequence of VH12. In some embodiments, the antibody molecule comprises a VH comprising an amino acid sequence having at least 80%, 85%, 90%, 95%, 96%, 97%, 98%, 99%, or 100% sequence identity to the amino acid sequence of VH13. In some embodiments, the antibody molecule comprises a VH comprising an amino acid sequence having at least 80%, 85%, 90%, 95%, 96%, 97%, 98%, 99%, or 100% sequence identity to the amino acid sequence of VH14. In some embodiments, the antibody molecule comprises a VH comprising an amino acid sequence having at least 80%, 85%, 90%, 95%, 96%, 97%, 98%, 99%, or 100% sequence identity to the amino acid sequence of VH15. In some embodiments, the antibody molecule comprises a VH comprising an amino acid sequence having at least 80%, 85%, 90%, 95%, 96%, 97%, 98%, 99%, or 100% sequence identity to the amino acid sequence of VH16. In some embodiments, the antibody molecule comprises a VH comprising an amino acid sequence having at least 80%, 85%, 90%, 95%, 96%, 97%, 98%, 99%, or 100% sequence identity to the amino acid sequence of VH17. In some embodiments, the antibody molecule comprises a VH comprising an amino acid sequence having at least 80%, 85%, 90%, 95%, 96%, 97%, 98%, 99%, or 100% sequence identity to the amino acid sequence of VH18. In some embodiments, the antibody molecule comprises a VH comprising an amino acid sequence having at least 80%, 85%, 90%, 95%, 96%, 97%, 98%, 99%, or 100% sequence identity to the amino acid sequence of VH19. In some embodiments, the antibody molecule comprises a VH comprising an amino acid sequence having at least 80%, 85%, 90%, 95%, 96%, 97%, 98%, 99%, or 100% sequence identity to the amino acid sequence of VH20. In some embodiments, the antibody molecule comprises a VH comprising an amino acid sequence having at least 80%, 85%, 90%, 95%, 96%, 97%, 98%, 99%, or 100% sequence identity to the amino acid sequence of VH21. In some embodiments, the antibody molecule comprises a VH comprising an amino acid sequence having at least 80%, 85%, 90%, 95%, 96%, 97%, 98%, 99%, or 100% sequence identity to the amino acid sequence of VH22. In some embodiments, the antibody molecule comprises a VH comprising an amino acid sequence having at least 80%, 85%, 90%, 95%, 96%, 97%, 98%, 99%, or 100% sequence identity to the amino acid sequence of VH23. In some embodiments, the antibody molecule comprises a VH comprising an amino acid sequence having at least 80%, 85%, 90%, 95%, 96%, 97%, 98%, 99%, or 100% sequence identity to the amino acid sequence of VH24. In some embodiments, the antibody molecule comprises a VH comprising an amino acid sequence having at least 80%, 85%, 90%, 95%, 96%, 97%, 98%, 99%, or 100% sequence identity to the amino acid sequence of VH25. In some embodiments, the antibody molecule comprises a VH comprising an amino acid sequence having at least 80%, 85%, 90%, 95%, 96%, 97%, 98%, 99%, or 100% sequence identity to the amino acid sequence of VH26. In some embodiments, the antibody molecule comprises a VH comprising an amino acid sequence having at least 80%, 85%, 90%, 95%, 96%, 97%, 98%, 99%, or 100% sequence identity to the amino acid sequence of VH27. In some embodiments, the antibody molecule comprises a VH comprising an amino acid sequence having at least 80%, 85%, 90%, 95%, 96%, 97%, 98%, 99%, or 100% sequence identity to the amino acid sequence of VH28. In some embodiments, the antibody molecule comprises a VH comprising an amino acid sequence having at least 80%, 85%, 90%, 95%, 96%, 97%, 98%, 99%, or 100% sequence identity to the amino acid sequence of VH29. In some embodiments, the antibody molecule comprises a VH comprising an amino acid sequence having at least 80%, 85%, 90%, 95%, 96%, 97%, 98%, 99%, or 100% sequence identity to the amino acid sequence of VH30. In some embodiments, the antibody molecule comprises a VH comprising an amino acid sequence having at least 80%, 85%, 90%, 95%, 96%, 97%, 98%, 99%, or 100% sequence identity to the amino acid sequence of VH31. In some embodiments, the antibody molecule comprises a VH comprising an amino acid sequence having at least 80%, 85%, 90%, 95%, 96%, 97%, 98%, 99%, or 100% sequence identity to the amino acid sequence of VH32. In some embodiments, the antibody molecule comprises a VH comprising an amino acid sequence having at least 80%, 85%, 90%, 95%, 96%, 97%, 98%, 99%, or 100% sequence identity to the amino acid sequence of VH33. In some embodiments, the antibody molecule comprises a VH comprising an amino acid sequence having at least 80%, 85%, 90%, 95%, 96%, 97%, 98%, 99%, or 100% sequence identity to the amino acid sequence of VH34. In some embodiments, the antibody molecule comprises a VH comprising an amino acid sequence having at least 80%, 85%, 90%, 95%, 96%, 97%, 98%, 99%, or 100% sequence identity to the amino acid sequence of VH35. In some embodiments, the antibody molecule comprises a VH comprising an amino acid sequence having at least 80%, 85%, 90%, 95%, 96%, 97%, 98%, 99%, or 100% sequence identity to the amino acid sequence of VH36. In some embodiments, the antibody molecule comprises a VH comprising an amino acid sequence having at least 80%, 85%, 90%, 95%, 96%, 97%, 98%, 99%, or 100% sequence identity to the amino acid sequence of VH37. In some embodiments, the antibody molecule comprises a VH comprising an amino acid sequence having at least 80%, 85%, 90%, 95%, 96%, 97%, 98%, 99%, or 100% sequence identity to the amino acid sequence of VH38. In some embodiments, the antibody molecule comprises a VH comprising an amino acid sequence having at least 80%, 85%, 90%, 95%, 96%, 97%, 98%, 99%, or 100% sequence identity to the amino acid sequence of VH39. In some embodiments, the antibody molecule comprises a VH comprising an amino acid sequence having at least 80%, 85%, 90%, 95%, 96%, 97%, 98%, 99%, or 100% sequence identity to the amino acid sequence of VH40. In some embodiments, the antibody molecule comprises a VH comprising an amino acid sequence having at least 80%, 85%, 90%, 95%, 96%, 97%, 98%, 99%, or 100% sequence identity to the amino acid sequence of VH41. In some embodiments, the antibody molecule comprises a VH comprising an amino acid sequence having at least 80%, 85%, 90%, 95%, 96%, 97%, 98%, 99%, or 100% sequence identity to the amino acid sequence of VH42. In some embodiments, the antibody molecule comprises a VH comprising an amino acid sequence having at least 80%, 85%, 90%, 95%, 96%, 97%, 98%, 99%, or 100% sequence identity to the amino acid sequence of VH43. In some embodiments, the antibody molecule comprises a VH comprising an amino acid sequence having at least 80%, 85%, 90%, 95%, 96%, 97%, 98%, 99%, or 100% sequence identity to the amino acid sequence of VH44. In some embodiments, the antibody molecule comprises a VH comprising an amino acid sequence having at least 80%, 85%, 90%, 95%, 96%, 97%, 98%, 99%, or 100% sequence identity to the amino acid sequence of VH45. In some embodiments, the antibody molecule comprises a VH comprising an amino acid sequence having at least 80%, 85%, 90%, 95%, 96%, 97%, 98%, 99%, or 100% sequence identity to the amino acid sequence of VH46. In some embodiments, the antibody molecule comprises a VH comprising an amino acid sequence having at least 80%, 85%, 90%, 95%, 96%, 97%, 98%, 99%, or 100% sequence identity to the amino acid sequence of VH47. In some embodiments, the antibody molecule comprises a VH comprising an amino acid sequence having at least 80%, 85%, 90%, 95%, 96%, 97%, 98%, 99%, or 100% sequence identity to the amino acid sequence of VH48. In some embodiments, the antibody molecule comprises a VH comprising an amino acid sequence having at least 80%, 85%, 90%, 95%, 96%, 97%, 98%, 99%, or 100% sequence identity to the amino acid sequence of VH49. In some embodiments, the antibody molecule comprises a VH comprising an amino acid sequence having at least 80%, 85%, 90%, 95%, 96%, 97%, 98%, 99%, or 100% sequence identity to the amino acid sequence of VH50. In some embodiments, the antibody molecule comprises a VH comprising an amino acid sequence having at least 80%, 85%, 90%, 95%, 96%, 97%, 98%, 99%, or 100% sequence identity to the amino acid sequence of VH51. In some embodiments, the antibody molecule comprises a VH comprising an amino acid sequence having at least 80%, 85%, 90%, 95%, 96%, 97%, 98%, 99%, or 100% sequence identity to the amino acid sequence of VH52. In some embodiments, the antibody molecule comprises a VH comprising an amino acid sequence having at least 80%, 85%, 90%, 95%, 96%, 97%, 98%, 99%, or 100% sequence identity to the amino acid sequence of VH53. In some embodiments, the antibody molecule comprises a VH comprising an amino acid sequence having at least 80%, 85%, 90%, 95%, 96%, 97%, 98%, 99%, or 100% sequence identity to the amino acid sequence of VH54. In some embodiments, the antibody molecule comprises a VH comprising an amino acid sequence having at least 80%, 85%, 90%, 95%, 96%, 97%, 98%, 99%, or 100% sequence identity to the amino acid sequence of VH55. In some embodiments, the antibody molecule comprises a VH comprising an amino acid sequence having at least 80%, 85%, 90%, 95%, 96%, 97%, 98%, 99%, or 100% sequence identity to the amino acid sequence of VH56. In some embodiments, the antibody molecule comprises a VH comprising an amino acid sequence having at least 80%, 85%, 90%, 95%, 96%, 97%, 98%, 99%, or 100% sequence identity to the amino acid sequence of VH57. In some embodiments, the antibody molecule comprises a VH comprising an amino acid sequence having at least 80%, 85%, 90%, 95%, 96%, 97%, 98%, 99%, or 100% sequence identity to the amino acid sequence of VH58. In some embodiments, the antibody molecule comprises a VH comprising an amino acid sequence having at least 80%, 85%, 90%, 95%, 96%, 97%, 98%, 99%, or 100% sequence identity to the amino acid sequence of VH59. In some embodiments, the antibody molecule comprises a VH comprising an amino acid sequence having at least 80%, 85%, 90%, 95%, 96%, 97%, 98%, 99%, or 100% sequence identity to the amino acid sequence of VH60. In some embodiments, the antibody molecule comprises a VH comprising an amino acid sequence having at least 80%, 85%, 90%, 95%, 96%, 97%, 98%, 99%, or 100% sequence identity to the amino acid sequence of VH61. In some embodiments, the antibody molecule comprises a VH comprising an amino acid sequence having at least 80%, 85%, 90%, 95%, 96%, 97%, 98%, 99%, or 100% sequence identity to the amino acid sequence of VH62. In some embodiments, the antibody molecule comprises a VH comprising an amino acid sequence having at least 80%, 85%, 90%, 95%, 96%, 97%, 98%, 99%, or 100% sequence identity to the amino acid sequence of VH63. In some embodiments, the antibody molecule comprises a VH comprising an amino acid sequence having at least 80%, 85%, 90%, 95%, 96%, 97%, 98%, 99%, or 100% sequence identity to the amino acid sequence of VH64. In some embodiments, the antibody molecule comprises a VH comprising an amino acid sequence having at least 80%, 85%, 90%, 95%, 96%, 97%, 98%, 99%, or 100% sequence identity to the amino acid sequence of VH65. In some embodiments, the antibody molecule comprises a VH comprising an amino acid sequence having at least 80%, 85%, 90%, 95%, 96%, 97%, 98%, 99%, or 100% sequence identity to the amino acid sequence of VH66. In some embodiments, the antibody molecule comprises a VH comprising an amino acid sequence having at least 80%, 85%, 90%, 95%, 96%, 97%, 98%, 99%, or 100% sequence identity to the amino acid sequence of VH67. In some embodiments, the antibody molecule comprises a VH comprising an amino acid sequence having at least 80%, 85%, 90%, 95%, 96%, 97%, 98%, 99%, or 100% sequence identity to the amino acid sequence of VH68. In some embodiments, the antibody molecule comprises a VH comprising an amino acid sequence having at least 80%, 85%, 90%, 95%, 96%, 97%, 98%, 99%, or 100% sequence identity to the amino acid sequence of VH69. In some embodiments, the antibody molecule comprises a VH comprising an amino acid sequence having at least 80%, 85%, 90%, 95%, 96%, 97%, 98%, 99%, or 100% sequence identity to the amino acid sequence of VH70. In some embodiments, the antibody molecule comprises a VH comprising an amino acid sequence having at least 80%, 85%, 90%, 95%, 96%, 97%, 98%, 99%, or 100% sequence identity to the amino acid sequence of VH71. In some embodiments, the antibody molecule comprises a VH comprising an amino acid sequence having at least 80%, 85%, 90%, 95%, 96%, 97%, 98%, 99%, or 100% sequence identity to the amino acid sequence of VH72. In some embodiments, the antibody molecule comprises a VH comprising an amino acid sequence having at least 80%, 85%, 90%, 95%, 96%, 97%, 98%, 99%, or 100% sequence identity to the amino acid sequence of VH73. In some embodiments, the antibody molecule comprises a VH comprising an amino acid sequence having at least 80%, 85%, 90%, 95%, 96%, 97%, 98%, 99%, or 100% sequence identity to the amino acid sequence of VH74. In some embodiments, the antibody molecule comprises a VH comprising an amino acid sequence having at least 80%, 85%, 90%, 95%, 96%, 97%, 98%, 99%, or 100% sequence identity to the amino acid sequence of VH75. In some embodiments, the antibody molecule comprises a VH comprising an amino acid sequence having at least 80%, 85%, 90%, 95%, 96%, 97%, 98%, 99%, or 100% sequence identity to the amino acid sequence of VH76. In some embodiments, the antibody molecule comprises a VH comprising an amino acid sequence having at least 80%, 85%, 90%, 95%, 96%, 97%, 98%, 99%, or 100% sequence identity to the amino acid sequence of VH77. In some embodiments, the antibody molecule comprises a VH comprising an amino acid sequence having at least 80%, 85%, 90%, 95%, 96%, 97%, 98%, 99%, or 100% sequence identity to the amino acid sequence of VH78. In some embodiments, the antibody molecule comprises a VH comprising an amino acid sequence having at least 80%, 85%, 90%, 95%, 96%, 97%, 98%, 99%, or 100% sequence identity to the amino acid sequence of VH79. In some embodiments, the antibody molecule comprises a VH comprising an amino acid sequence having at least 80%, 85%, 90%, 95%, 96%, 97%, 98%, 99%, or 100% sequence identity to the amino acid sequence of VH80. In some embodiments, the antibody molecule comprises a VH comprising an amino acid sequence having at least 80%, 85%, 90%, 95%, 96%, 97%, 98%, 99%, or 100% sequence identity to the amino acid sequence of VH81. In some embodiments, the antibody molecule comprises a VH comprising an amino acid sequence having at least 80%, 85%, 90%, 95%, 96%, 97%, 98%, 99%, or 100% sequence identity to the amino acid sequence of VH82. In some embodiments, the antibody molecule comprises a VH comprising an amino acid sequence having at least 80%, 85%, 90%, 95%, 96%, 97%, 98%, 99%, or 100% sequence identity to the amino acid sequence of VH83. In some embodiments, the antibody molecule comprises a VH comprising an amino acid sequence having at least 80%, 85%, 90%, 95%, 96%, 97%, 98%, 99%, or 100% sequence identity to the amino acid sequence of VH84. In some embodiments, the antibody molecule comprises a VH comprising an amino acid sequence having at least 80%, 85%, 90%, 95%, 96%, 97%, 98%, 99%, or 100% sequence identity to the amino acid sequence of VH85. In some embodiments, the antibody molecule comprises a VH comprising an amino acid sequence having at least 80%, 85%, 90%, 95%, 96%, 97%, 98%, 99%, or 100% sequence identity to the amino acid sequence of VH86. In some embodiments, the antibody molecule comprises a VH comprising an amino acid sequence having at least 80%, 85%, 90%, 95%, 96%, 97%, 98%, 99%, or 100% sequence identity to the amino acid sequence of VH87. In some embodiments, the antibody molecule comprises a VH comprising an amino acid sequence having at least 80%, 85%, 90%, 95%, 96%, 97%, 98%, 99%, or 100% sequence identity to the amino acid sequence of VH88. In some embodiments, the antibody molecule comprises a VH comprising an amino acid sequence having at least 80%, 85%, 90%, 95%, 96%, 97%, 98%, 99%, or 100% sequence identity to the amino acid sequence of VH89. In some embodiments, the antibody molecule comprises a VH comprising an amino acid sequence having at least 80%, 85%, 90%, 95%, 96%, 97%, 98%, 99%, or 100% sequence identity to the amino acid sequence of VH90. In some embodiments, the antibody molecule comprises a VH comprising an amino acid sequence having at least 80%, 85%, 90%, 95%, 96%, 97%, 98%, 99%, or 100% sequence identity to the amino acid sequence of VH91. In some embodiments, the antibody molecule comprises a VH comprising an amino acid sequence having at least 80%, 85%, 90%, 95%, 96%, 97%, 98%, 99%, or 100% sequence identity to the amino acid sequence of VH92. In some embodiments, the antibody molecule comprises a VH comprising an amino acid sequence having at least 80%, 85%, 90%, 95%, 96%, 97%, 98%, 99%, or 100% sequence identity to the amino acid sequence of VH93. In some embodiments, the antibody molecule comprises a VH comprising an amino acid sequence having at least 80%, 85%, 90%, 95%, 96%, 97%, 98%, 99%, or 100% sequence identity to the amino acid sequence of VH94. In some embodiments, the antibody molecule comprises a VH comprising an amino acid sequence having at least 80%, 85%, 90%, 95%, 96%, 97%, 98%, 99%, or 100% sequence identity to the amino acid sequence of VH95. In some embodiments, the antibody molecule comprises a VH comprising an amino acid sequence having at least 80%, 85%, 90%, 95%, 96%, 97%, 98%, 99%, or 100% sequence identity to the amino acid sequence of VH96. In some embodiments, the antibody molecule comprises a VH comprising an amino acid sequence having at least 80%, 85%, 90%, 95%, 96%, 97%, 98%, 99%, or 100% sequence identity to the amino acid sequence of VH97. In some embodiments, the antibody molecule comprises a VH comprising an amino acid sequence having at least 80%, 85%, 90%, 95%, 96%, 97%, 98%, 99%, or 100% sequence identity to the amino acid sequence of VH98. In some embodiments, the antibody molecule comprises a VH comprising an amino acid sequence having at least 80%, 85%, 90%, 95%, 96%, 97%, 98%, 99%, or 100% sequence identity to the amino acid sequence of VH99. In some embodiments, the antibody molecule comprises a VH comprising an amino acid sequence having at least 80%, 85%, 90%, 95%, 96%, 97%, 98%, 99%, or 100% sequence identity to the amino acid sequence of VH100. In some embodiments, the antibody molecule comprises a VH comprising an amino acid sequence having at least 80%, 85%, 90%, 95%, 96%, 97%, 98%, 99%, or 100% sequence identity to the amino acid sequence of VH101. In some embodiments, the antibody molecule comprises a VH comprising an amino acid sequence having at least 80%, 85%, 90%, 95%, 96%, 97%, 98%, 99%, or 100% sequence identity to the amino acid sequence of VH102. In some embodiments, the antibody molecule comprises a VH comprising an amino acid sequence having at least 80%, 85%, 90%, 95%, 96%, 97%, 98%, 99%, or 100% sequence identity to the amino acid sequence of VH103. In some embodiments, the antibody molecule comprises a VH comprising an amino acid sequence having at least 80%, 85%, 90%, 95%, 96%, 97%, 98%, 99%, or 100% sequence identity to the amino acid sequence of VH104. In some embodiments, the antibody molecule comprises a VH comprising an amino acid sequence having at least 80%, 85%, 90%, 95%, 96%, 97%, 98%, 99%, or 100% sequence identity to the amino acid sequence of VH105. In some embodiments, the antibody molecule comprises a VH comprising an amino acid sequence having at least 80%, 85%, 90%, 95%, 96%, 97%, 98%, 99%, or 100% sequence identity to the amino acid sequence of VH106. In some embodiments, the antibody molecule comprises a VH comprising an amino acid sequence having at least 80%, 85%, 90%, 95%, 96%, 97%, 98%, 99%, or 100% sequence identity to the amino acid sequence of VH107. In some embodiments, the antibody molecule comprises a VH comprising an amino acid sequence having at least 80%, 85%, 90%, 95%, 96%, 97%, 98%, 99%, or 100% sequence identity to the amino acid sequence of VH108. In some embodiments, the antibody molecule comprises a VH comprising an amino acid sequence having at least 80%, 85%, 90%, 95%, 96%, 97%, 98%, 99%, or 100% sequence identity to the amino acid sequence of VH109. In some embodiments, the antibody molecule comprises a VH comprising an amino acid sequence having at least 80%, 85%, 90%, 95%, 96%, 97%, 98%, 99%, or 100% sequence identity to the amino acid sequence of VH110. In some embodiments, the antibody molecule comprises a VH comprising an amino acid sequence having at least 80%, 85%, 90%, 95%, 96%, 97%, 98%, 99%, or 100% sequence identity to the amino acid sequence of VH111. In some embodiments, the antibody molecule comprises a VH comprising an amino acid sequence having at least 80%, 85%, 90%, 95%, 96%, 97%, 98%, 99%, or 100% sequence identity to the amino acid sequence of VH112. In some embodiments, the antibody molecule comprises a VH comprising an amino acid sequence having at least 80%, 85%, 90%, 95%, 96%, 97%, 98%, 99%, or 100% sequence identity to the amino acid sequence of VH113. In some embodiments, the antibody molecule comprises a VH comprising an amino acid sequence having at least 80%, 85%, 90%, 95%, 96%, 97%, 98%, 99%, or 100% sequence identity to the amino acid sequence of VH114. In some embodiments, the antibody molecule comprises a VH comprising an amino acid sequence having at least 80%, 85%, 90%, 95%, 96%, 97%, 98%, 99%, or 100% sequence identity to the amino acid sequence of VH115. In some embodiments, the antibody molecule comprises a VH comprising an amino acid sequence having at least 80%, 85%, 90%, 95%, 96%, 97%, 98%, 99%, or 100% sequence identity to the amino acid sequence of VH116. In some embodiments, the antibody molecule comprises a VH comprising an amino acid sequence having at least 80%, 85%, 90%, 95%, 96%, 97%, 98%, 99%, or 100% sequence identity to the amino acid sequence of VH117. In some embodiments, the antibody molecule comprises a VH comprising an amino acid sequence having at least 80%, 85%, 90%, 95%, 96%, 97%, 98%, 99%, or 100% sequence identity to the amino acid sequence of VH118. In some embodiments, the antibody molecule comprises a VH comprising an amino acid sequence having at least 80%, 85%, 90%, 95%, 96%, 97%, 98%, 99%, or 100% sequence identity to the amino acid sequence of VH119. In some embodiments, the antibody molecule comprises a VH comprising an amino acid sequence having at least 80%, 85%, 90%, 95%, 96%, 97%, 98%, 99%, or 100% sequence identity to the amino acid sequence of VH1120. In some embodiments, the antibody molecule comprises a VH comprising an amino acid sequence having at least 80%, 85%, 90%, 95%, 96%, 97%, 98%, 99%, or 100% sequence identity to the amino acid sequence of VH121. In some embodiments, the antibody molecule comprises a VH comprising an amino acid sequence having at least 80%, 85%, 90%, 95%, 96%, 97%, 98%, 99%, or 100% sequence identity to the amino acid sequence of VH122. In some embodiments, the antibody molecule comprises a VH comprising an amino acid sequence having at least 80%, 85%, 90%, 95%, 96%, 97%, 98%, 99%, or 100% sequence identity to the amino acid sequence of VH123. In some embodiments, the antibody molecule comprises a VH comprising an amino acid sequence having at least 80%, 85%, 90%, 95%, 96%, 97%, 98%, 99%, or 100% sequence identity to the amino acid sequence of VH124. In some embodiments, the antibody molecule comprises a VH comprising an amino acid sequence having at least 80%, 85%, 90%, 95%, 96%, 97%, 98%, 99%, or 100% sequence identity to the amino acid sequence of VH125. In some embodiments, the antibody molecule comprises a VH comprising an amino acid sequence having at least 80%, 85%, 90%, 95%, 96%, 97%, 98%, 99%, or 100% sequence identity to the amino acid sequence of VH126. In some embodiments, the antibody molecule comprises a VH comprising an amino acid sequence having at least 80%, 85%, 90%, 95%, 96%, 97%, 98%, 99%, or 100% sequence identity to the amino acid sequence of VH127. In some embodiments, the antibody molecule comprises a VH comprising an amino acid sequence having at least 80%, 85%, 90%, 95%, 96%, 97%, 98%, 99%, or 100% sequence identity to the amino acid sequence of VH128. In some embodiments, the antibody molecule comprises a VH comprising an amino acid sequence having at least 80%, 85%, 90%, 95%, 96%, 97%, 98%, 99%, or 100% sequence identity to the amino acid sequence of VH129. In some embodiments, the antibody molecule comprises a VH comprising an amino acid sequence having at least 80%, 85%, 90%, 95%, 96%, 97%, 98%, 99%, or 100% sequence identity to the amino acid sequence of VH130. In some embodiments, the antibody molecule comprises a VH comprising an amino acid sequence having at least 80%, 85%, 90%, 95%, 96%, 97%, 98%, 99%, or 100% sequence identity to the amino acid sequence of VH131. In some embodiments, the antibody molecule comprises a VH comprising an amino acid sequence having at least 80%, 85%, 90%, 95%, 96%, 97%, 98%, 99%, or 100% sequence identity to the amino acid sequence of VH132. In some embodiments, the antibody molecule comprises a VH comprising an amino acid sequence having at least 80%, 85%, 90%, 95%, 96%, 97%, 98%, 99%, or 100% sequence identity to the amino acid sequence of VH133. In some embodiments, the antibody molecule comprises a VH comprising an amino acid sequence having at least 80%, 85%, 90%, 95%, 96%, 97%, 98%, 99%, or 100% sequence identity to the amino acid sequence of VH134. In some embodiments, the antibody molecule comprises a VH comprising an amino acid sequence having at least 80%, 85%, 90%, 95%, 96%, 97%, 98%, 99%, or 100% sequence identity to the amino acid sequence of VH135. In some embodiments, the antibody molecule comprises a VH comprising an amino acid sequence having at least 80%, 85%, 90%, 95%, 96%, 97%, 98%, 99%, or 100% sequence identity to the amino acid sequence of VH136. In some embodiments, the antibody molecule comprises a VH comprising an amino acid sequence having at least 80%, 85%, 90%, 95%, 96%, 97%, 98%, 99%, or 100% sequence identity to the amino acid sequence of VH137. In some embodiments, the antibody molecule comprises a VH comprising an amino acid sequence having at least 80%, 85%, 90%, 95%, 96%, 97%, 98%, 99%, or 100% sequence identity to the amino acid sequence of VH138. In some embodiments, the antibody molecule comprises a VH comprising an amino acid sequence having at least 80%, 85%, 90%, 95%, 96%, 97%, 98%, 99%, or 100% sequence identity to the amino acid sequence of VH139. In some embodiments, the antibody molecule comprises a VH comprising an amino acid sequence having at least 80%, 85%, 90%, 95%, 96%, 97%, 98%, 99%, or 100% sequence identity to the amino acid sequence of VH140. In some embodiments, the antibody molecule comprises a VH comprising an amino acid sequence having at least 80%, 85%, 90%, 95%, 96%, 97%, 98%, 99%, or 100% sequence identity to the amino acid sequence of VH141. In some embodiments, the antibody molecule comprises a VH comprising an amino acid sequence having at least 80%, 85%, 90%, 95%, 96%, 97%, 98%, 99%, or 100% sequence identity to the amino acid sequence of VH142. In some embodiments, the antibody molecule comprises a VH comprising an amino acid sequence having at least 80%, 85%, 90%, 95%, 96%, 97%, 98%, 99%, or 100% sequence identity to the amino acid sequence of VH143. In some embodiments, the antibody molecule comprises a VH comprising an amino acid sequence having at least 80%, 85%, 90%, 95%, 96%, 97%, 98%, 99%, or 100% sequence identity to the amino acid sequence of VH144. In some embodiments, the antibody molecule comprises a VH comprising an amino acid sequence having at least 80%, 85%, 90%, 95%, 96%, 97%, 98%, 99%, or 100% sequence identity to the amino acid sequence of VH145. In some embodiments, the antibody molecule comprises a VH comprising an amino acid sequence having at least 80%, 85%, 90%, 95%, 96%, 97%, 98%, 99%, or 100% sequence identity to the amino acid sequence of VH146. In some embodiments, the antibody molecule comprises a VH comprising an amino acid sequence having at least 80%, 85%, 90%, 95%, 96%, 97%, 98%, 99%, or 100% sequence identity to the amino acid sequence of VH147. In some embodiments, the antibody molecule comprises a VH comprising an amino acid sequence having at least 80%, 85%, 90%, 95%, 96%, 97%, 98%, 99%, or 100% sequence identity to the amino acid sequence of VH148. In some embodiments, the antibody molecule comprises a VH comprising an amino acid sequence having at least 80%, 85%, 90%, 95%, 96%, 97%, 98%, 99%, or 100% sequence identity to the amino acid sequence of VH149. In some embodiments, the antibody molecule comprises a VH comprising an amino acid sequence having at least 80%, 85%, 90%, 95%, 96%, 97%, 98%, 99%, or 100% sequence identity to the amino acid sequence of VH150. In some embodiments, the antibody molecule comprises a VH comprising an amino acid sequence having at least 80%, 85%, 90%, 95%, 96%, 97%, 98%, 99%, or 100% sequence identity to the amino acid sequence of VH151. In some embodiments, the antibody molecule comprises a VH comprising an amino acid sequence having at least 80%, 85%, 90%, 95%, 96%, 97%, 98%, 99%, or 100% sequence identity to the amino acid sequence of VH152. In some embodiments, the antibody molecule comprises a VH comprising an amino acid sequence having at least 80%, 85%, 90%, 95%, 96%, 97%, 98%, 99%, or 100% sequence identity to the amino acid sequence of VH153. In some embodiments, the antibody molecule comprises a VH comprising an amino acid sequence having at least 80%, 85%, 90%, 95%, 96%, 97%, 98%, 99%, or 100% sequence identity to the amino acid sequence of VH154. In some embodiments, the antibody molecule comprises a VH comprising an amino acid sequence having at least 80%, 85%, 90%, 95%, 96%, 97%, 98%, 99%, or 100% sequence identity to the amino acid sequence of VH155. In some embodiments, the antibody molecule comprises a VH comprising an amino acid sequence having at least 80%, 85%, 90%, 95%, 96%, 97%, 98%, 99%, or 100% sequence identity to the amino acid sequence of VH156. In some embodiments, the antibody molecule comprises a VH comprising an amino acid sequence having at least 80%, 85%, 90%, 95%, 96%, 97%, 98%, 99%, or 100% sequence identity to the amino acid sequence of VH157. In some embodiments, the antibody molecule comprises a VH comprising an amino acid sequence having at least 80%, 85%, 90%, 95%, 96%, 97%, 98%, 99%, or 100% sequence identity to the amino acid sequence of VH158. In some embodiments, the antibody molecule comprises a VH comprising an amino acid sequence having at least 80%, 85%, 90%, 95%, 96%, 97%, 98%, 99%, or 100% sequence identity to the amino acid sequence of VH159. In some embodiments, the antibody molecule comprises a VH comprising an amino acid sequence having at least 80%, 85%, 90%, 95%, 96%, 97%, 98%, 99%, or 100% sequence identity to the amino acid sequence of VH160. In some embodiments, the antibody molecule comprises a VH comprising an amino acid sequence having at least 80%, 85%, 90%, 95%, 96%, 97%, 98%, 99%, or 100% sequence identity to the amino acid sequence of VH161. In some embodiments, the antibody molecule comprises a VH comprising an amino acid sequence having at least 80%, 85%, 90%, 95%, 96%, 97%, 98%, 99%, or 100% sequence identity to the amino acid sequence of VH162. In some embodiments, the antibody molecule comprises a VH comprising an amino acid sequence having at least 80%, 85%, 90%, 95%, 96%, 97%, 98%, 99%, or 100% sequence identity to the amino acid sequence of VH163. In some embodiments, the antibody molecule comprises a VH comprising an amino acid sequence having at least 80%, 85%, 90%, 95%, 96%, 97%, 98%, 99%, or 100% sequence identity to the amino acid sequence of VH164. In some embodiments, the antibody molecule comprises a VH comprising an amino acid sequence having at least 80%, 85%, 90%, 95%, 96%, 97%, 98%, 99%, or 100% sequence identity to the amino acid sequence of VH165. In some embodiments, the antibody molecule comprises a VH comprising an amino acid sequence having at least 80%, 85%, 90%, 95%, 96%, 97%, 98%, 99%, or 100% sequence identity to the amino acid sequence of VH166. In some embodiments, the antibody molecule comprises a VH comprising an amino acid sequence having at least 80%, 85%, 90%, 95%, 96%, 97%, 98%, 99%, or 100% sequence identity to the amino acid sequence of VH167. In some embodiments, the antibody molecule comprises a VH comprising an amino acid sequence having at least 80%, 85%, 90%, 95%, 96%, 97%, 98%, 99%, or 100% sequence identity to the amino acid sequence of VH168. In some embodiments, the antibody molecule comprises a VH comprising an amino acid sequence having at least 80%, 85%, 90%, 95%, 96%, 97%, 98%, 99%, or 100% sequence identity to the amino acid sequence of VH169. In some embodiments, the antibody molecule comprises a VH comprising an amino acid sequence having at least 80%, 85%, 90%, 95%, 96%, 97%, 98%, 99%, or 100% sequence identity to the amino acid sequence of VH170. In some embodiments, the antibody molecule comprises a VH comprising an amino acid sequence having at least 80%, 85%, 90%, 95%, 96%, 97%, 98%, 99%, or 100% sequence identity to the amino acid sequence of VH171. In some embodiments, the antibody molecule comprises a VH comprising an amino acid sequence having at least 80%, 85%, 90%, 95%, 96%, 97%, 98%, 99%, or 100% sequence identity to the amino acid sequence of VH172. In some embodiments, the antibody molecule comprises a VH comprising an amino acid sequence having at least 80%, 85%, 90%, 95%, 96%, 97%, 98%, 99%, or 100% sequence identity to the amino acid sequence of VH173. In some embodiments, the antibody molecule comprises a VH comprising an amino acid sequence having at least 80%, 85%, 90%, 95%, 96%, 97%, 98%, 99%, or 100% sequence identity to the amino acid sequence of VH174. In some embodiments, the antibody molecule comprises a VH comprising an amino acid sequence having at least 80%, 85%, 90%, 95%, 96%, 97%, 98%, 99%, or 100% sequence identity to the amino acid sequence of VH175. In some embodiments, the antibody molecule comprises a VH comprising an amino acid sequence having at least 80%, 85%, 90%, 95%, 96%, 97%, 98%, 99%, or 100% sequence identity to the amino acid sequence of VH176. In some embodiments, the antibody molecule comprises a VH comprising an amino acid sequence having at least 80%, 85%, 90%, 95%, 96%, 97%, 98%, 99%, or 100% sequence identity to the amino acid sequence of VH177. In some embodiments, the antibody molecule comprises a VH comprising an amino acid sequence having at least 80%, 85%, 90%, 95%, 96%, 97%, 98%, 99%, or 100% sequence identity to the amino acid sequence of VH178. In some embodiments, the antibody molecule comprises a VH comprising an amino acid sequence having at least 80%, 85%, 90%, 95%, 96%, 97%, 98%, 99%, or 100% sequence identity to the amino acid sequence of VH179. In some embodiments, the antibody molecule comprises a VH comprising an amino acid sequence having at least 80%, 85%, 90%, 95%, 96%, 97%, 98%, 99%, or 100% sequence identity to the amino acid sequence of VH180. In some embodiments, the antibody molecule comprises a VH comprising an amino acid sequence having at least 80%, 85%, 90%, 95%, 96%, 97%, 98%, 99%, or 100% sequence identity to the amino acid sequence of VH181. In some embodiments, the antibody molecule comprises a VH comprising an amino acid sequence having at least 80%, 85%, 90%, 95%, 96%, 97%, 98%, 99%, or 100% sequence identity to the amino acid sequence of VH182. In some embodiments, the antibody molecule comprises a VH comprising an amino acid sequence having at least 80%, 85%, 90%, 95%, 96%, 97%, 98%, 99%, or 100% sequence identity to the amino acid sequence of VH183. In some embodiments, the antibody molecule comprises a VH comprising an amino acid sequence having at least 80%, 85%, 90%, 95%, 96%, 97%, 98%, 99%, or 100% sequence identity to the amino acid sequence of VH184.

In some embodiments, the antibody molecule comprises a VL comprising an amino acid sequence having at least 80%, 85%, 90%, 95%, 96%, 97%, 98%, 99%, or 100% sequence identity to the amino acid sequence of VK-1. In some embodiments, the antibody molecule comprises a VL comprising an amino acid sequence having at least 80%, 85%, 90%, 95%, 96%, 97%, 98%, 99%, or 100% sequence identity to the amino acid sequence of VK-2. In some embodiments, the antibody molecule comprises a VL comprising an amino acid sequence having at least 80%, 85%, 90%, 95%, 96%, 97%, 98%, 99%, or 100% sequence identity to the amino acid sequence of VK-3. In some embodiments, the antibody molecule comprises a VL comprising an amino acid sequence having at least 80%, 85%, 90%, 95%, 96%, 97%, 98%, 99%, or 100% sequence identity to the amino acid sequence of VK-4. In some embodiments, the antibody molecule comprises a VL comprising an amino acid sequence having at least 80%, 85%, 90%, 95%, 96%, 97%, 98%, 99%, or 100% sequence identity to the amino acid sequence of VK-5. In some embodiments, the antibody molecule comprises a VL comprising an amino acid sequence having at least 80%, 85%, 90%, 95%, 96%, 97%, 98%, 99%, or 100% sequence identity to the amino acid sequence of VK-6. In some embodiments, the antibody molecule comprises a VL comprising an amino acid sequence having at least 80%, 85%, 90%, 95%, 96%, 97%, 98%, 99%, or 100% sequence identity to the amino acid sequence of VK-7. In some embodiments, the antibody molecule comprises a VL comprising an amino acid sequence having at least 80%, 85%, 90%, 95%, 96%, 97%, 98%, 99%, or 100% sequence identity to the amino acid sequence of VK-8. In some embodiments, the antibody molecule comprises a VL comprising an amino acid sequence having at least 80%, 85%, 90%, 95%, 96%, 97%, 98%, 99%, or 100% sequence identity to the amino acid sequence of VK-9. In some embodiments, the antibody molecule comprises a VL comprising an amino acid sequence having at least 80%, 85%, 90%, 95%, 96%, 97%, 98%, 99%, or 100% sequence identity to the amino acid sequence of VK-10. In some embodiments, the antibody molecule comprises a VL comprising an amino acid sequence having at least 80%, 85%, 90%, 95%, 96%, 97%, 98%, 99%, or 100% sequence identity to the amino acid sequence of VK-11. In some embodiments, the antibody molecule comprises a VL comprising an amino acid sequence having at least 80%, 85%, 90%, 95%, 96%, 97%, 98%, 99%, or 100% sequence identity to the amino acid sequence of VK-12. In some embodiments, the antibody molecule comprises a VL comprising an amino acid sequence having at least 80%, 85%, 90%, 95%, 96%, 97%, 98%, 99%, or 100% sequence identity to the amino acid sequence of VK-13. In some embodiments, the antibody molecule comprises a VL comprising an amino acid sequence having at least 80%, 85%, 90%, 95%, 96%, 97%, 98%, 99%, or 100% sequence identity to the amino acid sequence of VK-14. In some embodiments, the antibody molecule comprises a VL comprising an amino acid sequence having at least 80%, 85%, 90%, 95%, 96%, 97%, 98%, 99%, or 100% sequence identity to the amino acid sequence of VK-15. In some embodiments, the antibody molecule comprises a VL comprising an amino acid sequence having at least 80%, 85%, 90%, 95%, 96%, 97%, 98%, 99%, or 100% sequence identity to the amino acid sequence of VK-16. In some embodiments, the antibody molecule comprises a VL comprising an amino acid sequence having at least 80%, 85%, 90%, 95%, 96%, 97%, 98%, 99%, or 100% sequence identity to the amino acid sequence of VK-17. In some embodiments, the antibody molecule comprises a VL comprising an amino acid sequence having at least 80%, 85%, 90%, 95%, 96%, 97%, 98%, 99%, or 100% sequence identity to the amino acid sequence of VK-18. In some embodiments, the antibody molecule comprises a VL comprising an amino acid sequence having at least 80%, 85%, 90%, 95%, 96%, 97%, 98%, 99%, or 100% sequence identity to the amino acid sequence of VK-19. In some embodiments, the antibody molecule comprises a VL comprising an amino acid sequence having at least 80%, 85%, 90%, 95%, 96%, 97%, 98%, 99%, or 100% sequence identity to the amino acid sequence of VK-20. In some embodiments, the antibody molecule comprises a VL comprising an amino acid sequence having at least 80%, 85%, 90%, 95%, 96%, 97%, 98%, 99%, or 100% sequence identity to the amino acid sequence of VK-21. In some embodiments, the antibody molecule comprises a VL comprising an amino acid sequence having at least 80%, 85%, 90%, 95%, 96%, 97%, 98%, 99%, or 100% sequence identity to the amino acid sequence of VK-22. In some embodiments, the antibody molecule comprises a VL comprising an amino acid sequence having at least 80%, 85%, 90%, 95%, 96%, 97%, 98%, 99%, or 100% sequence identity to the amino acid sequence of VK-23. In some embodiments, the antibody molecule comprises a VL comprising an amino acid sequence having at least 80%, 85%, 90%, 95%, 96%, 97%, 98%, 99%, or 100% sequence identity to the amino acid sequence of VK-24. In some embodiments, the antibody molecule comprises a VL comprising an amino acid sequence having at least 80%, 85%, 90%, 95%, 96%, 97%, 98%, 99%, or 100% sequence identity to the amino acid sequence of VK-25. In some embodiments, the antibody molecule comprises a VL comprising an amino acid sequence having at least 80%, 85%, 90%, 95%, 96%, 97%, 98%, 99%, or 100% sequence identity to the amino acid sequence of VK-26. In some embodiments, the antibody molecule comprises a VL comprising an amino acid sequence having at least 80%, 85%, 90%, 95%, 96%, 97%, 98%, 99%, or 100% sequence identity to the amino acid sequence of VK-27. In some embodiments, the antibody molecule comprises a VL comprising an amino acid sequence having at least 80%, 85%, 90%, 95%, 96%, 97%, 98%, 99%, or 100% sequence identity to the amino acid sequence of VK-28. In some embodiments, the antibody molecule comprises a VL comprising an amino acid sequence having at least 80%, 85%, 90%, 95%, 96%, 97%, 98%, 99%, or 100% sequence identity to the amino acid sequence of VK-29. In some embodiments, the antibody molecule comprises a VL comprising an amino acid sequence having at least 80%, 85%, 90%, 95%, 96%, 97%, 98%, 99%, or 100% sequence identity to the amino acid sequence of VK-30. In some embodiments, the antibody molecule comprises a VL comprising an amino acid sequence having at least 80%, 85%, 90%, 95%, 96%, 97%, 98%, 99%, or 100% sequence identity to the amino acid sequence of VK-31. In some embodiments, the antibody molecule comprises a VL comprising an amino acid sequence having at least 80%, 85%, 90%, 95%, 96%, 97%, 98%, 99%, or 100% sequence identity to the amino acid sequence of VK-32. In some embodiments, the antibody molecule comprises a VL comprising an amino acid sequence having at least 80%, 85%, 90%, 95%, 96%, 97%, 98%, 99%, or 100% sequence identity to the amino acid sequence of VK-33. In some embodiments, the antibody molecule comprises a VL comprising an amino acid sequence having at least 80%, 85%, 90%, 95%, 96%, 97%, 98%, 99%, or 100% sequence identity to the amino acid sequence of VK-34. In some embodiments, the antibody molecule comprises a VL comprising an amino acid sequence having at least 80%, 85%, 90%, 95%, 96%, 97%, 98%, 99%, or 100% sequence identity to the amino acid sequence of VK-35. In some embodiments, the antibody molecule comprises a VL comprising an amino acid sequence having at least 80%, 85%, 90%, 95%, 96%, 97%, 98%, 99%, or 100% sequence identity to the amino acid sequence of VK-36. In some embodiments, the antibody molecule comprises a VL comprising an amino acid sequence having at least 80%, 85%, 90%, 95%, 96%, 97%, 98%, 99%, or 100% sequence identity to the amino acid sequence of VK-37. In some embodiments, the antibody molecule comprises a VL comprising an amino acid sequence having at least 80%, 85%, 90%, 95%, 96%, 97%, 98%, 99%, or 100% sequence identity to the amino acid sequence of VK-38. In some embodiments, the antibody molecule comprises a VL comprising an amino acid sequence having at least 80%, 85%, 90%, 95%, 96%, 97%, 98%, 99%, or 100% sequence identity to the amino acid sequence of VK-39. In some embodiments, the antibody molecule comprises a VL comprising an amino acid sequence having at least 80%, 85%, 90%, 95%, 96%, 97%, 98%, 99%, or 100% sequence identity to the amino acid sequence of VK-40. In some embodiments, the antibody molecule comprises a VL comprising an amino acid sequence having at least 80%, 85%, 90%, 95%, 96%, 97%, 98%, 99%, or 100% sequence identity to the amino acid sequence of VK-41. In some embodiments, the antibody molecule comprises a VL comprising an amino acid sequence having at least 80%, 85%, 90%, 95%, 96%, 97%, 98%, 99%, or 100% sequence identity to the amino acid sequence of VK-42. In some embodiments, the antibody molecule comprises a VL comprising an amino acid sequence having at least 80%, 85%, 90%, 95%, 96%, 97%, 98%, 99%, or 100% sequence identity to the amino acid sequence of VK-43. In some embodiments, the antibody molecule comprises a VL comprising an amino acid sequence having at least 80%, 85%, 90%, 95%, 96%, 97%, 98%, 99%, or 100% sequence identity to the amino acid sequence of VK-44. In some embodiments, the antibody molecule comprises a VL comprising an amino acid sequence having at least 80%, 85%, 90%, 95%, 96%, 97%, 98%, 99%, or 100% sequence identity to the amino acid sequence of VK-45. In some embodiments, the antibody molecule comprises a VL comprising an amino acid sequence having at least 80%, 85%, 90%, 95%, 96%, 97%, 98%, 99%, or 100% sequence identity to the amino acid sequence of VK-46. In some embodiments, the antibody molecule comprises a VL comprising an amino acid sequence having at least 80%, 85%, 90%, 95%, 96%, 97%, 98%, 99%, or 100% sequence identity to the amino acid sequence of VK-47. In some embodiments, the antibody molecule comprises a VL comprising an amino acid sequence having at least 80%, 85%, 90%, 95%, 96%, 97%, 98%, 99%, or 100% sequence identity to the amino acid sequence of VK-48. In some embodiments, the antibody molecule comprises a VL comprising an amino acid sequence having at least 80%, 85%, 90%, 95%, 96%, 97%, 98%, 99%, or 100% sequence identity to the amino acid sequence of VK-49. In some embodiments, the antibody molecule comprises a VL comprising an amino acid sequence having at least 80%, 85%, 90%, 95%, 96%, 97%, 98%, 99%, or 100% sequence identity to the amino acid sequence of VK-50. In some embodiments, the antibody molecule comprises a VL comprising an amino acid sequence having at least 80%, 85%, 90%, 95%, 96%, 97%, 98%, 99%, or 100% sequence identity to the amino acid sequence of VK-51. In some embodiments, the antibody molecule comprises a VL comprising an amino acid sequence having at least 80%, 85%, 90%, 95%, 96%, 97%, 98%, 99%, or 100% sequence identity to the amino acid sequence of VK-52. In some embodiments, the antibody molecule comprises a VL comprising an amino acid sequence having at least 80%, 85%, 90%, 95%, 96%, 97%, 98%, 99%, or 100% sequence identity to the amino acid sequence of VK-53. In some embodiments, the antibody molecule comprises a VL comprising an amino acid sequence having at least 80%, 85%, 90%, 95%, 96%, 97%, 98%, 99%, or 100% sequence identity to the amino acid sequence of VK-54. In some embodiments, the antibody molecule comprises a VL comprising an amino acid sequence having at least 80%, 85%, 90%, 95%, 96%, 97%, 98%, 99%, or 100% sequence identity to the amino acid sequence of VK-55. In some embodiments, the antibody molecule comprises a VL comprising an amino acid sequence having at least 80%, 85%, 90%, 95%, 96%, 97%, 98%, 99%, or 100% sequence identity to the amino acid sequence of VK-56. In some embodiments, the antibody molecule comprises a VL comprising an amino acid sequence having at least 80%, 85%, 90%, 95%, 96%, 97%, 98%, 99%, or 100% sequence identity to the amino acid sequence of VK-57. In some embodiments, the antibody molecule comprises a VL comprising an amino acid sequence having at least 80%, 85%, 90%, 95%, 96%, 97%, 98%, 99%, or 100% sequence identity to the amino acid sequence of VK-58. In some embodiments, the antibody molecule comprises a VL comprising an amino acid sequence having at least 80%, 85%, 90%, 95%, 96%, 97%, 98%, 99%, or 100% sequence identity to the amino acid sequence of VK-59. In some embodiments, the antibody molecule comprises a VL comprising an amino acid sequence having at least 80%, 85%, 90%, 95%, 96%, 97%, 98%, 99%, or 100% sequence identity to the amino acid sequence of VK-60. In some embodiments, the antibody molecule comprises a VL comprising an amino acid sequence having at least 80%, 85%, 90%, 95%, 96%, 97%, 98%, 99%, or 100% sequence identity to the amino acid sequence of VK-61. In some embodiments, the antibody molecule comprises a VL comprising an amino acid sequence having at least 80%, 85%, 90%, 95%, 96%, 97%, 98%, 99%, or 100% sequence identity to the amino acid sequence of VK-62. In some embodiments, the antibody molecule comprises a VL comprising an amino acid sequence having at least 80%, 85%, 90%, 95%, 96%, 97%, 98%, 99%, or 100% sequence identity to the amino acid sequence of VK-63. In some embodiments, the antibody molecule comprises a VL comprising an amino acid sequence having at least 80%, 85%, 90%, 95%, 96%, 97%, 98%, 99%, or 100% sequence identity to the amino acid sequence of VK-64. In some embodiments, the antibody molecule comprises a VL comprising an amino acid sequence having at least 80%, 85%, 90%, 95%, 96%, 97%, 98%, 99%, or 100% sequence identity to the amino acid sequence of VK-65. In some embodiments, the antibody molecule comprises a VL comprising an amino acid sequence having at least 80%, 85%, 90%, 95%, 96%, 97%, 98%, 99%, or 100% sequence identity to the amino acid sequence of VK-66. In some embodiments, the antibody molecule comprises a VL comprising an amino acid sequence having at least 80%, 85%, 90%, 95%, 96%, 97%, 98%, 99%, or 100% sequence identity to the amino acid sequence of VK-67. In some embodiments, the antibody molecule comprises a VL comprising an amino acid sequence having at least 80%, 85%, 90%, 95%, 96%, 97%, 98%, 99%, or 100% sequence identity to the amino acid sequence of VK-68. In some embodiments, the antibody molecule comprises a VL comprising an amino acid sequence having at least 80%, 85%, 90%, 95%, 96%, 97%, 98%, 99%, or 100% sequence identity to the amino acid sequence of VK-69. In some embodiments, the antibody molecule comprises a VL comprising an amino acid sequence having at least 80%, 85%, 90%, 95%, 96%, 97%, 98%, 99%, or 100% sequence identity to the amino acid sequence of VK-70. In some embodiments, the antibody molecule comprises a VL comprising an amino acid sequence having at least 80%, 85%, 90%, 95%, 96%, 97%, 98%, 99%, or 100% sequence identity to the amino acid sequence of VK-71. In some embodiments, the antibody molecule comprises a VL comprising an amino acid sequence having at least 80%, 85%, 90%, 95%, 96%, 97%, 98%, 99%, or 100% sequence identity to the amino acid sequence of VK-72. In some embodiments, the antibody molecule comprises a VL comprising an amino acid sequence having at least 80%, 85%, 90%, 95%, 96%, 97%, 98%, 99%, or 100% sequence identity to the amino acid sequence of VK-73. In some embodiments, the antibody molecule comprises a VL comprising an amino acid sequence having at least 80%, 85%, 90%, 95%, 96%, 97%, 98%, 99%, or 100% sequence identity to the amino acid sequence of VK-74. In some embodiments, the antibody molecule comprises a VL comprising an amino acid sequence having at least 80%, 85%, 90%, 95%, 96%, 97%, 98%, 99%, or 100% sequence identity to the amino acid sequence of VK-75. In some embodiments, the antibody molecule comprises a VL comprising an amino acid sequence having at least 80%, 85%, 90%, 95%, 96%, 97%, 98%, 99%, or 100% sequence identity to the amino acid sequence of VK-76. In some embodiments, the antibody molecule comprises a VL comprising an amino acid sequence having at least 80%, 85%, 90%, 95%, 96%, 97%, 98%, 99%, or 100% sequence identity to the amino acid sequence of VK-77. In some embodiments, the antibody molecule comprises a VL comprising an amino acid sequence having at least 80%, 85%, 90%, 95%, 96%, 97%, 98%, 99%, or 100% sequence identity to the amino acid sequence of VK-78. In some embodiments, the antibody molecule comprises a VL comprising an amino acid sequence having at least 80%, 85%, 90%, 95%, 96%, 97%, 98%, 99%, or 100% sequence identity to the amino acid sequence of VK-79. In some embodiments, the antibody molecule comprises a VL comprising an amino acid sequence having at least 80%, 85%, 90%, 95%, 96%, 97%, 98%, 99%, or 100% sequence identity to the amino acid sequence of VK-80. In some embodiments, the antibody molecule comprises a VL comprising an amino acid sequence having at least 80%, 85%, 90%, 95%, 96%, 97%, 98%, 99%, or 100% sequence identity to the amino acid sequence of VK-81. In some embodiments, the antibody molecule comprises a VL comprising an amino acid sequence having at least 80%, 85%, 90%, 95%, 96%, 97%, 98%, 99%, or 100% sequence identity to the amino acid sequence of VK-82. In some embodiments, the antibody molecule comprises a VL comprising an amino acid sequence having at least 80%, 85%, 90%, 95%, 96%, 97%, 98%, 99%, or 100% sequence identity to the amino acid sequence of VK-83. In some embodiments, the antibody molecule comprises a VL comprising an amino acid sequence having at least 80%, 85%, 90%, 95%, 96%, 97%, 98%, 99%, or 100% sequence identity to the amino acid sequence of VK-84. In some embodiments, the antibody molecule comprises a VL comprising an amino acid sequence having at least 80%, 85%, 90%, 95%, 96%, 97%, 98%, 99%, or 100% sequence identity to the amino acid sequence of VK-85. In some embodiments, the antibody molecule comprises a VL comprising an amino acid sequence having at least 80%, 85%, 90%, 95%, 96%, 97%, 98%, 99%, or 100% sequence identity to the amino acid sequence of VK-86. In some embodiments, the antibody molecule comprises a VL comprising an amino acid sequence having at least 80%, 85%, 90%, 95%, 96%, 97%, 98%, 99%, or 100% sequence identity to the amino acid sequence of VK-87. In some embodiments, the antibody molecule comprises a VL comprising an amino acid sequence having at least 80%, 85%, 90%, 95%, 96%, 97%, 98%, 99%, or 100% sequence identity to the amino acid sequence of VK-88. In some embodiments, the antibody molecule comprises a VL comprising an amino acid sequence having at least 80%, 85%, 90%, 95%, 96%, 97%, 98%, 99%, or 100% sequence identity to the amino acid sequence of VK-89. In some embodiments, the antibody molecule comprises a VL comprising an amino acid sequence having at least 80%, 85%, 90%, 95%, 96%, 97%, 98%, 99%, or 100% sequence identity to the amino acid sequence of VK-90. In some embodiments, the antibody molecule comprises a VL comprising an amino acid sequence having at least 80%, 85%, 90%, 95%, 96%, 97%, 98%, 99%, or 100% sequence identity to the amino acid sequence of VK-91. In some embodiments, the antibody molecule comprises a VL comprising an amino acid sequence having at least 80%, 85%, 90%, 95%, 96%, 97%, 98%, 99%, or 100% sequence identity to the amino acid sequence of VK-92. In some embodiments, the antibody molecule comprises a VL comprising an amino acid sequence having at least 80%, 85%, 90%, 95%, 96%, 97%, 98%, 99%, or 100% sequence identity to the amino acid sequence of VK-93. In some embodiments, the antibody molecule comprises a VL comprising an amino acid sequence having at least 80%, 85%, 90%, 95%, 96%, 97%, 98%, 99%, or 100% sequence identity to the amino acid sequence of VK-94. In some embodiments, the antibody molecule comprises a VL comprising an amino acid sequence having at least 80%, 85%, 90%, 95%, 96%, 97%, 98%, 99%, or 100% sequence identity to the amino acid sequence of VK-95. In some embodiments, the antibody molecule comprises a VL comprising an amino acid sequence having at least 80%, 85%, 90%, 95%, 96%, 97%, 98%, 99%, or 100% sequence identity to the amino acid sequence of VK-96. In some embodiments, the antibody molecule comprises a VL comprising an amino acid sequence having at least 80%, 85%, 90%, 95%, 96%, 97%, 98%, 99%, or 100% sequence identity to the amino acid sequence of VK-97. In some embodiments, the antibody molecule comprises a VL comprising an amino acid sequence having at least 80%, 85%, 90%, 95%, 96%, 97%, 98%, 99%, or 100% sequence identity to the amino acid sequence of VK-98. In some embodiments, the antibody molecule comprises a VL comprising an amino acid sequence having at least 80%, 85%, 90%, 95%, 96%, 97%, 98%, 99%, or 100% sequence identity to the amino acid sequence of VK-99. In some embodiments, the antibody molecule comprises a VL comprising an amino acid sequence having at least 80%, 85%, 90%, 95%, 96%, 97%, 98%, 99%, or 100% sequence identity to the amino acid sequence of VK-100. In some embodiments, the antibody molecule comprises a VL comprising an amino acid sequence having at least 80%, 85%, 90%, 95%, 96%, 97%, 98%, 99%, or 100% sequence identity to the amino acid sequence of VK-101. In some embodiments, the antibody molecule comprises a VL comprising an amino acid sequence having at least 80%, 85%, 90%, 95%, 96%, 97%, 98%, 99%, or 100% sequence identity to the amino acid sequence of VK-102. In some embodiments, the antibody molecule comprises a VL comprising an amino acid sequence having at least 80%, 85%, 90%, 95%, 96%, 97%, 98%, 99%, or 100% sequence identity to the amino acid sequence of VK-103. In some embodiments, the antibody molecule comprises a VL comprising an amino acid sequence having at least 80%, 85%, 90%, 95%, 96%, 97%, 98%, 99%, or 100% sequence identity to the amino acid sequence of VK-104. In some embodiments, the antibody molecule comprises a VL comprising an amino acid sequence having at least 80%, 85%, 90%, 95%, 96%, 97%, 98%, 99%, or 100% sequence identity to the amino acid sequence of VK-105. In some embodiments, the antibody molecule comprises a VL comprising an amino acid sequence having at least 80%, 85%, 90%, 95%, 96%, 97%, 98%, 99%, or 100% sequence identity to the amino acid sequence of VK-106. In some embodiments, the antibody molecule comprises a VL comprising an amino acid sequence having at least 80%, 85%, 90%, 95%, 96%, 97%, 98%, 99%, or 100% sequence identity to the amino acid sequence of VK-107. In some embodiments, the antibody molecule comprises a VL comprising an amino acid sequence having at least 80%, 85%, 90%, 95%, 96%, 97%, 98%, 99%, or 100% sequence identity to the amino acid sequence of VK-108. In some embodiments, the antibody molecule comprises a VL comprising an amino acid sequence having at least 80%, 85%, 90%, 95%, 96%, 97%, 98%, 99%, or 100% sequence identity to the amino acid sequence of VK-109. In some embodiments, the antibody molecule comprises a VL comprising an amino acid sequence having at least 80%, 85%, 90%, 95%, 96%, 97%, 98%, 99%, or 100% sequence identity to the amino acid sequence of VK-110. In some embodiments, the antibody molecule comprises a VL comprising an amino acid sequence having at least 80%, 85%, 90%, 95%, 96%, 97%, 98%, 99%, or 100% sequence identity to the amino acid sequence of VK-111.

In some embodiments, the antibody molecule comprises a VH comprising an amino acid sequence having at least 80%, 85%, 90%, 95%, 96%, 97%, 98%, 99%, or 100% sequence identity to the amino acid sequence of VH107. In some embodiments, the antibody molecule comprises a VH comprising an amino acid sequence having at least 80%, 85%, 90%, 95%, 96%, 97%, 98%, 99%, or 100% sequence identity to the amino acid sequence of VH123. In some embodiments, the antibody molecule comprises a VH comprising an amino acid sequence having at least 80%, 85%, 90%, 95%, 96%, 97%, 98%, 99%, or 100% sequence identity to the amino acid sequence of VH148. In some embodiments, the antibody molecule comprises a VH comprising an amino acid sequence having at least 80%, 85%, 90%, 95%, 96%, 97%, 98%, 99%, or 100% sequence identity to the amino acid sequence of VH175. In some embodiments, the antibody molecule comprises a VH comprising an amino acid sequence having at least 80%, 85%, 90%, 95%, 96%, 97%, 98%, 99%, or 100% sequence identity to the amino acid sequence of VH176.

In some embodiments, the antibody molecule comprises a VL comprising an amino acid sequence having at least 80%, 85%, 90%, 95%, 96%, 97%, 98%, 99%, or 100% sequence identity to the amino acid sequence of VK-24. In some embodiments, the antibody molecule comprises a VL comprising an amino acid sequence having at least 80%, 85%, 90%, 95%, 96%, 97%, 98%, 99%, or 100% sequence identity to the amino acid sequence of VK-65. In some embodiments, the antibody molecule comprises a VL comprising an amino acid sequence having at least 80%, 85%, 90%, 95%, 96%, 97%, 98%, 99%, or 100% sequence identity to the amino acid sequence of VK-83. In some embodiments, the antibody molecule comprises a VL comprising an amino acid sequence having at least 80%, 85%, 90%, 95%, 96%, 97%, 98%, 99%, or 100% sequence identity to the amino acid sequence of VK-107. In some embodiments, the antibody molecule comprises a VL comprising an amino acid sequence having at least 80%, 85%, 90%, 95%, 96%, 97%, 98%, 99%, or 100% sequence identity to the amino acid sequence of VK-110. In some embodiments, the antibody molecule comprises a VL comprising an amino acid sequence having at least 80%, 85%, 90%, 95%, 96%, 97%, 98%, 99%, or 100% sequence identity to the amino acid sequence of VK-111.

In some embodiments, the antibody molecule comprises: (i) a VH comprising an amino acid sequence having at least 80%, 85%, 90%, 95%, 96%, 97%, 98%, 99%, or 100% sequence identity to the amino acid sequence of VH123; and (ii) a VL comprising an amino acid sequence having at least 80%, 85%, 90%, 95%, 96%, 97%, 98%, 99%, or 100% sequence identity to the amino acid sequence of VK-65. In some embodiments, the antibody molecule further comprises one or more Fc mutations, e.g., one, two, or all three mutations of FcMut215, as described herein (e.g., T307Q, Q311V, and/or A378V).

In some embodiments, the antibody molecule comprises: (i) a VH comprising an amino acid sequence having at least 80%, 85%, 90%, 95%, 96%, 97%, 98%, 99%, or 100% sequence identity to the amino acid sequence of VH148; and (ii) a VL comprising an amino acid sequence having at least 80%, 85%, 90%, 95%, 96%, 97%, 98%, 99%, or 100% sequence identity to the amino acid sequence of VK-65. In some embodiments, the antibody molecule further comprises one or more Fc mutations, e.g., one, two, or all three mutations of FcMut215, as described herein (e.g., T307Q, Q311V, and/or A378V).

In some embodiments, the antibody molecule comprises: (i) a VH comprising an amino acid sequence having at least 80%, 85%, 90%, 95%, 96%, 97%, 98%, 99%, or 100% sequence identity to the amino acid sequence of VH175; and (ii) a VL comprising an amino acid sequence having at least 80%, 85%, 90%, 95%, 96%, 97%, 98%, 99%, or 100% sequence identity to the amino acid sequence of VK-65. In some embodiments, the antibody molecule further comprises one or more Fc mutations, e.g., one, two, or all three mutations of FcMut215, as described herein (e.g., T307Q, Q311V, and/or A378V).

In some embodiments, the antibody molecule comprises an Fc region described herein, e.g., an Fc region comprising one or more (e.g., 2, 3, 4, 5, 6, 7, 8, 9, 10, 11, 12, 13, 14, 15, 16, 17, 18, 19, 20, 21, 22, 23, 24, 25, or more) Fc mutations listed in Table 2. In some embodiments, the antibody molecule comprises an Fc region described herein, e.g., an Fc region comprising a combination of Fc mutations listed in a single row of Table 2. In some embodiments, the antibody molecule comprises one or more (e.g., 1, 2, or all 3 of) Fc mutations at positions selected from Q307, V311, and V378. In some embodiments, the antibody molecule comprises one or more (e.g., 1, 2, or all 3 of) Fc mutations selected from T307Q, Q311V, and A378V. In some embodiments, the antibody molecule comprises the Fc mutations T307Q, Q311V, and A378V.

In some embodiments, the antibody molecule comprising a heavy chain variable region (VH) comprising one or more of CDR1, CDR2, and CDR3 of VH123, and a light chain variable region (VL) comprising one or more of CDR1, CDR2, and CDR3 of VK-65. In some embodiments, the antibody molecule comprising a heavy chain variable region (VH) comprising one or more of CDR1, CDR2, and CDR3 of VH123, a light chain variable region (VL) comprising one or more of CDR1, CDR2, and CDR3 of VK-65, and an Fc region described herein, e.g., an Fc region comprising FcMut215.

In some embodiments, the antibody molecule comprises a heavy chain variable region (VH) with at least 90% sequence identity to VH123. In some embodiments, the antibody molecule comprises a light chain variable region (VL) with at least 90% sequence identity to VK-65. In some embodiments, the antibody molecule comprises an Fc region with at least 90% sequence identity to an Fc region described herein, e.g., an Fc region comprising FcMut215. In some embodiments, the antibody molecule comprises a heavy chain variable region (VH) with at least 90% sequence identity to VH123 and a light chain variable region (VL) with at least 90% sequence identity to VK-65. In some embodiments, the antibody molecule comprises a heavy chain variable region (VH) with at least 90% sequence identity to VH123, a light chain variable region (VL) with at least 90% sequence identity to VK-65, and an Fc region with at least 90% sequence identity to an Fc region described herein, e.g., an Fc region comprising FcMut215.

In some embodiments, the antibody molecule comprises one or more Fc mutations that enhances the half-life of the antibody molecule (e.g., in circulation and/or in serum) relative to a corresponding antibody molecule comprising a wild-type Fc region. In some embodiments, the Fc mutations enhance the interaction between the Fc region and FcRn. In some embodiments, antibody molecule comprises one or more Fc mutations that enhances an effector function (e.g., ADCC and/or CDC).

In some embodiments, the antibody molecule exhibits reduced polyreactivity compared to a reference antibody (e.g., FX-0-1-m3), e.g., according to an assay as described in Example 2. In some embodiments, the antibody molecule exhibits reduced self-interaction proclivity compared to a reference antibody (e.g., FX-0-1-m3), e.g., according to an assay as described in Example 2.

In an aspect, the disclosure features a composition (e.g., a pharmaceutical composition) comprising an anti-HA antibody molecule as described herein. In some embodiments, the pharmaceutical composition further comprises a pharmaceutically acceptable excipient or carrier.

In an aspect, the disclosure features a kit comprising an anti-HA antibody molecule as described herein. In some embodiments, the kit further comprises instructions for use of the anti-HA antibody molecule. In some embodiments, the kit further comprises instructions for administering the antibody molecule to a subject in need thereof (e.g., a subject having, or at risk of having, an influenza virus infection), e.g., according to a method as described herein.

In an aspect, the disclosure features a nucleic acid molecule encoding an anti-HA antibody molecule described herein, or a portion thereof. In some embodiments, the nucleic acid molecule encodes a VH of an antibody molecule described herein (or a functional fragment thereof) and/or a VL of an antibody molecule described herein (or a functional fragment thereof). In some embodiments, the nucleic acid molecule encodes a VH comprising the VH CDR sequences of an antibody molecule described herein (or a functional fragment thereof) and/or a VL comprising the VL CDR sequences of an antibody molecule described herein (or a functional fragment thereof).

In an aspect, the disclosure features a vector comprising a nucleic acid molecule as described herein.

In an aspect, the disclosure features a host cell comprising a nucleic acid molecule or vector as described herein.

In an aspect, the disclosure feature a method of making an anti-HA antibody molecule, the method comprising incubating a host cell as described herein (e.g., a host cell comprising a nucleic acid molecule or vector as described herein) under conditions suitable for production of the anti-HA antibody molecule.

In an aspect, the disclosure features a method of treating or preventing an influenza virus infection, or a symptom thereof, in a subject, comprising administering to the subject an effective amount of an anti-HA antibody molecule described herein.

In some embodiments, the method prevents an influenza virus infection. In some embodiments, the subject is at risk of having an influenza infection. In some embodiments, the antibody molecule is administered before the subject is exposed to an influenza virus. In some embodiments, the antibody molecule the antibody molecule is administered subcutaneously or intramuscularly. In some embodiments, the method prevents an influenza virus infection for at least 5, 10, 15, 20, 25, 30, 40, 45, 50, 60, 70, 80, 90, 100, 150, 200 days or more. In some embodiments, the antibody molecule is administered once during an influenza season. In some embodiments, the subject is a human subject (e.g., a human subject described herein).

In some embodiments, the subject is prevented from being infected with an influenza virus for at least 1 week, 2 weeks, 3 weeks, 4 weeks, 1 month, 2 months, 3 months, 4 months, 5 months, 6 months, or more (e.g., season-long prevention).

In an aspect, the disclosure features an antibody molecule as described herein for use in treating or preventing an influenza virus infection, or a symptom thereof, in a subject (e.g., a subject described herein), e.g., in accordance with a method described herein.

In an aspect, the disclosure features use of an antibody molecule as described herein in treating or preventing an influenza virus infection, or a symptom thereof, in a subject (e.g., a subject described herein), e.g., in accordance with a method described herein.

In an aspect, the disclosure features use of an antibody molecule as described herein in the manufacture of a medicament for treating or preventing an influenza virus infection, or a symptom thereof, in a subject (e.g., a subject described herein), e.g., in accordance with a method described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments featured in the disclosure are set forth in the accompanying drawings and the description below. Other features, objects, and advantages featured in the disclosure will be apparent from the description and drawings, and from the claims.

### BRIEF DESCRIPTION OF THE DRAWINGS

FIG. 1 depicts the binding and neutralization activity by antibody molecule FX-0-1-215 and an exemplary anti-HA antibody molecule described herein.

FIG. 2 depicts the pharmacokinetics of FX-0-1-m3, FX-0-1-215, and an exemplary anti-HA antibody molecule described herein.

FIG. 3 depicts the effector functions of FX-0-1-m3, FX-0-1-215, and an exemplary anti-HA antibody molecule described herein.

FIG. 4A depicts the pharmacokinetics of FX-0-1-215 (control) and an exemplary anti-HA antibody (FX-122-4-215) that comprises FcMut215.

FIG. 4B depicts the polyreactivity of FX-0-1-m3 (control) and an exemplary anti-HA antibody molecule (FX-122-4-m3) that comprises FcMut215.

FIG. 5A depicts four potential aggregation sites with surface hydrophobic residues that were identified by in silico analysis.

FIG. 5B depicts the convergence of the four sites with hydrophobic residues in the HCDR3 and LCDR1 regions of the antibody molecule.

FIG. 6 depicts, in the top panel, a representation of FX-0-1-m3 binding to H3 HA. N76 of FX-0-1-m3 VH contacts N278 of HA1. Insertion of leucine at position 76 (bottom) is predicted to provide better hydrophobic surface complementarity.

FIG. 7 depicts the percentage of IgG remaining in serum (normalized to 1-hour concentration) in Tg276 mice after a 2 mg/kg injection with the indicated exemplary anti-HA antibody molecules.

FIG. 8 depicts SE-HPLC analysis of two lots of the exemplary anti-HA antibody molecule FX-0-0-m17 on a Phenomenex Bio-Sep S3000 column.

FIGS. 9A-9C show expression and binding properties of exemplary anti-HA antibody molecules. All data is organized with heavy chain designs in each column (3-12) and light chain designs in each row (2-7). FIG. 9A shows small-scale expression of exemplary engineered antibodies in Expi293 cells. Data is reported as µg/mL of antibody in cell culture supernatant. FIGS. 9B-9C show cell culture supernatant binding to H1 (A/CA/09/2007) (FIG. 9B) and H3 (A/Brisbane/10/2007) (FIG. 9C). Data is shown as EC50 or c value in nanomolar resulting from a four-parameter logistical regression of an antibody titration series. All data is color-coded with red shading to indicate either poor expression or poor binding to HA.

FIG. 10 is a diagram showing a homology model of the HCDR3 loop of an exemplary anti-HA antibody molecule and the HA2 α-helix. Highlighted in red are the residues contributing to site I SAP and highlighted in dark blue are residues contributing to site II.

FIG. 11 is a diagram showing a homology model of the LCDR1 loop of FX-0-1-m3 and the HA2 α-helix. Highlighted in dark blue are the residues contributing to site III SAP (F27d, Y29) and highlighted in red are residues Q27, S27a. Both sites were re-engineered for improved PK properties.

FIG. 12 is a table showing the affinity of exemplary anti-HA antibodies having mutations of S74, K75, and N76 to hydrophobic or aromatic residues.

FIG. 13 is a series of graphs showing the results of pharmacokinetic studies performed in Tg276 mice for the indicated exemplary anti-HA antibody molecules. Percent of IgG remaining in serum (normalized to 1-hour Cmax) in Tg276 mice after a 2 mg/kg intravenous injection. It is to be noted, in JAX10 study the control antibody (FX-0-1-215) had lower persistence as compared to previous studies.

FIG. 14 is a series of graphs showing abbreviated pharmacokinetic studies performed in Tg276 mice for the indicated exemplary anti-HA antibody molecules. Percent of IgG remaining in serum (normalized to 1- hour Cmax) in Tg276 mice after a 2 mg/kg intravenous injection.

FIG. 15 is a series of graphs showing that an exemplary anti-HA antibody molecule containing Q27D/S27aE affinity-enhancing mutations in LCDR1 exhibited enhanced properties. (left) Binding, (middle) in vitro neutralization and (right) ADCC activity of FX-123-24 and FX-0-1 against A/Hong Kong/4801/2014 (H3N2) HA and virus.

FIG. 16 is a series of graphs showing ADCC (left panel) and ADCP (right panel) activity of exemplary anti-HA antibodies against H3 infected MDCK London cells.

### DETAILED DESCRIPTION

The disclosure is based, at least in part, on the design and synthesis of antibody molecules that can bind an epitope that is conserved across multiple hemagglutinin subtypes of influenza viruses (e.g., influenza A viruses). For example, the antibody molecules described herein are useful as broad-spectrum therapy against disease caused by at least one influenza A strain belonging to Group 1 and one influenza A strain belonging to Group 2 to neutralize infectivity of viruses belonging to both Group 1 and Group 2 (at least one subtype of each). Without wising to be bound by theory, it is believed that in some embodiments, the antibody molecules described herein have broad activity against seasonal strains (H1N1 and H3N2 subtype), are available in the target organs for the entire season (e.g., at least about 5 to 6 months) at efficacious concentrations, and/or are able to be formulated at high concentrations (e.g., greater than about 100 mg/mL), to support the use of the antibody molecules as a single administration (e.g., intramuscular or subcutaneous administration) prophylactic against influenza.

Based on the data from 3,254 children and adults enrolled in the U.S. Influenza Vaccine Effectiveness Network (U.S. Flu VE Network) during Nov. 23, 2018-Feb. 2, 2019, the overall adjusted vaccine effectiveness against all influenza virus infection associated with medically attended acute respiratory illness (ARI) was 47% (Doyle et al., MMWR Morb Mortal Wkly Rep. 2019; 68(6): 135-139). Vaccination is insufficient to effectively protect all people at risk of having influenza, particularly high-risk populations.

Without wishing to be bound by theory, it is believed that in some embodiments, the antibody molecules described herein are effective for the prophylaxis of influenza. For example, the antibody molecules described herein can result in higher protection against severe infection than vaccination, such as protection from pandemic strains, and/or can confer protection for immunocompromised and other high-risk groups for which vaccines are less effective. The high-risk populations include, but are not limited to, immunocompromised patients (e.g., cancer patients on a chemotherapy, transplant recipients, or HIV/AIDS patients), patients with chronic obstructive pulmonary disease (COPD) (e.g., mold-to-moderate, or severe COPD), patients with asthma (e.g., mild-to-moderate, or severe asthma), patients with diabetes, patients with a heart disease, patients with a chronic kidney disease, cancer patients not on a chemotherapy, or healthy elderly people (e.g., age 65 year or older).

Without wishing to be bound by theory, it is believed that in some embodiments, the antibody molecules described herein target a conserved and constrained epitope in hemagglutinin (HA). In some embodiments, the antibody molecule targets a highly constrained region on the stem or stalk region of HA. For example, the conserved and constrained epitope can be associated with the structural and functional integrity, common across multiple influenza strains, and/or resistant to mutations. In some embodiments, the antibody molecule is a modified human IgG1 monoclonal antibody.

Without wishing to be bound by theory, it is believed that in some embodiments, multiple mechanisms of action can allow for potent anti-viral activity, including distinct mechanisms from small molecule antivirals. For example, the antibody molecules described herein can inhibit viral/endosomal membrane fusion, have an antibody-dependent cellular cytotoxicity (ADCC) activity, and/or have an antibody-dependent cellular phagocytosis (ADCP) activity. In some embodiments, the antibody molecule is a human IgG1 monoclonal antibody, e.g., modified to improve the inhibition of viral/endosomal membrane fusion, the ADCC activity, and/or the ADCP activity. In some embodiments, the antibody molecule has broad coverage across Groups 1 and 2 influenza A viruses. In some embodiments, the antibody molecule has picomolar (pM) binding affinity to HA. In some embodiments, the antibody molecule has an enhanced binding affinity for H3 HA protein.

The antibody molecules described herein can have a half-life that is suitable for prophylactic use. IgG can be internalized into vascular endothelial cells through pinocytosis. IgG (and albumin) can bind to the neonatal Fc receptor (FcRn) in low pH environment of the endosome. FcRn-bound IgG can be processed in one of two ways: recycling back to the apical cell membrane, or transcytosis from the apical to basolateral side. IgG not associated with FcRn is degraded by lysosomes. The antibody molecules described herein can comprise an Fc variant, for example, an Fc variant having one or more (e.g., two, three, or all) of the following properties: (a) enhancing circulatory half-life when combined with different Fabs; (b) maintaining robust biophysical properties and favorable developability characteristics; (c) having effector functions that are comparable to wild-type Fc, or that in some cases are enhanced compared to wild-type Fc; or (d) enhancing half-life in non-human primates (NHP) by about 3-4 fold or more. Exemplary Fc variants are described, e.g., in PCT Application Publication No. WO2018/052556, U.S. Pat. Application Publication No. US 2018/0037634, and in Booth et al., MAbs. 2018; 10(7): 1098-1110, the contents of the aforesaid publications are incorporated by reference in their entirety. In some embodiments, the Fc region, the Fab region, or both, of the antibody molecule is engineered for enhanced circulatory half-life and developability characteristics.

The antibody molecules described herein can be used for prevention of influenza infection in high-risk individuals. In some embodiments, a single subcutaneous dose can be effective for season-long protection. In some embodiments, the antibody molecule has equivalent safety and efficacy in neutralizing multiple influenza A strains (e.g., from both groups, or of multiple or all subtypes), e.g., as a result of retaining one or more (e.g., 2, 3, 4, 5, or all) of the CDR regions of a reference anti-HA antibody, e.g., FX-0-1-m3. FX-0-1-m3 (also known as Ab 044) is described, e.g., in PCT Application Publication Nos. WO 2013/170139 and WO 2017/083627, U.S. Pat. Nos. 8,877,200, 9,096,657, 9,969,794, and 10,513,553, the contents of the aforesaid publications are incorporated by reference in their entirety.

Without wishing to be bound by theory, it is believed that in some embodiments, the antibody molecules described herein target a highly networked, mutationally constrained epitope. In some embodiments, the antibody molecules described herein can be used for treatment of severe influenza. In some embodiments, the antibody molecules described herein comprise a modification in the Fab and Fc region for use in prophylaxis of influenza.

Without wishing to be bound by theory, it is believed that, in some embodiments, the antibody molecules, e.g., the anti-HA antibody molecules, described herein comprise mutations resulting in greater binding affinity and/or in vitro neutralization activity than a reference anti-HA antibody molecule, e.g., FX-0-1-m3. In some embodiments, the antibody molecules, e.g., the anti-HA antibody molecules, described herein comprise mutations resulting in improved half-life and/or effector functions, e.g., ADCC and ADCP activity, to a reference anti-HA antibody molecule, e.g., FX-0-1-m3. In some embodiments, an antibody molecule, e.g., an anti-HA antibody molecule described herein, comprising a variable heavy chain region described herein, e.g., VH123, a variable light chain described herein, e.g., VK-65, and an Fc region described herein, e.g., FcMut215, has a binding affinity and/or in vitro neutralization capacity greater than a reference anti-HA antibody, e.g., FX-0-1-m3. In some embodiments, an antibody molecule, e.g., an anti-HA antibody molecule described herein comprising a variable heavy chain described herein, e.g., VH123, a variable light chain described herein, e.g., VK-65, and an Fc region described herein, e.g., FcMut215, has improved half-life and/or effector function, e.g., ADCC and/or ADCP activity, compare to a reference anti-HA antibody, e.g., FX-0-1-m3. In some embodiments, the antibody molecule has a circulating half-life of at least 30 days, e.g., at least 35, 40, 45, 50, 55, or 60 days. In some embodiments, the antibody molecule has a circulating half-life of at least 45 days.

### Definitions

As used herein, the term “antibody molecule” refers to a polypeptide that comprises sufficient sequence from an immunoglobulin heavy chain variable region and/or sufficient sequence from an immunoglobulin light chain variable region, to provide antigen specific binding. It comprises full length antibodies as well as fragments thereof, e.g., Fab fragments, that support antigen binding. Typically, an antibody molecule will comprise heavy chain CDR1, CDR2, and CDR3 and light chain CDR1, CDR2, and CDR3 sequence. Antibody molecules include human, humanized, CDR-grafted antibodies and antigen binding fragments thereof. In some embodiments, an antibody molecule comprises a protein that comprises at least one immunoglobulin variable region segment, e.g., an amino acid sequence that provides an immunoglobulin variable domain or immunoglobulin variable domain sequence.

The VH or VL chain of the antibody molecule can further include all or part of a heavy or light chain constant region, to thereby form a heavy or light immunoglobulin chain, respectively. In one embodiment, the antibody molecule is a tetramer of two heavy immunoglobulin chains and two light immunoglobulin chains.

An antibody molecule can comprise one or both of a heavy (or light) chain immunoglobulin variable region segment. As used herein, the term “heavy (or light) chain immunoglobulin variable region segment,” refers to an entire heavy (or light) chain immunoglobulin variable region, or a fragment thereof, that is capable of binding antigen. The ability of a heavy or light chain segment to bind antigen is measured with the segment paired with a light or heavy chain, respectively. In some embodiment, a heavy or light chain segment that is less than a full length variable region will, when paired with the appropriate chain, bind with an affinity that is at least 20, 30, 40, 50, 60, 70, 80, 90, or 95% of what is seen when the full length chain is paired with a light chain or heavy chain, respectively.

An immunoglobulin variable region segment may differ from a reference or consensus sequence. As used herein, to “differ,” means that a residue in the reference sequence or consensus sequence is replaced with either a different residue or an absent or inserted residue.

An antibody molecule can comprise a heavy (H) chain variable region (abbreviated herein as VH), and a light (L) chain variable region (abbreviated herein as VL). In another example, an antibody comprises two heavy (H) chain variable regions and two light (L) chain variable regions or antibody binding fragments thereof. The light chains of the immunoglobulin may be of type kappa or lambda. In one embodiment, the antibody molecule is glycosylated. An antibody molecule can be functional for antibody dependent cytotoxicity and/or complement-mediated cytotoxicity, or may be non-functional for one or both of these activities. An antibody molecule can be an intact antibody or an antigen-binding fragment thereof.

Antibody molecules include “antigen-binding fragments” of a full-length antibody, e.g., one or more fragments of a full-length antibody that retain the ability to specifically bind to an HA target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′) or F(ab′)₂ fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See, e.g., Bird et al. (1988) Science 242:423-426; and Huston et al. (1988) Proc. Natl. Acad. Sci. USA 85:5879-5883. Antibody molecules include diabodies.

As used herein, an antibody refers to a polypeptide, e.g., a tetrameric or single chain polypeptide, comprising the structural and functional characteristics, particularly the antigen binding characteristics, of an immunoglobulin. Typically, a human antibody comprises two identical light chains and two identical heavy chains. Each chain comprises a variable region.

The variable heavy (VH) and variable light (VL) regions can be further subdivided into regions of hypervariability, termed “complementarity determining regions” (“CDR”), interspersed with regions that are more conserved, termed “framework regions” (FR). Human antibodies have three VH CDRs and three VL CDRs, separated by framework regions FR1-FR4. The extent of the FRs and CDRs has been precisely defined (see, Kabat, E.A., et al. (1991) Sequences of Proteins of Immunological Interest, Fifth Edition, U.S. Department of Health and Human Services, NIH Publication No. 91-3242; and Chothia, C. et al. (1987) J. Mol. Biol. 196:901-917). Kabat definitions are used herein. Each VH and VL is typically composed of three CDRs and four FRs, arranged from amino-terminus to carboxyl-terminus in the following order: FR1, CDR1, FR2, CDR2, FR3, CDR3, FR4.

The heavy and light immunoglobulin chains can be connected by disulfide bonds. The heavy chain constant region typically comprises three constant domains, CH1, CH2 and CH3. The light chain constant region typically comprises a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system.

The term “immunoglobulin” comprises various broad classes of polypeptides that can be distinguished biochemically. Those skilled in the art will appreciate that heavy chains are classified as gamma, mu, alpha, delta, or epsilon (γ, µ, α, δ, ε) with some subclasses among them (e.g., γ1-γ4). It is the nature of this chain that determines the “class” of the antibody as IgG, IgM, IgA IgD, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure. Light chains are classified as either kappa or lambda (κ, λ). Each heavy chain class may be bound with either a kappa or lambda light chain.

Suitable antibody molecules include, but are not limited to, monoclonal antibodies, monospecific antibodies, polyclonal antibodies, polyspecific antibodies, human antibodies, primatized antibodies, chimeric antibodies, bi-specific antibodies, humanized antibodies, conjugated antibodies (i.e., antibodies conjugated or fused to other proteins, radiolabels, cytotoxins), Small Modular ImmunoPharmaceuticals (“SMIPs™”), single chain antibodies, cameloid antibodies, and antibody fragments.

In some embodiments, an antibody molecule is a humanized antibody. A humanized antibody refers to an immunoglobulin comprising a human framework region and one or more CDR’s from a non-human, e.g., mouse or rat, immunoglobulin. The immunoglobulin providing the CDR’s is often referred to as the “donor” and the human immunoglobulin providing the framework often called the “acceptor,” though in some embodiments, no source or no process limitation is implied. Typically, a humanized antibody comprises a humanized light chain and a humanized heavy chain immunoglobulin.

An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay (1988) Ann. Rev. Immunol. 6:381-405).

As used herein, an “immunoglobulin variable domain sequence” refers to an amino acid sequence that can form the structure of an immunoglobulin variable domain. For example, the sequence may include all or part of the amino acid sequence of a naturally-occurring variable domain. For instance, the sequence may omit one, two or more N- or C-terminal amino acids, internal amino acids, may include one or more insertions or additional terminal amino acids, or may include other alterations. In one embodiment, a polypeptide that comprises an immunoglobulin variable domain sequence can associate with another immunoglobulin variable domain sequence to form a target binding structure (or “antigen binding site”), e.g., a structure that interacts with the target antigen.

As used herein, the term antibodies comprises intact monoclonal antibodies, polyclonal antibodies, single domain antibodies (e.g., shark single domain antibodies (e.g., IgNAR or fragments thereof)), multispecific antibodies (e.g., bi-specific antibodies) formed from at least two intact antibodies, and antibody fragments so long as they exhibit the desired biological activity. Antibodies for use herein may be of any type (e.g., IgA, IgD, IgE, IgG, or IgM).

The antibody molecule can be derived from a mammal, e.g., a rodent, e.g., a mouse or rat, horse, pig, or goat. In some embodiments, the antibody molecule is produced using a recombinant cell. In some embodiments, the antibody molecule is a chimeric antibody, for example, from mouse, rat, horse, pig, or other species, bearing human constant and/or variable regions domains.

A binding agent, as used herein, is an agent that bind, e.g., specifically binds, a target antigen, e.g., HA. Binding agents of the invention share sufficient structural relationship with anti-HA antibody molecules disclosed herein to support specific binding to HA, and in some embodiments, other functional properties of an anti-HA antibody molecule disclosed herein. In some embodiments, a binding agent will exhibit a binding affinity at of at least 10, 20, 30, 40, 50, 60, 70, 80, or 90% of an antibody molecule disclosed herein, e.g., an antibody molecule with which it shares, significant structural homology, e.g., CDR sequences. Binding agents can be naturally occurring, e.g., as are some antibodies, or synthetic. In an embodiment, the binding agent is a polypeptide, e.g., an antibody molecule. While some binding agents are antibody molecules, other molecules, e.g., other polypeptides, can also function as binding agents. Polypeptide binding agents can be monomeric or multimeric, e.g., dimeric, trimeric, or tetrameric and can be stabilized by intra- or interchain bonds, e.g., disulfide bonds. They can contain natural or non-naturally occurring amino acid residues. In some embodiments, binding agents are antibody molecules, or other polypeptides, that present one or more CDRs of antibody molecules disclosed herein or that otherwise mimic the structure of an antibody molecule disclosed herein. Binding agents can also comprise aptamers, nucleic acids or other molecular entities. A binding agent can be developed in a variety of ways, e.g., by immunization, by rational design, screening of random structures, or a combination of those or other approaches. Typically, a binding agent will act by making contact with substantially the same epitope as an antibody molecule disclosed herein, e.g., an antibody molecule with which it shares, significant structural homology, e.g., CDR sequences. A binding agent can interact with amino acids, saccharides, or combinations thereof. Polypeptides other than antibodies can be used as a scaffold to present sequence, e.g., one or more, or a complete set of heavy chain and/or light chain CDRs, disclosed herein. Exemplary scaffolds include adnectin, zinc finger DNA-binding proteins. protein A, lipoclins, ankryin consensus repeat domain, thioredoxin, anticalins, centyrin, avimer domains, ubiquitin, peptidomimetics, stapled peptides, cystine-knot miniproteins, and IgNARs. In some embodiments, a binding agent is or comprises a nucleic acid, e.g., DNA, RNA or mixtures thereof. In some embodiments, a binding agent, e.g., a nucleic acid, shows secondary, tertiary, or quaternary structure. In some embodiments a binding agent, e.g., a nucleic acid, forms a structure that mimics the structure of an antibody molecule disclosed herein.

A broad-spectrum binding agent, e.g., antibody molecule, as used herein, binds, a plurality of different HA molecules, and optionally neutralizes viruses comprising the different HA molecules. In an embodiment, it binds a first HA and binds a second HA from influenza A Group 1, and optionally neutralizes viruses comprising the first or second HA molecules. In an embodiment, it binds a first HA from an influenza A Group 1 virus and binds a second HA from an influenza A Group 2 virus, and optionally neutralizes viruses comprising the different HA molecules. In an embodiment, it binds, and in some embodiments neutralizes, at least two different clades or clusters of viruses, e.g., from different Groups. In some embodiments, it binds, and in some embodiments neutralizes, all or substantially all strains of Group 1 an/or Group 2 disclosed herein. In an embodiment, a binding agent, e.g., antibody molecule, binds, and in some embodiments, neutralizes: at least one strain from the Group 1 H1, e.g., H1a or H1b, cluster and at least one strain from the Group 2 H3 or H7 cluster. In some embodiments, binding agent, e.g., antibody molecule, binds, and optionally neutralizes or mediate infection of particular hosts, e.g., avian, camel, canine, cat, civet, equine, human, mouse, swine, tiger, or other mammal or bird.

The term “combination therapy”, as used herein, refers to administration of a plurality of agents, e.g., wherein at least one binding agent, e.g., antibody molecule, disclosed herein is administered to a subject, e.g., a human subject. The introduction of the agents into the subject can be at different times. In some embodiments, the agents are administered in overlapping regimens, or such that the subject is simultaneously exposed to both agents, or such that the response of the subject is better than would be seen with either agent administered alone.

As used herein, an “escape mutant” is a mutated influenza strain that is resistant to neutralization by an anti-HA antibody molecule described herein. In some embodiments, an escape mutant is resistant to neutralization with a binding agent, e.g., antibody molecule, but its parent strain is neutralized by the binding agent, e.g., antibody molecule. Resistance can be tested by various methods, including, but not limited to, genotypic testing (e.g., Sanger sequencing/nested PCR -baseline and last qPCR sample (Ct<32)), and phenotypic testing (e.g., plaque reduction on primary sample, e.g., ViroSpot™ assay (e.g., virus titration - last post-baseline ≥ 2 Log₁₀ TCID₅₀/mL) or IC50 single passage sample (e.g., antibody titration - last post-baseline ≥ 1 Log₁₀ TCID₅₀/mL).

As used herein, “pandemic influenza” refers to a new viral strain that arises due to human adaptation of an influenza strain by mutation or by emergence of a strain by reassortment of different strains of influenza A. The resulting pandemic strain is significantly different from previous strains and most people will have little or no pre-existing immunity. Symptoms and complications may be more severe and more frequent than those typical of seasonal influenza. Examples of past pandemic flu viruses include, e.g., the 2009 H1N1 ‘swine flu,’ the 1957-58 H2N2 ‘Asian flu’ and the 1968 H3N2 influenza strains.

The terms “purified” and “isolated” as used herein in the context of an antibody molecule, e.g., an antibody, or generally a polypeptide, obtained from a natural source, refers to a molecule which is substantially free of contaminating materials from the natural source, e.g., cellular materials from the natural source, e.g., cell debris, membranes, organelles, the bulk of the nucleic acids, or proteins, present in cells. Thus, a polypeptide, e.g., an antibody molecule, that is isolated includes preparations of a polypeptide having less than about 30%, 20%, 10%, 5%, 2%, or 1% (by dry weight) of cellular materials and/or contaminating materials. The terms “purified” and “isolated” when used in the context of a chemically synthesized species, e.g., an antibody molecule, refers to the species which is substantially free of chemical precursors or other chemicals which are involved in the syntheses of the molecule.

A preparation of binding agents, e.g., antibody molecules, as used herein, comprises a plurality of molecules of a binding agent, e.g., antibody molecule, described herein. In some embodiments, the binding agent, e.g., antibody molecule, makes up at least 60, 70, 80, 90, 95, 98, 99, 99.5 or 99.9%, of the preparation, or of the active ingredients of the preparation, by weight or number. In some embodiments, that binding agent is an antibody molecule which makes up at least 60, 70, 80, 90, 95, 98, 99, 99.5 or 99.9%, of the preparation, or of the active ingredients, or polypeptide ingredients, or antibody molecules, of the preparation, by weight or number. In some embodiments, the binding agent is an antibody molecule and the preparation contains no more than 30, 20, 10, 5, 2, 1, or 0.5%, by weight or number, of a contaminant, e.g., a reactant, solvent, precursor or other species, from the source, or used in the preparation, of the antibody molecule, e.g., a species from a cell, reaction mixture, or other system used to produce the antibody molecule.

As used herein, the term “preventing an influenza,” “prevent an influenza,” “preventing an influenza virus infection,” or “prevent an influenza virus infection” means that a subject (e.g., a human) is less likely to be infected by influenza if the subject receives the antibody molecule prior to (e.g., 1 day, 2 days, 1 week, 2 weeks, 3 weeks, 4 weeks, 1 month, 2 months, 3 months, or more before) being exposed to an influenza virus.

As used herein, “seasonal influenza virus” is a strain that is identical or closely related to strains that have been circulating in the human population in recent years and therefore most people are at least partially immune to it. Such a strain is not likely to cause severe disease. Symptoms can include fever, cough, runny nose, and muscle pain, and in rare cases, death can result from complications, such as pneumonia. Outbreaks follow predictable seasonal patterns, annually, and usually in fall and winter and in temperate climates. Infection due to seasonal influenza virus is commonly referred to as the flu.

As used herein, specific binding, means that a binding agent, e.g., an antibody molecule, binds its antigen with a K_{D} of equal to or less than 10⁻⁵. In some embodiments, the antibody binds its antigen with a K_{D} of equal to or less than 10⁻⁶, 10⁻⁷, 10⁻⁸, 10⁻⁹, 10⁻¹⁰, 10⁻¹¹, or 10⁻¹².

As used herein, the term “therapeutically effective amount” refers to an amount of a therapeutic agent, e.g., a binding agent, e.g., an antibody molecule, which results in a positive outcome for the subject. In some embodiments, it can be statistically correlated with therapeutic effect or benefit, e.g., the lessening or prevention of a manifestation of an effect or a symptom, when administered to a population of subjects. In some embodiments, it is an amount that also provides a preselected, or reasonable, benefit/risk ratio. In some embodiments, it is an amount effective to reduce the incidence and/or severity of and/or to delay onset of one or more features, symptoms, or characteristics of a disease, disorder, or condition. A therapeutically effective amount is can be administered in a dosing regimen that may comprise one or multiple unit doses.

As used herein, the term “treating an influenza,” “treat an influenza,” “treating an influenza virus infection,” or “treat an influenza virus infection” means that a subject (e.g., a human) who has been infected with an influenza virus and experiences symptoms of the influenza (e.g., the flu), will in some embodiments, suffer less severe symptoms and/or will recover faster when the antibody molecule is administered than if the antibody is never administered. In some embodiments, when an infection is treated, an assay to detect virus in the subject will detect less virus after effective treatment for the infection. For example, a diagnostic assay using an antibody molecule, such as an antibody molecule described herein, will detect less or no virus in a biological sample of a patient after administration of an antibody molecule for the effective treatment of the viral infection. Other assays, such as PCR (e.g., qPCR) can also be used to monitor treatment in a patient, to detect the presence, e.g., decreased presence (or absence) after treatment of viral infection in the patient. Treatment can, e.g., partially or completely alleviate, ameliorate, relive, inhibit, reduce the severity of, and/or reduces incidence and optionally, delay onset of, one or more manifestations of the effects or symptoms, features, and/or causes of a particular disease, disorder, and/or condition (e.g., influenza). In some embodiments, treatment is of a subject who does not exhibit signs of the relevant disease, disorder and/or condition and/or of a subject who exhibits only early signs of the disease, disorder, and/or condition. In some embodiments, treatment is of a subject who exhibits one or more established signs of the relevant disease, disorder and/or condition. In some embodiments, treatment is of a subject diagnosed as suffering from influenza.

Calculations of “homology” or “sequence identity” or “identity” between two sequences (the terms are used interchangeably herein) can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

### Hemagglutinin (HA) Polypeptides and Influenza

Influenza viruses are negative sense, single-stranded, segmented RNA envelope viruses. Two glycoproteins, a hemagglutinin (HA) polypeptide and a neuraminidase (NA) polypeptide, are displayed on the outer surface of the viral envelope. There are several Influenza A subtypes, labeled according to an H number (for the type of hemagglutinin) and an N number (for the type of neuraminidase). There are 17 different H antigens (H1 to H17) and nine different N antigens (N1 to N9). Influenza strains are identified by a nomenclature based on the number of the strain’s HA polypeptide and NA polypeptide subtypes, for example, H1N1, H1N2, H1N3, H1N4, H1N5, and the like.

HA is the major viral surface glycoprotein that mediates binding and entry of the virus into host cells and is a primary target of neutralizing antibody responses. HA is a trimer of three identical monomers. Each monomer is synthesized as a precursor, HA₀, that is proteolytically processed into two disulfide-bonded polypeptide chains, HA₁ and HA₂. The ectodomain of this protein has (i) a globular head domain possessing receptor binding activity and major antigenic determinants, (ii) a hinge region, and (iii) a stem region where a sequence critical for fusion, the fusion peptide, is located. The viral replication cycle is initiated when the virion attaches via its surface hemagglutinin proteins to sialylated glycan receptors on the host cell and enters the cell by endocytosis. The acidic environment in the endosome induces conformational changes in HA that expose the fusion peptide hidden within the stem region of the trimer. The exposed fusion peptide mediates the fusion of the viral and target cell membranes resulting in the release of the viral ribonucleoprotein into the cell cytoplasm.

Influenza A hemagglutinin subtypes have been divided into two main groups and four smaller clades, and these are further divided into clusters. Group 1 influenza A strains are divided into 3 clades: (i) H8, H9 and H12 (“the H9 cluster”); (ii) H1, H2, H5, H6 and H17 (“the H1a cluster”); and (iii) H11, H13 and H16 (“the H1b cluster”). Group 2 strains are divided into 2 clades: (i) H3, H4 and H14 (“the H3 cluster”); and (ii) H7, H10 and H15 (“the H7 cluster”). The H1b and the H1a clusters are classified together as the H1 cluster. The different HA subtypes do not necessarily share strong amino acid sequence identity, but their overall 3D structures are similar.

Of the 17 HA polypeptide subtypes, only 3 (H1, H2 and H3) have adapted for human infection. These subtypes have in common an ability to bind alpha 2,6 sialylated glycans. In contrast, their avian counterparts preferentially bind to alpha 2,3 sialylated glycans. HA polypeptides that have adapted to infect humans (e.g., of HA polypeptides from the pandemic H1N1 (1918) and H3N2 (1967-68) influenza subtypes) have been characterized by an ability to preferentially bind to α2,6 sialylated glycans in comparison with their avian progenitors that preferentially bind to α2,3 sialylated glycans (see, e.g., Skehel & Wiley, Annu Rev Biochem, 69:531, 2000; Rogers, & Paulson, Virology, 127:361, 1983; Rogers et al., Nature, 304:76, 1983; Sauter et al., Biochemistry, 31:9609, 1992

Further, HA polypeptides that mediate infection of humans preferentially bind to umbrella topology glycans over cone topology glycans (see, e.g., U.S. 2011/0201547). Without wishing to be bound by any particular theory, it has been proposed that the ability to infect human hosts correlates less with binding to glycans of a particular linkage, and more with binding to glycans of a particular topology, even though cone-topology glycans may be α2,6 sialylated glycans. In has been demonstrated that HA polypeptides that mediate infection of humans bind to umbrella topology glycans, often showing preference for umbrella topology glycans over cone topology glycans (See, for example, U.S. Application Publication Nos. 2009/0269342, 2010/0061990, 2009/0081193, and 2008/0241918, and International Publication No. WO2008/073161).

Mature HA polypeptides include three domains, (i) a globular domain (a.k.a., the head domain) consists mainly of the HA1 peptide and contains the receptor (sialylated glycoproteins)-binding region, (ii) a stalk domain (HA1 and HA2) where the membrane fusion peptide resides, and (iii) a transmembrane domain (HA2) that anchors hemagglutinin to the viral envelope. A set of amino acids in the interface of the HA1 and HA2 peptides is highly conserved across all influenza subtypes. The HA1/HA2 membrane proximal region (MPER), including a canonical alpha-helix, is also highly conserved across influenza subtypes.

HA polypeptides interact with the surface of cells by binding to a glycoprotein receptor, known as the HA receptor. Binding of an HA polypeptide to an HA receptor is predominantly mediated by N-linked glycans on the HA receptors. HA polypeptides on the surface of flu virus particles recognize sialylated glycans that are associated with HA receptors on the surface of the cellular host. Following replication of viral proteins and genome by the cellular machinery, new viral particles bud from the host to infect neighboring cells.

Currently, vaccines are administered to subjects, e.g., humans, to prevent the flu, e.g., to prevent infection or to minimize the effects of an infection with influenza virus. Traditional vaccines contain a cocktail of antigens from various strains of influenza and are administered to humans to prevent the human from getting infected with the virus. HA is the main target of influenza A-neutralizing antibodies, and HA undergoes continuous evolution driven by the selective pressure of the antibody response, which is primarily directed against the membrane-distal receptor-binding subdomain of the HA polypeptide. The subject, however, is protected only from strains that are identical to, or closely related to, the strains from which the antigens in the cocktail were derived. The human is still most vulnerable to infection by other strains of the flu that were not included in the cocktail. One of the advantages of the antibodies provided herein is their ability to bind an epitope of HA that is conserved across multiple strains of influenza A. Thus, administration of an anti-HA antibody described herein will be more effective to protect an individual from infection from a broader spectrum of influenza (e.g., influenza A) and conditions associate thereof (e.g., secondary infections, e.g., secondary bacterial infections). Further, the antibodies are effective in treating a subject after infection has occurred.

### Anti-HA Antibody Molecules

Binding agents, and in particular, the antibody molecules described herein (e.g., anti-HA antibody molecules described herein), can bind to influenza A viruses from both Group 1 and Group 2. For example, the antibody molecules described herein can bind to an hemagglutinin (HA) polypeptide on at least 1, 2, 3, 4, 5, 6, 7, 8, 9, 10 or 11 strains from Group 1, and can also bind to an HA polypeptide on at least 1, 2, 3, 4, 5, or 6 strains from Group 2. In another example, the antibody molecules described herein can bind to an HA polypeptide on an influenza strain from at least 1, 2 or 3 clades from Group 1, and can also bind to an HA polypeptide on an influenza strain from one or both clades of Group 2. The antibody molecules described herein inhibit viral/endosome membrane fusion, and thus targeting an early step in the infection process.

The binding agents, and in particular, the antibody molecules featured in the disclosure, can be effective to treat or prevent infection by seasonal or pandemic influenza strains. The binding agents, and in particular the antibody molecules described herein, can be characterized by their ability to prevent or treat a Group 1 or a Group 2 strain of influenza A viruses. The binding agents, and in particular the antibody molecules featured in the disclosure, are effective to prevent or treat infection by one or more strains of Group 1, one or more strains of Group 2. In an embodiment, the binding agent is used to treat or prevent an influenza virus infection caused by an influenza virus chose from an H1N1 virus, an H3N2 virus, an H7N9 virus, or a combination thereof.

The binding agents, and in particular, the antibody molecules featured in the disclosure, can be effective to treat or prevent an influenza virus infection, when administered prior to exposure to an influenza virus, e.g., 1 day, 2 days, 3 days, 4 days, 1 week, 2 weeks, 3 weeks, 4 weeks, 1 month, 2 months, 3 months, or more, or later after infection, or upon a first symptom experienced by the patient.

### Strains

The antibody molecules described herein are effective to treat one or more influenza strains of Group 1, and one or more influenza strains of Group 2, and specific isolates within these strains. Certain antibody molecules may be more effective for treatment of certain isolates than other isolates. Exemplary influenza strains and isolates are described in the below Table 1. Affinity can also be in reference to a particular isolate of a given Group 1 or Group 2 strain for influenza A viruses. Exemplary isolates are as provided in the above Table 1.

TABLE 1

Exemplary Influenza Strains and Isolates

| Type | Group | HA type | Isolate |
|---|---|---|---|
| A | 1 | H1N1 | A/PR/8/34 (aka PR-8) |
| | | | A/Solomon Islands/03/06 |
| | | | A/Solomon Islands/20/1999 |
| | | | A/California/07/2009 |
| | | | A/New Caledonia/20/99 |
| | | | A/Bangkok/10/83 |
| | | | A/Yamagata/120/86 |
| | | | A/Osaka/930/88 |
| | | | A/Suita/1/89 |
| | | | A/California/04/2009 |
| A | 1 | H2N2 | A/Okuda/57 |
| | | | A/Adachi/2/57 |
| | | | A/Kumamoto/1/65 |
| | | | A/Kaizuka/2/65 |
| | | | A/Izumi/5/65 |
| | | | A/Chicken/PA/2004 |
| A | 1 | H5N1 | A/Vietnam/1203/04 |
| | | | A/Duck/Singapore/3/97 |
| | | | A/Duck/MN/1525/81 |
| A | 1 | H9N2 | A/Hong Kong/1073/2004 |
| | | | A/Swine/Hong Kong/9/98 |
| | | | A/Guinea fowl/HK/WF10/99 |
| A | 2 | H3N2 | A/black headedgull/Mongolia/1756/2006 |
| | | | X-31 |
| | | | A/Victoria/3/75 |
| | | | A/Wyoming/03/2003 |
| | | | A/Wisconsin/67/2005 |
| | | | A/Brisbane/10/2007 |
| | | | A/California/7/2004 |
| | | | A/New York/55/2004 |
| | | | A/Moscow/10/1999 |
| | | | A/Aichi/2/68 |
| | | | A/Beijing/32/92/X-117 |
| | | | A/Fukuoka/C29/85 |
| | | | A/Sichuan/2/87 |
| | | | A/Ibaraki/1/90 |
| | | | A/Suita/1/90 |
| | | | A/Perth/16/2009 |
| | | | A/Uruguay/716/2007 |
| | | | A/Fujian/411/2003 |
| | | | A/Panama/2007/99 |
| | | | A/Shangdong/09/93 |
| | | | A/Hong Kong/4801/2014 |
| A | 2 | H7N7 | A/Netherlands/219/2003 |

### Mechanisms of Inhibition

While not being limited by a specific mechanism, HA specific antibodies can inhibit infection by numerous methods, such as by blocking viral attachment to sialic acid residues on surface proteins on host cells, by interfering with the structural transition of HA that triggers fusion activity in the endosome, or by simultaneously inhibiting attachment and virus-cell fusion. In some embodiments, antibody molecules featured herein bind an epitope at the HA trimer interface. Structural changes at the trimer interface are important for fusion of the viral membrane and the endocytic membrane, and the antibody molecules described herein interfere with this critical step of infection. Assays to measure fusogenic activity of HA are known in the art. For example, one fusion assay measures syncytia formation, which occurs in cell-cell fusion events. Cells that express and display an influenza viral strain HA can be used in the assay. Membrane-anchored hemagglutinin in these cells is induced to convert to the fusion conformation by a brief (e.g., 3 minute) exposure to low pH (e.g., pH 5). A 2-3-hour incubation period follows to allow the cells to recover and fuse to form syncytia. A nuclear stain can be used to aid in the visualization of these fusion products, and their count is used as a gauge of fusion activity. A candidate anti-HA antibody can be added either before or after the low pH treatment to determine at which stage of the fusion process the antibody interferes.

Another type of fusion assay monitors content mixing. To measure content mixing, host cells (e.g., erythrocytes) are loaded with a dye (e.g., Lucifer yellow) to determine whether the contents of HA-bound host cells could be delivered to HA-expressing cells after exposure to fusion-inducing conditions (e.g., low pH, such as pH less than 6 or pH less than 5). If the dye fails to mix with the contents of the host cells, then the conclusion can be made that fusion is inhibited. See, e.g., Kemble et al., J. Virol. 66:4940-4950, 1992. In another example, a fusion assay is performed by monitoring lipid mixing. The lipid mixing assay can be performed by labeling host cells (e.g., erythrocytes) with a fluorescent dye (e.g., R18 (octadecylrhodamine)) or dye pairs (e.g., CPT-PC/DABS-PC) (for fluorescence resonance energy transfer), exposing the host cells and HA-expressing cells to fusion-inducing conditions, and assaying for fluorescence dequenching (FDQ). Lipid mixing leads to dilution of the label into the viral envelope and a consequent dequenching. A lag in dequenching or the absence of dequenching is indicative of membrane fusion inhibition. See, e.g., Kemble et al., J. Virol. 66:4940-4950, 1992; and Carr et al., Proc. Natl. Acad. Sci. 94:14306-14313, 1997.

### Escape Mutants

In some embodiments, influenza strains will rarely if ever produce escape mutants when contacted with the featured antibody molecules. Escape mutants can be identified by methods known in the art. For example, an antibody featured in the disclosure will not produce an escape mutant when the cells are infected with the virus under prolonged or repeated exposure to anti-HA antibodies featured in the disclosure.

One exemplary method includes infection of cells (e.g. MDCK cells) with a fixed amount of influenza A viral particles in the presence of the antibody at a concentration known to attenuate infection rates by 50%. Viral progeny collected after each passaging is used to infect a fresh cell culture in the presence of the same or greater concentration of the antibody. After multiple cycles of infection, e.g., after 15 cycles, 12 cycles, 11 cycles, 10 cycles, 9 cycles, 8 cycles, 7 cycles, 6 cycles, or 5 cycles, of infection under these conditions, the HA nucleotide sequence extracted from 20 viral plaque picks is evaluated for enrichment for mutations that renders the viral isolate resistant to neutralization by the antibody (an escape mutant). If no mutants with reduced sensitivity to the antibody are detected after the multiple rounds of selection, e.g., after 11 rounds, 10 rounds, or 9 rounds of selection, the antibody is determined to be resistant to escape mutations (see, e.g., Throsby et al. (2008) PLoS One, volume 3, e3942).

In another example, an assay that measures minimum inhibitory concentration (MIC) of the neutralizing antibody can be used to identify escape mutants. The MIC of an antibody molecule is the lowest concentration of an antibody molecule that can be mixed with virus to prevent infection of cell culture with influenza. If escape mutants arise within a viral population, then the MIC of a particular antibody will be observed to increase with increased rounds of propagation under the antibody selective pressure, as the proportion of the viral particles that carry the resistance mutation within the population increased. Influenza escape mutants rarely if ever evolve in response to an anti-HA antibody molecule described herein, and therefore the MIC will stay the same over time.

Another assay suitable for monitoring for the development of escape mutants is a Cytopathic Effect (CPE) assay. A CPE assay monitors the ability of an antibody to neutralize (i.e., prevent infection by) an influenza strain. A CPE assay provides the minimal concentration of antibody required in cell culture to neutralize the virus. If escape mutants arise, than the CPE of a particular antibody will increase over time, as the antibody becomes less effective at neutralizing the virus. Viral strains rarely if ever produce escape mutants in response to an anti-HA antibody molecule described herein, and therefore the CPE will stay essentially the same over time.

Quantitative polymerase chain reaction (qPCR) can also be used to monitor for the development of escape mutants. qPCR is useful to monitor the ability of an antibody to neutralize (i.e., prevent infection by) an influenza strain. If an antibody effectively neutralizes a virus, then qPCR performed on cell culture samples will not detect presence of viral genomic nucleic acid. If escape mutants arise, than over time, qPCR will amplify more and more viral genomic nucleic acid. Escape mutants rarely if ever develop in response to an anti-HA antibody molecule described herein, and therefore qPCR will rarely if ever detect viral genomic nucleic acid, even after the passage of time.

### Binding and Affinity

In some embodiments, the binding agents, particularly antibody molecules, featured herein bind to two or more of the following: at least one HA polypeptide from a Group 1 influenza strain (e.g., an H1, H2, H5, H6, H8, H9 H12, H11, H13, H16 or H17 polypeptide); and at least one HA polypeptide from a Group 2 influenza strain (e.g., an H3, H4, H14, H7, H10, or H15 polypeptide). In an embodiment, a binding agent, e.g., an antibody molecule, has a K_{D} for an HA from a Group 1 influenza strain (e.g., an H1, H2, H5, H6, H8, H9 H12, H11, H13, H16 or H17 polypeptide) of equal to or less than 10⁻⁶, 10⁻⁷, 10⁻⁸, 10⁻⁹, 10⁻¹⁰, 10⁻¹¹, or 10⁻¹². In an embodiment, a binding agent, e.g., an antibody molecule, has a K_{D} for an HA from a Group 2 influenza strain (e.g., an H3, H4, H14, H7, H10, or H15 polypeptide) of equal to or less than 10⁻⁶, 10⁻⁷, 10⁻⁸, 10⁻⁹, 10⁻¹⁰, 10⁻¹¹, or 10⁻¹².In an embodiment, a binding agent, e.g., an antibody molecule, has: a) a first K_{D} (representing an affinity for an HA from a Group 1 influenza strain, e.g., an H1, H2, H5, H6, H8, H9 H12, H11, H13, H16 or H17 polypeptide); and b) a second K_{D} (representing an affinity for an HA from a Group 2 influenza strain, e.g., an H3, H4, H14, H7, H10, or H15 polypeptide), wherein the first and second K_{D} are one or both of: both equal to or less than 10⁻⁸; and within 10 or 100 fold of each other.

In an embodiment, a binding agent, e.g., an antibody molecule, has a) a first K_{D} (representing an affinity for an H1, e.g., the H1 from an H1N1 strain, e.g., A/South Carolina/1/1918, A/Puerto Rico/08/1934, or A/California/04/2009, or an H5N1 strain, e.g., A/Indonesia/5/2005 or A/Vietnam/1203/2004); and b) a second K_{D} (representing an affinity for an H3 polypeptide, e.g., the H3 from an H3N2 strain, e.g., A/Brisbane/59/2007), wherein the first and second K_{D} are one or both of: both equal to or less than 10⁻⁸; and within 10 or 100 fold of each other. In an embodiment, a binding agent, e.g., an antibody molecule, has: a) a first K_{D} (representing an affinity for an H1, e.g., the H1 from an H1N1 strain, e.g., A/South Carolina/1/1918, A/Puerto Rico/08/1934, or A/California/04/2009, or an H5N1 strain, e.g., A/Indonesia/5/2005 or A/Vietnam/1203/2004); and b) a second K_{D} (representing an affinity for an H3 polypeptide, e.g., the H3 from an H3N2 strain, e.g., A/Brisbane/59/2007), wherein the first and second K_{D} are one or both of: both equal to or less than 10⁻⁸; and within 10 or 100 fold of each other.

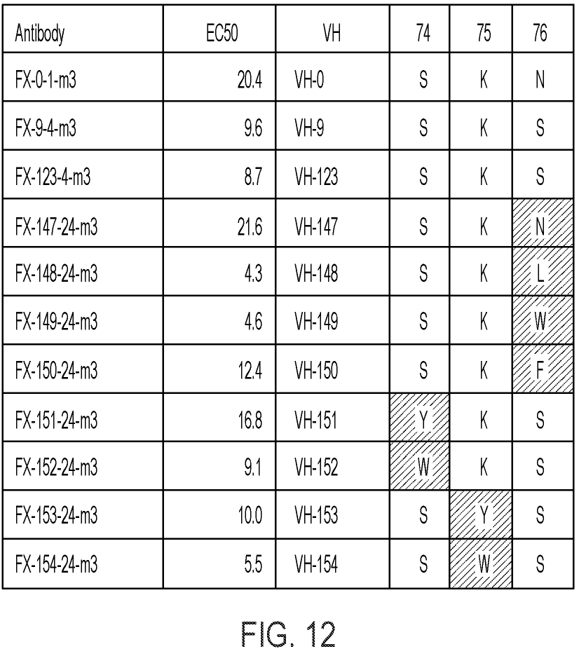

In one embodiment, the antibody molecule binds to at least one HA polypeptide from a Group 1 influenza strain with a higher affinity than a reference anti-HA antibody, and to at least one HA polypeptide from a Group 2 influenza strain with a higher affinity than a reference anti-HA antibody. Exemplary reference HA antibodies include an anti-HA antibody disclosed in PCT Application Publication Nos. WO 2013/170139 (e.g., Ab 044), an anti-HA antibody disclosed in PCT Application Publication No. WO 2013/169377, FI6 (FI6, as used herein, refers to any specifically disclosed FI6 sequence in U.S. Application Publication No. 2010/0080813, US Application Publication No. 2011/0274702, International Publication No. WO2013/011347 or Corti et al., Science 333:850-856, 2011, published online Jul. 28, 2011; FIGS. 12A to 12C of International Publication No. WO2013/170139 or U.S. Application Publication No. 2013/0302349), FI28 (U.S. Application Publication No. 2010/0080813), and C179 (Okuno et al., J. Virol. 67:2552-1558, 1993), F10 (Sui et al., Nat. Struct. Mol. Biol. 16:265, 2009), CR9114 (Dreyfus et al., Science. 2012; 337(6100):1343-1348; published online Aug. 9, 2012), and CR6261 (Ekiert et al., Science 324:246-251, 2009; published online Feb. 26, 2009).

In an embodiment, the binding agent, e.g., an antibody molecule, described herein, comprises a mutation that results one or more of additional contact with the HA polypeptide, improved binding, and/or improved neutralization capability. In an embodiment, the mutation is at position 75 and/or position 76 of the VH of the binding agent, e.g., an antibody molecule described herein. In an embodiment, a mutation in the VH at position 75 of a binding agent, e.g., antibody molecule, described herein, results in one or more of additional contact with the HA polypeptide, improved binding, and/or improved neutralization capability. In an embodiment, a mutation in the VH at position 76 of a binding agent, e.g., antibody molecule, described herein, results in one or more of additional contact with the HA polypeptide, improved binding, and/or improved neutralization capability.

Affinity, or relative affinity or aviditiy, can be measured by methods known in the art, such as by ELISA assay (Enzyme Linked Immunosorbent Assay), Surface Plasmon Resonance (SPR, e.g., by a Biacore™ Assay), or KinExA® assay (Sapidyne, Inc.). Relative binding affinity is expressed herein according to ELISA assay. As used herein, an anti-HA antibody that binds with “high affinity” to a Group 1 HA, and to a Group 2 HA, can bind a Group 1 HA with a Kd less than or equal to 200 pM, e.g., less than or equal to 100 pM, as measured by ELISA, and can bind a Group 2 HA with a Kd less than or equal to 200 pM, e.g., less than or equal to 100 pM, as measured by ELISA.

### Exemplary Anti-HA Antibody Molecules

The amino acid sequences of exemplary heavy and light chain variable regions, as well as heavy and light chain CDRs, are shown below (bold and underline = Kabat-defined CDRs).

Exemplary Heavy Chain Variable Regions

>VH0
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSLLYFEWLSQGYFNP WGQGTTLTVSS (SEQ ID NO: 1)

>VH1
QVQLVESGGGVVQPGRSLRLSCAASGFTFSSYAMH WVRQAPGKGLEWVAV
VSYDGNYKYYADSVQG RFTISRDNSKNTLYLQMNSLRAEDTAVYYCAKDS
RLRSLLYFEWLSQGYFNP WGQGTTVTVSS (SEQ ID NO: 2)

>VH2
QVQLVESGGGWQPGRSLRLSCAASGFTFSSYAMH WVRQAPGKGLEWVAVV
SYDGNYKYYADSVKG RFTISRDNSKNTLYLQMNSLRAEDTAVYYCAKDSR
LRSLLYFEWLSQGYFNP WGQGTTVTVSS (SEQ ID NO: 3)

>VH3
QVQLVESGGGVVQPGRSLRLSCAASGFTFSTYAMH WVRQAPGKGLEWVAV
ISYDANYKYYADSVKG RFTISRDNSKNTLYLQMNSLRAEDTAVYYCAKDS
QLRSLLYFEWLSQGYFDY WGQGTLVTVSS (SEQ ID NO: 4)

>VH4
QVQLVESGGGWQPGRSLRLSCAASGFDFSTYAMH WVRQAPGKGLEWVAVI
SYDANYKYYADSVKG RFTISRDNSKNTLYLQMNSLRAEDTAVYYCARDSR
LRSLLYFEWLSQGYFDY WGQGTLVTVSS (SEQ ID NO: 5)

>VH5
QVQLVESGGGWQPGRSLRLSCAASGFTFSTYAMH WVRQAPGKGLEWVCVI
SYDANYKYYADSVKG RFTCSRDNSKNTLYLQMNSLRAEDTAVYYCARDSR
LRSLLYFEWLSQGYFDY WGQGTLVTVSS (SEQ ID NO: 6)

>VH6
QVQLVESGGGWQPGRSLRLSCAASGFTFDTYAMH WVRQAPGKGLEWVAVI
SYDANYKYYADSVKG RFTISRDNSKNTLYLQMNSLRAEDTAVYYCARDSR
LRSLLYFEWLSQGYFDY WGQGTLVTVSS (SEQ ID NO: 7)

>VH7
EVQLLESGGGLVQPGGSLRLSCAASGFTFSSYAMH WVRQAPGKGLEWVSV
ISYDANYKYYADSVKG RFTISRDNSKNTLYLQMNSLRAEDTAVYYCAKDS
QLRSLLYFEWLSQGYFDY WGQGTTLTVSS (SEQ ID NO: 8)

>VH8
EVQLLESGGGLVQPGGSLRLSCAASGFTFSSYAMH WVRQAPGKGLEWVAV
ISYDANYKYYADSVKG RFTISRDNSKNTLYLQMNSLRAEDTAVYYCAKDS
RLRSLLYFEWLSQGYFDY WGQGTLVTVSS (SEQ ID NO: 9)

>VH9
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
QLRSLLYFEWLSQGYFDY WGQGTTLTVSS (SEQ ID NO: 10)

>VH10
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCARDS
RLRSLLYFEWLSQGYFDY WGQGTTLTVSS (SEQ ID NO: 11)

>VH11
QVQLVQSGAEVKKPGASVKVSCKASGFTFSTYAIN WVRQATGQGLEWMGW
ISYDANYKYYAQKFQG RVTMTRDTSISTAYMELSSLRSEDTAVYYCAKDS
QLRSLLYFEWLSQGYFDY WGQGTTLTVSS (SEQ ID NO: 12)

>VH12
QVQLVQSGAEVKKPGASVKVSCKASGFTFSTYAIN WVRQATGQGLEWMGV
ISYDANYKYYAQKFQG RVTMTRDTSISTAYMELSSLRSEDTAVYYCARDS
QLRSLLYFEWLSQGYFDY WGQGTTLTVSS (SEQ ID NO: 13)

>VH13
QVQLLETGGGLVKPGQSLKLSCAASGFTFTSYAMH WVRQPPGKGLEWVAV
VCYDGNYKYYADSVQG RFTISRDNSKNTLYLQMNSLRAEDTAVYYCAKDS
RLRSLCYFEWLSQGYFNP WGQGTTLTVSS (SEQ ID NO: 14)

>VH14
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSLLYCEWCSQGYFNP WGQGTTLTVSS (SEQ ID NO: 15)

>VH15
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSLLYFEWLSCGYFNP WGQGTTLTVSS (SEQ ID NO: 16)

>VH16
QVQLLETGGGLVKPGQSLKLSCAASGFTFTSYAMH WVRQPPGKGLEWVAV
VSYDGNCKYYADSVQG RFTISRDNSKNTLYLQMNSLRAEDTAVYYCAKDS
RLRSLCYFEWLSQGYFNP WGQGTTLTVSS (SEQ ID NO: 17)

>VH17
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSLLYFECLSQGYFNP WGQGTTLTVSS (SEQ ID NO: 18)

>VH18
QVQLLETGGGLVKPGQSLKLSCAASGFTFTSYSMH WVRQPPGKGLEWVAV
VSYDGNYKYYADSVQG RFTISRDNSKNTLYLQMNSLRAEDTAVYYCAKDS
RLRSLHYFEWLSQGYFNP WGQGTTLTVSS (SEQ ID NO: 19)

>VH19
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSLLYFEWMNQGYFNP WGQGTTLTVSS (SEQ ID NO: 20)

>VH20
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSLLYQEWLSQGYFNP WGQGTTLTVSS (SEQ ID NO: 21)

>VH21
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSLLYFDWLSQGYFNP WGQGTTLTVSS (SEQ ID NO: 22)

>VH22
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSLQYFEWLSQGYFNP WGQGTTLTVSS (SEQ ID NO: 23)

>VH23
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSLNYFEWLSQGYFNP WGQGTTLTVSS (SEQ ID NO: 24)

>VH24
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RNRSLLMFEWLSQGYFNP WGQGTTLTVSS (SEQ ID NO: 25)

>VH25
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RQRSLLMFEWLSQGYFNP WGQGTTLTVSS (SEQ ID NO: 26)

>VH26
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSLLYFEWNSQGYFNP WGQGTTLTVSS (SEQ ID NO: 27)

>VH27
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RNRSLLMFEWNNQGYFNP WGQGTTLTVSS (SEQ ID NO: 28)

>VH28
QVQLVESGGGVVQPGRSLRLSCAASGFTFSSYAMH WVRQAPGKGLEWVAI
VSYDGNYKYYADSVKG RFTISRDNSKNTLYLQMNSLRAEDTAVYYCAKDS
RLRSLLYFEWLSQGYFNP WGQGTTVTVSS (SEQ ID NO: 29)

>VH29
QVQLVESGGGVVQPGRSLRLSCAASGFTFSSYAMH WVRQAPGKGLEWVAV
VSYDGNYKYYADSVKG RFTISRDNSKNTLYLQMNSLRAEDTAVYYCAKDS
RNRVLLYFEWLSQGYFNP WGQGTTVTVSS (SEQ ID NO: 30)

>VH30
QVQLVESGGGVVQPGRSLRLSCAASGFTFSSYAMH WVRQAPGKGLEWVAV
VSYDGNYKYYADSVKG RFTISRDNSKNTLYLQMNSLRAEDTAVYYCAKDS
RLRSLLYFEWLSLGYFNP WGQGTTVTVSS (SEQ ID NO: 31)

>VH31
QVQLVESGGGVVQPGRSLRLSCAASGFTFSSYAMH WVRQAPGKGLEWVAV
VSYDGNYKYYADSVKG RFTISRDNSKNTLYLQMNSLRAEDTAVYYCAKDS
RLRSLQYFEWLSQGYFNP WGQGTTVTVSS (SEQ ID NO: 32)

>VH32
QVQLVESGGGVVQPGRSLRLSCAASGFTFSSYAMH WVRQAPGKGLEWVAV
VSYDGNYKYYADSVKG RFTISRDNSKNTLYLQMNSLRAEDTAVYYCAKDS
RLRLLLYFEWLSQGYFNP WGQGTTVTVSS (SEQ ID NO: 33)

>VH33
QVQLVESGGGVVQPGRSLRLSCAASGFTFSSYAMH WVRQAPGKGLEWVAV
VSYDGNYKYYADSVKG RFTISRDNSKNTLYLQMNSLRAEDTAVYYCAKDS
RLRSLLYFEWLSQGRFNP WGQGTTVTVSS (SEQ ID NO: 34)

>VH34
QVQLVESGGGVVQPGRSLRLSCAASGFTFSSYAMH WVRQAPGKGLEWVAV
VSYDGNYKYYADSVRG RFTISRDNSKNTLYLQMNSLRAEDTAVYYCAKDS
RLRSLLYFEWLSQGYFNP WGQGTTVTVSS (SEQ ID NO: 35)

>VH35
QVQLVESGGGVVQPGRSLRLSCAASGFTFSSYAMH WVRQAPGKGLEWVAV
VSYDGNYKYYADSVKG RFTISRDNSKNTLYLQMNSLRAEDTAVYYCAKDS
ELRSLLYFEWLSQGYFNP WGQGTTVTVSS (SEQ ID NO: 36)

>VH36
QVQLEESGGGVVQPGRSLRLSCAASGFTFSSYAMH WVRQAPGKGLEWVAV
VSYDSNYKYYADSVKG RFTISRDNSKNTLYLQMNSLRAEDTAVYYCAKDS
RLRSLLYFEWLSQGYFNP WGQGTTVTVSS (SEQ ID NO: 37)

>VH37
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSNLYYEWLSQGYFNP WGQGTTLTVSS (SEQ ID NO: 38)

>VH38
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSQLYYEWLSQGYFNP WGQGTTLTVSS (SEQ ID NO: 39)

>VH39
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSLLYFEHLSQGYFNP WGQGTTLTVSS (SEQ ID NO: 41)

>VH40
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSLLYFERLSQGYFNP WGQGTTLTVSS (SEQ ID NO: 42)

>VH41
QVQLLETGGGLVKPGQSLKLSCAASGFTFTSYAMH WVRQPPGKGLEWVAV
VSYDGNHKYYADSVQG RFTISRDNSKNTLYLQMNSLRAEDTAVYYCAKDS
RLRSLLYFDWLSQGYFNP WGQGTTLTVSS (SEQ ID NO: 43)

>VH42
QVQLLETGGGLVKPGQSLKLSCAASGFTFTSYAMH WVRQPPGKGLEWVAV
VSYDGNNKYYADSVQG RFTISRDNSKNTLYLQMNSLRAEDTAVYYCAKDS
RLRSLLYFDWLSQGYFNP WGQGTTLTVSS (SEQ ID NO: 45)

>VH43
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKES
RLRSLLYFEWLSQGYFNP WGQGTTLTVSS (SEQ ID NO: 46)

>VH44
QVQLLETGGGLVKPGQSLKLSCAASGFTFTSYGMH WVRQPPGKGLEWVAV
VSYDGNYKYYADSVQG RFTISRDNSKNTLYLQMNSLRAEDTAVYYCAKDS
RLRLLLYFEWLSQGYFNP WGQGTTLTVSS (SEQ ID NO: 47)

>VH45
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSLRYFEWLSGGYFNP WGQGTTLTVSS (SEQ ID NO: 48)

>VH46
EVQLVESGGGAVQPGESLKLSCAASGFTFSNYGMH WVRQAPGKGLEWVAV
ISYDGSNKYYADSVKG RFTISRDNSKDTLYLQMNSLRAEDTALFYCAKER
PLRLLRYFDWLSGGANDY WGQGTLVTVSS (SEQ ID NO: 49)

>VH47
QVQLLETGGGLVKPGQSLKLSCAASGFTFTNYGMH WVRQPPGKGLEWVAV
VSYDGSYKYYADSVQG RFTISRDNSKNTLYLQMNSLRAEDTAVYYCAKER
PLRLLRYFDWLSGGANDY WGQGTTLTVSS (SEQ ID NO: 50)

>VH48
QVQLLETGGGLVKPGQSLKLSCAASGFTFTNYGMH WVRQPPGKGLEWVAV
VSYDGNYKYYADSVQG RFTISRDNSKNTLYLQMNSLRAEDTAVYYCAKER
PLRLLRYFDWLSGGANDY WGQGTTLTVSS (SEQ ID NO: 51)

>VH49
QVQLLETGGGLVKPGQSLKLSCAASGFTFTNYGMH WVRQPPGKGLEWVAV
VSYDGNNKYYADSVQG RFTISRDNSKNTLYLQMNSLRAEDTAVYYCAKER
PLRLLRYFDWLSGGANDY WGQGTTLTVSS (SEQ ID NO: 52)

>VH50
EVQLVESGGGAVQPGESLKLSCAASGFTFSNYGMH WVRQAPGKGLEWVAV
ISYDGSYKYYADSVKG RFTISRDNSKDTLYLQMNSLRAEDTALFYCAKER
PLRLLRYFDWLSGGANDY WGQGTLVTVSS (SEQ ID NO: 53)

>VH51
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSLLMFEWLSQGYFNP WGQGTTLTVSS (SEQ ID NO: 54)

>VH52
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RNRSLLYFEWLSQGYFNP WGQGTTLTVSS (SEQ ID NO: 55)

>VH53
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RQRSLLYFEWLSQGYFNP WGQGTTLTVSS (SEQ ID NO: 56)

>VH54
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RTRSLLYFEWLSQGYFNP WGQGTTLTVSS (SEQ ID NO: 57)

>VH55
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RVRSLLYFEWLSQGYFNP WGQGTTLTVSS (SEQ ID NO: 58)

>VH56
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RIRSLLYFEWLSQGYFNP WGQGTTLTVSS (SEQ ID NO: 59)

>VH57
QVQLLETGGGLVKPGQSLKLSCAASGFTFTSYSMH WVRQPPGKGLEWVAV
VSYDGNYKYYADSVQG RFTISRDNSKNTLYLQMNSLRAEDTAVYYCAKDS
RLRSLLYFEWLSQGY FNPWGQGTTLTVSS (SEQ ID NO: 60)

>VH58
QVQLVESGGGVVQPGRSLRLSCAASGFTFSTYAMH WVRQAPGKGLEWVAV
ISYDANYKYYADSVKG RFTISRDNSKNTLYLQMNSLRAEDTAVYYCAKDS
QNRSLLYFEWLSQGYFDY WGQGTLVTVSS (SEQ ID NO: 61)

>VH59
QVQLVESGGGVVQPGRSLRLSCAASGFTFSTYAMH WVRQAPGKGLEWVAV
ISYDANYKYYADSVKG RFTISRDNSKNTLYLQMNSLRAEDTAVYYCAKDS
QLRSLLMFEWLSQGYFDY WGQGTLVTVSS (SEQ ID NO: 62)

>VH60
QVQLVESGGGVVQPGRSLRLSCAASGFTFSTYAMH WVRQAPGKGLEWVAV
ISYDANYKYYADSVKG RFTISRDNSKNTLYLQMNSLRAEDTAVYYCAKDS
QNRSLLMFEWLSQGYFDY WGQGTLVTVSS (SEQ ID NO: 63)

>VH61
QVQLLETGGGLVKPGQSLKLSCAASGFTFTSYSMH WVRQPPGKGLEWVAV
VSYDGNYKYYADSVQG RFTISRDNSKNTLYLQMNSLRAEDTAVYYCAKDS
RNRSLLYFEWLSQGYFNP WGQGTTLTVSS (SEQ ID NO: 64)

>VH62
QVQLLETGGGLVKPGQSLKLSCAASGFTFTSYSMH WVRQPPGKGLEWVAV
VSYDGNYKYYADSVQG RFTISRDNSKNTLYLQMNSLRAEDTAVYYCAKDS
RLRSLLMFEWLSQGYFNP WGQGTTLTVSS (SEQ ID NO: 65)

>VH63
QVQLVESGGGVVQPGRSLRLSCAASGFTFSSYAMH WVRQAPGKGLEWVAV
VSYDGNYKYYADSVKG RFTISRDNSKNTLYLQMNSLRAEDTAVYYCAKDS
QLRSLLYFEWLSQGYFNP WGQGTTVTVSS (SEQ ID NO: 66)

>VH64
QVQLVESGGGVVQPGRSLRLSCAASGFTFSSYAMH WVRQAPGKGLEWVAV
VSYDGNYKYYADSVKG RFTISRDNSKNTLYLQMNSLRAEDTAVYYCAKDS
RNRSLLYFEWLSQGYFNP WGQGTTVTVSS (SEQ ID NO: 67)

>VH65
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RARSLLYFEWLSQGYFNP WGQGTTLTVSS (SEQ ID NO: 68)

>VH66
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RSRSLLYFEWLSQGYFNP WGQGTTLTVSS (SEQ ID NO: 69)

>VH67
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RHRSLLYFEWLSQGYFNP WGQGTTLTVSS (SEQ ID NO: 70)

>VH68
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RYRSLLYFEWLSQGYFNP WGQGTTLTVSS (SEQ ID NO: 71)

>VH69
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RMRSLLYFEWLSQGYFNP WGQGTTLTVSS (SEQ ID NO: 72)

>VH70
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RRRSLLYFEWLSQGYFNP WGQGTTLTVSS (SEQ ID NO: 73)

>VH71
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RDRSLLYFEWLSQGYFNP WGQGTTLTVSS (SEQ ID NO: 74)

>VH72
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RKRSLLYFEWLSQGYFNP WGQGTTLTVSS (SEQ ID NO: 75)

>VH73
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RPRSLLYFEWLSQGYFNP WGQGTTLTVSS (SEQ ID NO: 76)

>VH74
QVQLLETGGGLVKPGQSLKLSCAASGFTFTSFAMH WVRQPPGKGLEWVAV
VSYDGNYKYYADSVQG RFTISRDNSKNTLYLQMNSLRAEDTAVYYCAKDS
RNRSLLYFEWLSQGYFNP WGQGTTLTVSS (SEQ ID NO: 77)

>VH75
QVQLLETGGGLVKPGQSLKLSCAASGFTFTSFAMH WVRQPPGKGLEWVAV
VSYDGNYKYYADSVQG RFTISRDNSKNTLYLQMNSLRAEDTAVYYCAKDS
RQRSLLYFEWLSQGYFNP WGQGTTLTVSS (SEQ ID NO: 78)

>VH76
QVQLLETGGGLVKPGQSLKLSCAASGFTFTSFAMH WVRQPPGKGLEWVAV
VSYDGNYKYYADSVQG RFTISRDNSKNTLYLQMNSLRAEDTAVYYCAKDS
RRRSLLYFEWLSQGYFNP WGQGTTLTVSS (SEQ ID NO: 79)

>VH77
QVQLLETGGGLVKPGQSLKLSCAASGFTFTSHAMH WVRQPPGKGLEWVAV
VSYDGNYKYYADSVQG RFTISRDNSKNTLYLQMNSLRAEDTAVYYCAKDS
RNRSLLYFEWLSQGYFNP WGQGTTLTVSS (SEQ ID NO: 80)

>VH78
QVQLLETGGGLVKPGQSLKLSCAASGFTFTSHAMH WVRQPPGKGLEWVAV
VSYDGNYKYYADSVQG RFTISRDNSKNTLYLQMNSLRAEDTAVYYCAKDS
RQRSLLYFEWLSQGYFNP WGQGTTLTVSS (SEQ ID NO: 81)

>VH79
QVQLLETGGGLVKPGQSLKLSCAASGFTFTSHAMH WVRQPPGKGLEWVAV
VSYDGNYKYYADSVQG RFTISRDNSKNTLYLQMNSLRAEDTAVYYCAKDS
RRRSLLYFEWLSQGYFNP WGQGTTLTVSS (SEQ ID NO: 82)

>VH80
QVQLLETGGGLVKPGQSLKLSCAASGFTFTNYAMH WVRQPPGKGLEWVAV
VSYDGNYKYYADSVQG RFTISRDNSKNTLYLQMNSLRAEDTAVYYCAKDS
RLRSLLYFEWLSQGYFNP WGQGTTLTVSS (SEQ ID NO: 83)

>VH81
QVQLLETGGGLVKPGQSLKLSCAASGFTFTLYAMH WVRQPPGKGLEWVAV
VSYDGNYKYYADSVQG RFTISRDNSKNTLYLQMNSLRAEDTAVYYCAKDS
RLRSLLYFEWLSQGYFNP WGQGTTLTVSS (SEQ ID NO: 84)

>VH82
QVQLLETGGGLVKPGQSLKLSCAASGFTFTDYAMH WVRQPPGKGLEWVAV
VSYDGNYKYYADSVQG RFTISRDNSKNTLYLQMNSLRAEDTAVYYCAKDS
RLRSLLYFEWLSQGYFNP WGQGTTLTVSS (SEQ ID NO: 85)

>VH83
QVQLLETGGGLVKPGQSLKLSCAASGFTFTQYAMH WVRQPPGKGLEWVAV
VSYDGNYKYYADSVQG RFTISRDNSKNTLYLQMNSLRAEDTAVYYCAKDS
RLRSLLYFEWLSQGYFNP WGQGTTLTVSS (SEQ ID NO: 86)

>VH84
QVQLLETGGGLVKPGQSLKLSCAASGFTFTHYAMH WVRQPPGKGLEWVAV
VSYDGNYKYYADSVQG RFTISRDNSKNTLYLQMNSLRAEDTAVYYCAKDS
RLRSLLYFEWLSQGYFNP WGQGTTLTVSS (SEQ ID NO: 87)

>VH85
QVQLLETGGGLVKPGQSLKLSCAASGFTFTYYAMH WVRQPPGKGLEWVAV
VSYDGNYKYYADSVQG RFTISRDNSKNTLYLQMNSLRAEDTAVYYCAKDS
RLRSLLYFEWLSQGYFNP WGQGTTLTVSS (SEQ ID NO: 88)

>VH86
QVQLLETGGGLVKPGQSLKLSCAASGFTFTNYAMH WVRQPPGKGLEWVAV
VSYDGNYKYYADSVQG RFTISRDNSKNTLYLQMNSLRAEDTAVYYCAKDS
RNRSLLYFEWLSQGYFNP WGQGTTLTVSS (SEQ ID NO: 89)

>VH87
QVQLLETGGGLVKPGQSLKLSCAASGFTFTLYAMH WVRQPPGKGLEWVAV
VSYDGNYKYYADSVQG RFTISRDNSKNTLYLQMNSLRAEDTAVYYCAKDS
RNRSLLYFEWLSQGYFNP WGQGTTLTVSS (SEQ ID NO: 90)

>VH88
QVQLLETGGGLVKPGQSLKLSCAASGFTFTDYAMH WVRQPPGKGLEWVAV
VSYDGNYKYYADSVQG RFTISRDNSKNTLYLQMNSLRAEDTAVYYCAKDS
RNRSLLYFEWLSQGYFNP WGQGTTLTVSS (SEQ ID NO: 91)

>VH89
QVQLLETGGGLVKPGQSLKLSCAASGFTFTQYAMH WVRQPPGKGLEWVAV
VSYDGNYKYYADSVQG RFTISRDNSKNTLYLQMNSLRAEDTAVYYCAKDS
RNRSLLYFEWLSQGYFNP WGQGTTLTVSS (SEQ ID NO: 92)

>VH90
QVQLLETGGGLVKPGQSLKLSCAASGFTFTHYAMH WVRQPPGKGLEWVAV
VSYDGNYKYYADSVQG RFTISRDNSKNTLYLQMNSLRAEDTAVYYCAKDS
RNRSLLYFEWLSQGYFNP WGQGTTLTVSS (SEQ ID NO: 93)

>VH91
QVQLLETGGGLVKPGQSLKLSCAASGFTFTYYAMH WVRQPPGKGLEWVAV
VSYDGNYKYYADSVQG RFTISRDNSKNTLYLQMNSLRAEDTAVYYCAKDS
RNRSLLYFEWLSQGYFNP WGQGTTLTVSS (SEQ ID NO: 94)

>VH92
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLHSLLYFEWLSQGYFNP WGQGTTLTVSS (SEQ ID NO: 95)

>VH93
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLYSLLYFEWLSQGYFNP WGQGTTLTVSS (SEQ ID NO: 96)

>VH94
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RNHSLLYFEWLSQGYFNP WGQGTTLTVSS (SEQ ID NO: 97)

>VH95
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RNYSLLYFEWLSQGYFNP WGQGTTLTVSS (SEQ ID NO: 98)

>VH96
QVQLLETGGGLVKPGQSLKLSCAASGFQFTSFAMH WVRQPPGKGLEWVAV
VSYDGNYKYYADSVQG RFTISRDNSKNTLYLQMNSLRAEDTAVYYCAKDS
RNRSLLYFEWLSQGYFNP WGQGTTLTVSS (SEQ ID NO: 99)

>VH97
QVQLLETGGGLVKPGQSLKLSCAASGFNFTSFAMH WVRQPPGKGLEWVAV
VSYDGNYKYYADSVQG RFTISRDNSKNTLYLQMNSLRAEDTAVYYCAKDS
RNRSLLYFEWLSQGYFNP WGQGTTLTVSS (SEQ ID NO: 100)

>VH98
QVQLLETGGGLVKPGQSLKLSCAASGFRFTSFAMH WVRQPPGKGLEWVAV
VSYDGNYKYYADSVQG RFTISRDNSKNTLYLQMNSLRAEDTAVYYCAKDS
RNRSLLYFEWLSQGYFNP WGQGTTLTVSS (SEQ ID NO: 101)

>VH99
QVQLLETGGGLVKPGQSLKLSCAASGFTFTSYAMH WVRQPPGKGLEWVAV
VSYDANYKYYADSVQG RFTISRDNSKNTLYLQMNSLRAEDTAVYYCAKDS
RLRSQLYFEWLSQGYFNP WGQGTTLTVSS (SEQ ID NO: 102)

>VH100
QVQLLETGGGLVKPGQSLKLSCAASGFTFTSYAMH WVRQPPGKGLEWVAV
VSYDANYKYYADSVQG RFTISRDNSKNTLYLQMNSLRAEDTAVYYCAKDS
RLRSHLYFEWLSQGYFNP WGQGTTLTVSS (SEQ ID NO: 103)

>VH101
QVQLLETGGGLVKPGQSLKLSCAASGFTFTSYAMH WVRQPPGKGLEWVAV
VSYDANYKYYADSVQG RFTISRDNSKNTLYLQMNSLRAEDTAVYYCAKDS
RLRSKLYFEWLSQGYFNP WGQGTTLTVSS (SEQ ID NO: 104)

>VH102
QVQLLETGGGLVKPGQSLKLSCAASGFTFTSYAMH WVRQPPGKGLEWVAV
VSYDANYKYYADSVQG RFTISRDNSKNTLYLQMNSLRAEDTAVYYCAKDS
RLRSRLYFEWLSQGYFNP WGQGTTLTVSS (SEQ ID NO: 105)

>VH103
QVQLLETGGGLVKPGQSLKLSCAASGFTFTSYAMH WVRQPPGKGLEWVAV
VSYDANYKYYADSVQG RFTISRDNSKNTLYLQMNSLRAEDTAVYYCAKDS
RLRSFLYFEWLSQGYFNP WGQGTTLTVSS (SEQ ID NO: 106)

>VH104
QVQLLETGGGLVKPGQSLKLSCAASGFTFTSYAMH WVRQPPGKGLEWVAV
VSYDANYKYYADSVQG RFTISRDNSKNTLYLQMNSLRAEDTAVYYCAKDS
RLRSLLKFEWLSQGYFNP WGQGTTLTVSS (SEQ ID NO: 107)

>VH105
QVQLLETGGGLVKPGQSLKLSCAASGFTFTSYAMH WVRQPPGKGLEWVAV
VSYDANYKYYADSVQG RFTISRDNSKNTLYLQMNSLRAEDTAVYYCAKDS
RLRSLLRFEWLSQGYFNP WGQGTTLTVSS (SEQ ID NO: 108)

>VH106
EVQLLESGGGLVQPGGSLRLSCAASGFTFSSYAMH WVRQAPGKGLEWVAV
ISYDANYKYYADSVKG RFTISRDNSKNTLYLQMNSLRAEDTAVYYCAKDS
QLRSLLYFEWLSQGYFDY WGQGTLVTVSS (SEQ ID NO: 109)

>VH107
EVQLLESGGGLVQPGGSLRLSCAASGFTFSSYAMH WVRQAPGKGLEWVAV
ISYDANYKYYADSVKG RFTISRDNSKNTLYLQMNSLRAEDTAVYYCAKDS
RLRSLLYFEWLSQGYFNP WGQGTLVTVSS (SEQ ID NO: 110)

>VH108
EVQLLESGGGLVQPGGSLRLSCAASGFTFSSYAMH WVRQAPGKGLEWVAV
ISYDANYKYYADSVKG RFTISRDNSKNTLYLQMNSLRAEDTAVYYCAKDS
RLRSLLYFEWLSQGYFGV WGQGTLVTVSS (SEQ ID NO: 111)

>VH109
EVQLLESGGGLVQPGGSLRLSCAASGFTFSSYAMH WVRQAPGKGLEWVAV
ISYDANYKYYADSVKG RFTISRDNSKNTLYLQMNSLRAEDTAVYYCAKDS
RLRSLLYFEWLSQGYFDY WGQGTTLTVSS (SEQ ID NO: 112)

>VH110
EVQLLESGGGLVQPGGSLRLSCAASGFTFSSYAMH WVRQAPGKGLEWVAV
VSYDGNYKYYADSVKG RFTISRDNSKNTLYLQMNSLRAEDTAVYYCAKDS
RLRSLLYFEWLSQGYFDY WGQGTLVTVSS (SEQ ID NO: 113)

>VH111
EVQLLESGGGLVQPGGSLRLSCAASGFTFTSYAMH WVRQAPGKGLEWVAV
ISYDANYKYYADSVKG RFTISRDNSKNTLYLQMNSLRAEDTAVYYCAKDS
RLRSLLYFEWLSQGYFDY WGQGTLVTVSS (SEQ ID NO: 114)

>VH112
EVQLLESGGGLVQPGGSLRLSCAASGFTFSDYAMH WVRQAPGKGLEWVAV
ISYDANYKYYADSVKG RFTISRDNSKNTLYLQMNSLRAEDTAVYYCAKDS
RLRSLLYFEWLSQGYFDY WGQGTLVTVSS (SEQ ID NO: 115)

>VH113
EVQLLESGGGLVQPGGSLRLSCAASGFTFSSRAMH WVRQAPGKGLEWVAV
ISYDANYKYYADSVKG RFTISRDNSKNTLYLQMNSLRAEDTAVYYCAKDS
RLRSLLYFEWLSQGYFDY WGQGTLVTVSS (SEQ ID NO: 116)

>VH114
EVQLLESGGGLVQPGGSLRLSCAASGFTFSSHAMH WVRQAPGKGLEWVAV
ISYDANYKYYADSVKG RFTISRDNSKNTLYLQMNSLRAEDTAVYYCAKDS
RLRSLLYFEWLSQGYFDY WGQGTLVTVSS (SEQ ID NO: 117)

>VH115
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
QLRSLLYFEWLSQGYFDY WGQGTTVTVSS (SEQ ID NO: 118)

>VH116
QVQLVQSGAEVKKPGSSVKVSCKASGFTFSTYAMH WVRQAPGQGLEWMGV
ISYDANYKYYAQKFQG RVTITRDNSKSTLYMELSSLRSEDTAVYYCAKDS
QLRSLLYFEWLSQGYFDY WGQGTTLTVSS (SEQ ID NO: 119)

>VH117
QVQLVQSGAEVKKPGSSVKVSCKASGFTFSTYAMH WVRQAPGQGLEWMGV
ISYDANYKYYAQKFQG RVTITRDNSKNTLYMELSSLRSEDTAVYYCAKDS
QLRSLLYFEWLSQGYFDY WGQGTTLTVSS (SEQ ID NO: 120)

>VH118
QVQLVQSGAEVKKPGSSVKVSCAASGFTFSTYAIH WVRQAPGQGLEWMGV
ISYDANYKYYAQKFQG RVTITRDNSKSTAYMELSSLRSEDTAVYYCAKDS
QLRSLLYFEWLSQGYFDY WGQGTTLTVSS (SEQ ID NO: 121)

>VH119
QVQLVQSGAEVKKPGSSVKVSCKASGFTFSTYAIH WVRQAPGQGLEWMAV
ISYDANYKYYAQKFQG RVTITRDNSKSTAYMELSSLRSEDTAVYYCAKDS
QLRSLLYFEWLSQGYFDY WGQGTTLTVSS (SEQ ID NO: 122)

>VH120
QVQLVQSGAEVKKPGSSLKSCKASGFTFSTYAIH WVRQAPGQGLEWVAV
ISYDANYKYYAQKVQG RFTITRDNSKSTAYLEMSSLRSEDTAVYYCAKDS
QLRSLLYFEWLSQGYFDY WGQGTTVTVSS (SEQ ID NO: 123)

>VH121
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
QLRSLLYFEWLSQGYFNP WGQGTTLTVSS (SEQ ID NO: 124)

>VH122
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGYFDY WGQGTTLTVSS (SEQ ID NO: 125)

>VH123
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGYFNP WGQGTTLTVSS (SEQ ID NO: 126)

>VH124
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGYFGV WGQGTTLTVSS (SEQ ID NO: 127)

>VH125
QVQLVQSGAEVKKPGSSVKVSCKASGFTFTSYAIH WVRQAPGQGLEWMGV
ISYDANYKYYAQKFQG RVTITRDNSKSTAYMELSSLRSEDTAVYYCAKDS
QLRSLLYFEWLSQGY FDYWGQGTTLTVSS (SEQ ID NO: 128)

>VH126
QVQLVQSGAEVKKPGSSVKVSCKASGFTFSDYAIH WVRQAPGQGLEWMGV
ISYDANYKYYAQKFQG RVTITRDNSKSTAYMELSSLRSEDTAVYYCAKDS
QLRSLLYFEWLSQGYFDY WGQGTTLTVSS (SEQ ID NO: 129)

>VH127
QVQLVQSGAEVKKPGSSVKVSCKASGFTFSTRAIH WVRQAPGQGLEWMGV
ISYDANYKYYAQKFQG RVTITRDNSKSTAYMELSSLRSEDTAVYYCAKDS
QLRSLLYFEWLSQGY FDYWGQGTTLTVSS (SEQ ID NO: 130)

>VH128
QVQLVQSGAEVKKPGSSVKVSCKASGFTFSTHAIH WVRQAPGQGLEWMGV
ISYDANYKYYAQKFQG RVTITRDNSKSTAYMELSSLRSEDTAVYYCAKDS
QLRSLLYFEWLSQGYFDY WGQGTTLTVSS (SEQ ID NO: 131)

>VH129
QVQLLETGGGLVKPGQSLKLSCAASGFTFTSYAMH WVRQPPGKGLEWVAV
VSYDANYKYYADSVQG RFTISRDNSKNTLYLQMNSLRAEDTAVYYCAKDS
RLRSLLYFEWLSQGYFNP WGQGTTLTVSS (SEQ ID NO: 132)

>VH130
QVQLLETGGGLVKPGQSLKLSCAASGFTFTSYAMH WVRQPPGKGLEWVAV
VSYDADYKYYADSVQG RFTISRDNSKNTLYLQMNSLRAEDTAVYYCAKDS
RLRSLLYFEWLSQGYFNP WGQGTTLTVSS (SEQ ID NO: 133)

>VH131
QVQLLETGGGLVKPGQSLKLSCAASGFTFTSYAMH WVRQPPGKGLEWVAV
VSYDAEYKYYADSVQG RFTISRDNSKNTLYLQMNSLRAEDTAVYYCAKDS
RLRSLLYFEWLSQGYFNP WGQGTTLTVSS (SEQ ID NO: 134)

>VH132
QVQLLETGGGLVKPGQSLKLSCAASGFTFTSYAMH WVRQPPGKGLEWVAV
VSYDAHYKYYADSVQG RFTISRDNSKNTLYLQMNSLRAEDTAVYYCAKDS
RLRSLLYFEWLSQGYFNP WGQGTTLTVSS (SEQ ID NO: 135)

>VH133
QVQLLETGGGLVKPGQSLKLSCAASGFTFTSYAMH WVRQPPGKGLEWVAV
VSYDGHYKYYADSVQG RFTISRDNSKNTLYLQMNSLRAEDTAVYYCAKDS
RLRSLLYFEWLSQGYFNP WGQGTTLTVSS (SEQ ID NO: 136)

>VH134
QVQLLETGGGLVKPGQSLKLSCAASGFTFTSYAMH WVRQPPGKGLEWVAV
VSYDGFYKYYADSVQG RFTISRDNSKNTLYLQMNSLRAEDTAVYYCAKDS
RLRSLLYFEWLSQGYFNP WGQGTTLTVSS (SEQ ID NO: 137)

>VH135
QVQLLETGGGLVKPGQSLKLSCAASGFTFTSYAMH WVRQPPGKGLEWVAV
VSYDGYYKYYADSVQG RFTISRDNSKNTLYLQMNSLRAEDTAVYYCAKDS
RLRSLLYFEWLSQGYFNP WGQGTTLTVSS (SEQ ID NO: 138)

>VH136
QVQLLETGGGLVKPGQSLKLSCAASGFTFTSYAMH WVRQPPGKGLEWVAV
VSYDGSYKYYADSVQG RFTISRDNSKNTLYLQMNSLRAEDTAVYYCAKDS
RLRSLLYFEWLSQGYFNP WGQGTTLTVSS (SEQ ID NO: 139)

>VH137
QVQLLETGGGLVKPGQSLKLSCAASGFTFTSYAMH WVRQPPGKGLEWVAV
VSFDGNYKYYADSVQG RFTISRDNSKNTLYLQMNSLRAEDTAVYYCAKDS
RLRSLLYFEWLSQGYFNP WGQGTTLTVSS (SEQ ID NO: 140)

>VH138
QVQLLETGGGLVKPGQSLKLSCAASGFTFTSYAMH WVRQPPGKGLEWVAV
VSWDGNYKYYADSVQG RFTISRDNSKNTLYLQMNSLRAEDTAVYYCAKDS
RLRSLLYFEWLSQGYFNP WGQGTTLTVSS (SEQ ID NO: 141)

>VH139
QVQLLETGGGLVKPGQSLKLSCAASGFTFTSYAMH WVRQPPGKGLEWVAV
VSYNGNYKYYADSVQG RFTISRDNSKNTLYLQMNSLRAEDTAVYYCAKDS
RLRSLLYFEWLSQGYFNP WGQGTTLTVSS (SEQ ID NO: 142)

>VH140
QVQLLETGGGLVKPGQSLKLSCAASGFTFTSYAMH WVRQPPGKGLEWVAV
VSYSGNYKYYADSVQG RFTISRDNSKNTLYLQMNSLRAEDTAVYYCAKDS
RLRSLLYFEWLSQGYFNP WGQGTTLTVSS (SEQ ID NO: 143)

>VH141
QVQLLETGGGLVKPGQSLKLSCAASGFTFTSYAMH WVRQPPGKGLEWVAV
ISYDGNYKYYADSVQG RFTISRDNSKNTLYLQMNSLRAEDTAVYYCAKDS
RLRSLLYFEWLSQGYFNP WGQGTTLTVSS (SEQ ID NO: 144)

>VH142
QVQLLETGGGLVKPGQSLKLSCAASGFTFTSYAMH WVRQPPGKGLEWVAV
VSYDGNYKYYADSVQG RFTISVDNSKNTLYLQMNSLRAEDTAVYYCAKDS
RLRSLLYFEWLSQGYFNP WGQGTTLTVSS (SEQ ID NO: 145)

>VH143
QVQLLETGGGLVKPGQSLKLSCAASGFTFTSYAMH WVRQPPGKGLEWVAV
VSYDGNYKYYADSVQG RFTISRDSSKNTLYLQMNSLRAEDTAVYYCAKDS
RLRSLLYFEWLSQGYFNP WGQGTTLTVSS (SEQ ID NO: 146)

>VH144
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSFLYFEWLSQGYFNP WGQGTTLTVSS (SEQ ID NO: 147)

>VH145
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSLLFFEWLSQGYFNP WGQGTTLTVSS (SEQ ID NO: 148)

>VH146
QVQLLETGGGLVKPGQSLKLSCAASGFTFTSYAMH WVRQPPGKGLEWVAV
VSYDGNYKYYADSVQG RFTISRDNSKNTLYLQMNSLRAEDTAVYYCAKDS
RLRSFLFFEWLSQGYFNP WGQGTTLTVSS (SEQ ID NO: 149)

>VH147
QVQLVQSGAEVKKPGSSVKVSCKASGFTFSTYAIH WVRQAPGQGLEWMGV
ISYDANYKYYAQKFQG RVTITRDNSKNTAYMELSSLRSEDTAVYYCAKDS
RLRSLLYFEWLSQGYFNP WGQGTTLTVSS (SEQ ID NO: 150)

>VH148
QVQLVQSGAEVKKPGSSVKVSCKASGFTFSTYAIH WVRQAPGQGLEWMGV
ISYDANYKYYAQKFQG RVTITRDNSKLTAYMELSSLRSEDTAVYYCAKDS
RLRSLLYFEWLSQGYFNP WGQGTTLTVSS (SEQ ID NO: 151)

>VH149
QVQLVQSGAEVKKPGSSVKVSCKASGFTFSTYAIH WVRQAPGQGLEWMGV
ISYDANYKYYAQKFQG RVTITRDNSKWTAYMELSSLRSEDTAVYYCAKDS
RLRSLLYFEWLSQGYFNP WGQGTTLTVSS (SEQ ID NO: 152)

>VH150
QVQLVQSGAEVKKPGSSVKVSCKASGFTFSTYAIH WVRQAPGQGLEWMGV
ISYDANYKYYAQKFQG RVTITRDNSKFTAYMELSSLRSEDTAVYYCAKDS
RLRSLLYFEWLSQGYFNP WGQGTTLTVSS (SEQ ID NO: 153)

>VH151
QVQLVQSGAEVKKPGSSVKVSCKASGFTFSTYAIH WVRQAPGQGLEWMGV
ISYDANYKYYAQKFQG RVTITRDNYKSTAYMELSSLRSEDTAVYYCAKDS
RLRSLLYFEWLSQGYFNP WGQGTTLTVSS (SEQ ID NO: 154)

>VH152
QVQLVQSGAEVKKPGSSVKVSCKASGFTFSTYAIH WVRQAPGQGLEWMGV
ISYDANYKYYAQKFQG RVTITRDNWKSTAYMELSSLRSEDTAVYYCAKDS
RLRSLLYFEWLSQGYFNP WGQGTTLTVSS (SEQ ID NO: 155)

>VH153
QVQLVQSGAEVKKPGSSVKVSCKASGFTFSTYAIH WVRQAPGQGLEWMGV
ISYDANYKYYAQKFQG RVTITRDNSYSTAYMELSSLRSEDTAVYYCAKDS
RLRSLLYFEWLSQGYFNP WGQGTTLTVSS (SEQ ID NO: 156)

>VH154
QVQLVQSGAEVKKPGSSVKVSCKASGFTFSTYAIH WVRQAPGQGLEWMGV
ISYDANYKYYAQKFQG RVTITRDNSWSTAYMELSSLRSEDTAVYYCAKDS
RLRSLLYFEWLSQGYFNP WGQGTTLTVSS (SEQ ID NO: 157)

>VH155
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCARDS
RLRSLLYFEWLSQGYFNP WGQGTTLTVSS (SEQ ID NO: 158)

>VH156
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCARDS
RLRSLLYFEWLSQGYFGV WGQGTTLTVSS (SEQ ID NO: 159)

>VH157
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGYFDP WGQGTTLTVSS (SEQ ID NO: 160)

>VH158
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCARDS
RLRSLLYFEWLSQGYFDP WGQGTTLTVSS (SEQ ID NO: 161)

>VH159
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGYFQP WGQGTTLTVSS (SEQ ID NO: 162)

>VH160
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGYFND WGQGTTLTVSS (SEQ ID NO: 163)

>VH161
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGYFDD WGQGTTLTVSS (SEQ ID NO: 164)

>VH162
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGYFQH WGQGTLVTVSS (SEQ ID NO: 165)

>VH163
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGYFDL WGRGTLVTVSS (SEQ ID NO: 166)

>VH164
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGYFDI WGQGTMVTVSS (SEQ ID NO: 167)

>VH165
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGY FDYWGQGTLVTVSS (SEQ ID NO: 168)

>VH166
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGYFDP WGQGTLVTVSS (SEQ ID NO: 169)

>VH167
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGYMDV WGQGTTVTVSS (SEQ ID NO: 170)

>VH168
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGYFDF WGQGTTLTVSS (SEQ ID NO: 171)

>VH169
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGYFDV WGQGTTLTVSS (SEQ ID NO: 172)

>VH170
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCARDS
RLRSLLYFEWLSQGYFEI WGQGTTLTVSS (SEQ ID NO: 173)

>VH171
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCARDS
RLRSLLYFEWLSQGYFEY WGQGTTLTVSS (SEQ ID NO: 174)

>VH172
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGYFSY WGQGTTLTVSS (SEQ ID NO: 175)

>VH173
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RLRSLLYFEWLSQGYMGV WGQGTTLTVSS (SEQ ID NO: 176)

>VH174
QVQLVQSGAEVKKPGSSVKVSCKASGFTFSTYAIH WVRQAPGQGLEWMGV
ISYDANYKYYAQKFQG RVTITRDNSKSTAYMELSSLRSEDTAVYYCAKDS
RNRSLLYFEWLSQGYFNP WGQGTTLTVSS (SEQ ID NO: 177)

>VH175
QVQLVQSGAEVKKPGSSVKVSCKASGFTFSTYAIH WVRQAPGQGLEWMGV
ISYDANYKYYAQKFQG RVTITRDNSWLTAYMELSSLRSEDTAVYYCAKDS
RLRSLLYFEWLSQGYFNP WGQGTTLTVSS (SEQ ID NO: 178)

>VH176
EVQLLESGGGLVQPGGSLRLSCAASGFTFSSYAMH WVRQAPGKGLEWVAV
ISYDANYKYYADSVKG RFTISRDNSWLTLYLQMNSLRAEDTAVYYCAKDS
RLRSLLYFEWLSQGYFNP WGQGTLVTVSS (SEQ ID NO: 179)

>VH177
EVQLLESGGGLVQPGGSLRLSCAASGFTFSSYAMH WVRQAPGKGLEWVAV
ISYDANYKYYADSVKG RFTISRDNSKSTLYLQMNSLRAEDTAVYYCAKDS
RLRSLLYFEWLSQGYFNP WGQGTLVTVSS (SEQ ID NO: 180)

>VH178
EVQLLESGGGLVQPGGSLRLSCAASGFTFSSYAMH WVRQAPGKGLEWVAV
ISYDANYKYYADSVKG RFTISRDNSKLTLYLQMNSLRAEDTAVYYCAKDS
RLRSLLYFEWLSQGYFNP WGQGTLVTVSS (SEQ ID NO: 181)

>VH179
EVQLLESGGGLVQPGGSLRLSCAASGFTFSSYAMH WVRQAPGKGLEWVAV
ISYDANYKYYADSVKG RFTISRDNSWNTLYLQMNSLRAEDTAVYYCAKDS
RLRSLLYFEWLSQGYFNP WGQGTLVTVSS (SEQ ID NO: 182)

>VH180
QVQLVQSGAEVKKPGSSVKVSCKASGFTFTSYAIH WVRQAPGQGLEWMGV
ISYDGNYKYYAQKFQG RVTITRDNSKLTAYMELSSLRSEDTAVYYCAKDS
RLRSLLYFEWLSQGYFNP WGQGTTLTVSS (SEQ ID NO: 183)

>VH181
QVQLVQSGAEVKKPGSSVKVSCKASGFTFTSYAIH WVRQAPGQGLEWMGV
ISYDGNYKYYAQKFQG RVTITRDNSWLTAYMELSSLRSEDTAVYYCAKDS
RLRSLLYFEWLSQGYFNP WGQGTTLTVSS (SEQ ID NO: 184)

>VH182
QVQLVQSGAEVKKPGSSVKVSCKASGFTFTSYAIH WVRQAPGQGLEWMGV
ISYDGNYKYYAQKFQG RVTITRDNSKSTAYMELSSLRSEDTAVYYCAKDS
RLRSLLYFEWLSQGYFNP WGQGTTLTVSS (SEQ ID NO: 185)

>VH183
EVQLLESGGGLVQPGGSLRLSCAASGFTFTSYAMH WVRQAPGKGLEWVAV
ISYDGNYKYYADSVKG RFTISRDNSKNTLYLQMNSLRAEDTAVYYCAKDS
RLRSLLYFEWLSQGYFNP WGQGTLVTVSS (SEQ ID NO: 186)

>VH184
EVQLLESGGGLVQPGGSLRLSCAASGFTFTSYAMH WVRQAPGKGLEWVAV
ISYDGNYKYYADSVKG RFTISRDNSWLTLYLQMNSLRAEDTAVYYCAKDS
RLRSLLYFEWLSQGYFNP WGQGTLVTVSS (SEQ ID NO: 187)

Exemplary Light Chain Variable Regions

>VK-0
DIQMTQSPSSLSASVGDRVTITCRSSQSITFDYKNYLA WYQQKPGKAPKL
LIYWGSYLES GVPSRFSGSGSGTDFTLTISSLQPEDFATYYCQQHYRTPP
S FGQGTKVEIK (SEQ ID NO: 188)

>VK-1
DIQMTQSPSSLSASVGDRVTITCRSSQSITFDYKNYLA WYQQKPGKAPKL
LIYWGSYLES GVPSRFSGSGSGTDFTLTISSLQPEDFATYYCQQHYRTPP
S FGQGTKVEIK (SEQ ID NO: 188)

>VK-2
DIQMTQSPSSLSASVGDRVTITCRSSQSITFDYKNYLA WYQQKPGKAPKL
LIYWGSSLQS GVPSRFSGSGSGTDFTLTISSLQPEDFATYYCQQHYRTPP
S FGQGTKVEIK (SEQ ID NO: 189)

>VK-3
DIQMTQSPSSLSASVGDRVTITCRSSQSITFDYKNYLA WYQQKPGKAPKL
LIYAASSLQS GVPSRFSGSGSGTDFTLTISSLQPEDFATYYCQQHYRTPP
S FGQGTKVEIK (SEQ ID NO: 190)

>VK-4
DIVMTQSPDSLAVSLGERATINCKSSQSVTFDYKNYLA WYQQKPGQPPKL
LIYWASTRES GVPDRFSGSGSGTDFTLTISSLQAEDVAVYYCQQHYRTPP
S FGQGTKVEIK (SEQ ID NO: 191)

>VK-5
DIVMTQSPDSLAVSLGERATINCKSSQSVTFDYKNYLA WYQQKPGQPPKL
LIYEASTRES GVPDRFSGSGSGTDFTLTISSLQAEDVAVYYCQQHYRTPP
S FGQGTKVEIK (SEQ ID NO: 192)

>VK-6
EIVMTQSPATLSVSPGERATLSCRSSQSITFDYKNYLA WYQQKPGQAPRL
LIYWGSTRAT GIPARFSGSGSGTEFTLTISSLQSEDFAVYYCQQHYRTPP
S FGQGTKVEIK (SEQ ID NO: 193)

>VK-7
EIVMTQSPATLSVSPGERATLSCRSSQSITFDYKNYLA WYQQKPGQAPRL
LIYSASTRAT GIPARFSGSGSGTEFTLTISSLQSEDFAVYYCQQHYRTPP
S FGQGTKVEIK (SEQ ID NO: 194)

>VK-8
DIQMTQSPSSLSASVGDRVTITCRSSQSITFDYKNYLA WYQQKPGKAPKL
LIYWGSYLES GVPSRFSGSGSGTDFTLTISSLQPEDFATYYCQQCYRTPP
S FGQGTKVEIK (SEQ ID NO: 195)

>VK-9
DIQMTQSPSSLSASVGDRVTITCRSSQSITFDYKNYLA WYQQKPGKAPKL
LIYWGSYLES GVPSRFSGSGSGTDFTLTISSLQPEDFATYYCQQHYCTPP
S FGQGTKVEIK (SEQ ID NO: 196)

>VK-10
DIQMTQSPSSLSASVGDRVTITCRSSQSITFDYQNYLA WYQQKPGKAPKL
LIYWGSYLES GVPSRFSGSGSGTDFTLTISSLQPEDFATYYCQQHYRTPP
S FGQGTKVEIK (SEQ ID NO: 197)

>VK-11
DIQMTQSPSSLSASVGDRVTITCRSSQSITFDYKNYLA WYQQKPGKAPKL
LIYWGSYLES GVPSRFSGSGSGTDFTLTISSLQPEDFATYYCQQFYRTPP
S FGQGTKVEIK (SEQ ID NO: 198)

>VK-12
QIQMTQSPSSLSASVGDRVTITCRSSQSITFDYKNYLA WYQQKPGKAPKL
LIYWGSYLES GVPSRFSGSGSGTDFTLTISSLQPEDFATYYCQQHYRTPP
S FGQGTKVEIK (SEQ ID NO: 199)

>VK-13
DIQMTQSPSSLSASVGDRVTITCRSSQSITFDYQNYLA WYQQKPGKAPKL
LIYWGSTLES GVPSRFSGSGSGTDFTLTISSLQPEDFATYYCQQHYRTPP
S FGQGTKVEIK (SEQ ID NO: 200)

>VK-14
DIQMTQSPSSLSASVGDRVTITCRSSQSITSDNQNYLA WYQQKPGKAPKL
LIYWGSYLES GVPSRFSGSGSGTDFTLTISSLQPEDFATYYCQQHYRTPP
S FGQGTKVEIK (SEQ ID NO: 201)

>VK-15
DIVMTQSPDSLAVSLGERATINCKSSQSVL YSSNNKNYLA WYQQKPGQP
PKLLIDWASTRES GVPDRFSGSGSGTDFTLTISNLQVEDVAVYYCQQYYR
S-PS FGQGTKLEIK (SEQ ID NO: 202)

>VK-16
DIQMTQSPSSLSASVGDRVTITCRSSQSITFDYKNYLA WYQQKPGKAPKL
LIDWGSYLES GVPSRFSGSGSGTDFTLTISSLQPEDFATYYCQQHYRTPP
S FGQGTKVEIK (SEQ ID NO: 203)

>VK-17
DIQMTQSPSSLSASVGDRVTITCRSSQSITFDYKNYLA WYQQKPGKAPKL
LIDWGSYLES GVPSRFSGSGSGTDFTLTISSLQPEDFATYYCQQYYRTPP
S FGQGTKVEIK (SEQ ID NO: 204)

>VK-18
DIVMTQSPDSLAVSLGERATINCKSSQSVSFNYKNYLA WYQQKPGQPPKL
LIDWASTRES GVPDRFSGSGSGTDFTLTISNLQVEDVAVYYCQQYYRSPP
S FGQGTKLEIK (SEQ ID NO: 205)

>VK-19
DIQMTQSPDSLAVSLGARATINCKSSQSVTFNYKNYLA WYQQKPGQPPKV
LIDWASARES GVPDRFSGSGSGTDFTLTISSLQAEDVAVYYCQQHYRTPP
T FGQGTKVEIK (SEQ ID NO: 206)

>VK-20
DIVMTQSPDSLAVSLGERATINCKSSQSVTFNYKNYLA WYQQKPGQPPKL
LIYWASTRES GVPDRFSGSGSGTDFTLTISSLQAEDVAVYYCQQHYRTPP
T FGQGTKVEIK (SEQ ID NO: 207)

>VK-21
DIQMTQSPSSLSASVGDRVTITCRSSQSITFDYKNYLA WYQQKPGKAPKL
LIYEGSYLES GVPSRFSGSGSGTDFTLTISSLQPEDFATYYCQQHYRTPP
S FGQGTKVEIK (SEQ ID NO: 208)

>VK-22
DIQMTQSPSSLSASVGDRVTITCRSSQSITFDYKNYLA WYQQKPGKAPKL
LIYSGSYLES GVPSRFSGSGSGTDFTLTISSLQPEDFATYYCQQHYRTPP
S FGQGTKVEIK (SEQ ID NO: 209)

>VK-23
DIQMTQSPSSLSASVGDRVTITCRSSQSITFDYKNRLA WYQQKPGKAPKL
LIYWGSYLES GVPSRFSGSGSGTDFTLTISSLQPEDFATYYCQQHYRTPP
S FGQGTKVEIK (SEQ ID NO: 210)

>VK-24
DIQMTQSPSSLSASVGDRVTITCRSSQSITSDNKNYLAW YQQKPGKAPKL
LIYWGSYLES GVPSRFSGSGSGTDFTLTISSLQPEDFATYYCQQHYRTPP
S FGQGTKVEIK (SEQ ID NO: 211)

>VK-25
DIQMTQSPSSLSASVGDRVTITCRSSQSITSDDKNYLA WYQQKPGKAPKL
LIYWGSYLES GVPSRFSGSGSGTDFTLTISSLQPEDFATYYCQQHYRTPP
S FGQGTKVEIK (SEQ ID NO: 212)

>VK-26
DIQMTQSPSSLSASVGDRVTITCRSSQSITSDEKNYLA WYQQKPGKAPKL
LIYWGSYLES GVPSRFSGSGSGTDFTLTISSLQPEDFATYYCQQHYRTPP
S FGQGTKVEIK (SEQ ID NO: 213)

>VK-27
LIDIQMTQSPSSLSASVGDRVTITCRSSQSITDDNKNYLA WYQQKPGKAP
KLYWGSYLES GVPSRFSGSGSGTDFTLTISSLQPEDFATYYCQQHYRTPP
S FGQGTKVEIK (SEQ ID NO: 214)

>VK-28
DIQMTQSPSSLSASVGDRVTITCRSSQSITDDDKNYLA WYQQKPGKAPKL
LIYWGSYLES GVPSRFSGSGSGTDFTLTISSLQPEDFATYYCQQHYRTPP
S FGQGTKVEIK (SEQ ID NO: 215)

>VK-29
DIQMTQSPSSLSASVGDRVTITCRSSQSITDDEKNYLA WYQQKPGKAPKL
LIYWGSYLES GVPSRFSGSGSGTDFTLTISSLQPEDFATYYCQQHYRTPP
S FGQGTKVEIK (SEQ ID NO: 216)

>VK-30
DIQMTQSPSSLSASVGDRVTITCRSSQSITEDNKNYLA WYQQKPGKAPKL
LIYWGSYLES GVPSRFSGSGSGTDFTLTISSLQPEDFATYYCQQHYRTPP
S FGQGTKVEIK (SEQ ID NO: 217)

>VK-31
DIQMTQSPSSLSASVGDRVTITCRSSQSITEDDKNYLA WYQQKPGKAPKL
LIYWGSYLES GVPSRFSGSGSGTDFTLTISSLQPEDFATYYCQQHYRTPP
S FGQGTKVEIK (SEQ ID NO: 218)

>VK-32
DIQMTQSPSSLSASVGDRVTITCRSSQSITEDEKNYLA WYQQKPGKAPKL
LIYWGSYLES GVPSRFSGSGSGTDFTLTISSLQPEDFATYYCQQHYRTPP
S FGQGTKVEIK (SEQ ID NO: 219)

>VK-33
DIQMTQSPSSLSASVGDRVTITCRSSQSITQDNKNYLA WYQQKPGKAPKL
LIYWGSYLES GVPSRFSGSGSGTDFTLTISSLQPEDFATYYCQQHYRTPP
S FGQGTKVEIK (SEQ ID NO: 220)

>VK-34
DIQMTQSPSSLSASVGDRVTITCRSSQSITQDDKNYLA WYQQKPGKAPKL
LIYWGSYLES GVPSRFSGSGSGTDFTLTISSLQPEDFATYYCQQHYRTPP
S FGQGTKVEIK (SEQ ID NO: 221)

>VK-35
DIQMTQSPSSLSASVGDRVTITCRSSQSITQDEKNYLA WYQQKPGKAPKL
LIYWGSYLES GVPSRFSGSGSGTDFTLTISSLQPEDFATYYCQQHYRTPP
S FGQGTKVEIK (SEQ ID NO: 222)

>VK-36
DIQMTQSPSSLSASVGDRVTITCRSSQSITHDNKNYLA WYQQKPGKAPKL
LIYWGSYLES GVPSRFSGSGSGTDFTLTISSLQPEDFATYYCQQHYRTPP
S FGQGTKVEIK (SEQ ID NO: 223)

>VK-37
DIQMTQSPSSLSASVGDRVTITCRSSQSITHDDKNYLA WYQQKPGKAPKL
LIYWGSYLES GVPSRFSGSGSGTDFTLTISSLQPEDFATYYCQQHYRTPP
S FGQGTKVEIK (SEQ ID NO: 224)

>VK-38
DIQMTQSPSSLSASVGDRVTITCRSSQSITHDEKNYLA WYQQKPGKAPKL
LIYWGSYLES GVPSRFSGSGSGTDFTLTISSLQPEDFATYYCQQHYRTPP
S FGQGTKVEIK (SEQ ID NO: 225)

>VK-39
DIQMTQSPSSLSASVGDRVTITCRSSQSITFDYKNYLAW YQQKPGKAPKL
LIYAASSRQS GVPDRFSGSGSGTDFTLTISSLQPEDFATYYCQQHYRTPP
S FGQGTKVEIK (SEQ ID NO: 226)

>VK-40
DIQMTQSPSSLSASVGDRVTITCRSSQSITFNYKNYLA WYQQKPGKAPKL
LIYAASSLQS GVPSRFSGSGSGTDFTLTISSLQPEDFATYYCQQHYRTPP
S FGQGTKVEIK (SEQ ID NO: 227)

>VK-41
DIQMTQSPSSLSASVGDRVTITCRSSQSITFDYKNYLA WYQQKPGKAPKL
LIYAASSLES GVPSRFSGSGSGTDFTLTISSLQPEDFATYYCQQHYRTPP
S FGQGTKVEIK (SEQ ID NO: 228)

>VK-42
DIQMTQSPSSLSASVGDRVTITCRSSQSITFDYKNYLA WYQQKPGKAPKL
LIYSASSLQS GVPSRFSGSGSGTDFTLTISSLQPEDFATYYCQQHYRTPP
S FGQGTKVEIK (SEQ ID NO: 229)

>VK-43
DIQMTQSPSSLSASVGDRVTITCRSSQSITFDYKNYLA WYQQKPGKAPKL
LIYSGSSLQS GVPSRFSGSGSGTDFTLTISSLQPEDFATYYCQQHYRTPP
S FGQGTKVEIK (SEQ ID NO: 230)

>VK-44
DIQMTQSPSSLSASVGDRVTITCRSSQSVTFDYKNYLA WYQQKPGKAPKL
LIYAASSLQS GVPSRFSGSGSGTDFTLTISSLQPEDFATYYCQQHYRTPP
S FGQGTKVEIK (SEQ ID NO: 231)

>VK-45
DIQMTQSPSSLSASVGDRVTITCRSSQSITDDNKNYLA WYQQKPGKAPKL
LIYAASSLQS GVPSRFSGSGSGTDFTLTISSLQPEDFATYYCQQHYRTPP
S FGQGTKVEIK (SEQ ID NO: 232)

>VK-46
DIQMTQSPSSLSASVGDRVTITCRSSQSITSDYKNYLA WYQQKPGKAPKL
LIYAASSLQS GVPSRFSGSGSGTDFTLTISSLQPEDFATYYCQQHYRTPP
S FGQGTKVEIK (SEQ ID NO: 233)

>VK-47
DIQMTQSPSSLSASVGDRVTITCRSSQSITSDNKNYLA WYQQKPGKAPKL
LIYAASSLQS GVPSRFSGSGSGTDFTLTISSLQPEDFATYYCQQHYRTPP
S FGQGTKVEIK (SEQ ID NO: 234)

>VK-48
DIQMTQSPSSLSASVGDRVTITCRSSQSITFDYKNYLA WYQQKPGKAPKL
LIYAASSLQS GVPSRFSGSGSGTDFTLTISSLQPEDFATYYCQQHYSTPP
S FGQGTKVEIK (SEQ ID NO: 235)

>VK-49
DIQMTQSPSSLSASVGDRVTITCRSSQSITFDYKNYLA WYQQKPGKAPKL
LIYAASSLQS GVPSRFSGSGSGTDFTLTISSLQPEDFATYYCQQHYQTPP
S FGQGTKVEIK (SEQ ID NO: 236)

>VK-50
DIQMTQSPDSLSASVGDRVTITCRSSQSITFDYKNYLA WYQQKPGKAPKL
LIYAASSLQS GVPSRFSGSGSGTDFTLTISSLQPEDFATYYCQQHYRTPP
T FGQGTKVEIK (SEQ ID NO: 237)

>VK-51
DIVMTQSPDSLAVSLGERATINCKSSQSVTFDYKNYLA WYQQKPGQPPKL
LIYWASTRES GVPDRFSGSGSGTDFTLTISSLQAEDVAVYYCQQHYRTPP
T FGQGTKVEIK (SEQ ID NO: 238)

>VK-52
DIVMTQSPDSLAVSLGERATINCKSSQSVTFNYKNYLA WYQQKPGQPPKL
LIYWASTRES GVPDRFSGSGSGTDFTLTISSLQAEDVAVYYCQQHYRTPP
S FGQGTKVEIK (SEQ ID NO: 239)

>VK-53
DIVMTQSPDSLAVSLGERATINCKSSQSVTFDYKNYLA WYQQKPGQPPKL
LIYSASTRES GVPDRFSGSGSGTDFTLTISSLQAEDVAVYYCQQHYRTPP
S FGQGTKVEIK (SEQ ID NO: 240)

>VK-54
DIVMTQSPDSLAVSLGERATINCKSSQSVTFDYKNYLA WYQQKPGQPPKL
LIYWASTRQS GVPDRFSGSGSGTDFTLTISSLQAEDVAVYYCQQHYRTPP
S FGQGTKVEIK (SEQ ID NO: 241)

>VK-55
DIVMTQSPDSLAVSLGERATINCKSSQSITFDYKNYLA WYQQKPGQPPKL
LIYWASTRES GVPDRFSGSGSGTDFTLTISSLQAEDVAVYYCQQHYRTPP
S FGQGTKVEIK (SEQ ID NO: 242)

>VK-56
DIVMTQSPDSLAVSLGERATINCKSSQSVTFDYKNYLA WYQQKPGQPPKL
LIYSGSTRES GVPDRFSGSGSGTDFTLTISSLQAEDVAVYYCQQHYRTPP
S FGQGTKVEIK (SEQ ID NO: 243)

>VK-57
DIQMTQSPDSLSVSLGERATINCKSSQSVTFDYKNYLA WYQQKPGQPPKL
LIYWASTRES GVPDRFSGSGSGTDFTLTISSLQAEDVAVYYCQQHYRTPP
S FGQGTKVEIK (SEQ ID NO: 244)

>VK-58
DIVMTQSPDSLAVSLGERATINCKSSQSVTFDYKNYLA WYQQKPGQPPKL
LIYWASTRES GVPDRFSGSGSGTDFTLTISSLQAEDVATYYCQQHYRTPP
S FGQGTKVEIK (SEQ ID NO: 245)

>VK-59
DIVMTQSPDSLAVSLGERATINCKSSQSVTDDNKNYLA WYQQKPGQPPKL
LIYWASTRES GVPDRFSGSGSGTDFTLTISSLQAEDVAVYYCQQHYRTPP
S FGQGTKVEIK (SEQ ID NO: 246)

>VK-60
DIVMTQSPDSLAVSLGERATINCKSSQSVTSDYKNYLA WYQQKPGQPPKL
LIYWASTRES GVPDRFSGSGSGTDFTLTISSLQAEDVAVYYCQQHYRTPP
S FGQGTKVEIK (SEQ ID NO: 247)

>VK-61
DIVMTQSPDSLAVSLGERATINCKSSQSVTSDNKNYLA WYQQKPGQPPKL
LIYWASTRES GVPDRFSGSGSGTDFTLTISSLQAEDVAVYYCQQHYRTPP
S FGQGTKVEIK (SEQ ID NO: 248)

>VK-62
DIVMTQSPDSLAVSLGERATINCKSSQSVTFDYKNYLA WYQQKPGQPPKL
LIYWASTRES GVPDRFSGSGSGTDFTLTISSLQAEDVAVYYCQQHYSTPP
S FGQGTKVEIK (SEQ ID NO: 249)

>VK-63
DIVMTQSPDSLAVSLGERATINCKSSQSVTFDYKNYLA WYQQKPGQPPKL
LIYWASTRES GVPDRFSGSGSGTDFTLTISSLQAEDVAVYYCQQHYQTPP
S FGQGTKVEIK (SEQ ID NO: 250)

>VK-64
DIQMTQSPSSLSASVGDRVTITCRSSESITFDYKNYLA WYQQKPGKAPKL
LIYWGSYLES GVPSRFSGSGSGTDFTLTISSLQPEDFATYYCQQHYRTPP
S FGQGTKVEIK (SEQ ID NO: 251)

>VK-65
DIQMTQSPSSLSASVGDRVTITCRSSEDITFDYKNYLA WYQQKPGKAPKL
LIYWGSYLES GVPSRFSGSGSGTDFTLTISSLQPEDFATYYCQQHYRTPP
S FGQGTKVEIK (SEQ ID NO: 252)

>VK-66
DIQMTQSPSSLSASVGDRVTITCRSSESITFDYKNYLA WYQQKPGKAPKL
LIYWGSYLES GVPSRFSGSGSGTDFTLTISSLQPEDFATYYCQQHYQTPP
S FGQGTKVEIK (SEQ ID NO: 253)

>VK-67
DIQMTQSPSSLSASVGDRVTITCRSSEDITFDYKNYLA WYQQKPGKAPKL
LIYWGSYLES GVPSRFSGSGSGTDFTLTISSLQPEDFATYYCQQHYQTPP
S FGQGTKVEIK (SEQ ID NO: 254)

>VK-68
DIQMTQSPSSLSASVGDRVTITCRSSQSITFSSDYKNYLA WYQQKPGKAP
KLLIYWGSYLES GVPSRFSGSGSGTDFTLTISSLQPEDFATYYCQQHYRT
PPS FGQGTKVEIK (SEQ ID NO: 255)

>VK-69
DIQMTQSPSSLSASVGDRVTITCRSSQSITFSPDYKNYLA WYQQKPGKAP
KLLIYWGSYLES GVPSRFSGSGSGTDFTLTISSLQPEDFATYYCQQHYRT
PPS FGQGTKVEIK (SEQ ID NO: 256)

>VK-70
DIQMTQSPSSLSASVGDRVTITCRSSQSITLSPDYKNYLA WYQQKPGKAP
KLLIYWGSYLES GVPSRFSGSGSGTDFTLTISSLQPEDFATYYCQQHYRT
PPS FGQGTKVEIK (SEQ ID NO: 257)

>VK-71
DIQMTQSPSSLSASVGDRVTITCRSSQSITISPDYKNYLA WYQQKPGKAP
KLLIYWGSYLES GVPSRFSGSGSGTDFTLTISSLQPEDFATYYCQQHYRT
PPS FGQGTKVEIK (SEQ ID NO: 258)

>VK-72
DIQMTQSPSSLSASVGDRVTITCRSSQSITFGGDYKNYLA WYQQKPGKAP
KLLIYWGSYLES GVPSRFSGSGSGTDFTLTISSLQPEDFATYYCQQHYRT
PPS FGQGTKVEIK (SEQ ID NO: 259)

>VK-73
DIQMTQSPSSLSASVGDRVTITCRSSQSITFSDYKNYLA WYQQKPGKAP
KLLIYWGSYLES GVPSRFSGSGSGTDFTLTISSLQPEDFATYYCQQHYRT
PPS FGQGTKVEIK (SEQ ID NO: 260)

>VK-74
DIQMTQSPSSLSASVGDRVTITCRSSQSITFGDYKNYLA WYQQKPGKAP
KLLIYWGSYLES GVPSRFSGSGSGTDFTLTISSLQPEDFATYYCQQHYRT
PPS FGQGTKVEIK (SEQ ID NO: 261)

>VK-75
DIQMTQSPSSLSASVGDRVTITCRSSQSITFGPDYKNYLA WYQQKPGKAP
KLLIYWGSYLES GVPSRFSGSGSGTDFTLTISSLQPEDFATYYCQQHYRT
PPS FGQGTKVEIK (SEQ ID NO: 262)

>VK-76
DIQMTQSPSSLSASVGDRVTITCRSSQSITYDYKNYLA WYQQKPGKAPKL
LIYWGSYLES GVPSRFSGSGSGTDFTLTISSLQPEDFATYYCQQHYRTPP
S FGQGTKVEIK (SEQ ID NO: 263)

>VK-77
DIQMTQSPSSLSASVGDRVTITCRSSQSITWDYKNYLA WYQQKPGKAPKL
LIYWGSYLES GVPSRFSGSGSGTDFTLTISSLQPEDFATYYCQQHYRTPP
S FGQGTKVEIK (SEQ ID NO: 264)

>VK-78
DIQMTQSPSSLSASVGDRVTITCRSSQSITFWYKNYLA WYQQKPGKAPKL
LIYWGSYLES GVPSRFSGSGSGTDFTLTISSLQPEDFATYYCQQHYRTPP
S FGQGTKVEIK (SEQ ID NO: 265)

>VK-79
DIQMTQSPSSLSASVGDRVTITCRSSQSITFLDYKNYLA WYQQKPGKAP
KLLIYWGSYLES GVPSRFSGSGSGTDFTLTISSLQPEDFATYYCQQHYRT
PPS FGQGTKVEIK (SEQ ID NO: 266)

>VK-80
DIQMTQSPSSLSASVGDRVTITCRSSQSITLSPWYKNYLA WYQQKPGKAP
KLLIYWGSYLES GVPSRFSGSGSGTDFTLTISSLQPEDFATYYCQQHYRT
PPS FGQGTKVEIK (SEQ ID NO: 267)

>VK-81
DIQMTQSPSSLSASVGDRVTITCRSSQSITLSPYYKNYLA WYQQKPGKAP
KLLIYWGSYLES GVPSRFSGSGSGTDFTLTISSLQPEDFATYYCQQHYRT
PPS FGQGTKVEIK (SEQ ID NO: 268)

>VK-82
DIQMTQSPSSLSASVGDRVTITCRSSQSITLSPYDKNYLA WYQQKPGKAP
KLLIYWGSYLES GVPSRFSGSGSGTDFTLTISSLQPEDFATYYCQQHYRT
PPS FGQGTKVEIK (SEQ ID NO: 269)

>VK-83
DIQMTQSPSSLSASVGDRVTITCRSSQSITFDNKNYLA WYQQKPGKAPKL
LIYWGSYLES GVPSRFSGSGSGTDFTLTISSLQPEDFATYYCQQHYRTPP
S FGQGTKVEIK (SEQ ID NO: 270)

>VK-84
DIQMTQSPSSLSASVGDRVTITCRSSQSITSDNKNYLA WYQQKPGKAPKL
LIYWGSELES GVPSRFSGSGSGTDFTLTISSLQPEDFATYYCQQHYRTPP
S FGQGTKVEIK (SEQ ID NO: 271)

>VK-85
DIQMTQSPSSLSASVGDRVTITCRSSQSITADNKNYLA WYQQKPGKAPKL
LIYWGSYLES GVPSRFSGSGSGTDFTLTISSLQPEDFATYYCQQHYRTPP
S FGQGTKVEIK (SEQ ID NO: 272)

>VK-86
DIQMTQSPSSLSASVGDRVTITCRSSQSITNDNKNYLA WYQQKPGKAPKL
LIYWGSYLES GVPSRFSGSGSGTDFTLTISSLQPEDFATYYCQQHYRTPP
S FGQGTKVEIK (SEQ ID NO: 273)

>VK-87
DIQMTQSPSSLSASVGDRVTITCRSSQSITMDNKNYLA WYQQKPGKAPKL
LIYWGSYLES GVPSRFSGSGSGTDFTLTISSLQPEDFATYYCQQHYRTPP
S FGQGTKVEIK (SEQ ID NO: 274)

>VK-88
DIQMTQSPSSLSASVGDRVTITCRSSQSITRDNKNYLA WYQQKPGKAPKL
LIYWGSYLES GVPSRFSGSGSGTDFTLTISSLQPEDFATYYCQQHYRTPP
S FGQGTKVEIK (SEQ ID NO: 275)

>VK-89
DIQMTQSPSSLSASVGDRVTITCRSSQSITSDSKNYLA WYQQKPGKAPKL
LIYWGSYLES GVPSRFSGSGSGTDFTLTISSLQPEDFATYYCQQHYRTPP
S FGQGTKVEIK (SEQ ID NO: 276)

>VK-90
DIQMTQSPSSLSASVGDRVTITCRSSQSITSDQKNYLA WYQQKPGKAPKL
LIYWGSYLES GVPSRFSGSGSGTDFTLTISSLQPEDFATYYCQQHYRTPP
S FGQGTKVEIK (SEQ ID NO: 277)

>VK-91
DIQMTQSPSSLSASVGDRVTITCRSSQSITSDRKNYLA WYQQKPGKAPKL
LIYWGSYLES GVPSRFSGSGSGTDFTLTISSLQPEDFATYYCQQHYRTPP
S FGQGTKVEIK (SEQ ID NO: 278)

>VK-92
DIQMTQSPSSLSASVGDRVTITCRSSQSITFDYKNYLA WYQQKPGKAPKL
LIYWGSDLES GVPSRFSGSGSGTDFTLTISSLQPEDFATYYCQQHYRTPP
S FGQGTKVEIK (SEQ ID NO: 279)

>VK-93
DIQMTQSPSSLSASVGDRVTITCRSSQSITFDYKNYLA WYQQKPGKAPKL
LIYWGSELES GVPSRFSGSGSGTDFTLTISSLQPEDFATYYCQQHYRTPP
S FGQGTKVEIK (SEQ ID NO: 280)

>VK-94
DIQMTQSPSSLSASVGDRVTITCRSSQSITFDYKNYLA WYQQKPGKAPKL
LIYWGEYLES GVPSRFSGSGSGTDFTLTISSLQPEDFATYYCQQHYRTPP
S FGQGTKVEIK (SEQ ID NO: 281)

>VK-95
DIQMTQSPSSLSASVGDRVTITCRSSQSITFDYKNYLA WYQQKPGKAPKL
LIYWGSYLES GVPSRFSGSGSGTDFTLTISSLQPEDFATYYCQQHYRTP
S FGQGTKVEIK (SEQ ID NO: 282)

>VK-96
DIVMTQSPDSLAVSLGERATINCKSSQSVTFDYKNYLA WYQQKPGQPPKL
LIYAASTRES GVPDRFSGSGSGTDFTLTISSLQAEDVAVYYCQQHYRTPP
S FGQGTKVEIK (SEQ ID NO: 283)

>VK-97
DIQMTQSPSSLSASVGDRVTITCRSSEDITFWYKNYLA WYQQKPGKAPKL
LIYWGSYLES GVPSRFSGSGSGTDFTLTISSLQPEDFATYYCQQHYRTPP
S FGQGTKVEIK (SEQ ID NO: 284)

>VK-98
DIQMTQSPSSLSASVGDRVTITCRSSQSITFWEKNYLA WYQQKPGKAPKL
LIYWGSYLES GVPSRFSGSGSGTDFTLTISSLQPEDFATYYCQQHYRTPP
S FGQGTKVEIK (SEQ ID NO: 285)

>VK-99
DIQMTQSPSSLSASVGDRVTITCRSSQSITSWNKNYLA WYQQKPGKAPKL
LIYWGSYLES GVPSRFSGSGSGTDFTLTISSLQPEDFATYYCQQHYRTPP
S FGQGTKVEIK (SEQ ID NO: 286)

>VK-100
DIQMTQSPSSLSASVGDRVTITCRSSEDITSWNKNYLA WYQQKPGKAPKL
LIYWGSYLES GVPSRFSGSGSGTDFTLTISSLQPEDFATYYCQQHYRTPP
S FGQGTKVEIK (SEQ ID NO: 287)

>VK-101
DIQMTQSPSSLSASVGDRVTITCRSSEDITSWDKNYLA WYQQKPGKAPKL
LIYWGSYLES GVPSRFSGSGSGTDFTLTISSLQPEDFATYYCQQHYRTPP
S FGQGTKVEIK (SEQ ID NO: 288)

>VK-102
DIQMTQSPSSLSASVGDRVTITCRSSQSITFYYKNYLA WYQQKPGKAPKL
LIYWGSYLES GVPSRFSGSGSGTDFTLTISSLQPEDFATYYCQQHYRTPP
S FGQGTKVEIK (SEQ ID NO: 289)

>VK-103
DIQMTQSPSSLSASVGDRVTITCRSSQSITFRYKNYLA WYQQKPGKAPKL
LIYWGSYLES GVPSRFSGSGSGTDFTLTISSLQPEDFATYYCQQHYRTPP
S FGQGTKVEIK (SEQ ID NO: 290)

>VK-104
DIQMTQSPSSLSASVGDRVTITCRSSQSITFRDKNYLA WYQQKPGKAPKL
LIYWGSYLES GVPSRFSGSGSGTDFTLTISSLQPEDFATYYCQQHYRTPP
S FGQGTKVEIK (SEQ ID NO: 291)

>VK-105
DIQMTQSPSSLSASVGDRVTITCRSSQSITDRYKNYLA WYQQKPGKAPKL
LIYWGSYLES GVPSRFSGSGSGTDFTLTISSLQPEDFATYYCQQHYRTPP
S FGQGTKVEIK (SEQ ID NO: 292)

>VK-106
DIQMTQSPSSLSASVGDRVTITCRSSESITFYYKNYLA WYQQKPGKAPKL
LIYWGSYLES GVPSRFSGSGSGTDFTLTISSLQPEDFATYYCQQHYRTPP
S FGQGTKVEIK (SEQ ID NO: 293)

>VK-107
DIQMTQSPSSLSASVGDRVTITCRSSQSITSDYKNYLA WYQQKPGKAPKL
LIYWGSYLES GVPSRFSGSGSGTDFTLTISSLQPEDFATYYCQQHYRTPP
S FGQGTKVEIK (SEQ ID NO: 294)

>VK-108
DIVMTQSPDSLAVSLGERATINCKSSQSVTFWYKNYLA WYQQKPGQPPKL
LIYWASTRES GVPDRFSGSGSGTDFTLTISSLQAEDVAVYYCQQHYRTPP
T FGQGTKVEIK (SEQ ID NO: 295)

>VK-109
DIQMTQSPSSLSASVGDRVTITCRSSEDITSDNQNYLA WYQQKPGKAPKL
LIYWGSYLES GVPSRFSGSGSGTDFTLTISSLQPEDFATYYCQQHYRTPP
S FGQGTKVEIK (SEQ ID NO: 296)

>VK-110
DIQMTQSPSSLSASVGDRVTITCRSSEDITSDNKNYLA WYQQKPGKAPKL
LIYWGSYLES GVPSRFSGSGSGTDFTLTISSLQPEDFATYYCQQHYRTPP
S FGQGTKVEIK (SEQ ID NO: 297)

>VK-111
DIQMTQSPSSLSASVGDRVTITCRSSQSITWDNKNYLA WYQQKPGKAPKL
LIYWGSYLES GVPSRFSGSGSGTDFTLTISSLQPEDFATYYCQQHYRTPP
S FGQGTKVEIK (SEQ ID NO: 298)

TABLE 15

Exemplary Heavy Chain CDRs (HCDRs)

| HCDR | Amino Acid Sequence | SEQ ID NO |
|---|---|---|
| HCDR1 | DYAIH | 299 |
| | DYAMH | 300 |
| | HYAMH | 301 |
| | LYAMH | 302 |
| | NYAMH | 303 |
| | NYGMH | 304 |
| | QYAMH | 305 |
| | SFAMH | 306 |
| | SHAMH | 307 |
| | SRAMH | 308 |
| | SYAIH | 309 |
| | SYAMH | 310 |
| | SYGMH | 311 |
| | SYSMH | 312 |
| | THAIH | 313 |
| | TRAIH | 314 |
| | TYAIH | 315 |
| | TYAIN | 316 |
| | TYAMH | 317 |
| | YYAMH | 318 |
| HCDR2 | IVSYDGNYKYYADSVKG | 319 |
| | VISYDANYKYYADSVKG | 320 |
| | VISYDANYKYYAQKFQG | 321 |
| | VISYDANYKYYAQKVQG | 322 |
| | VISYDGNYKYYADSVKG | 323 |
| | VISYDGNYKYYADSVQG | 324 |
| | VISYDGNYKYYAQKFQG | 325 |
| | VISYDGSNKYYADSVKG | 326 |
| | VISYDGSYKYYADSVKG | 327 |
| | VVCYDGNYKYYADSVQG | 328 |
| | WSFDGNYKYYADSVQG | 329 |
| | WSWDGNYKYYADSVQG | 330 |
| | VVSYDADYKYYADSVQG | 331 |
| | WSYDAEYKYYADSVQG | 332 |
| | WSYDAEYKYYADSVQG | 333 |
| | WSYDANYKYYADSVQG | 334 |
| | WSYDGFYKYYADSVQG | 335 |
| | WSYDGHYKYYADSVQG | 336 |
| | VVSYDGNCKYYADSVQG | 337 |
| | WSYDGNHKYYADSVQG | 338 |
| | VVSYDGNNKYYADSVQG | 339 |
| | WSYDGNYKYYADSVKG | 340 |
| | VVSYDGNYKYYADSVQG | 341 |
| | VVSYDGNYKYYADSVRG | 342 |
| | VVSYDGSYKYYADSVQG | 343 |
| | WSYDGYYKYYADSVQG | 344 |
| | VVSYDSNYKYYADSVKG | 345 |
| | VVSYNGNYKYYADSVQG | 346 |
| | VVSYSGNYKYYADSVQG | 347 |
| | WISYDANYKYYAQKFQG | 348 |
| HCDR3 | DSELRSLLYFEWLSQGYFNP | 349 |
| | DSQLRSLLMFEWLSQGYFDY | 350 |
| | DSQLRSLLYFEWLSQGYFDY | 351 |
| | DSQLRSLLYFEWLSQGYFNP | 352 |
| | DSQNRSLLMFEWLSQGYFDY | 353 |
| | DSQNRSLLYFEWLSQGYFDY | 354 |
| | DSRARSLLYFEWLSQGYFNP | 355 |
| | DSRDRSLLYFEWLSQGYFNP | 356 |
| | DSRHRSLLYFEWLSQGYFNP | 357 |
| | DSRIRSLLYFEWLSQGYFNP | 358 |
| | DSRKRSLLYFEWLSQGYFNP | 359 |
| | DSRLHSLLYFEWLSQGYFNP | 360 |
| | DSRLRLLLYFEWLSQGYFNP | 361 |
| | DSRLRSFLFFEWLSQGYFNP | 362 |
| | DSRLRSFLYFEWLSQGYFNP | 363 |
| | DSRLRSHLYFEWLSQGYFNP | 364 |
| | DSRLRSKLYFEWLSQGYFNP | 365 |
| | DSRLRSLCYFEWLSQGYFNP | 366 |
| | DSRLRSLHYFEWLSQGYFNP | 367 |
| | DSRLRSLLFFEWLSQGYFNP | 368 |
| | DSRLRSLLKFEWLSQGYFNP | 369 |
| | DSRLRSLLMFEWLSQGYFNP | 370 |
| | DSRLRSLLRFEWLSQGYFNP | 371 |
| | DSRLRSLLYCEWCSQGYFNP | 372 |
| | DSRLRSLLYFDWLSQGYFNP | 373 |
| | DSRLRSLLYFECLSQGYFNP | 374 |
| | DSRLRSLLYFEHLSQGYFNP | 375 |
| | DSRLRSLLYFERLSQGYFNP | 376 |
| | DSRLRSLLYFEWLSCGYFNP | 377 |
| | DSRLRSLLYFEWLSLGYFNP | 378 |
| | DSRLRSLLYFEWLSQGRFNP | 379 |
| | DSRLRSLLYFEWLSQGYFDD | 380 |
| | DSRLRSLLYFEWLSQGYFDF | 381 |
| | DSRLRSLLYFEWLSQGYFDI | 382 |
| | DSRLRSLLYFEWLSQGYFDL | 383 |
| | DSRLRSLLYFEWLSQGYFDP | 384 |
| | DSRLRSLLYFEWLSQGYFDV | 385 |
| | DSRLRSLLYFEWLSQGYFDY | 386 |
| | DSRLRSLLYFEWLSQGYFEY | 387 |
| | DSRLRSLLYFEWLSQGYFGV | 388 |
| | DSRLRSLLYFEWLSQGYFND | 389 |
| | DSRLRSLLYFEWLSQGYFNP | 390 |
| | DSRLRSLLYFEWLSQGYFQH | 391 |
| | DSRLRSLLYFEWLSQGYFQP | 392 |
| | DSRLRSLLYFEWLSQGYFSY | 393 |
| | DSRLRSLLYFEWLSQGYMDV | 394 |
| | DSRLRSLLYFEWLSQGYMGV | 395 |
| | DSRLRSLLYFEWMNQGYFNP | 396 |
| | DSRLRSLLYFEWNSQGYFNP | 397 |
| | DSRLRSLLYQEWLSQGYFNP | 398 |
| | DSRLRSLNYFEWLSQGYFNP | 399 |
| | DSRLRSLQYFEWLSQGYFNP | 400 |
| | DSRLRSLRYFEWLSGGYFNP | 401 |
| | DSRLRSNLYYEWLSQGYFNP | 402 |
| | DSRLRSQLYFEWLSQGYFNP | 403 |
| | DSRLRSQLYYEWLSQGYFNP | 404 |
| | DSRLRSRLYFEWLSQGYFNP | 405 |
| | DSRLYSLLYFEWLSQGYFNP | 406 |
| | DSRMRSLLYFEWLSQGYFNP | 407 |
| | DSRNHSLLYFEWLSQGYFNP | 408 |
| | DSRNRSLLMFEWLSQGYFNP | 409 |
| | DSRNRSLLMFEWNNQGYFNP | 410 |
| | DSRNRSLLYFEWLSQGYFNP | 411 |
| | DSRNRVLLYFEWLSQGYFNP | 412 |
| | DSRNYSLLYFEWLSQGYFNP | 413 |
| | DSRPRSLLYFEWLSQGYFNP | 414 |
| | DSRQRSLLMFEWLSQGYFNP | 415 |
| | DSRQRSLLYFEWLSQGYFNP | 416 |
| | DSRRRSLLYFEWLSQGYFNP | 417 |
| | DSRSRSLLYFEWLSQGYFNP | 418 |
| | DSRTRSLLYFEWLSQGYFNP | 419 |
| | DSRVRSLLYFEWLSQGYFNP | 420 |
| | DSRYRSLLYFEWLSQGYFNP | 421 |
| | ERPLRLLRYFDWLSGGANDY | 422 |
| | ESRLRSLLYFEWLSQGYFNP | 423 |

TABLE 16

Exemplary Light Chain CDRs (LCDRs)

| LCDR | Amino Acid Sequence | SEQ ID NO |
|---|---|---|
| LCDR1 | RSSQSITFDYKNYLA | 424 |
| | KSSQSVTFDYKNYLA | 425 |
| | RSSQSITFDYQNYLA | 426 |
| | RSSQSITSDNQNYLA | 427 |
| | KSSQSVLYSSNNKNYLA | 428 |
| | KSSQSVSFNYKNYLA | 429 |
| | KSSQSVTFNYKNYLA | 430 |
| | RSSQSITFDYKNRLA | 431 |
| | RSSQSITSDNKNYLA | 432 |
| | RSSQSITSDDKNYLA | 433 |
| | RSSQSITSDEKNYLA | 434 |
| | RSSQSITDDNKNYLA | 435 |
| | RSSQSITDDDKNYLA | 436 |
| | RSSQSITDDEKNYLA | 437 |
| | RSSQSITEDNKNYLA | 438 |
| | RSSQSITEDDKNYLA | 439 |
| | RSSQSITEDEKNYLA | 440 |
| | RSSQSITQDNKNYLA | 441 |
| | RSSQSITQDDKNYLA | 442 |
| | RSSQSITQDEKNYLA | 443 |
| | RSSQSITHDNKNYLA | 444 |
| | RSSQSITHDDKNYLA | 445 |
| | RSSQSITHDEKNYLA | 446 |
| | RSSQSITFNYKNYLA | 447 |
| | RSSQSVTFDYKNYLA | 448 |
| | RSSQSITSDYKNYLA | 449 |
| | KSSQSITFDYKNYLA | 450 |
| | KSSQSVTDDNKNYLA | 451 |
| | KSSQSVTSDYKNYLA | 452 |
| | KSSQSVTSDNKNYLA | 453 |
| | RSSESITFDYKNYLA | 454 |
| | RSSEDITFDYKNYLA | 455 |
| | RSSQSITFSSDYKNYLA | 456 |
| | RSSQSITFSPDYKNYLA | 457 |
| | RSSQSITLSPDYKNYLA | 458 |
| | RSSQSITISPDYKNYLA | 459 |
| | RSSQSITFGGDYKNYLA | 460 |
| | RSSQSITFSDYKNYLA | 461 |
| | RSSQSITFGDYKNYLA | 462 |
| | RSSQSITFGPDYKNYLA | 463 |
| | RSSQSITYDYKNYLA | 464 |
| | RSSQSITWDYKNYLA | 465 |
| | RSSQSITFWYKNYLA | 466 |
| | RSSQSITFLDYKNYLA | 467 |
| | RSSQSITLSPWYKNYLA | 468 |
| | RSSQSITLSPYYKNYLA | 469 |
| | RSSQSITLSPYDKNYLA | 470 |
| | RSSQSITFDNKNYLA | 471 |
| | RSSQSITADNKNYLA | 472 |
| | RSSQSITNDNKNYLA | 473 |
| | RSSQSITNDNKNYLA | 474 |
| | RSSQSITRDNKNYLA | 475 |
| | RSSQSITSDSKNYLA | 476 |
| | RSSQSITSDQKNYLA | 477 |
| | RSSQSITSDRKNYLA | 478 |
| | RSSEDITFWYKNYLA | 479 |
| | RSSQSITFWEKNYLA | 480 |
| | RSSQSITSWNKNYLA | 481 |
| | RSSEDITSWNKNYLA | 482 |
| | RSSEDITSWDKNYLA | 483 |
| | RSSQSITFYYKNYLA | 484 |
| | RSSQSITFRYKNYLA | 485 |
| | RSSQSITFRDKNYLA | 486 |
| | RSSQSITDRYKNYLA | 487 |
| | RSSESITFYYKNYLA | 488 |
| | KSSQSVTFWYKNYLA | 489 |
| | RSSEDITSDNQNYLA | 490 |
| | RSSEDITSDNKNYLA | 491 |
| | RSSQSITWDNKNYLA | 492 |
| LCDR2 | WGSYLES | 493 |
| | WGSSLQS | 494 |
| | AASSLQS | 495 |
| | WASTRES | 496 |
| | EASTRES | 497 |
| | WGSTRAT | 498 |
| | SASTRAT | 499 |
| | WGSTLES | 500 |
| | WASARES | 501 |
| | EGSYLES | 502 |
| | SGSYLES | 503 |
| | AASSRQS | 504 |
| | AASSLES | 505 |
| | SASSLQS | 506 |
| | SGSSLQS | 507 |
| | SASTRES | 508 |
| | WASTRQS | 509 |
| | SGSTRES | 510 |
| | WGSELES | 511 |
| | WGSDLES | 512 |
| | WGEYLES | 513 |
| | AASTRES | 514 |
| LCDR3 | QQHYRTPPS | 515 |
| | QQCYRTPPS | 516 |
| | QQHYCTPPS | 517 |
| | QQFYRTPPS | 518 |
| | QQYYRSPS | 519 |
| | QQYYRTPPS | 520 |
| | QQYYRSPPS | 521 |
| | QQHYRTPPT | 522 |
| | QQHYSTPPS | 523 |
| | QQHYQTPPS | 524 |
| | QQHYRTPS | 525 |

An anti-HA antibody molecule described herein can have any combination of the heavy chain variable region and the light chain variable region described above.

In another embodiment, the binding agent, e.g., an anti-HA antibody molecule, is a full-length tetrameric antibody, a single chain antibody (scFv), a F(ab′)₂ fragment, a Fab fragment, or an Fd fragment. In another embodiment, the heavy chain of the antibody molecule is a γ1 heavy chain, and in yet another embodiment, the light chain of the antibody molecule is a κ light chain or a λ light chain. In yet another embodiment, the anti-HA antibody molecule featured in the disclosure is an IgG1 antibody.

In an embodiment, the antibody molecule binds an epitope that has one, two, three, four, five, or all of, the following properties a)-f): a) it includes one, two, or all of, H3 HA1 residues N38, 1278, and D291; b) it includes H3 HA2 residue N12; c) it does not include one, two or all of, H3 HA1 residues Q327, T328, and R329; d) it does not include one, two, three, four, or all of, H3 HA2 residues G1, L2, F3, G4, and D46; e) it includes one, two, or all of, H3 HA1 residues T318, R321, and V323; or f) it includes 1, 2, 3, 4, 5, 6, 7, 8, 9, 10, 11, 12, 13, 14, 15, 16, 17, or all of, H3 HA2 residues A7, E11, 118, D19, G20, W21, L38, K39, T41, Q42, A43, 145, 148, N49, L52, N53, 156,and E57.

In an embodiment, the antibody molecule further binds an epitope that comprises one or more (2, 3, 4, 5, or all) of the following residues: Group 1 HA1 residues N41, D277, C278, T280, A288, or P290. In an embodiment, the antibody molecule further binds an epitope that comprises one or more (2, 3, 4, 5, or all) of the following residues: Group 2 HA1 residues T48, T276, C277, S279, S287, or P289

In an embodiment, the antibody molecule has properties: a) and b). In an embodiment, the antibody molecule has properties: c) and d). In an embodiment, the antibody molecule has properties: a); and c) or d). In an embodiment, the antibody molecule has properties: b); and c) or d). In an embodiment, the antibody molecule has properties: c); and a) or b). In an embodiment, the antibody molecule has properties: d); and a) or b). In an embodiment, the antibody molecule has properties: a), b), c) and d). In an embodiment, the antibody molecule has properties: a), b), c), d), e), and f).

In an embodiment, the antibody molecule has a K_{D} for H3 of equal to or less than 10⁻⁶, wherein said K_{D} is increased by at least 2, 5,10, or 100 fold, by a mutation or mutations in any of: a) H3 HA1 residues N38, 1278, or D291; b) H3 HA2 residue N12; c) H3 HA1 residues T318, R321, or V323; or d) H3 HA2 residues A7, E11, 118, D19, G20, W21, L38, K39, T41, Q42, A43, 145, 148, N49, L52, N53, 156, or E57. In an embodiment, the antibody molecule has a K_{D} for H3 of equal to or less than 10⁻⁶, wherein said K_{D} is increased by no more than 2, or 5 fold, by a mutation or mutations in any of: c) H3 HA1 residues Q327, T328, or R329; or d) H3 HA2 residues G1, L2, F3, G4, or D46.

In an embodiment, the antibody molecule binds an epitope that has one, two, three, four, five, or all of, the following properties aa)-ff): aa) it includes one, two, or all of, H1 HA1 residues H31, N279, and S292; bb) it includes H1 HA2 residue G12; cc) it does not include one or both of H1 HA1 residues Q328 and S329; dd) it does not include one, two, three, or all of, H1 HA2 residues G1, L2, F3, or G4; ee) it includes one, two, or all of, H1 HA1 residues T319, R322, and 1324 are bound by both Ab 044 and FI6; or ff) it includes 1, 2, 3, 4, 5, 6, 7, 8, 9, 10, 11, 12, 13, 14, 15, 16, 17, or all of, H1 HA2 residues A7, E11, 118, D19, G20, W21, Q38, K39, T41, Q42, N43, I45, I48, T49, V52, N53, 156, or E57. In an embodiment, the antibody molecule has properties: aa) and bb). In an embodiment, the antibody molecule has properties: cc) and dd). In an embodiment, the antibody molecule has properties: aa); and cc) or dd). In an embodiment, the antibody molecule has properties: bb); and cc) or dd). In an embodiment, the antibody molecule has properties: cc); and aa) or bb). In an embodiment, the antibody molecule has properties: dd); and aa) or bb). In an embodiment, the antibody molecule has properties: aa), bb), cc) and dd). In an embodiment, the antibody molecule has properties: aa), bb), cc), dd), ee), and ff).

In an embodiment, the antibody molecule has a K_{D} for H1 of equal to or less than 10⁻⁶, wherein said K_{D} is increased by at least 2, 5, 10, or 100 fold, by a mutation or mutations in any of: aa) H1 HA1 residues H31, N279, and S292; bb) H1 HA2 residue G12; cc) H1 HA1 residues T319, R322, and 1324; or dd) H1 HA2 residues A7, E11, 118, D19, G20, W21, Q38, K39, T41, Q42, N43, I45, I48, T49, V52, N53, 156, and E57. In an embodiment, the antibody molecule has a K_{D} for H1 of equal to or less than 10⁻⁶, wherein said K_{D} is increased by no more than 2, or 5-fold, by a mutation or mutations in any of: cc) H1 HA1 residues Q328 and S329; or dd) H1 HA2 residues G1, L2, F3, and G4. In an embodiment, the antibody molecule has one, two, three or all of the following properties: a) and aa); b) and bb); c) and cc); or d) and dd). In an embodiment, the molecule has properties c), cc), d), and dd).

In an embodiment, the binding agent, e.g., an antibody molecule, comprises one or both of: a heavy chain variable region comprising at least, or more than, 60, 65, 70, 75, 80, 85, 87, 90, 95, 98 or 99 percent homology with a heavy chain variable region described herein; and a light chain variable region comprising at least, or more than, 60, 65, 70, 75, 80, 85, 87, 90, 95, 98 or 99 percent homology with light chain variable region described herein.

In an embodiment, the antibody molecule comprises one or both of: a) one or more framework regions (FRs) from heavy chain disclosed herein. E.g., the antibody molecule comprises one or more (e.g., 2 or 3) or all of FR1, FR2, FR3, or FR4, or FR sequences that differ individually, or collectively, by no more than 1, 2, 3, 4, of 5 amino acid residues, e.g., conservative residues, from a heavy chain disclosed herein; and b) one or more framework regions (FRs) from light chain disclosed herein. E.g., the antibody molecule comprises one or more or all of FR1, FR2, FR3, or FR4, or FR sequences that differ individually, or collectively, by no more than 1, 2, 3, 4, of 5 amino acid residues, e.g., conservative residues, from light chain disclosed herein.

In one aspect, an anti-HA antibody molecule featured in the disclosure, or preparation, or isolated preparation thereof, comprises: (a) a heavy chain immunoglobulin variable domain comprising a sequence at least 60, 70, 80, 85, 87, 90, 95, 97, 98, or 99, e.g., 90%, homologous, to a heavy chain consensus sequence provided herein; and (b) a light chain immunoglobulin variable domain comprising a sequence at least 60, 70, 80, 85, 87, 90, 95, 97, 98, or 99, e.g., 95%, homologous, to a light chain consensus sequence provided herein.

In one embodiment, the 1, 2, 3, 4, 5, 6, 8, 10, 11, 12, 13, 14, 15 or 16 amino acid differences, e.g., conservative amino acid differences, in the heavy chain immunoglobulin variable region are in the FR regions of the heavy chain immunoglobulin variable domain. In another embodiment, the 1, 2, 3, 4 or 5 amino acid differences, e.g., conservative amino acid differences, in the light chain immunoglobulin variable domain are in the FR regions of the light chain immunoglobulin variable domain. In one embodiment, the amino acid differences in the heavy chain immunoglobulin variable region, or in the light chain immunoglobulin variable region, are conservative amino acid changes.

In an embodiment, the binding agent, e.g., an antibody molecule, binds to an epitope, e.g., it has an epitope that overlaps with or is the same as, of an antibody disclosed herein, e.g., as determined by mutational analysis or crystal structure analysis.

In an embodiment, the antibody molecule comprises one or both of: a) one or more framework regions (FRs) from heavy chain consensus sequence disclosed herein, e.g., the antibody molecule comprises one or more or all of FR1, FR2, FR3, or FR4, or sequences that differ individually, or collectively, by no more than 1, 2, 3, 4, of 5 amino acid residues, e.g., conservative residues, from heavy chain consensus sequence disclosed herein; and b) one or more framework regions (FRs) from light chain consensus sequence disclosed herein, e.g., the antibody molecule comprises one or more or all of FR1, FR2, FR3, or FR4, or sequences that differ individually, or collectively, by no more than 1, 2, 3, 4, of 5 amino acid residues, e.g., conservative residues, from light chain consensus disclosed herein. In an embodiment, the binding agent, e.g., an antibody molecule, specifically binds the HA antigen.

### Variants

In an embodiment, an antibody molecule, e.g., an antibody featured in the disclosure has a variable heavy chain immunoglobulin domain that is at least 85%, 87%, 88%, 89%, 90%, 92%, 94%, 95%, 96%, 97%, 98%, or 99% homologous, or at least 85%, 87%, 88%, 89%, 90%, 92%, 94%, 95%, 96%, 97%, 98%, or 99% identical, to a heavy chain disclosed herein, and has a variable light chain immunoglobulin domain that is at least 85%, 87%, 88%, 89%, 90%, 92%, 94%, 95%, 96%, 97%, 98%, or 99% homologous, or at least 85%, 87%, 88%, 89%, 90%, 92%, 94%, 95%, 96%, 97%, 98%, or 99% identical, to a light chain disclosed herein.

An exemplary anti-HA binding antibody has one or more CDRs, e.g., all three heavy chain (HC) CDRs and/or all three light chain (LC) CDRs of a particular antibody disclosed herein, or CDRs that are, in sum, at least 85%, 87%, 88%, 89%, 90%, 92%, 94%, 95%, 96%, 97%, 98%, or 99% homologous, or at least 85%, 87%, 88%, 89%, 90%, 92%, 94%, 95%, 96%, 97%, 98%, or 99% identical, to such an antibody. In one embodiment, the H1 and H2 hypervariable loops have the same canonical structure as those of an antibody described herein. In one embodiment, the L1 and L2 hypervariable loops have the same canonical structure as those of an antibody described herein.

In one embodiment, the amino acid sequence of the HC and/or LC variable domain sequence is at least 85%, 87%, 88%, 89%, 90%, 92%, 94%, 95%, 96%, 97%, 98%, or 99% homologous, or at least 85%, 87%, 88%, 89%, 90%, 92%, 94%, 95%, 96%, 97%, 98%, or 99% identical, to the amino acid sequence of the HC and/or LC variable domain of an antibody described herein. The amino acid sequence of the HC and/or LC variable domain sequence can differ by at least one amino acid, but no more than ten, eight, six, five, four, three, or two amino acids from the corresponding sequence of an antibody described herein. For example, the differences may be primarily or entirely in the framework regions.

In certain embodiments, the amino acid differences are conservative amino acid differences (e.g., conservative amino acid substitutions). A “conservative” amino acid substitution is one in which the amino acid residue is replaced with an amino acid residue comprising a similar side chain. Families of amino acid residues comprising similar side chains have been defined in the art. These families include, e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The amino acid sequences of the HC and LC variable domain sequences can be encoded by a nucleic acid sequence that hybridizes under high stringency conditions to a nucleic acid sequence described herein or one that encodes a variable domain or an amino acid sequence described herein. In one embodiment, the amino acid sequences of one or more (e.g., 2, 3, or 4) framework regions (e.g., FR1, FR2, FR3, and/or FR4) of the HC and/or LC variable domain are at least 85%, 87%, 88%, 89%, 90%, 92%, 94%, 95%, 96%, 97%, 98%, or 99% homologous, or at least 85%, 87%, 88%, 89%, 90%, 92%, 94%, 95%, 96%, 97%, 98%, or 99% identical, to corresponding framework regions of the HC and LC variable domains of an antibody described herein. In one embodiment, one or more heavy or light chain framework regions (e.g., HC FR1, FR2, FR3, and FR4, or LC FR1, FR2, FR3, and FR4) are at least 85%, 87%, 88%, 89%, 90%, 92%, 94%, 95%, 96%, 97%, 98%, or 99% homologous, or at least 85%, 87%, 88%, 89%, 90%, 92%, 94%, 95%, 96%, 97%, 98%, or 99% identical, to the sequence of corresponding framework regions from a human germline antibody.

### Validation of Epitopes

In one embodiment, the antibodies featured in the disclosure are useful for validating a vaccine based on a particular epitope. For example, an epitope that is the target of an antibody featured in the disclosure can be assessed by computation methods to identify a peptide framework suitable for supporting the epitope conformation, such as to stabilize an epitope that is transient or minimally accessible in nature. Computational abstraction of the epitope and framework properties allows automated screening of databases to identify candidate acceptor peptide scaffolds. The acceptor scaffold can have a particular tertiary structure that includes, for example, one or more of a beta sheet, a beta sandwich, a loop, or an alpha or beta helix. The candidate epitope-scaffold antigens can be assayed in vitro, such as to identify binding properties with an antibody featured in the disclosure, e.g., binding affinity or structure analysis of the epitope-scaffold/antibody complex, or in vitro neutralization. The ability of the epitope-scaffold to generate an immune response (e.g., to generate antibodies) can be tested by administering the epitope-scaffold to an animal (e.g., in a mammal, such as a rat, a mouse, a guinea pig, or a rabbit), and then testing sera for the presence of anti-epitope-scaffold antibodies, e.g., by ELISA assay. The ability of the epitope-scaffold to elicit protection against infection by an influenza A Group 1 or Group 2 strain, or by both types of influenza strains, can be assessed in vivo, such as in an animal (e.g., in a mammal). Thus, an antibody featured in the disclosure can provide validation that the epitope is functionally important and that targeting the epitope will provide protection from infection with a Group 1 or Group 2 influenza strain, or both types of strains.

### Production of Antibody Molecules

The nucleic acids (e.g., the genes) encoding an antibody molecule generated by a method described herein can be sequenced, and all or part of the nucleic acids can be cloned into a vector that expresses all or part of the nucleic acids. For example, the nucleic acids can include a fragment of the gene encoding the antibody, such as a single chain antibody (scFv), a F(ab′)₂ fragment, a Fab fragment, or an Fd fragment. The disclosure also provides host cells comprising the nucleic acids encoding an antibody or fragment thereof as described herein. The host cells can be, for example, prokaryotic or eukaryotic cells, e.g., mammalian cells, or yeast cells, e.g., Pichia (see, e.g., Powers et al. (2001) J. Immunol. Methods 251:123-35), Hanseula, or Saccharomyces.

Antibody molecules, particularly full-length antibody molecules, e.g., IgGs, can be produced in mammalian cells. Exemplary mammalian host cells for recombinant expression include Chinese Hamster Ovary (CHO) cells (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, K562, and a cell from a transgenic animal, e.g., a transgenic mammal. For example, the cell is a mammary epithelial cell. In addition to the nucleic acid sequence encoding the immunoglobulin domain, the recombinant expression vectors may carry additional nucleic acid sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216; 4,634,665; and 5,179,017). Exemplary selectable marker genes include the dihydrofolate reductase (DHFR) gene (for use in dhfr host cells with methotrexate selection/amplification) and the neo gene (for G418 selection).

In an exemplary system for recombinant expression of an antibody molecule (e.g., a full-length antibody or an antigen-binding portion thereof), a recombinant expression vector encoding both the antibody heavy chain and the antibody light chain is introduced into dhfr- CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody molecule is recovered from the culture medium. Standard molecular biology techniques are used to prepare the recombinant expression vector, to transfect the host cells, to select for transformants, to culture the host cells, and to recover the antibody from the culture medium. For example, some antibodies can be isolated by affinity chromatography with a Protein A or Protein G. For example, purified antibodies can be concentrated to about 100 mg/mL to about 200 mg/mL using protein concentration techniques that are known in the art.

Antibody molecules can also be produced by a transgenic animal. For example, U.S. Pat. No. 5,849,992 describes a method for expressing an antibody molecule in the mammary gland of a transgenic mammal. A transgene is constructed that includes a milk-specific promoter and nucleic acid sequences encoding the antibody molecule of interest, e.g., an antibody described herein, and a signal sequence for secretion. The milk produced by females of such transgenic mammals includes, secreted therein, the antibody of interest, e.g., an antibody described herein. The antibody molecule can be purified from the milk, or for some applications, used directly. Antibody molecules can also be expressed in vivo, following administration of a vector containing nucleic acids encoding the antibody heavy chain and the antibody light chain. Vector mediated gene-transfer is then used to engineer secretion of the anti-HA antibody into circulation. For example, an anti-HA antibody heavy chain and an anti-HA antibody light chain as described herein are cloned into an adeno-associated virus (AAV)-based vector, and each of the anti-HA antibody heavy chain and the anti-HA antibody light chain are under control of a promoter, such as a cytomegalovirus (CMV) promoter. Administration of the vector to a subject, such as to a patient, e.g., a human patient, such as by intramuscular injection, results in expression of an anti-HA antibody, and secretion into the circulation.

### Modifications of Binding Agents

Binding, agents, e.g., antibody molecules can be modified to have numerous properties, e.g., to have altered, e.g., extended half-life, to be associated with, e.g., covalently bound to detectable moieties, e.g., labels, to be associated with, e.g., covalently bound to toxins, or to have other properties, e.g., altered immune functions. Antibody molecules may include modifications, e.g., modifications that alter Fc function, e.g., to decrease or remove interaction with an Fc receptor or with C1q, or both. In one example, the human IgG1 constant region can be mutated at one or more residues.

For some antibody molecules that include an Fc domain, the antibody production system may be designed to synthesize antibody molecules in which the Fc region is glycosylated. The Fc domain can be produced in a mammalian expression system that appropriately glycosylates the residue corresponding to asparagine 297. The Fc domain can also include other eukaryotic post-translational modifications. Other suitable Fc domain modifications include those described in WO2004/029207. For example, the Fc domain can be an XmAb® Fc (Xencor, Monrovia, CA). The Fc domain, or a fragment thereof, can have a substitution in an Fcy Receptor (FcyR) binding region, such as the domains and fragments described in WO05/063815. In some embodiments, the Fc domain, or a fragment thereof, has a substitution in a neonatal Fc Receptor (FcRn) binding region, such as the domains and fragments described in WO05047327. In other embodiments, the Fc domain is a single chain, or fragment thereof, or modified version thereof, such as those described in WO2008143954. Other suitable Fc modifications are known and described in the art.

Antibody molecules can be modified, e.g., with a moiety that improves its stabilization and/or retention in circulation, e.g., in blood, serum, lymph, bronchoalveolar lavage, or other tissues, e.g., by at least 1.5, 2, 5, 10, or 50-fold. For example, an antibody molecule generated by a method described herein can be associated with a polymer, e.g., a substantially non-antigenic polymer, such as a polyalkylene oxide or a polyethylene oxide. Suitable polymers will vary substantially by weight. Polymers comprising molecular number average weights ranging from about 200 to about 35,000 Daltons (or about 1,000 to about 15,000, and 2,000 to about 12,500) can be used.

For example, an antibody molecule generated by a method described herein can be conjugated to a water-soluble polymer, e.g., a hydrophilic polyvinyl polymer, e.g. polyvinylalcohol or polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides that comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparan.

Binding agents, e.g., antibody molecules, as disclosed herein, can by conjugated to another entity or moiety (e.g., to a cytotoxic or cytostatic moiety, a label or detectable moiety, or a therapeutic moiety). Exemplary moieties include: a cytotoxic or cytostatic agent, e.g., a therapeutic agent, a drug, a compound emitting radiation, molecules of plant, fungal, or bacterial origin, or a biological protein (e.g., a protein toxin) or particle (e.g., a recombinant viral particle, e.g., via a viral coat protein), a detectable agent; a pharmaceutical agent, and/or a protein or peptide that can mediate association of the antibody or antibody portion with another molecule (such as a streptavidin core region or a polyhistidine tag). A binding agent, e.g., an antibody molecule, as disclosed herein, can be functionally linked by any suitable method (e.g., chemical coupling, genetic fusion, covalent binding, noncovalent association or otherwise) to one or more other molecular entities.

Binding agents, e.g., antibody molecules, disclosed herein can be conjugated with a detectable moiety, e.g., a label or imaging agent. Such moieties can include enzymes (e.g., horseradish peroxidase, beta-galactosidase, luciferase, alkaline phosphatase, acetylcholinesterase, glucose oxidase and the like), radiolabels (e.g., ³H, ¹⁴C, ¹⁵N, ³⁵S, ⁹⁰Y, ⁹⁹Tc, ¹¹¹In, ¹²⁵I,¹³¹I and the like), haptens, fluorescent labels (e.g., FITC, rhodamine, lanthanide phosphors, fluorescein, fluorescein isothiocyanate, rhodamine, 5-dimethylamine-1-napthalenesulfonyl chloride, phycoerythrin and the like), phosphorescent molecules, chemiluminescent molecules, chromophores, luminescent molecules, photoaffinity molecules, colored particles or affinity ligands, such as biotin, predetermined polypeptide epitopes recognized by a secondary reporter (e.g., leucine zipper pair sequences, or binding sites for secondary antibodies, metal binding domains, epitope tags). In some embodiments, a moiety, e.g., a detectable moiety, e.g., a label, is attached by spacer arms of various lengths to reduce potential steric hindrance.

In some embodiments, a binding agent, e.g., antibody molecule, disclosed herein, is derivatized with a detectable enzyme and is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. A binding agent, e.g., antibody molecule, disclosed herein, may also be derivatized with a prosthetic group (e.g., streptavidin/biotin and avidin/biotin). For example, an antibody may be derivatized with biotin, and detected through indirect measurement of avidin or streptavidin binding.

In some embodiments, the moiety comprises paramagnetic ions and NMR-detectable substances, among others. For example, in some embodiments, a paramagnetic ion is one or more of chromium (III), manganese (II), iron (III), iron (II), cobalt (II), nickel (II), copper (II),neodymium (III), samarium (III), ytterbium (III), gadolinium (III), vanadium (II), terbium (III), dysprosium (III), holmium (III), erbium (III), lanthanum (III), gold (III), lead (II), and/or bismuth (III). Binding agents, e.g., antibody molecules, as disclosed herein, can be modified to be associated with, e.g., conjugated to, a therapeutic agent, e.g., an agent comprising anti-viral activity, anti-inflammatory activity, or cytotoxic activity, etc. In some embodiments, therapeutic agents can treat symptoms or causes of influenza infection (e.g., for example, anti-viral, pain-relief, anti-inflammatory, immunomodulatory, sleep-inducing activities, etc.).

### Fc Region

The present disclosure provides binding agents, and in particular antibody molecules (e.g., anti-hemagglutinin (HA) antibody molecules) comprising an Fc region or a fragment thereof, e.g., an Fc region, or a fragment thereof (e.g., a functional fragment thereof), described herein.

In an embodiment, the binding agent, particularly the antibody molecule, e.g., the anti-hemagglutinin (HA) antibody described herein comprises an Fc region described herein. In an embodiment, the anti-HA antibody described herein comprises an Fc region described herein. In an embodiment, the Fc region comprises one or more mutations described herein.

A fragment crystallizable region, or Fc region, refers to a region of an immunoglobulin that interacts with an Fc receptor. In an embodiment, the Fc region interacts with a protein of the complement system. Without wishing to be bound by theory, it is believed that in an embodiment, the interaction between the Fc region with an Fc receptor, allows for activation of the immune system.

In IgG, IgA and IgD antibody isotypes, the naturally-occurring Fc region generally comprises two identical protein fragments, derived from the second and third constant domains of the antibody’s two heavy chains. Naturally-occurring IgM and IgE Fc regions generally comprise three heavy chain constant domains (C_{H} domains 2-4) in each polypeptide chain. The Fc regions of IgGs can contain a highly conserved N-glycosylation site (Stadlmann et al. (2008). Proteomics 8 (14): 2858-2871; Stadlmann (2009) Proteomics 9 (17): 4143-4153). While not wishing to be bound by theory, it is believed that in an embodiment, glycosylation of the Fc fragment contributes to Fc receptor-mediated activities (Peipp et al. (2008) Blood 112 (6): 2390-2399). In an embodiment, the N-glycans attached to this site are predominantly core-fucosylated diantennary structures of the complex type. In another embodiment, small amounts of these N-glycans also contain bisecting GlcNAc and/or α-2,6 linked sialic acid residues.

An exemplary fragment of an Fc region amino acid sequence from human IgG1 is provided in SEQ ID NO: 40 and is shown below:

DKTHTCPPCPAPELLGGPSVFLFPPKPKDTLMISRTPEVTCVVVDVSHED
PEVKFNWYVDGVEVHNAKTKPREEQYGSTYRVVSVLTVLH QDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALH NH YTQKSLSLSPGK (SEQ ID NO: 40)

In SEQ ID NO: 40, the first amino acid residue in this sequence is referred to as position 221 herein. The three histidine residues shown in bold and underlined are positions 310, 433, and 435, respectively.

A binding agent, e.g., an antibody, e.g., an anti-hemagglutinin (HA) antibody comprising an Fc region or fragment thereof described herein can have one or more (e.g., 2, 3, 4, 5, 6, 7, 8, 9, 10, 11, 12, 13, 14, 15, 16, 17, 18, 19, 20, 21, 22, 23, 24, 25, or more) of mutations or combinations of mutations described in Table 2 (based on EU numbering, e.g., as described in www.imgt.org/IMGTScientificChart/Numbering/Hu_IGHGnber.html).

TABLE 2

Exemplary Fc mutations

| | |
|---|---|
| Name | Mutation |
| FcMut001 | I253M |
| FcMut002 | L309H_D312A_N315D |
| FcMut003 | L309N |
| FcMut004 | M252E_S254R |
| FcMut005 | M252E_S254R_R255Y |
| FcMut006 | S254H |
| FcMut007 | S254M |
| FcMut008 | T256D_T307R |
| FcMut009 | T256L_N286I_T307I |
| FcMut010 | T256I_N286I_T307I |
| FcMut011 | K248S_D376Q |
| FcMut012 | K248S_D376N |
| FcMut013 | D376Q_E380A |
| FcMut014 | D376N_E380A |
| FcMut015 | D376Q_M428L |
| FcMut016 | K248S_A378I |
| FcMut017 | L314K |
| FcMut018 | T250Q_M428L |
| FcMut019 | M428L_N434A |
| FcMut020 | N434A |
| FcMut021 | T307A_E380A_N434A |
| FcMut022 | M252W |
| FcMut023 | V308F |
| FcMut024 | V308F_N434Y |
| FcMut026 | T256D_T307R_D376N |
| FcMut027 | L309R_D312E |
| FcMut028 | L309R_Q311P_D312E |
| FcMut029 | K246N_P247A |
| FcMut030 | K246N_P247A_D376N |
| FcMut031 | T256E_T307R |
| FcMut032 | T256R_T307D |
| FcMut033 | T256R_T307E |
| FcMut034 | Q311P |
| FcMut035 | D376Q |
| FcMut036 | L234A_L235A |
| FcMut037 | L235V_G236A |
| FcMut038 | L234P_L235P |
| FcMut039 | L235P |
| FcMut040 | P329G |
| FcMut041 | P329E |
| FcMut042 | E233K |
| FcMut043 | T256D_N286D_A287S_T307R |
| FcMut044 | T256D_P257L_T307R |
| FcMut045 | T256D_T307R_Q311V |
| FcMut046 | P247D_T256D_T307R |
| FcMut047 | P247D_N286D_A287S_Q311V |
| FcMut048 | P257M_V308N |
| FcMut049 | V279I_Q311L_N315T |
| FcMut050 | M428L_N434S |
| FcMut051 | N434S |
| FcMut052 | H433G_N434P |
| FcMut053 | V259I_V308F_M428L |
| FcMut067 | T256D_N286D_T307R |
| FcMut068 | T256D_N286E_T307R |
| FcMut069 | T256D_N286Q_T307R |
| FcMut070 | T256D_P257T_T307R |
| FcMut071 | T256D_P257V_T307R |
| FcMut072 | T256D_T307R_Q311I |
| FcMut073 | T256D_T307R_Q311L |
| FcMut074 | T256D_T307R_Q311M |
| FcMut075 | T256D_P257L_N286D_T307R_Q311V |
| FcMut076 | T256D_T307R_M428L |
| FcMut077 | M428L |
| FcMut078 | M252Y_S254T_T256Q |
| FcMut079 | M252Y_S254T_T256E_K288E |
| FcMut080 | T256K_K288E |
| FcMut081 | T256D_E258T |
| FcMut082 | E283Q_H285E |
| FcMut083 | R344D_D401R |
| FcMut084 | K248E_E380K |
| FcMut085 | K248E_E380R |
| FcMut086 | K246H |
| FcMut087 | K248H |
| FcMut088 | T250I |
| FcMut089 | T250V |
| FcMut090 | L251F |
| FcMut091 | L251M |
| FcMut093 | P257V |
| FcMut094 | N276D |
| FcMut095 | H285N |
| FcMut096 | H285D |
| FcMut097 | K288H |
| FcMut098 | K288Q |
| FcMut099 | K288E |
| FcMut100 | T307E |
| FcMut101 | T307Q |
| FcMut102 | V308P |
| FcMut103 | V308I |
| FcMut104 | V308L |
| FcMut105 | L309H |
| FcMut106 | L309M |
| FcMut107 | Q311H |
| FcMut108 | L314F |
| FcMut109 | Y319H |
| FcMut110 | I336T |
| FcMut111 | P343D |
| FcMut112 | P343V |
| FcMut113 | E345Q |
| FcMut114 | P346V |
| FcMut115 | P374T |
| FcMut116 | D376N |
| FcMut117 | A378S |
| FcMut118 | A431T |
| FcMut119 | A431P |
| FcMut120 | A431G |
| FcMut121 | L432V |
| FcMut122 | L432I |
| FcMut123 | L432Q |
| FcMut124 | N434T |
| FcMut125 | H435N |
| FcMut126 | Y436H |
| FcMut127 | K439Q |
| FcMut128 | T256D |
| FcMut129 | T307R |
| FcMut130 | A378T |
| FcMut131 | A378D |
| FcMut132 | A378H |
| FcMut133 | A378Y |
| FcMut134 | A378V |
| FcMut135 | D376R |
| FcMut136 | D376F |
| FcMut137 | D376W |
| FcMut138 | L314H |
| FcMut139 | L432E_T437Q |
| FcMut140 | D376Q_A378T |
| FcMut141 | D376Q_I377M_A378T |
| FcMut142 | P244Q_D376Q |
| FcMut143 | P247T_A378T |
| FcMut144 | P247N_A378T |
| FcMut145 | T256D_T307R_L309T |
| FcMut146 | A339T_S375E_F404Y |
| FcMut147 | L235V_G236A_T256D_T307R |
| FcMut148 | L235V_G236A_D376Q_M428L |
| FcMut149 | L314N |
| FcMut150 | N315D |
| FcMut151 | A378T |
| FcMut152 | T437Q |
| FcMut153 | L432E |
| FcMut154 | Y436R |
| FcMut155 | L314M |
| FcMut156 | L234A_L235A_T256D_T307R_Q311V |
| FcMut157 | L234A_L235A_T256D_P257V_T307R |
| FcMut158 | L234A_L235A_T256D_P257L_N286D_T307R_Q311V |
| FcMut159 | L235V_G236A_T256D_T307R_Q311V |
| FcMut160 | L235V_G236A_T256D_P257V_T307R |
| FcMut161 | L235V_G236A_T256D_P257L_N286D_T307R_Q311V |
| FcMut162 | S267T_A327N_A330M |
| FcMut163 | S267T_A327N |
| FcMut164 | L235V_G236A_S267T_A327N_A330M |
| FcMut165 | L235V_G236A_S267T_A327N |
| FcMut166 | M252Y_S254T |
| FcMut167 | T256E |
| FcMut168 | G236A_1332E |
| FcMut169 | S239D_1332E |
| FcMut170 | G236A_S239D_I332E |
| FcMut171 | T256D_N286D_T307R_Q311V |
| FcMut172 | T256D_E258T_T307R |
| FcMut173 | T256D_E258T_T307R_Q311V |
| FcMut174 | T256D_P257V_E258T_T307R |
| FcMut175 | T256D_P257L_E258T_N286D_T307R_Q311V |
| FcMut176 | T256D_E258T_N286D_T307R_Q311V |
| FcMut177 | A378V_M428L |
| FcMut178 | A378V_M428I |
| FcMut179 | A378V_M428V |
| FcMut180 | T256D_N286D |
| FcMut181 | T256D_A378V |
| FcMut182 | T256D_Q311V |
| FcMut183 | T256D_Q311V_A378V |
| FcMut184 | T256D_T307R_A378V |
| FcMut185 | T256D_N286D_T307R_A378V |
| FcMut186 | T256D_T307R_Q311V_A378V |
| FcMut187 | H285D_A378V |
| FcMut188 | H285D_Q311V |
| FcMut189 | T256D_H285D |
| FcMut190 | T256D_H285D_Q311V |
| FcMut191 | T256D_H285D_T307R |
| FcMut192 | T256D_H285D_T307R_A378V |
| FcMut193 | H285D_L314M_A378V |
| FcMut194 | T256D_E258T_H285D_Q311H |
| FcMut195 | T256D_E258T_H285D |
| FcMut196 | H285D_N315D |
| FcMut197 | H285N_T307Q_N315D |
| FcMut198 | H285D_L432E_T437Q |
| FcMut199 | T256D_E258T_N315D |
| FcMut200 | P257V_H285N |
| FcMut201 | H285N_L432F |
| FcMut202 | H285N_T437I |
| FcMut203 | T256D_E258T_L314M |
| FcMut204 | T256D_E258T_T307Q |
| FcMut205 | T256D_E258T_A378V |
| FcMut206 | V308P_A378V |
| FcMut207 | P257V_A378T |
| FcMut208 | P257V_V308P_A378V |
| FcMut209 | N315D_A378T |
| FcMut210 | H285N_L314M |
| FcMut211 | L314M_L432E_T437Q |
| FcMut212 | T307Q_N315D |
| FcMut213 | H285D_T307Q_A378V |
| FcMut214 | L314M_N315D |
| FcMut215 | T307Q_Q311V_A378V |
| FcMut216 | H285D_Q311V_A378V |
| FcMut217 | Q311V_N315D_A378V |
| FcMut218 | T256D_E258T_Q311V |
| FcMut219 | T256D_N315D_A378V |
| FcMut220 | T256D_Q311V_N315D |
| FcMut221 | T256D_T307Q_A378V |
| FcMut222 | T256D_T307Q_Q311V |
| FcMut223 | T256D_H285D_A378V |
| FcMut224 | T256D_H285D_T307R_Q311V |
| FcMut225 | T256D_H285D_N286D_T307R |
| FcMut226 | T256D_H285D_N286D_T307R_Q311V |
| FcMut227 | T256D_H285D_N286D_T307R_A378V |
| FcMut228 | T256D_N286D_T307R_Q311V_A378V |
| FcMut229 | T256D_H285D_T307R_Q311V_A378V |
| FcMut230 | V308P_Q311V_A378V |
| FcMut231 | T256D_V308P_A378V |
| FcMut232 | T256D_V308P_Q311V |
| FcMut233 | T256D_E258T_V308P |
| FcMut234 | H285D_V308P_Q311V |
| FcMut242 | E258T |
| FcMut243 | N286D |
| FcMut244 | Q311V |
| YTE | M252Y_S254T_T256E |

In an embodiment, the Fc region comprises FcMut001. In an embodiment, the Fc region comprises FcMut002. In an embodiment, the Fc region comprises FcMut003. In an embodiment, the Fc region comprises FcMut004. In an embodiment, the Fc region comprises FcMut005. In an embodiment, the Fc region comprises FcMut006. In an embodiment, the Fc region comprises FcMut007. In an embodiment, the Fc region comprises FcMut008. In an embodiment, the Fc region comprises FcMut009. In an embodiment, the Fc region comprises FcMut010. In an embodiment, the Fc region comprises FcMut011. In an embodiment, the Fc region comprises FcMut012. In an embodiment, the Fc region comprises FcMut013. In an embodiment, the Fc region comprises FcMut014. In an embodiment, the Fc region comprises FcMut015. In an embodiment, the Fc region comprises FcMut016. In an embodiment, the Fc region comprises FcMut017. In an embodiment, the Fc region comprises FcMut018. In an embodiment, the Fc region comprises FcMut019. In an embodiment, the Fc region comprises FcMut020. In an embodiment, the Fc region comprises FcMut021. In an embodiment, the Fc region comprises FcMut022. In an embodiment, the Fc region comprises FcMut023. In an embodiment, the Fc region comprises FcMut024. In an embodiment, the Fc region comprises FcMut026. In an embodiment, the Fc region comprises FcMut027. In an embodiment, the Fc region comprises FcMut028. In an embodiment, the Fc region comprises FcMut029. In an embodiment, the Fc region comprises FcMut030. In an embodiment, the Fc region comprises FcMut031. In an embodiment, the Fc region comprises FcMut032. In an embodiment, the Fc region comprises FcMut033. In an embodiment, the Fc region comprises FcMut034. In an embodiment, the Fc region comprises FcMut035. In an embodiment, the Fc region comprises FcMut036. In an embodiment, the Fc region comprises FcMut037. In an embodiment, the Fc region comprises FcMut038. In an embodiment, the Fc region comprises FcMut039. In an embodiment, the Fc region comprises FcMut040. In an embodiment, the Fc region comprises FcMut041. In an embodiment, the Fc region comprises FcMut042. In an embodiment, the Fc region comprises FcMut043. In an embodiment, the Fc region comprises FcMut044. In an embodiment, the Fc region comprises FcMut045. In an embodiment, the Fc region comprises FcMut046. In an embodiment, the Fc region comprises FcMut047. In an embodiment, the Fc region comprises FcMut048. In an embodiment, the Fc region comprises FcMut049. In an embodiment, the Fc region comprises FcMut050. In an embodiment, the Fc region comprises FcMut051. In an embodiment, the Fc region comprises FcMut052. In an embodiment, the Fc region comprises FcMut053. In an embodiment, the Fc region comprises FcMut067. In an embodiment, the Fc region comprises FcMut068. In an embodiment, the Fc region comprises FcMut069. In an embodiment, the Fc region comprises FcMut070. In an embodiment, the Fc region comprises FcMut071. In an embodiment, the Fc region comprises FcMut072. In an embodiment, the Fc region comprises FcMut073. In an embodiment, the Fc region comprises FcMut074. In an embodiment, the Fc region comprises FcMut075. In an embodiment, the Fc region comprises FcMut076. In an embodiment, the Fc region comprises FcMut077. In an embodiment, the Fc region comprises FcMut078. In an embodiment, the Fc region comprises FcMut079. In an embodiment, the Fc region comprises FcMut080. In an embodiment, the Fc region comprises FcMut081. In an embodiment, the Fc region comprises FcMut082. In an embodiment, the Fc region comprises FcMut083. In an embodiment, the Fc region comprises FcMut084. In an embodiment, the Fc region comprises FcMut085. In an embodiment, the Fc region comprises FcMut086. In an embodiment, the Fc region comprises FcMut087. In an embodiment, the Fc region comprises FcMut088. In an embodiment, the Fc region comprises FcMut089. In an embodiment, the Fc region comprises FcMut090. In an embodiment, the Fc region comprises FcMut091. In an embodiment, the Fc region comprises FcMut093. In an embodiment, the Fc region comprises FcMut094. In an embodiment, the Fc region comprises FcMut095. In an embodiment, the Fc region comprises FcMut096. In an embodiment, the Fc region comprises FcMut097. In an embodiment, the Fc region comprises FcMut098. In an embodiment, the Fc region comprises FcMut099. In an embodiment, the Fc region comprises FcMut100. In an embodiment, the Fc region comprises FcMut101. In an embodiment, the Fc region comprises FcMut102. In an embodiment, the Fc region comprises FcMut103. In an embodiment, the Fc region comprises FcMut104. In an embodiment, the Fc region comprises FcMut105. In an embodiment, the Fc region comprises FcMut106. In an embodiment, the Fc region comprises FcMut107. In an embodiment, the Fc region comprises FcMut108. In an embodiment, the Fc region comprises FcMut109. In an embodiment, the Fc region comprises FcMut110. In an embodiment, the Fc region comprises FcMut111. In an embodiment, the Fc region comprises FcMut112. In an embodiment, the Fc region comprises FcMut113. In an embodiment, the Fc region comprises FcMut114. In an embodiment, the Fc region comprises FcMut115. In an embodiment, the Fc region comprises FcMut116. In an embodiment, the Fc region comprises FcMut117. In an embodiment, the Fc region comprises FcMut118. In an embodiment, the Fc region comprises FcMut119. In an embodiment, the Fc region comprises FcMut120. In an embodiment, the Fc region comprises FcMut121. In an embodiment, the Fc region comprises FcMut122. In an embodiment, the Fc region comprises FcMut123. In an embodiment, the Fc region comprises FcMut124. In an embodiment, the Fc region comprises FcMut125. In an embodiment, the Fc region comprises FcMut126. In an embodiment, the Fc region comprises FcMut127. In an embodiment, the Fc region comprises FcMut128. In an embodiment, the Fc region comprises FcMut129. In an embodiment, the Fc region comprises FcMut130. In an embodiment, the Fc region comprises FcMut131. In an embodiment, the Fc region comprises FcMut132. In an embodiment, the Fc region comprises FcMut133. In an embodiment, the Fc region comprises FcMut134. In an embodiment, the Fc region comprises FcMut135. In an embodiment, the Fc region comprises FcMut136. In an embodiment, the Fc region comprises FcMut137. In an embodiment, the Fc region comprises FcMut138. In an embodiment, the Fc region comprises FcMut139. In an embodiment, the Fc region comprises FcMut140. In an embodiment, the Fc region comprises FcMut141. In an embodiment, the Fc region comprises FcMut142. In an embodiment, the Fc region comprises FcMut143. In an embodiment, the Fc region comprises FcMut144. In an embodiment, the Fc region comprises FcMut145. In an embodiment, the Fc region comprises FcMut146. In an embodiment, the Fc region comprises FcMut147. In an embodiment, the Fc region comprises FcMut148. In an embodiment, the Fc region comprises FcMut149. In an embodiment, the Fc region comprises FcMut150. In an embodiment, the Fc region comprises FcMut151. In an embodiment, the Fc region comprises FcMut152. In an embodiment, the Fc region comprises FcMut153. In an embodiment, the Fc region comprises FcMut154. In an embodiment, the Fc region comprises FcMut155. In an embodiment, the Fc region comprises FcMut156. In an embodiment, the Fc region comprises FcMut157. In an embodiment, the Fc region comprises FcMut158. In an embodiment, the Fc region comprises FcMut159. In an embodiment, the Fc region comprises FcMut160. In an embodiment, the Fc region comprises FcMut161. In an embodiment, the Fc region comprises FcMut162. In an embodiment, the Fc region comprises FcMut163. In an embodiment, the Fc region comprises FcMut164. In an embodiment, the Fc region comprises FcMut165. In an embodiment, the Fc region comprises FcMut166. In an embodiment, the Fc region comprises FcMut167. In an embodiment, the Fc region comprises FcMut168. In an embodiment, the Fc region comprises FcMut169. In an embodiment, the Fc region comprises FcMut170. In an embodiment, the Fc region comprises FcMut171. In an embodiment, the Fc region comprises FcMut172. In an embodiment, the Fc region comprises FcMut173. In an embodiment, the Fc region comprises FcMut174. In an embodiment, the Fc region comprises FcMut175. In an embodiment, the Fc region comprises FcMut176. In an embodiment, the Fc region comprises FcMut177. In an embodiment, the Fc region comprises FcMut178. In an embodiment, the Fc region comprises FcMut179. In an embodiment, the Fc region comprises FcMut180. In an embodiment, the Fc region comprises FcMut181. In an embodiment, the Fc region comprises FcMut182. In an embodiment, the Fc region comprises FcMut183. In an embodiment, the Fc region comprises FcMut184. In an embodiment, the Fc region comprises FcMut185. In an embodiment, the Fc region comprises FcMut186. In an embodiment, the Fc region comprises FcMut187. In an embodiment, the Fc region comprises FcMut188. In an embodiment, the Fc region comprises FcMut189. In an embodiment, the Fc region comprises FcMut190. In an embodiment, the Fc region comprises FcMut191. In an embodiment, the Fc region comprises FcMut192. In an embodiment, the Fc region comprises FcMut193. In an embodiment, the Fc region comprises FcMut194. In an embodiment, the Fc region comprises FcMut195. In an embodiment, the Fc region comprises FcMut196. In an embodiment, the Fc region comprises FcMut197. In an embodiment, the Fc region comprises FcMut198. In an embodiment, the Fc region comprises FcMut199. In an embodiment, the Fc region comprises FcMut200. In an embodiment, the Fc region comprises FcMut201. In an embodiment, the Fc region comprises FcMut202. In an embodiment, the Fc region comprises FcMut203. In an embodiment, the Fc region comprises FcMut204. In an embodiment, the Fc region comprises FcMut205. In an embodiment, the Fc region comprises FcMut206. In an embodiment, the Fc region comprises FcMut207. In an embodiment, the Fc region comprises FcMut208. In an embodiment, the Fc region comprises FcMut209. In an embodiment, the Fc region comprises FcMut210. In an embodiment, the Fc region comprises FcMut211. In an embodiment, the Fc region comprises FcMut212. In an embodiment, the Fc region comprises FcMut213. In an embodiment, the Fc region comprises FcMut214. In an embodiment, the Fc region comprises FcMut215. In an embodiment, the Fc region comprises FcMut216. In an embodiment, the Fc region comprises FcMut217. In an embodiment, the Fc region comprises FcMut218. In an embodiment, the Fc region comprises FcMut219. In an embodiment, the Fc region comprises FcMut220. In an embodiment, the Fc region comprises FcMut221. In an embodiment, the Fc region comprises FcMut222. In an embodiment, the Fc region comprises FcMut223. In an embodiment, the Fc region comprises FcMut224. In an embodiment, the Fc region comprises FcMut225. In an embodiment, the Fc region comprises FcMut226. In an embodiment, the Fc region comprises FcMut227. In an embodiment, the Fc region comprises FcMut228. In an embodiment, the Fc region comprises FcMut229. In an embodiment, the Fc region comprises FcMut230. In an embodiment, the Fc region comprises FcMut231. In an embodiment, the Fc region comprises FcMut232. In an embodiment, the Fc region comprises FcMut233. In an embodiment, the Fc region comprises FcMut234. In an embodiment, the Fc region comprises FcMut242. In an embodiment, the Fc region comprises FcMut243. In an embodiment, the Fc region comprises FcMut244.

In an embodiment, the Fc region comprises one or more (e.g., 2, 3, 4, 5, 6, 7, 8, 9, or more) of mutations or combinations of mutations chosen from FcMut045, FcMut171, FcMut183, FcMut186, FcMut190, FcMut197, FcMut213, FcMut215, FcMut216, FcMut219, FcMut222, FcMut223, FcMut224, FcMut226, FcMut227, FcMut228, or FcMut229. In an embodiment, the Fc region comprises one or more (e.g., 2, 3, 4, 5, 6, or all) of mutations or combinations of mutations chosen from FcMut045, FcMut183, FcMut197, FcMut213, FcMut215, FcMut228, or FcMut156. In another embodiment, the Fc region comprises one or more (e.g., 2, 3, 4, 5, or all) of mutations or combinations of mutations chosen from FcMut183, FcMut197, FcMut213, FcMut215, FcMut228, or FcMut229.

In an embodiment, the Fc region does not comprise one or more (e.g., 2, 3, 4, or all) of mutations or combinations of mutations chosen from FcMut018, FcMut021, FcMut050, FcMut102, or YTE. In an embodiment, the Fc region comprises one or more (e.g., 2, 3, 4, or all) of mutations or combinations of mutations chosen from FcMut018, FcMut021, FcMut050, FcMut102, or YTE, and one or more other mutations or combinations of mutations described in Table 2.

In an embodiment, the Fc region comprises one or more (e.g., 2, 3, 4, 5, 6, 7, 8, 9, 10, 11, 12, 13, 14, 15, 16, 17, 18, 19, 20, or more) of mutations or combinations of mutations described in Table 2 that result in a synergistic effect (e.g., binding affinity or circulating half-life) as described herein.

In an embodiment, the Fc region comprises one or more (e.g., 2, 3, 4, 5, 6, or 7) mutations in residues chosen from T256, H285, N286, T307, Q311, N315, or A378. In an embodiment, the Fc region comprises one or more (e.g., 2, 3, 4, 5, 6, or 7) mutations chosen from T256D, H285N, N286D, T307Q, Q311V, N315D, or A378V. In an embodiment, the Fc region comprises mutations in the residues T307, Q311, and A378. In an embodiment, the Fc region comprises the mutations T307Q, Q311V, and A378V.

In an embodiment, the Fc region comprises a half-life enhancing mutation, a mutation that is capable of enhancing Fc effector function, or both. In an embodiment, the Fc region comprises a half-life enhancing mutation, a mutation that is capable of maintaining Fc effector function, or both. In an embodiment, the Fc region comprises one or more mutations or combinations of mutations described herein, e.g., chosen from M252W, V308F/N434Y, R255Y, P257L/N434Y, V308F, P257N/M252Y, G385N, P257N/V308Y, N434Y, M252Y/S254T/T256E (“YTE”), M428L/N434S (“LS”), or any combination thereof. Alternatively, or additionally, in an embodiment, the Fc region comprises (a) one or more (e.g., 2, 3, 4, 5, or all) combinations of mutations chosen from: T256D/Q311V/A378V, H285N/T307Q/N315D, H285D/T307Q/A378V, T307Q/Q311V/A378V, T256D/N286D/T307R/Q311V/A378V, or T256D/T307R/Q311V. Additional Fc enhancements are described, for example, in PCT Publication No. WO 2018/052556 (incorporated by reference herein in its entirety).

Any of the mutations in the Fc region that extend half-life described herein can be used in combination with any Fc mutation capable of enhancing or maintaining an Fc effector function.

In an embodiment the Fc region comprises the Fc region of human IgG4, human IgG4 containing S228P mutation, and/or R409K mutation, and/or other mutations of the Fc region of human IgG4, or a fragment thereof. An exemplary fragment of an Fc region amino acid sequence from human IgG4 is provided in SEQ ID NO: 44 and is shown below:

E₂₁₉SKYGPPCPP₂₂₈ CPAPEFLGGPSV₂₄₀FLFPPKPKDT₂₅₀LMISRT
PEVT₂₆₀CVVVDVSQED₂₇₀PEVQFNWYVD₂₈₀GVEVHNAKTK₂₉₀PREE
QFNSTY₃₀₀RVVSVLT₃₀₇ VLHQ₃₁₁ DWLNGKEYK₃₂₀CKVSNKGLPS₃₃₀
SIEKTISKAK ₃₄₀GQPREPQ VYT₃₅₀LPPS QEEMTK₃₆₀N QVSLTCLVK₃₇₀
GFYPSDIA₃₇₈ VEWESNGQPENNYKTTPPVLDSDGSFFLYSRLTVDKSRW
QEGNVFSCSVMHEALHNHYTQKSLSLSLGK (SEQ ID NO: 44)

In SEQ ID NO: 44, the first amino acid residue in this sequence is referred to as position 219 herein. Mutations described to extend the half-life of human IgG1 can be applied to human IgG4 Fc. For example, Mut215 corresponds to mutations T307Q/Q311V/A378V in SEQ ID NO: 44.

The Fc region can bind to various cell receptors (e.g., Fc receptors) and complement proteins. The Fc region can also mediate different physiological effects of antibody molecules, e.g., detection of opsonized particles; cell lysis; degranulation of mast cells, basophils, and eosinophils; and other processes.

There are several different types of Fc receptors (FcR), which can be classified based on the type of antibody that they recognize.

Fcγ receptors (FcyR) belong to the immunoglobulin superfamily, and are involved, e.g., in inducing phagocytosis of opsonized microbes. This family includes several members, FcyRI (CD64), FcyRIIA (CD32), FcyRIIB (CD32), FcyRIIIA (CD16a), FcyRIIIB (CD16b), which differ in their antibody affinities due to their different molecular structure. For instance, FcyRI can bind to IgG more strongly than FcyRII or FcyRIII does. FcyRI also has an extracellular portion comprising three immunoglobulin (Ig)-like domains, one more domain than FcγRII or FcγRIII has. This property allows FcyRI to bind a sole IgG molecule (or monomer), but Fcγ receptors generally need to bind multiple IgG molecules within an immune complex to be activated.

The Fcγ receptors differ in their affinity for IgG and the different IgG subclasses can have unique affinities for each of the Fcγ receptors. These interactions can be further tuned by the glycan (oligosaccharide) at certain position of IgG. For example, by creating steric hindrance, fucose containing CH2-84.4 glycans reduce IgG affinity for FcyRIIIA, whereas G0 glycans, which lack galactose and terminate instead with GlcNAc moieties, have increased affinity for FcyRIIIA (Maverakis et al. (2015) Journal of Autoimmunity 57 (6): 1-13).

The neonatal Fc receptor (FcRn) is expressed on multiple cell types and is similar in structure to MHC class I. This receptor also binds IgG and is involved in preservation of this antibody (Zhu et al. (2001). Journal of Immunology 166 (5): 3266-76.). FcRn is also involved in transferring IgG from a mother either via the placenta to her fetus or in milk to her suckling infant. This receptor may also play a role in the homeostasis of IgG serum levels.

FcαRI (or CD89) belongs to the FcαR subgroup. FcαRI is found on the surface of neutrophils, eosinophils, monocytes, macrophages (including Kupffer cells), and dendritic cells. It comprises two extracellular Ig-like domains and is a member of both the immunoglobulin superfamily and the multi-chain immune recognition receptor (MIRR) family. It signals by associating with two FcRy signaling chains.

Fc-alpha/mu receptor (Fcα/µR) is a type I transmembrane protein. It can bind IgA, although it has higher affinity for IgM (Shibuya and Honda (2006) Springer Seminars in Immunopathology 28 (4): 377-82). With one Ig-like domain in its extracellular portion, this Fc receptor is also a member of the immunoglobulin superfamily.

There are two known types of FcεR. The high-affinity receptor FcsRI is a member of the immunoglobulin superfamily (it has two Ig-like domains). FcsRI is found on epidermal Langerhans cells, eosinophils, mast cells and basophils. This receptor can play a role in controlling allergic responses. FcsRI is also expressed on antigen-presenting cells, and controls the production of immune mediators, e.g., cytokines that promote inflammation (von Bubnoff et al. (2003) Clinical and Experimental Dermatology 28 (2): 184-7). The low-affinity receptor FcsRII (CD23) is a C-type lectin. FcsRII has multiple functions as a membrane-bound or soluble receptor. It can also control B cell growth and differentiation and blocks IgE-binding of eosinophils, monocytes, and basophils (Kikutani et al. (1989) Ciba Foundation Symposium 147: 23-31).

In an embodiment, the Fc region can be engineered to contain an antigen-binding site to generate an Fcab fragment (Wozniak-Knopp et al. (2010) Protein Eng Des 23 (4): 289-297). Fcab fragments can be inserted into a full immunoglobulin by swapping the Fc region, thus obtaining a bispecific antibody (with both Fab and Fcab regions containing distinct binding sites).

The binding and recycling of FcRn can be illustrated below. For example, IgG and albumin are internalized into vascular endothelial cells through pinocytosis. The pH of the endosome is 6.0, facilitating association with membrane-bound FcRn. The contents of endosomes can be processed in one of two ways: either recycling back to the apical cell membrane or transcytosis from the apical to the basolateral side. IgG not associated with FcRn is degraded by lysosomes.

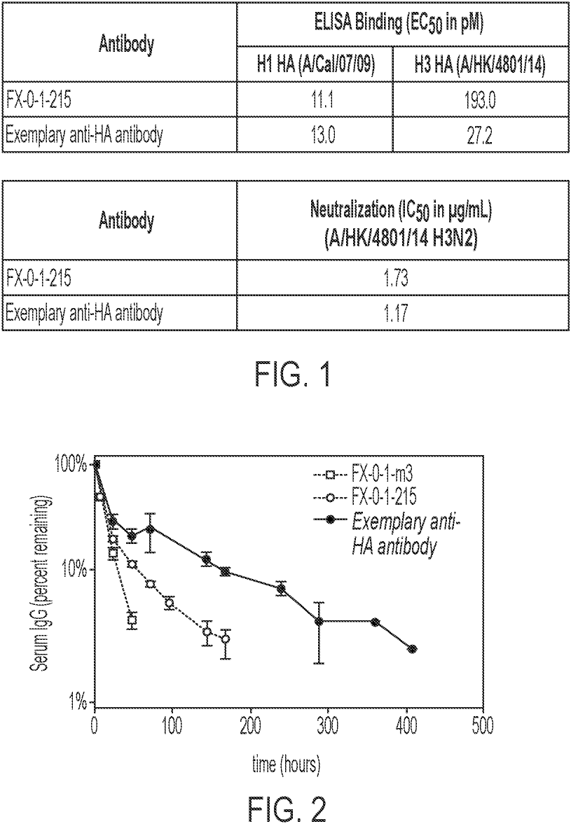

While not wishing to be bound by theory, it is believed that FcRn interaction with IgG is mediated through Fc. The binding of Fc to FcRn is pH specific, e.g., no significant binding at pH 7.4 and strong binding in acidic environment. Structure of FcRn in complex with Fc domain of IgG1 molecule is described, e.g., in FIG. 1 of International Application Publication No. WO2018/052556 or U.S. Application Publication No. US2018/0037634. Each FcRn molecule generally binds to an Fc-monomer. In an embodiment, Fab domains can also influence binding of IgG to FcRn, e.g., have either a negative or no influence on the affinity of the IgG for FcRn.

There can be multiple considerations when an Fc region is engineered to enhance half-life of a polypeptide. For example, prolonging half-life and efficient recirculation of antibody molecules or fusion proteins often requires pH specific affinity enhancement (e.g., only at low pH of the endosome). FcRn binds proximal to the linker region between CH2 and CH3 domains of a Fc region. Modifications to the linker can impact Fc engagement with Fcγ receptors. Modifications on the Fc region can impact thermal stability and aggregation properties of the polypeptide.

In an embodiment, the polypeptide (e.g., antibody molecule or fusion protein) described herein has the same affinity function, or does not substantially alter (e.g., decrease by more than 10%, 20%, 30%, 40%, 50%, 60%, 70%, 80%, or 90%) an effector function (e.g., an effector function described herein). In an embodiment, the effector function is not associated with the binding between an Fc region and an FcRn. The amino acid residues to be mutated can be selected, at least in part, based on the structural or functional properties of one or more binding sites on the Fc region. These binding sites include, but are not limited to, a Protein A binding site, a C1q binding site, an FcyRI binding site, an FcyRIIa binding site, an FcyRIIIa binding site, or an FcRn binding site. The binding sites can also include a TRIM21 binding site, e.g., one or more residues chosen from loop 308-316, loop 252-256, or loop 429-436 of an IgG. In an embodiment, the linker region between the CH2 and CH3 domain can influence the dynamics of the CH2 domain which impinges on FcyR binding.

In an embodiment, the polypeptide increases an effector function, e.g., by more than 10%, 20%, 30%, 40%, 50%, 60%, 70%, 80%, or 90%. In an embodiment, the polypeptide increases an effector function, e.g., by at least 1.5, 2, 3, 4, 5, 6, 7, 8, 9, 10, 15, 20, or 50-fold. In an embodiment, the increased effector function comprises, e.g., one or more (e.g., two, three, or all) of a complement dependent cytotoxicity (CDC), an antibody dependent cell mediated cytotoxicity (ADCC), an antibody dependent cell mediated phagocytosis (ADCP), or an antibody dependent intracellular neutralization (ADIN), e.g., compared to a reference polypeptide.

In an embodiment, the polypeptide has the same effector function, or does not substantially alter (e.g., decreases or increases by more than 10%, 20%, 30%, 40%, 50%, 60%, 70%, 80%, or 90%) an effector function, or increases an effector function (e.g., by at least 1.5, 2, 3, 4, 5, 6, 7, 8, 9, 10, 15, 20, or 50-fold), e.g., one or more (e.g., two, three, or all) of a complement dependent cytotoxicity (CDC), an antibody dependent cell mediated cytotoxicity (ADCC), an antibody dependent cell mediated phagocytosis (ADCP), or an antibody dependent intracellular neutralization (ADIN), compared to a reference polypeptide.

### Structural Basis for pH Specific Engagement of FcRn

Without wishing to be bound by theory, it is believed that low pH of the endosome leads to protonation of surface histidines on the CH2 and CH3 domains. For example, protonation of residue H310 on CH2 and/or H433 on CH3 can be important for FcRn engagement, e.g., at low pH (e.g., at pH 6.0). Protonation can also lead to change in the conformational dynamics of the region, such as better exposure or shielding of the linker region for solvent or ligand molecule binding. Accordingly, in an embodiment, the polypeptide (e.g., antibody molecule or fusion protein) comprises a mutation in residue H310, a mutation in residue H433, or both. One or more residues adjacent to residues H310 and/or H433 can also be mutated. The polypeptide can also include a compensating or beneficial mutation, e.g., a mutation that compensates, or beneficial, for any of the aforesaid mutations, e.g., to reduce a negative consequence of that mutation (e.g., polar vs. non-polar, charged vs. no charge, positively-charged (basic) vs. negatively charged (acidic), or hydrophobic vs. hydrophilic). For example, P247D can be a compensating or beneficial mutation.

In an embodiment, protonation of histidine can result in additional conformational changes including, e.g., movement/displacement of the linker/CH2/CH3 interface residues.

### Design Considerations for Optimizing FcRn Binding

In an embodiment, the polypeptide (e.g., antibody molecule or fusion protein) described herein can be designed for optimizing Fc-FcRn binding.

In an embodiment, the polypeptide having a mutation in the Fc region has a pH-specific affinity enhancement, compared to a reference polypeptide (e.g., an otherwise identical polypeptide without the mutation). In an embodiment, affinity enhancement is achieved by increasing van der Waal interaction. In an embodiment, affinity enhancement is not achieved by introduction of hydrogen bonds and/or electrostatic interaction. In an embodiment, the mutation does not alter, or has reduced or minimal perturbation to, the conformation of the linker region between the CH2 and CH3 domains. In an embodiment, the polypeptide comprises a plurality of mutations across both domains (four quadrants). In an embodiment, the polypeptide does not contain a large cluster of hydrophobic or aromatic residues on the surface.

In an embodiment, the polypeptide comprises a mutation that enhances the strength of interaction between an Fc region and FcRn or reduces the dissociation constant (K_{d}) for FcRn, e.g., at an acidic pH. In an embodiment, the polypeptide comprises a mutation that reduces the rate of dissociation (k_{off}) for FcRn, e.g., at an acidic pH. In an embodiment, the polypeptide comprises a mutation that increases the rate of association (kₒₙ) for FcRn, e.g., at an acidic pH. In an embodiment, the polypeptide comprises a mutation that reduces the rate of dissociation (k_{off}) for FcRn, and increases the rate of association (kₒₙ) for FcRn, e.g., at an acidic pH. In an embodiment, the polypeptide comprises a mutation that reduces the rate of dissociation (k_{off}) for FcRn, and does not, or does not significantly, affect the rate of association (kₒₙ) for FcRn, e.g., at an acidic pH. Without wishing to be bound by theory, it is believed that in an embodiment, the reduction of the dissociation constant K_{d} for FcRn is primarily resulted from the reduction of the rate of dissociation (k_{off}) for FcRn, rather than the increase of the rate of association (kₒₙ).

### Treatment/Prevention Methods and Administration

The binding agents, e.g., antibody molecules, featured in the disclosure, can be used to treat a subject, e.g., a subject, e.g., a human subject, infected with, or at risk for becoming infected with, an influenza virus. In some embodiments, the antibody molecule is for therapeutic use. In some embodiments, the antibody molecule is for prophylactic use.

Any human is candidate to receive an antibody molecule featured in the disclosure for treatment or prevention of an infection by an influenza virus. Humans at high risk of infection, such as immunocompromised individuals, and humans who are at high risk of exposure to influenza virus are particularly suited to receive treatment with the antibody molecule. Immunocompromised individuals include the elderly (65 years and older) and children (e.g., 6 months to 18 years old), and people with chronic medical conditions. People at high risk of exposure include heath care workers, teachers and emergency responders (e.g., firefighters, policemen). In an embodiment, the subject is hospitalized. In an embodiment, the subject is not hospitalized.

The antibody molecules described herein can also be used to prevent or reduce (e.g., minimize) secondary infection (e.g., secondary bacterial infection) or a risk of comprising secondary infection associated with influenza, or any effects (e.g., symptoms or complications) thereof on a subject. Opportunistic secondary bacterial infections (e.g., secondary bacterial pneumonia, e.g., primarily with Streptococcus pneumonia) contribute significantly to the overall morbidity and mortality associated with seasonal and pandemic influenza infections. The antibody molecules described herein can be used to prevent or reduce (e.g., minimize) the complications from secondary, opportunistic infections (e.g., bacterial infections) in a subject.

An antibody molecule can be administered to a subject, e.g., a human subject, by a variety of methods. For many applications, the route of administration is one of: intravenous injection or infusion, subcutaneous injection, or intramuscular injection. An antibody molecule can be administered as a fixed dose, or in a mg/kg dose. The antibody molecule can be administered intravenously (IV) or subcutaneously (SC). For example, the antibody molecule can be administered at a fixed unit dose of between about 50-600 mg IV, e.g., every 4 weeks, or between about 50-100 mg SC (e.g., 75 mg), e.g., at least once a week (e.g., twice a week). In one embodiment, the antibody molecule is administered IV at a fixed unit dose of 50 mg, 60 mg, 80 mg, 100 mg, 120 mg, 130 mg, 140 mg, 150 mg, 160 mg, 180 mg, 200 mg, 300 mg, 400 mg, 500 mg, or 600 mg or more. Administration of the IV dose can be once or twice or three times or more per week, or once every two, three, four, or five weeks, or less frequently.

An anti-HA antibody molecule featured in the disclosure can also be administered intravenously, such as a fixed unit dose between 500 mg and 5000 mg, e.g., between 500 mg and 4000 mg, between 500 mg and 3000 mg, between 1000 mg and 3000 mg, between 1500 mg and 3000 mg, between 2000 mg and 3000 mg, between 1800 mg and 2500 mg, between 2500 mg and 3000 mg, between 500 mg and 2500 mg, between 500 mg and 2000 mg, between 500 mg and 1500 mg, between 500 mg and 1000 mg, between 1000 mg and 2500 mg, between 1500 mg and 2000 mg, or between 2000 mg and 2500 mg, e.g., 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, or 4000 mg. In an embodiment, the antibody molecule is administered intravenously over a period of 1-3 hours, e.g., 1-2 hours or 2 to 3 hours, e.g., 2 hours. In an embodiment, the antibody molecule is administered as a single dose. In one embodiment, the antibody molecule is administered subcutaneously at a fixed unit dose of 50 mg, 60 mg, 70 mg, 75 mg, 80 mg, 100 mg, or 120 mg or more. Administration of the SC dose can be once or twice or three times or more per week, or once every two, three, four, or five weeks, or less frequently. An anti-HA antibody molecule featured in the disclosure can also be administered by inhalation, such as by intranasal or by oral inhalation, such as at a fixed unit dose of 50 mg, 60 mg, 80 mg, 100 mg, 120 mg, 130 mg, 140 mg, 150 mg, 160 mg, 180 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, or more.

In an embodiment, the antibody molecule is administered in an amount that does not cause an ADE in the subject, e.g., as determined by a method described herein. In an embodiment, the antibody molecule is administered in an amount that does not cause viral resistance, e.g., as determined by a method described herein. In one embodiment, an anti-HA antibody is administered to a subject via vector-mediated gene transfer, such as through the delivery of a vector encoding the heavy chain and the light chain of an anti-HA antibody, and the antibody is expressed from the heavy chain and light chain genes in the body. For example, nucleic acids encoding a heavy chain and a light chain can be cloned in a AAV vector, such as a self-complementary AAV vector, the scAAV vector administered to a human by injection, such as by IM injection, and the antibody is expressed and secreted into the circulation of the human.

An antibody molecule can also be administered in a bolus at a dose of between about 1 and 50 mg/kg, e.g., between about 1 and 10 mg/kg, between about 1 and 25 mg/kg or about 25 and 50 mg/kg, e.g., about 50 mg/kg, 25 mg/kg, 10 mg/kg, 6.0 mg/kg, 5.0 mg/kg, 4.0 mg/kg, 3.0 mg/kg, 2.0 mg/kg, 1.0 mg/kg, or less. Modified dose ranges include a dose that is less than about 3000 mg/subject, about 1500 mg, about 1000 mg/subject, about 600 mg/subject, about 500 mg/subject, about 400 mg/subject, about 300 mg/subject, about 250 mg/subject, about 200 mg/subject, or about 150 mg/subject, typically for administration every fourth week or once a month. The antibody molecule can be administered, for example, every three to five weeks, e.g., every fourth week, or monthly.

Dosing can be adjusted according to a patient’s rate of clearance of a prior administration of the antibody. For example, a patient may not be administered a second or follow-on dose before the level of antibodies in the patient’s system has dropped below a pre-determined level. In one embodiment, a sample from a patient (e.g., plasma, serum, blood, urine, or cerebrospinal fluid (CSF)) is assayed for the presence of antibodies, and if the level of antibodies is above a pre-determined level, the patient will not be administered a second or follow-on dose. If the level of antibodies in the patient’s system is below a pre-determined level, then the patient is administered a second or follow-on dose. A patient whose antibody levels are determined to be too high (above the pre-determined level) can be tested again after one or two or three days, or a week, and if the level of antibody in the patient samples has dropped below the pre-determined level, the patient may be administered a second or follow-on dose of antibody.

In certain embodiments, the antibody may be prepared with a carrier that will protect the drug against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Controlled Drug Delivery (Drugs and the Pharmaceutical Sciences), Second Edition, J. Robinson and V. H. L. Lee, eds., Marcel Dekker, Inc., New York, 1987.

Pharmaceutical compositions can be administered with a medical device. For example, pharmaceutical compositions can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules are discussed in, e.g., U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system comprising multi-chamber compartments; and U.S. Pat. No. 4,475,196, which discloses an osmotic drug delivery system. Of course, many other such implants, delivery systems, and modules are also known. In some embodiments, the binding agent, e.g., an antibody molecule, is administered buccally, orally, or by nasal delivery, e.g., as a liquid, spray, or aerosol, e.g., by topical application, e.g., by a liquid or drops, or by inhalation.

An antibody molecule described herein can be administered with one or more additional therapeutic agents, e.g., a second drug, for treatment of a viral infection, or a symptom of the infection. The antibody molecule and the one or more second or additional agents can be formulated together, in the same formulation, or they can be in separate formulations, and administered to a patient simultaneously or sequentially, in either order.

Dosage regimens are adjusted to provide the desired response, such as a therapeutic response or a combinatorial therapeutic effect. Generally, any combination of doses (either separate or co-formulated) of an antibody molecule and a second or additional agent can be used in order to provide a subject with both agents in bioavailable quantities. Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with another agent.

A pharmaceutical composition may include a “therapeutically effective amount” of an agent described herein. In some embodiments, where the antibody molecule is administered in combination with a second or additional agent, such effective amounts can be determined based on the combinatorial effect of the administered first and second or additional agent. A therapeutically effective amount of an agent may also vary according to factors such as the disease state, age, sex, and weight of the individual, and the ability of the compound to elicit a desired response in the individual, such as amelioration of at least one infection parameter, or amelioration of at least one symptom of the infection, such as chills, fever, sore throat, muscle pain, headache, coughing, weakness, fatigue and general discomfort. A therapeutically effective amount is also one in which any toxic or detrimental effects of the composition are outweighed by the therapeutically beneficial effects.

In an embodiment, administration of a binding agent, e.g., antibody molecule, provided, e.g., as a pharmaceutical preparation, is by one of the following routes: oral, intravenous, intramuscular, intra-arterial, subcutaneous, intraventricular, transdermal, intradermal, rectal, intravaginal, intraperitoneal, topical (as by liquids, powders, ointments, creams, sprays, or drops), mucosal, nasal, buccal, enteral, sublingual; intratracheal instillation, bronchial instillation, and/or inhalation; and/or as an oral spray, nasal spray, and/or aerosol. In an embodiment, the method described herein further comprises determining the presence or absence of an anti-drug antibody (ADA) in the subject. In an embodiment, the subject is selected for administration of an antibody molecule described herein on the basis of the absence of an ADA in the subject. ADA can be detected, e.g., by ELISA, in a sample from the subject.

### Combination Treatments and Exemplary Second or Additional Agents

Binding agents, e.g., antibody molecules, provided e.g., as pharmaceutical compositions, can be administered either alone or in combination with one or more other therapy, e.g., the administration of a second or additional therapeutic agent.

In some embodiments, the combination can result in a lower dose of the antibody molecule or of the other therapy being needed, which, in some embodiments, can reduce side effects. In some embodiments, the combination can result in enhanced delivery or efficacy of one or both agents. The agents or therapies can be administered at the same time (e.g., as a single formulation that is administered to a patient or as two separate formulations administered concurrently) or sequentially in any order. Such second or additional agents include vaccines, anti-viral agents, and/or additional antibodies. In typical embodiments the second or additional agent is not co-formulated with the binding agent, e.g., antibody molecule, though in others it is. In some embodiments, the binding agent, e.g., antibody molecule, and the second or additional agent are administered such that one or more of the following is achieved: therapeutic levels, or therapeutic effects, of one overlap the other; detectable levels of both are present at the same time; or the therapeutic effect is greater than what would be seen in the absence of either the binding agent, e.g., antibody molecule, or the second or additional agent. In some embodiments, each agent will be administered at a dose and on a time schedule determined for that agent.

The second or additional agent can be, for example, for treatment or prevention of influenza. For example, the binding agents, e.g., antibody molecules, e.g., therapeutic antibodies, provided herein can be administered in combination with a vaccine, e.g., a vaccine described herein or a mixture (a.k.a. a cocktail) of influenza peptides to stimulate the patient’s immune system to prevent infection with particular strains of influenza A. In other examples, the second or additional agent is an anti-viral agent (e.g., an anti-NA or anti-M2 agent), a pain reliever, an anti-inflammatory, an antibiotic, a steroidal agent, a second therapeutic antibody molecule (e.g., an anti-HA antibody), an adjuvant, a protease or glycosidase (e.g., sialidase), etc.

Four drugs have been approved for the treatment of acute influenza: three drugs that target the viral neuraminidase (NA) activity (oseltamivir, peramivir, and zanamivir) and a drug targeting the PA subunit of the viral RNA polymerase (baloxavir-marboxil) that was recently approved in Japan and the U.S. in 2018. The neuraminidase inhibitors (NAIs) are used off label as standard-of-care for critically ill hospitalized patients with influenza. Baloxavir marboxil may also be used for treating hospitalized patients with influenza.

Exemplary anti-viral agents include, e.g., vaccines, neuraminidase inhibitors or nucleoside analogs. Exemplary anti-viral agents can include, e.g., zidovudine, gangcyclovir, vidarabine, idoxuridine, trifluridine, foscarnet, acyclovir, ribavirin, amantadine, remantidine, saquinavir, indinavir, ritonavir, alpha-interferons and other interferons, a neuraminidase inhibitor (e.g., zanamivir (Relenza®), oseltamivir (Tamiflu®), laninamivir, peramivir), rimantadine, a PB2 inhibitor (e.g., pimodivir), and an endonuclease inhibitor (e.g., the cap-dependent endonuclease inhibitor, e.g., baloxavir marboxil).

In an embodiment, the antiviral agent is an endonuclease (e.g., cap-dependent endonuclease (CEN) inhibitor or an PA (viral RNA polymerase PA subunit) inhibitor. In an embodiment, the endonuclease inhibitor or PA inhibitor is baloxavir. Baloxavir is described, e.g., in Antiviral Res. 2018; 160: 109-117, the content of which is incorporated by reference in its entirety. Cap-dependent endonuclease (CEN) resides in the PA subunit of the influenza virus and mediates the critical “cap-snatching” step of viral RNA transcription. Baloxavir acid (BXA) is generally considered to be an active form of baloxavir marboxil (BXM). Without wishing to be bound by theory, it is believed that in an embodiment, BXA can inhibit both viral RNA transcription via selective inhibition of CEN activity and viral replication.

In an embodiment, the antiviral agent is an inhibitor of influenza virus basic protein 2 (PB2), a component of the viral RNA replication complex. In an embodiment, the PB2 inhibitor is pimodivir. Pimodivir is described, e.g., in Nucleic Acids Res. 2018; 46(2): 956-971, the content of which is incorporated by reference in its entirety. Influenza RNA-dependent RNA polymerase is typically a heterotrimer with subunits PA, PB1 and PB2. Without wishing to be bound by theory, it is believed that it binds the conserved 3′ and 5′ ends of each of the eight negative-sense RNA genome segments and is responsible for transcription and replication of the genomic RNA in the nucleus of infected cells. Transcription is typically initiated by short capped primers originated from nascent host Pol II transcripts, and therefore a host sequence of 10-14 nucleotides in length precede the virally encoded sequences in the resultant chimeric viral mRNA.

Exemplary second antibody molecules include, for example, Ab 67-11 (U.S. Provisional Application Number 61/645,453, FI6 (U.S. Application Publication No. 2010/0080813), FI28 (U.S. Application Publication No. 2010/0080813), C179 (Okuno et al., J. Virol. 67:2552-8, 1993), F10 (Sui et al., Nat. Struct. Mol. Biol. 16:265, 2009), CR9114 (Dreyfus et al., Science 337:1343, 2012), or CR6261 (Ekiert et al., Science 324:246, 2009). Thus, Ab 044 can be used in combination of any of those antibodies. In other embodiments, two or more binding agents, e.g., antibody molecules disclosed herein, can be administered in combination, e.g., Ab 044 can be administered in combination with Ab 032. In the case of combinations, two agents can be administered as part of the same dosage unit or administered separately. Other exemplary agents useful for treating the symptoms associated with influenza infection are acetaminophen, ibuprofen, aspirin, and naproxen.

In one embodiment, the antibody molecule and the second or additional agent are provided as a co-formulation, and the co-formulation is administered to the subject. It is further possible, e.g., at least 24 hours before or after administering the co-formulation, to administer separately one dose of the antibody formulation and then one dose of a formulation containing a second or additional agent. In another implementation, the antibody molecule and the second or additional agent are provided as separate formulations, and the step of administering includes sequentially administering the antibody molecule and the second or additional agent. The sequential administrations can be provided on the same day (e.g., within one hour of one another or at least 3, 6, or 12 hours apart) or on different days.

In some embodiments, the antibody molecule and the second or additional agent are each administered as a plurality of doses separated in time. The antibody molecule and the second or additional agent are generally each administered according to a regimen. The regimen for one or both may have a regular periodicity. The regimen for the antibody molecule can have a different periodicity from the regimen for the second or additional agent, e.g., one can be administered more frequently than the other. In one implementation, one of the antibody molecule and the second or additional agent is administered once weekly and the other once monthly. In another implementation, one of the antibody molecule and the second or additional agent is administered continuously, e.g., over a period of more than 30 minutes but less than 1, 2, 4, or 12 hours, and the other is administered as a bolus. In some embodiments, sequential administrations are administered. The time between administration of the one agent and another agent can be minutes, hours, days, or weeks. The use of an antibody molecule described herein can also be used to reduce the dosage of another therapy, e.g., to reduce the side-effects associated with another agent that is being administered. Accordingly, a combination can include administering a second or additional agent at a dosage at least 10, 20, 30, or 50% lower than would be used in the absence of the antibody molecule. The antibody molecule and the second or additional agent can be administered by any appropriate method, e.g., subcutaneously, intramuscularly, or intravenously.

In some embodiments, each of the antibody molecule and the second or additional agent is administered at the same dose as each is prescribed for monotherapy. In other embodiments, the antibody molecule is administered at a dosage that is equal to or less than an amount required for efficacy if administered alone. Likewise, the second or additional agent can be administered at a dosage that is equal to or less than an amount required for efficacy if administered alone. In some cases, the formulations described herein, e.g., formulations containing an antibody molecule featured in the disclosure, include one or more second or additional agents, or are administered in combination with a formulation containing one or more second or additional agents. In an embodiment a binding agent, e.g., antibody molecule, provided, e.g., as a pharmaceutical preparation, is administered by inhalation or aerosol delivery of a plurality of particles, e.g., particles comprising a mean particle size of 4, 5, 6, 7, 8, 9, 10, 11, 12, or 13 microns.

### Pharmaceutical Compositions

The binding agents, e.g., antibody molecules, featured in the disclosure can be formulated as pharmaceutical compositions, such as for the treatment or prevention of influenza.

Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible.

A “pharmaceutically acceptable salt” refers to a salt that retains the desired biological activity of the parent compound and does not impart any undesired toxicological effects (see e.g., Berge, S.M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The compositions comprising antibody molecules can be formulated according to methods known in the art. Pharmaceutical formulation is a well-established art, and is further described in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

Pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form can depend on the intended mode of administration and therapeutic application. Typically, compositions for the agents described herein are in the form of injectable or infusible solutions. Such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular (IM), intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and by intrasternal injection or by infusion.

Pharmaceutical compositions may be provided in a sterile injectable form (e.g., a form that is suitable for subcutaneous injection or intravenous infusion). In some embodiments, pharmaceutical compositions are provided in a liquid dosage form that is suitable for injection or topical application. In some embodiments, pharmaceutical compositions are provided as in dry form, e.g., as powders (e.g. lyophilized and/or sterilized preparations). The Pharmaceutical composition can be provided under conditions that enhance stability, e.g., under nitrogen or under vacuum. Dry material can be reconstituted with an aqueous diluent (e.g., water, buffer, salt solution, etc.) prior to injection.

In one embodiment, the pharmaceutical composition containing an anti-HA antibody is administered intranasally. In another embodiment, the pharmaceutical composition containing an anti-HA antibody is administered by inhalation, such as by oral or by nasal inhalation. In some embodiments, the pharmaceutical composition is suitable for buccal, oral or nasal delivery, e.g., as a liquid, spray, or aerosol, e.g., by topical application, e.g., by a liquid or drops, or by inhalation). In some embodiments, a pharmaceutical preparation comprises a plurality of particles, suitable, e.g., for inhaled or aerosol delivery. In some embodiments, the mean particle size of 4, 5, 6, 7, 8, 9, 10, 11, 12, or 13 microns. In some embodiments, a pharmaceutical preparation is formulated as a dry powder, suitable, e.g., for inhaled or aerosol delivery. In some embodiments, a pharmaceutical preparation is formulated as a wet powder, through inclusion of a wetting agent, e.g., water, saline, or other liquid of physiological pH. In some embodiments, a pharmaceutical preparation is provided as drops, suitable, e.g., for delivery to the nasal or buccal cavity. In some embodiments, the pharmaceutical composition is disposed in a delivery device, e.g., a syringe, a dropper or dropper bottle, an inhaler, or a metered dose device, e.g., an inhaler.

In one embodiment, a pharmaceutical composition contains a vector, such as an adenovirus-associated virus (AAV)-based vector, that encodes a heavy chain of an anti-HA antibody molecule, and a light chain of an anti-HA antibody molecule featured in the disclosure. The composition containing the vector can be administered to a subject, such as a patient, such as by injection, e.g., IM injection. Genes encoding the anti-HA antibody under control of, for example, cytomegalovirus (CMV) promoters, are expressed in the body, and the recombinant anti-HA antibody molecule is introduced into the circulation. See, e.g., Balazs et al., Nature 30:481:81-84, 2011.

Pharmaceutical compositions typically should be sterile and stable under the conditions of manufacture and storage. A pharmaceutical composition can also be tested to insure it meets regulatory and industry standards for administration. The composition can be formulated as a solution, microemulsion, dispersion, liposome, or other ordered structure suitable to high drug concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, typical methods of preparation are vacuum drying and freeze-drying that yields a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

A pharmaceutical composition may be provided, prepared, packaged, and/or sold in bulk, as a single unit dose, and/or as a plurality of single unit doses. Typically, a bulk preparation will contain at least 2, 5, 10, 20, 50, or 100 unit doses. A unit dose is typically the amount introduced into the patient in a single administration. In some embodiments, only a portion of a unit dose is introduced. In some embodiments, a small multiple, e.g., as much as 1.5, 2, 3, 5, or 10 times a unit dose is administered. The amount of the active ingredient is generally equal to a dose which would be administered to a subject and/or a convenient fraction of such a dose such as, for example, one-half or one-third of such a dose.

In some embodiments, the pharmaceutical composition comprises an antibody molecule as described herein, e.g., at a concentration greater than about 100 mg/mL (e.g., greater than about 100, 110, 120, 130, 140, 148, 150, 160, 170, 180, 190, 200, 210, 220, 230, 240, or 250 mg/mL). In some embodiments, an antibody molecule as described herein is formulated at a concentration greater than about 100 mg/mL (e.g., greater than about 100, 110, 120, 130, 140, 148, 150, 160, 170, 180, 190, 200, 210, 220, 230, 240, or 250 mg/mL).

### Epitope

HAs exist in nature as homotrimers of proteolytically processed mature subunits. Each subunit of the trimer is synthesized as a precursor. A precursor molecule is proteolytically processed into two disulfide bonded polypeptide chains to form a mature HA polypeptide. The mature HA polypeptide includes two domains: (1) a core HA-1 domain that extends from the base of the molecule through the fibrous stem to the membrane distal head region that contains the glycan receptor binding domain, returning to fibrous region ending in the cleavage site, and (2) HA-2 domain that includes the stem region and the transmembrane domain of HA. HA-1 includes a glycan binding site. The glycan binding site may be responsible for mediating binding of HA to the HA-receptor. The HA-2 domain acts to present the HA-1 domain. The HA trimer can be stabilized by polar and non-polar interactions between the three long HA alpha-helices of the stem of HA monomers.

HA sequences from all influenza subtypes share a set of amino acids in the interface of the HA-1 and HA-2 domains that are well conserved. The HA-1/HA-2 interface membrane proximal epitope region (MPER) that includes the canonical α-helix and residues in its vicinity are also conserved across a broad spectrum of subtypes. (Ekiert et al., Science. 324(5924):246, 2009; Sui et al., Nat Struct Mol Biol. 16(3):265, 2009).

The antibody molecules described herein can have high affinity for HA’s from Group 1 and Group 2. They typically bind a conformational epitope that is broadly conserved across a plurality of influenza strains. Numerous amino acid residues distributed along the linear sequences of HA from different strains/subtypes contribute the conformational epitope. In some embodiments, the antibody molecule binds to a conserved and constrained epitope on HA (e.g., in the stem or stalk region), e.g., a region that is associated with the structural and functional integrity, common across multiple influenza strains, and/or resistant to mutations. In some embodiments, the antibody molecule binds to the same, or essentially the same, epitope as FX-0-1-m3. FX-0-1-m3 (also known as Ab 044) is described, e.g., in PCT Application Publication Nos. WO 2013/170139 and WO 2017/083627, U.S. Pat. Nos. 8,877,200, 9,096,657, 9,969,794, and 10,513,553, the contents of the aforesaid publications are incorporated by reference in their entirety.

### Diagnostic Methods

The binding agents, e.g., antibody molecules, provided herein are useful for identifying the presence of influenza in a biological sample, e.g., a patient sample, such as a fluid sample, e.g., a blood, serum, saliva, mucous, or urine sample, or a tissue sample, such as a biopsy. In one embodiment, a patient sample is contacted with a binding agent, e.g., an antibody molecule, featured in the disclosure, and binding is detected. Binding can be detected with a number of formats and means of detection, e.g., with an antigen capture assay, such as an ELISA assay or Western blot, or an immunohistochemistry assay. In some embodiments, the binding agent, e.g., an antibody molecule, is provided, e.g., coupled to an insoluble matrix, e.g., a bead or other substrate, and a detection molecule used to detect binding of HA.

Binding of binding agent, e.g., antibody molecule, to HA, can be detected with a reagent comprising a detectable moiety, e.g., a reagent, e.g., an antibody, which binds the binding agent, e.g., antibody molecule. In some embodiments, the binding agent, e.g., antibody molecule, has a detectable moiety. Suitable detectable moieties include enzymes (e.g., horseradish peroxidase, betagalactosidase, luciferase, alkaline phosphatase, acetylcholinesterase, glucose oxidase and the like), radiolabels (e.g., ³H, ¹⁴C, ¹⁵N, ³⁵S, ⁹⁰Y, ⁹⁹Tc, ¹¹¹ In, ¹²⁵I,¹³¹I), haptens, fluorescent labels (e.g., FITC, rhodamine, lanthanide phosphors, fluorescein, fluorescein isothiocyanate, rhodamine, 5-dimethylamine-1-napthalenesulfonyl chloride, phycoerythrin and the like), phosphorescent molecules, chemiluminescent molecules, chromophores, luminescent molecules, photoaffinity molecules, colored particles or affinity ligands, such as biotin, predetermined polypeptide epitopes recognized by a secondary reporter (e.g., leucine zipper pair sequences, or binding sites for secondary antibodies, metal binding domains, epitope tags). In some embodiments, labels are attached by spacer arms of various lengths to reduce potential steric hindrance.

In some embodiments, a human is tested for presence of influenza virus be a method described herein, and if the test is positive, binding agents, e.g., antibody molecules, e.g., an antibody provided herein, is administered. The binding agents, e.g., antibody molecules, e.g., an antibody, provided herein can be used for cytology assays, such as to identify an HA in a cell. The assay can be a colorimetric assay. A biological sample from a normal (non-infected) individual is used as a control. The diagnostic assay can be performed in vitro. The diagnostic assay can also be performed to determine infection of cells in culture, e.g., of mammalian cells in culture. The antibody molecules can be used in in vitro assays.

Because the antibody molecules featured herein bind a broad spectrum of HA subtypes, the diagnostic assays featured in the disclosure can detect the presence of influenza virus in patients infected with a variety of distinct strains of influenza. A patient sample can be further tested with subtype specific antibodies, or other assays (e.g., RFLP (Restriction Fragment Length Polymorphism), PCR (Polymerase Chain Reaction), RT-PCR (Reverse Transcription coupled to Polymerase Chain Reaction), Northern blot, Southern blot or DNA sequencing) to further determine the particular strain of virus. In one embodiment, a patient determined to be infected with influenza A can be further administered an antibody molecule featured in the disclosure, to treat the infection. Also provided are solid substrates, e.g., beads, dipsticks, arrays, and the like, on which is disposed a binding agent, e.g., antibody molecule.

### Kits

A binding agent, e.g., an antibody molecule, disclosed herein, e.g., generated by the methods described herein, can be provided in a kit, e.g., for use in a method described herein. The kit can include one or more other components, e.g., containers, buffers or other diluents, delivery devices, and the like.

In one embodiment, the kit includes materials for administering an antibody molecule to a subject, such as for treatment or prevention of infection by influenza viruses. For example, the kit can include one or more or all of: (a) a container that contains a composition that includes an antibody molecule, optionally (b) a container that contains a composition that includes a second therapeutic agent, and optionally (c) informational material. In another embodiment, the kit includes materials for using an antibody molecule in a diagnostic assay, such as for detection of HA in a biological sample. For example, the kit can include one or more or all of: (a) a container that contains a composition that includes an antibody molecule, optionally (b) a container that contains a reagents, e.g., labeled with a detectable moiety, to detect the antibody, e.g., for use in an ELISA or immunohistochemistry assay, and optionally (c) informational material. In other embodiments, the kit comprises a binding agent, e.g., antibody molecule, comprising a detectable moiety.

In an embodiment, the kit comprises a solid substrate, e.g., bead, dipstick, array, and the like, on which is disposed a binding agent, e.g., antibody molecule. The informational material can be descriptive, instructional, marketing or other material that relates to the methods described herein and/or the use of the agents for therapeutic benefit, or for a diagnostic assay. The informational material of the kits is not limited in its form. In one embodiment, the informational material can include information about production of the antibody, concentration, date of expiration, batch or production site information, and so forth. In one embodiment, the informational material relates to methods of administering the antibody, e.g., in a suitable dose, dosage form, or mode of administration (e.g., a dose, dosage form, or mode of administration described herein), to treat a subject who has an infection, e.g., viral infection or secondary infection (e.g., secondary bacterial infection). In another embodiment, the informational material relates to methods for using the antibody molecule for a diagnostic assay, e.g., to detect the presence of influenza viruses in a biological sample. The information can be provided in a variety of formats, including printed text, computer readable material, video recording, or audio recording, or information that provides a link or address to substantive material. In addition to the agent, the composition in the kit can include other ingredients, such as a solvent or buffer, a stabilizer, or a preservative. The agent can be provided in any form, e.g., a liquid, dried or lyophilized form, and substantially pure and/or sterile. When the agents are provided in a liquid solution, the liquid solution typically is an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more units of dosage forms (e.g., a dosage form described herein) of the agents. The containers can include a combination unit dosage, e.g., a unit that includes both the antibody molecule and the second or additional agent, such as in a desired ratio. For example, the kit can include a plurality of syringes, ampoules, foil packets, blister packs, or medical devices each containing, for example, a single combination unit dose. The containers of the kits can be airtight, waterproof (e.g., impermeable to changes in moisture or evaporation), and/or light-tight.

The kit optionally includes a device suitable for administering the composition, e.g., a syringe or device for delivering particles or aerosols, e.g., an inhaler, a spray device, or a dropper or other suitable delivery device. The device can be provided pre-loaded with one or both of the agents or can be empty but suitable for loading. The invention is further illustrated by the following examples, which should not be construed as further limiting.

### Other Embodiments

The antibody molecule described herein can be encoded by a nucleic acid molecule, e.g., an isolated nucleic acid molecule. In an embodiment, the nucleic acid molecule comprises a nucleotide sequence that encodes a heavy chain immunoglobulin variable region segment featured in the disclosure. In another embodiment, the nucleic acid molecule comprises a nucleotide sequence encoding a light chain immunoglobulin variable region segment featured in the disclosure. In yet another aspect, the nucleic acid molecule comprises a nucleotide sequence that encodes a heavy chain immunoglobulin variable region segment featured in the disclosure and a light chain immunoglobulin variable region segment featured in the disclosure. In an embodiment, the nucleic acid molecule is present in a vector, e.g., a recombinant vector (e.g., an expression vector). In an embodiment, the vector comprises a nucleic acid molecule that comprises a nucleotide sequence that encodes a heavy chain immunoglobulin variable region segment featured in the disclosure, a nucleotide sequence that encodes a light chain immunoglobulin variable region segment featured in the disclosure, or both.

In an embodiment, the antibody molecule described herein is produced from a cell containing a recombinant vector featured in the disclosure, such as a recombinant vector comprising a nucleic acid sequence that encodes a heavy chain immunoglobulin variable region, or a recombinant vector comprising a nucleic acid sequence that encodes a light chain immunoglobulin variable region. In one embodiment, the cell contains a recombinant vector comprising a nucleic acid sequence that encodes a heavy chain immunoglobulin variable region, and a recombinant vector comprising a nucleic acid sequence that encodes a light chain immunoglobulin variable region. In yet another embodiment, the cell contains a recombinant vector comprising a nucleic acid sequence that encodes a heavy chain immunoglobulin variable region, and a nucleic acid sequence that encodes a light chain immunoglobulin variable region. In an embodiment, the antibody molecule is produced, e.g., by providing a host cell comprising a nucleic acid sequence expressing a heavy chain segment and a nucleic acid sequence expressing a light chain segment and expressing the nucleic acids in the host cell. In one embodiment, the nucleic acid sequence expressing the heavy chain segment and the nucleic acid sequence expressing the light chain segment are on the same recombinant expression vector. In another embodiment, the nucleic acid sequence expressing the heavy chain segment and the nucleic acid sequence expressing the light chain segment are on separate recombinant expression vectors.

In an embodiment, a pharmaceutical composition containing an antibody molecule featured in the disclosure, and a pharmaceutically acceptable carrier, is used in a method described herein.

In an embodiment, the method described herein treats or prevents an infection with an influenza virus (e.g., an influenza A virus, e.g., a Group 1 strain, e.g., an H1N1 strain, e.g., A/South Carolina/1/1918, A/Puerto Rico/08/1934, or A/California/04/2009, or an H5N1 strain, e.g., A/Indonesia/5/2005 or A/Vietnam/1203/2004, in a subject, e.g., a human subject, that comprises: administering a binding agent, e.g., an antibody molecule, featured in the disclosure to a subject, e.g., human subject, in need thereof. In one embodiment, the influenza A virus is an H1, H5, H9, H3 or H7 strain, such as an H1N1 strain, an H3N2 strain, or an H5N1 strain of influenza A virus. In an embodiment, the administration results in, or correlates with, one or more of a reduction in the incidence or severity of a symptom or manifestation of an influenza infection, or the delay or onset of a symptom or manifestation of an influenza infection. In an embodiment, the administration results in, or correlates with, one or more of a reduction in the incidence or severity of a symptom or manifestation of a secondary infection, or the delay or onset of a symptom or manifestation of a secondary infection. In some embodiments, the subject, e.g., a human subject, has been administered, or the method comprises, administering, or recommending the administration of, a second or additional therapy.

In some embodiments, the antibody molecule is administered in combination with a second or additional agent or therapy. In some embodiments, the second or additional therapy comprises administration of a vaccine or an anti-viral therapy, e.g., an anti-NA or an anti-M2 therapy. In an embodiment, the second or additional therapy comprises an administration of a vaccine, e.g., a vaccine described herein or a mixture (a.k.a. a cocktail) of influenza peptides to stimulate the patient’s immune system to prevent infection with particular strains of influenza A. In an embodiment, the second or additional agent comprises administering an anti-viral agent, a pain reliever, an anti-inflammatory, an antibiotic, a steroidal agent, a second therapeutic antibody molecule (e.g., an anti-HA antibody), an adjuvant, a protease or glycosidase (e.g., sialidase). In an embodiment, the second or additional agent comprises, acyclovir, ribavirin, amantadine, rimantadine, a neuraminidase inhibitor (e.g., zanamivir (Relenza®), oseltamivir (Tamiflu®), laninamivir, peramivir), or rimantadine.

In an embodiment, the second or additional agent comprises a second antibody molecule, e.g., an anti-HA antibody disclosed in PCT Application Publication Nos. WO 2013/170139 (e.g., Ab 044), an anti-HA antibody disclosed in PCT Application Publication No. WO 2013/169377, FI6 (U.S. Application Publication No. 2010/0080813), FI28 (U.S. Application Publication No. 2010/0080813), C179 (Okuno et al., J. Virol. 67:2552-8, 1993), F10 (Sui et al., Nat. Struct. Mol. Biol. 16:265, 2009), CR9114 (Dreyfus et al., Science 337:1343, 2012), or CR6261 (see, e.g., Ekiert et al., Science 324:246, 2009). Thus, an antibody molecule described herein can be used in combination of any of those antibodies. In an embodiment, the second or additional agent comprises a second or additional binding agent, e.g., antibody molecule, e.g., an anti-HA antibody, e.g., an anti-HA antibody disclosed herein. In the case of combinations, two agents can be administered as part of the same dosage unit or administered separately. Other exemplary agents useful for treating the symptoms associated with influenza infection are acetaminophen, ibuprofen, aspirin, and naproxen.

In an embodiment, the binding agent, e.g., an antibody molecule, is administered to a human subject suffering from or susceptible to an influenza infection. In an embodiment, the binding agent, e.g., an antibody molecule, is administered prior to known exposure to influenza, or to particular influenza subtypes or strains. In an embodiment, the binding agent, e.g., an antibody molecule, is administered prior to manifestation of effects or symptoms of influenza infection, or to one or more particular effects manifestation of effects or symptoms of influenza infection. In an embodiment, the binding agent, e.g., an antibody molecule, is administered after known exposure to influenza, or to particular influenza subtypes or strains. In an embodiment, the binding agent, e.g., an antibody molecule, is administered after manifestation of effects or symptoms of influenza infection, or after observation of one or more particular effects manifestation of effects or symptoms of influenza infection. In an embodiment, the binding agent, e.g., an antibody molecule, is administered in response to, or to treat or prevent, a manifestation of an effect or a symptom of influenza infection, e.g., inflammation, fever, nausea, weight loss, loss of appetite, rapid breathing, increase heart rate, high blood pressure, body aches, muscle pain, eye pain, fatigue, malaise, dry cough, runny nose, and/or sore throat.

In an embodiment, the method further comprises, testing the human subject for the influenza virus, e.g., with a method disclosed herein. In some embodiments, the administration is responsive to a positive test for influenza.

In an embodiment, the method described herein treats a subject, e.g., a human subject, an infected with an influenza virus (e.g., an influenza A virus, e.g., a Group 1 strain, e.g., an H1N1 strain, e.g., A/South Carolina/1/1918, A/Puerto Rico/08/1934, or A/California/04/2009, or an H5N1 strain, e.g., A/Indonesia/5/2005 or A/Vietnam/1203/2004, by administering a binding agent, e.g., an antibody molecule, featured in the disclosure. For example, the influenza A virus is an H1, H5, H9, H3 or H7 strain, such as an H1N1 strain, an H3N2 strain, or an H5N1 strain of influenza A virus. In one embodiment, a binding agent, e.g., an anti-HA antibody, described herein is administered instead of a vaccine for prevention of influenza. In another embodiment, the binding agent, e.g., anti-HA antibody molecule, is administered in combination with (simultaneously or sequentially with) a vaccine for prevention of the flu.

In an embodiment, the method further comprises detecting influenza (e.g., influenza A) virions in a biological sample, such as by contacting the sample with a binding agent, e.g., an antibody molecule, featured in the disclosure, and then detecting the binding of the antibody molecule to the sample. In one embodiment, the method of detecting the influenza virus (e.g., influenza A virus) is performed in vitro.

In an embodiment, the method further includes: (a) providing a sample from a patient; (b) contacting the sample with a binding agent, e.g., an antibody molecule, featured in the disclosure, and (c) determining whether the binding agent, e.g., an antibody molecule, featured in the disclosure binds a polypeptide in the sample, where if the binding agent, e.g., an antibody molecule, binds a polypeptide in the sample, then the patient is determined to be infected with an influenza virus (e.g., an influenza A virus, e.g., a Group 1 strain, e.g., an H1N1 strain, e.g., A/South Carolina/1/1918, A/Puerto Rico/08/1934, or A/California/04/2009, or an H5N1 strain, e.g., A/Indonesia/5/2005 or A/Vietnam/1203/2004). In one embodiment, the patient is determined to be infected with an influenza virus (e.g., an influenza A virus, e.g., a Group 1 strain, e.g., an H1N1 strain, e.g., A/South Carolina/1/1918, A/Puerto Rico/08/1934, or A/California/04/2009, or an H5N1 strain, e.g., A/Indonesia/5/2005 or A/Vietnam/1203/2004), and the patient is further administered a binding agent, e.g., an antibody molecule, disclosed herein, e.g., the binding agent, e.g., an antibody molecule, with which the test was performed.

In an embodiment, the administration results in, or correlates with, one or more of: a reduction in the chance of an infection, a reduction in the incidence or severity of a symptom or manifestation of an influenza infection, or the delay or onset of a symptom or manifestation of an influenza infection. In an embodiment, the administration results in, or correlates with, one or more of: a reduction in the incidence or severity of a symptom or manifestation of a secondary infection, or the delay or onset of a symptom or manifestation of a secondary infection.

In some embodiments, the subject, e.g., a human subject, has been administered, or the method comprises, administering, or recommending the administration of, a second or additional therapy. In some embodiments, the broad range vaccine is administered in combination with a second or additional agent or therapy. In some embodiments, the second or additional agent comprises administration of another vaccine or another anti-viral therapy, e.g., an anti-NA or an anti-M2 therapy. In an embodiment, the second or additional agent comprises administration of a vaccine comprising a mixture (a.k.a. a cocktail) of influenza peptides to stimulate the patient’s immune system to prevent infection with particular strains of influenza A. In an embodiment, the second or additional agent comprises administering an anti-viral agent, a pain reliever, an anti-inflammatory, an antibiotic, a steroidal agent, a second therapeutic antibody molecule (e.g., an anti-HA antibody), an adjuvant, a protease or glycosidase (e.g., sialidase). In an embodiment, the second or additional agent comprises, acyclovir, ribavirin, amantadine, rimantadine, a neuraminidase inhibitor (e.g., zanamivir (Relenza®), oseltamivir (Tamiflu®), laninamivir, peramivir), or rimantadine. In an embodiment, the second or additional agent comprises an antibody molecule, e.g., an anti-HA antibody disclosed in PCT Application Publication Nos. WO 2013/170139 (e.g., Ab 044), an anti-HA antibody disclosed in PCT Application Publication No. WO 2013/169377, FI6 (U.S. Application Publication No. 2010/0080813), FI28 (U.S. Application Publication No. 2010/0080813), C179 (Okuno et al., J. Virol. 67:2552-8, 1993), F10 (Sui et al., Nat. Struct. Mol. Biol. 16:265, 2009), CR9114 (Dreyfus et al., Science 337:1343, 2012), or CR6261 (Ekiert et al., Science 324:246, 2009). In an embodiment, the second or additional agent comprises an antibody molecule disclosed herein, e.g., an antibody molecule selected from Ab-044, Ab 069, Ab 032, and Ab 031 antibody molecules. In the case of combinations, two agents can be administered as part of the same dosage unit or administered separately. Other exemplary second or additional agents useful for treating the symptoms associated with influenza infection are acetaminophen, ibuprofen, aspirin, and naproxen.

In an embodiment, the method further comprises, testing the human subject for the influenza virus, e.g., with a method disclosed herein. In some embodiments, the administration is responsive to a positive test for influenza. In an embodiment, the method further comprises reducing the severity of influenza in a population. The method includes administering a broad range vaccine, or broad range immunogen, to sufficient individuals in the population to prevent or decrease the chance of influenza virus transmission to another individual in the population.

### Additional Aspects and Embodiments Are Provided in the Numbered Paragraphs Below

1. An anti-hemagglutinin (HA) antibody molecule (e.g., an isolated anti-HA antibody molecule) comprising (a) one, two, or all of CDR1, CDR2, or CDR3 of a heavy chain variable region segment described herein (e.g., one, two, or all of CDR1, CDR2, or CDR3 of the heavy chain variable region of any one of VH1 through VH184); (b) one, two, or all of CDR1, CDR2, or CDR3 of a light chain variable region segment described herein (e.g., one, two, or all of CDR1, CDR2, or CDR3 of the light chain variable region of any one of VK-1 through VK-111); or (c) both (a) and (b), provided that the antibody molecule does not comprises all of CDR1, CDR2, or CDR3 of the heavy chain variable region VH0 and all of CDR1, CDR2, or CDR3 of a light chain variable region the light chain variable region VK-0.
2. The antibody molecule of paragraph 1, comprising CDR1, CDR2, and CDR3 of the heavy chain variable region segment and CDR1, CDR2, and CDR3 of the light chain variable region segment.
3. The antibody molecule of paragraph 1 or 2, comprising the heavy chain variable region segment, the light chain variable region segment, or both.
4. The antibody molecule of any of paragraphs 1-3, comprising the heavy chain variable region segment and the light chain variable region segment.
5. The antibody molecule of any of paragraphs 1-4, comprising (a) a heavy chain variable region segment comprising the CDR1, CDR2, and CDR3 of the heavy chain variable region VH123, VH148, or VH175; and (b) a light chain variable region segment comprising the CDR1, CDR2, or CDR3 of the light chain variable region VK-65.
6. The antibody molecule of any of paragraphs 1-5, further comprising an Fc region.
7. The antibody molecule of paragraph 6, wherein the Fc region comprises a mutation.
8. The antibody molecule of any of paragraphs 1-7, comprising an Fc region comprising the mutations of FcMut215.
9. The antibody molecule of any of paragraphs 1-8, wherein the heavy chain variable region segment comprises the CDR1, CDR2, and CDR3 of the heavy chain variable region VH123.
10. The antibody molecule of any of paragraphs 1-8, wherein the heavy chain variable region segment comprises the CDR1, CDR2, and CDR3 of the heavy chain variable region VH148.
11. The antibody molecule of any of paragraphs 1-8, wherein the heavy chain variable region segment comprises the CDR1, CDR2, and CDR3 of the heavy chain variable region VH175.
12. The antibody molecule of any of paragraphs 1-11, comprising the amino acid sequence of heavy chain variable region VH123, VH148, or VH175, or an amino acid sequence at least 85%, 90%, 95%, 98%, or 99% identical thereto.
13. The antibody molecule of paragraph 12, comprising the amino acid sequence of heavy chain variable region VH123, or an amino acid sequence at least 85%, 90%, 95%, 96%, 97%, 98%, or 99% identical thereto or differing by no more than 1, 2, 3, 4, 5, 6, 7, 8, 9, 10, 11, 12, 13, 14, or 15 amino acids therefrom.
14. The antibody molecule of paragraph 12, comprising the amino acid sequence of heavy chain variable region VH148, or an amino acid sequence at least 85%, 90%, 95%, 98%, or 99% identical thereto or differing by no more than 1, 2, 3, 4, 5, 6, 7, 8, 9, 10, 11, 12, 13, 14, or 15 amino acids therefrom.
15. The antibody molecule of paragraph 12, comprising the amino acid sequence of heavy chain variable region VH175, or an amino acid sequence at least 85%, 90%, 95%, 96%, 97%, 98%, or 99% identical thereto or differing by no more than 1, 2, 3, 4, 5, 6, 7, 8, 9, 10, 11, 12, 13, 14, or 15 amino acids therefrom.
16. The antibody molecule of any of paragraphs 1-15, comprising the amino acid sequence of light chain variable region VK-65, or an amino acid sequence at least 85%, 90%, 95%, 96%, 97%, 98%, or 99% identical thereto or differing by no more than 1, 2, 3, 4, 5, 6, 7, 8, 9, 10, 11, 12, 13, 14, or 15 amino acids therefrom.
17. An anti-hemagglutinin (HA) antibody molecule comprising (a) CDR1, CDR2, and CDR3 of the heavy chain variable region VH123; (b) CDR1, CDR2, or CDR3 of the light chain variable region VK-65; and optionally, (c) an Fc region comprising FcMut215.
18. An anti-hemagglutinin (HA) antibody molecule comprising (a) CDR1, CDR2, and CDR3 of the heavy chain variable region VH148; (b) CDR1, CDR2, or CDR3 of the light chain variable region VK-65; and optionally, (c) an Fc region comprising FcMut215.
19. An anti-hemagglutinin (HA) antibody molecule comprising (a) CDR1, CDR2, and CDR3 of the heavy chain variable region VH175; (b) CDR1, CDR2, or CDR3 of the light chain variable region VK-65; and optionally, (c) an Fc region comprising FcMut215.
20. An anti-hemagglutinin (HA) antibody molecule comprising (a) the heavy chain variable region VH123; (b) the light chain variable region VK-65; and optionally, (c) an Fc region comprising FcMut215.
21. An anti-hemagglutinin (HA) antibody molecule comprising (a) the heavy chain variable region VH148; (b) the light chain variable region VK-65; and optionally, (c) an Fc region comprising FcMut215.
22. An anti-hemagglutinin (HA) antibody molecule comprising (a) the heavy chain variable region VH175; (b) the light chain variable region VK-65; and optionally, (c) an Fc region comprising FcMut215.
23. A pharmaceutical composition comprising the antibody molecule of any of paragraphs 1-22 and a pharmaceutically acceptable carrier.
24. A nucleic acid (e.g., an isolated nucleic acid) encoding a heavy chain variable region segment, a light chain variable region segment, or both, of the antibody molecule of any of paragraphs 1-22.
25. A vector comprising the nucleic acid of paragraph 24.
26. A cell (e.g., an isolated cell) comprising the nucleic acid of paragraph 24 or the vector of paragraph 25.
27. A method of producing an antibody molecule, comprising culturing the cell of paragraph 26 under conditions that allow production of the antibody molecule, thereby producing the antibody molecule.
28. A kit comprising the antibody molecule of any of paragraphs 1-22 and instructions for use.
29. A method of treating or preventing an influenza virus infection, or a symptom thereof, in a subject, comprising administering to the subject an effective amount of the antibody molecule of any of paragraphs 1-22.
30. The method of paragraph 29, which prevents an influenza virus infection, optionally wherein the method prevents an influenza virus infection for at least 5, 10, 15, 20, 25, 30, 40, 45, 50, 60, 70, 80, 90, 100, 150, 200 days or more.
31. The method of paragraph 19 or 20, wherein the subject is at risk of having an influenza infection, e.g., a subject 65 years and older or younger than 2 years old, a subject having asthma, a neurologic and neurodevelopment condition, a blood disorder (e.g., sickle cell disease), a chronic lung disease (e.g., chronic obstructive pulmonary disease (COPD) or cystic fibrosis), an endocrine disorder (e.g., diabetes mellitus), a heart disease (e.g., a congenital heart disease, a congestive heart failure, or a coronary artery disease), a kidney disease, a liver disorder, or a metabolic disorder (e.g., an inherited metabolic disorder or a mitochondrial disorder), a subject who is obese with a body mass index (BMI) of 40 or higher, a subject younger than 19 years old on a long-term aspirin- or salicylate-containing medication, a subject with a weakened immune system due to disease (e.g., a subject with HIV or AIDS, or a cancers, e.g., a leukemia) or a medication (e.g., a subject receiving a chemotherapy or radiation treatment for cancer, or a subject with a chronic condition requiring a chronic corticosteroid that suppresses the immune system), or a subject who has had a stroke.
32. The method of any of paragraphs 29-31, wherein the antibody molecule is administered before the subject is exposed to an influenza virus.
33. The method of any of paragraph 29-32, wherein the antibody molecule is administered subcutaneously or intramuscularly.
34. The method of any of paragraphs 29-33, wherein the antibody molecule is administered once or no more than twice during an influenza season.
35. The method of any of paragraphs 29-34, wherein the influenza virus is an influenza A virus, optionally wherein the influenza virus is an H1 or H3 influenza virus.

### EXAMPLES

### Example 1: Design and Functional Characterization of an Exemplary Anti-HA Antibody Molecule

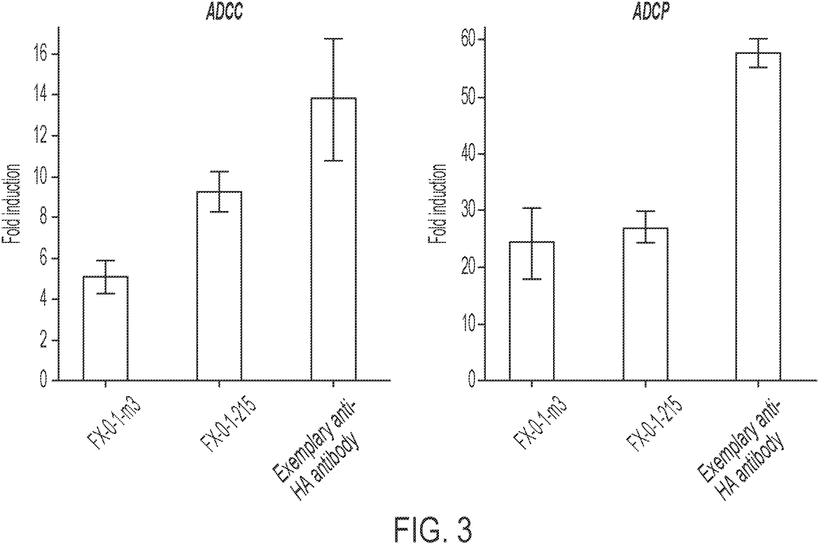

As shown in FIGS. 1-3, an exemplary anti-HA antibody molecule showed potent binding and neutralization activity, while significantly enhancing half-life in relevant animal model. In addition, Fc and Fab engineering (e.g., Fc mutations T307Q/Q311V/A378V) of the exemplary anti-HA antibody molecule enhanced effector functions such as ADCC and ADCP for further improvement in overall potency.

### Re-Scaffolding FX-0-1-m3 to Improve Half-Life for Prophylactic Use

### Grafting FX-0-1-m3 Specificity Determining Regions (SDRs) Into Alternative Germline Scaffolds

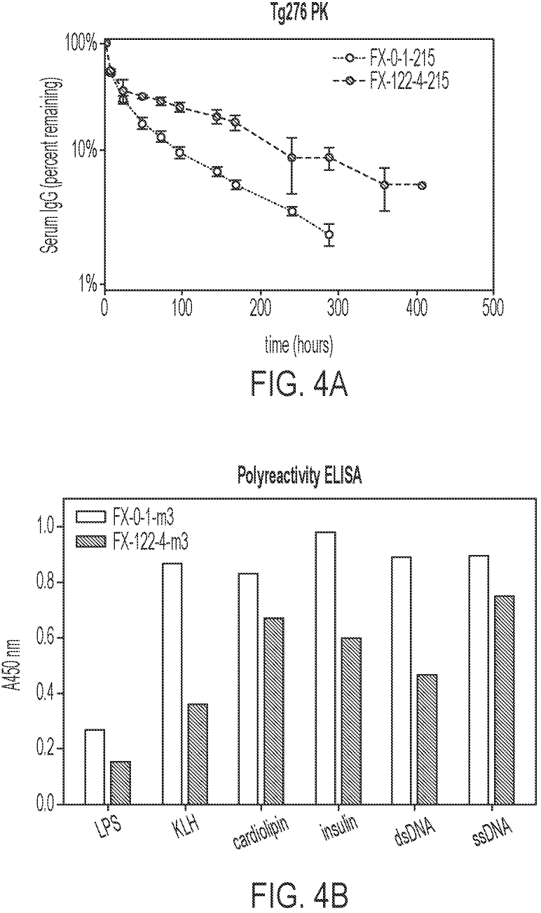

Because alternative germlines can improve antibody half-life, the SDRs of the anti-hemagglutinin (HA) antibody, FX-0-1-m3, were re-scaffolded. Grafting was performed in a manner similar to humanization of antibodies. Multiple scaffolds were identified that retained binding to H1 and H3 that also demonstrated improved biophysical properties. Re-scaffolding design based on the VH-169 germline significantly improved the PK of the antibody (FIGS. 4A-4B). Further experiments, revealed that changes to the base of HCDR3, encoded by the J gene, were responsible for the improved PK of the antibody in the new scaffold. As shown in FIG. 4A, the re-scaffolded antibody molecule, FX-122-4-215, demonstrated improvements in half-life in a relevant animal model and as depicted in FIG. 4B, it also showed improvements in polyreactivity assays. In conclusion, these data demonstrate that subtle changes to the framework of an antibody molecule can impact position and/or dynamics of CDR loops and impact the PK of the antibody molecule.

### Predicted Hydrophobic Clusters and CDR Sites on FX-0-1-m3 Surface

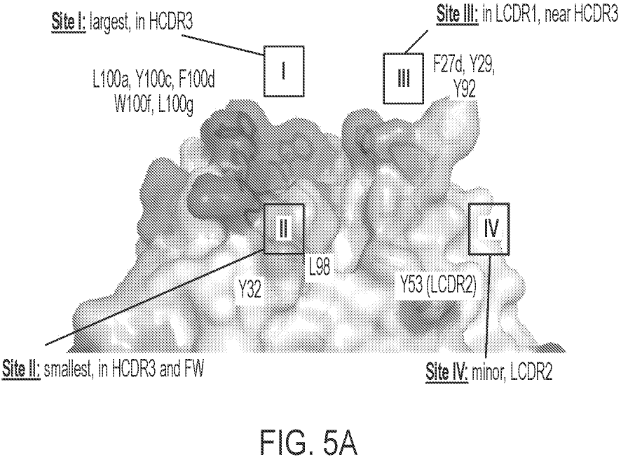

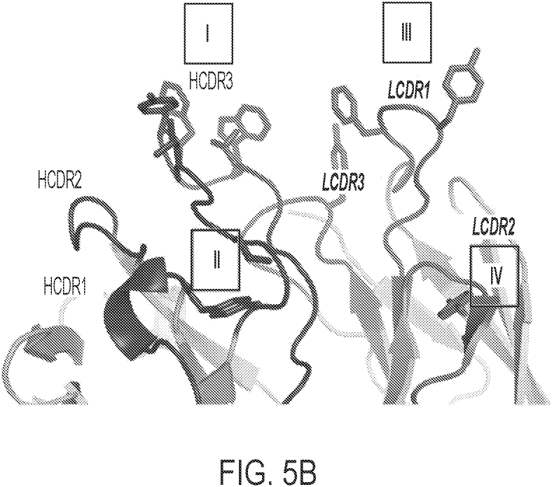

Four potential aggregation sites with surface hydrophobic residues were identified in FX-0-1-m3 by in silico analysis. As shown in FIG. 5A, Site I was the largest hydrophobic cluster found on the surface of FX-0-1-m3, composed of five amino acids in the HCDR3 and is important for HA binding. Site II was located at the HCDR3 torso domain formed by the L98 residue of HCDR3 and the Y32 residue of HCDR1. Site III was a hydrophobic cluster located on the light chain in LCDR1, and is composed of three residues (F27d, Y29, and Y92). Site IV (a minor site) was located on the light chain in LCDR2 and contains a single amino acid, Y53. As depicted in FIG. 5B, the four sites converge primarily in the HCDR3 and LCDR1 regions.

### Affinity Enhancement by Engineering Framework of the VH

The VH:FWR3 (framework 3) region of the FX-0-1-m3 antibody (residues 73-76, also called HCDR4) is in close proximity with the HA1 domain of the HA polypeptide. Increasing antibody interface area could improve hydrophobic shape and complementarity. It was observed that in all available H3 structures the C β atom at position 278 of the HA polypeptide faces towards the polar side of position N76 of the VH of the FX-0-1-m3 antibody. It was investigated to determine whether insertion of an aliphatic or aromatic amino acid at position 76 of the VH could provide improved hydrophobic complementarity with the HA polypeptide at position N278. Mutation of the HFWR3 residues S74, K75 and N76 (S74Y/W, K75Y/W, N76S/L/F/W) significantly improved the affinity. For instance, mutation to L or W at position 76 of the FX-0-1-m3 antibody increased binding to H3 HA and mutation to W at position 75 of the FX-0-1-m3 antibody increased binding to H3 HA.

### Example 2: Engineering a Human Antibody Molecule for Prophylactic Use Against Influenza A Virus Infection

In this example, FX-0-1-m3, an exemplary antibody molecule having particular designed attributes was engineered. These attributes included, e.g., targeting an epitope on hemagglutinin that is conserved across different influenza subtypes and strains and is constrained in its ability to mutate; strong binding affinity for HA (Kd ≤ 1 nM) of the various subtypes; potent neutralization activity (IC₅₀ ≤ 2 µg/mL); long in vivo circulating half-life (e.g., to provide season long protection); solubility and viscosity to support subcutaneous/intramuscular administration of the target dose; and mucosal transport leading to sufficient availability in the upper respiratory tract.

To identify an antibody with the above properties, FX-0-1-m3, an exemplary human antibody with broad neutralization activity against influenza A virus was engineered to have properties amenable to prophylactic administration. In this example, the Fab and Fc regions were engineered to extend half-life to at least 45 days and to enhance the affinity of the antibody for H3 HA protein. The engineered antibody was also evaluated for its amenability for subcutaneous administration, e.g., for season-long protection against influenza A infection.

### Overview of Antibody Engineering

To support the use of an antibody as a single administration prophylactic against influenza, it would be desirable for the antibody to have broad activity against seasonal strains (H1N1 and H3N2 subtype) and to be available in the target organs for the entire season (e.g., at least about 5 to 6 months) at efficacious concentrations. Further, to support intramuscular or subcutaneous (SubQ) administration, it was desirable for the antibody to be able to be formulated at high concentrations (e.g., greater than about 100 mg/mL).

Stability studies showed that the exemplary antibody FX-0-1-m3 was thermally stable for at least 24-36 months at 4° C., and remained active in sera for an extended period of time. Specifically, toxicology studies in cynomolgus monkeys showed that FX-0-1-m3 in sera retained binding to HA at least 42 days past final dosing. Further, formulation studies showed that it could be formulated at concentrations as high as 148 mg/mL while maintaining a low viscosity (< 10 cP), supporting SubQ/IM administration. It was readily soluble at high concentrations (>100 mg/mL), dynamic light scattering studies showed that the exemplary antibody exists as dimers at higher concentrations (>25 mg/mL).

FX1-m3 was modified to further enhance pharmacokinetics, physicochemical properties, and affinity for group 2 HA (in particular, H3), while maintaining the desirable properties of the exemplary antibody molecule. Modified antibody molecules were generated as described below. Candidate antibody molecules were assigned an identifier of the format “FX-XXX-YY-ZZZ,” in which the XXX indicated the heavy chain sequence (e.g., as described herein), YY indicated the light chain sequence (e.g., as described herein), and ZZZ indicated the Fc domain variant (e.g., as described herein).

### Fc Engineering

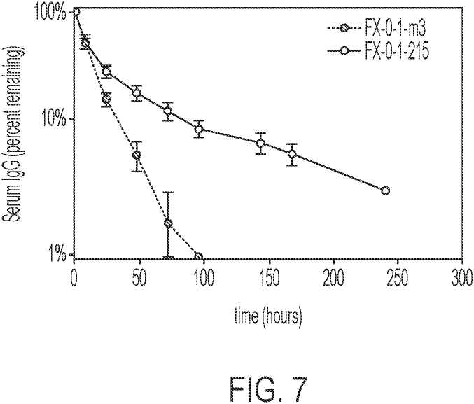

A structural- and network-based framework was developed to interrogate the interaction of the Fc domain with FcRn at neutral and acidic pH. Multiple Fc variants were identified that confer enhancement in half-life and retain or enhance effector functions such as ADCC and CDC. DF215 (also known as FcMut215 herein) is one such Fc domain variant (comprising Fc mutations Q307/V311/V378), which was shown to enhance half-life of a control antibody by greater than 3-fold in cynomolgus monkeys. DF215 was thermally stable and has been shown to enhance ADCC activity. Here, DF215 was combined with the Fab of the exemplary antibody to produce antibody molecule FX-0-1-215. When the pharmacokinetic properties of FX-0-1-215 were assessed in Tg276 transgenic mice, it persisted in sera for significantly longer than the parental FX-0-1-m3 mAb (FIG. 7). Thus, DF215 Fc domain was chosen for incorporation into lead candidates for half-life extension.

### Fab Engineering for Improved Biophysical Properties

The Fab domain of an antibody can influence circulating half-life through a variety of factors including, e.g., (i) interaction with serum and cell components, (ii) influencing the rate of fluid phase pinocytosis, (iii) receptor-mediated endocytosis (for antibodies targeting endogenous target), (iv) presence of anti-drug antibodies, and (v) influencing the Fc-FcRn interaction. As shown above, half-life can be extended by incorporating the DF215 mutation. Fab engineering provides an additional opportunity to further enhance half-life. Properties such as low melting temperature, charge, self-interaction, non-specific binding to tissues and general polyreactivity are all characteristics that could adversely impact half-life.

Many of these biophysical properties of the exemplary antibody were sought to be assessed through a panel of assays. These assays were performed using antibody molecule FX-0-1-m3 (comprising the heavy chain of FX-0-1-m3, GS->RT Cκ variant, and a heavy chain constant region G1m3 allotype). Thermal stability was measured by differential scanning fluorimetry (DSF) using SYPRO™ Orange. From the denaturation profile, the melting temperature (Tm) of the CH2, CH3, and Fab domains can be inferred by taking the first derivative of the fluorescence curve and identifying local maximum y values. The human CH2 domain unfolds at about 69° C., the human CH3 domain unfolds at about 85° C., and the Fab unfolding temperature varies widely between antibodies. To assess the melting temperature of Fab domain of the exemplary antibody, its Fab was purified post-papain treatment. DSF analysis revealed the unfolding of the Fab domain occurred at about 70° C., which is close to the CH2 unfolding temperature. As such, full-length FX-0-1-m3 showed a single unfolding signal at about 69° C. when assessed in DSF (Table 3).

TABLE 3

Tm values for FX-0-1-m3 Fab and full-length antibody FX-0-1-m3

| mAb | Tm (°C) (CH2) | Tm (°C) (CH3) | Tm (°C) (Fab) |
|---|---|---|---|
| FX-0-1-m3-Fab | N/A | N/A | 70.65 |
| FX-0-1-m3 | 69.51 | Not detectable | Not detectable |

An assessment of polyreactivity of FX-0-1-m3 was performed using a biolayer interferometry (BLI) polyreactivity assay. Briefly, polyclonal human IgG was immobilized onto anti-human CH1 biosensors followed by exposure to a panel of test antibodies. Excessive binding of the test antibodies to the loaded biosensors resulted in a shift in mass on the biosensor and thus an increase in BLI signal. An increase in BLI signal would indicate that the antibody nonspecifically binds to polyclonal human IgG. Table 4 summarizes the maximum signals for FX-0-1-m3 and a control antibody, where higher maximum signals suggest more nonspecific binding to human polyclonal antibody on the anti-human CH1 biosensors. FX-0-1-m3 exhibited relatively high polyreactivity in this assay.

TABLE 4

Maximum signal measurements (binding response) for polyreactivity for a variety of antibodies as measured by BLI

| mAb | Max Signal (nm) |
|---|---|
| Control Antibody | 0.046 |
| FX-0-1-m3 | 0.738 |

In addition to testing polyreactivity, an AC-SINS assay was carried out to quantify the propensity of the FX-0-1-m3 to self-aggregate. The AC-SINS assay involves concentrating dilute solutions of mAbs around gold nanoparticles pre-coated with polyclonal capture antibodies. Interaction between immobilized mAbs leads to reduced inter-particle separation distances and increased plasmon wavelengths, which in turn alters the color of the gold colloidal solution. This change in color, or change in maximum wavelength, can be quantified using a standard plate reader. Antibodies with the propensity to self-interact demonstrate red-shifted plasmon wavelengths (detected as change in wavelength maximum absorbance). Table 5 shows both the parabola vertex as well as vertex shift from baseline at 530 nm for FX-0-1-m3 and a control mAb. Antibodies with higher vertex shift values have higher self-interaction proclivity. FX-0-1-m3 exhibited a high propensity to self-aggregate with a vertex shift greater than 20 nm.

TABLE 5

AC-SINS assay comparing parabola vertex (nm) and vertex shift (nm) of various antibodies

| Antibody | Parabola vertex (nm) | | Mean vertex (nm) | Vertex shift (nm) -two experiments | | Mean vertex shift (nm) |
|---|---|---|---|---|---|---|
| Control mAb | 535.1 | 537.3 | 536.2 | 5.1 | 7.3 | 6.2 |
| FX-0-1-m3 | 556.5 | 550.8 | 553.7 | 26.5 | 20.8 | 23.7 |

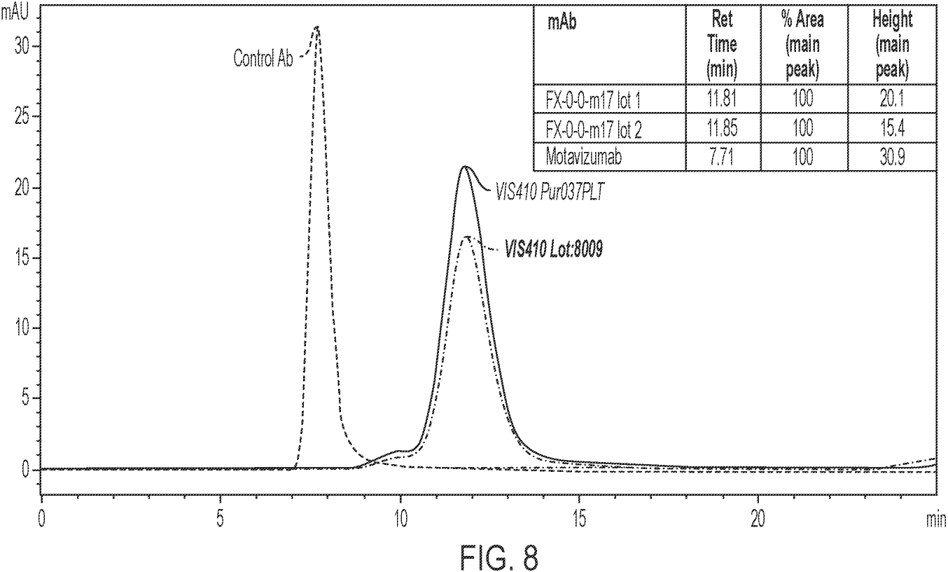

FX1-m3 was also assessed by size exclusion chromatography. FX1-m3, when resolved on a Phenomenex Bio-Sep S3000 column, eluted at about 19 minutes, as compared to most typical antibodies that eluted around 8 minutes (FIG. 8). This was indicative of association of the antibody to the column matrix.

The combination of the DSF, polyreactivity, AC-SINS, and SEC data suggested that FX-0-1-m3 had atypical biophysical properties and that engineering efforts to enhance these were beneficial for the development of a prophylactic antibody (e.g., by extending half-life). Both the framework region as well as the CDRs can influence many biophysical properties. As such, the impact of modifying both the FWRs and CDRs of FX-0-1-m3 was assessed with respect to the impact on biophysical and pharmacokinetic properties.

### Re-Scaffolding FX-0-1-m3 CDR Residues Onto Alternative Human Germlines

The CDRs of antibody molecule FX-0-1-m3 were rescaffolded onto alternate human VH and VL germlines and evaluated for their impact on antigen binding and biophysical properties.

The original VH and VL scaffolds of FX-0-1-m3 are from human VH3-30*01 and V_{K}4-1 germlines, respectively, which have generally good biophysical properties. Many HA stem binding antibodies belong to VH1-69*04 germline and the VH-CDRs of FX-0-1-m3 were grafted onto VH1-69*04 as well as the other germlines frameworks including VH3-30*01, VH3-30*02, VH3-30-03*03 and VH1-8*01. Similarly, the Vκ- CDRs were grafted on to Vκ1-39*01, Vκ4-1*01 and Vκ3-15*01 germline frameworks. In addition, variants of these germline frameworks with selected framework mutations at Vernier residues were also tested. Ten VHs and six VLs were designed representing three VH germlines and three VL germlines (Table 6).

TABLE 6

Germline usage for rescaffolded FX-0-1-m3 variants

| ID | Variant (Heavy) | Germline | Germline Identity (%) |
|---|---|---|---|
| VH-0 | Exemplary anti-HA | IGHV3-30-3*02 | 87.8 |
| VH-3 | des_ VH1_1 | IGHV3-30-3*02 | 95.9 |
| VH-4 | des_VHI_2 | IGHV3-30*01 | 94.9 |
| VH-5 | des_VH1_3 | IGHV3-30*01 | 93.9 |
| VH-6 | des_VH1_4 | IGHV3-30*01 | 94.9 |
| VH-7 | VH2 1 | IGHV3-30*02 | 91.8 |
| VH-8 | des_VH2_2 | IGHV3-30*02 | 92.9 |
| VH-9 | des_VH3_1 | IGHV1-69*04 | 83.7 |
| VH-10 | des_VH3_2 | IGHV1-69*04 | 84.7 |
| VH-11 | des_VH4_1 | IGHV1-8*01 | 84.7 |
| VH-12 | des_VH4_2 | IGHV1-8*01 | 84.7 |
| VK-0 | Exemplary anti-HA | IGKV1-39*01 | 85.9 |
| VK-2 | des_VL1_1 | IGKV1-39*01 | 87.9 |
| VK-3 | des_VL1_2 | IGKV1-39*01 | 89.9 |
| VK-4 | des_ VL2_1 | IGKV1-39*01 | 92.1 |
| VK-5 | des_VL2_2 | IGKV4-1*01 | 91.1 |
| VK-6 | des_VL3_1 | IGKV3-15*01 | 84.8 |
| VK-7 | des_VL3_2 | IGKV3-15*01 | 85.9 |

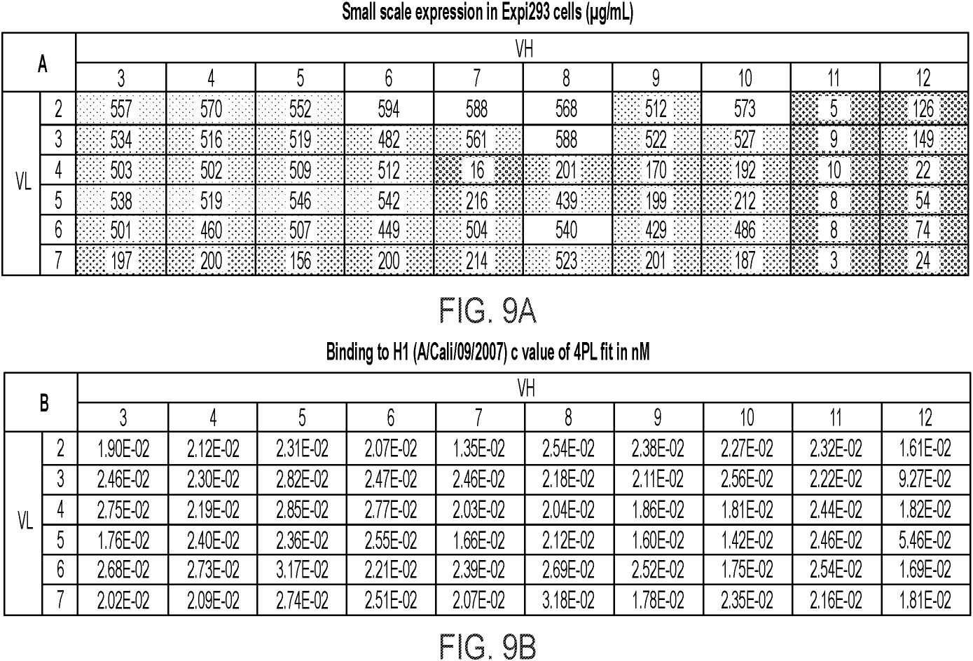

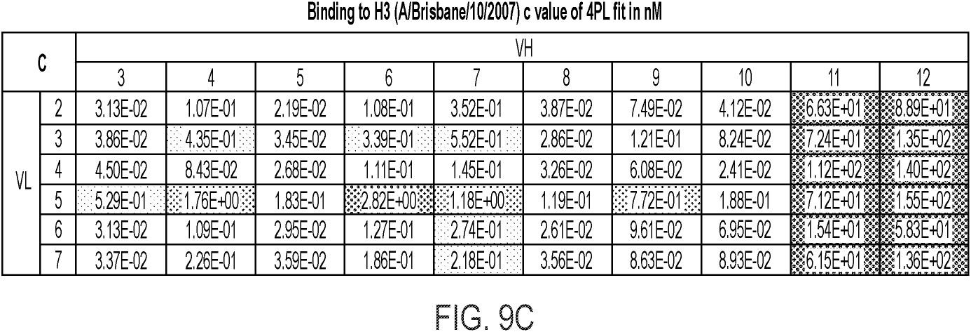

The VH- and VL-containing plasmids were assessed in a combinatorial manner and a total of 60 antibodies were expressed in small scale Expi293 cultures. Supernatant was harvested 4 days post-transfection and antibody expression was measured by Octet (FIG. 9A). Most combinations expressed well except for all antibodies containing heavy chain FX-VH-11 and most antibodies containing FX-VH-12. The supernatant was used in an HA binding ELISA to test for the binding properties of the newly designed antibodies against H1 (A/CA/09/2007) and H3 (A/Brisbane/10/2007) (FIGS. 9B-9C). All combinations bound to H1 HA; however, differential binding was observed with H3 HA. In particular, all antibodies containing heavy chains FX-VH- 11 and FX-VH-12, along with some antibodies containing light chain FX-VK-5, displayed reduced binding to H3.

From this initial set, a panel of 18 combinations were chosen to be assessed for large scale expression and purification and evaluation of binding and biophysical properties (Table 7). Multiple scaffolds were identified that retained binding to H1 and H3 and had improvements in biophysical properties. Rescaffolded designs based on the VH1-69 VH germline were considered of particular interest.

TABLE 7

Biophysical and functional data set for VIS-FLX scaffold designs

| Antibody | SEC elution time (minutes) | AC-SINS vertex x-coordinate (nm) | AC-SINS latus rectum (nm) | poly reactivity octet max binding response (nm) | TM (°C) |
|---|---|---|---|---|---|
| FX-0-1-m3 | 10.5 | 550.5 | 2.48E+05 | 0.775 | 68.9 |
| FX-3-2-m3 | 12.2 | 546.3 | 2.07E+05 | 0.521 | 68.8 |
| FX-3-3-m3 | 11.7 | 541.6 | 2.01E+05 | 0.485 | 68.5 |
| FX-3-6-m3 | 13.1 | 552.3 | 1.80E+05 | 0.774 | 68.5 |
| FX-4-2-m3 | 11.4 | 550.7 | 1.92E+05 | 0.758 | 68.5 |
| FX-5-2-m3 | 12.6 | 555.9 | 2.21E+05 | 0.992 | 68.8 |
| FX-5-4-m3 | 11.7 | 553.3 | 1.55E+05 | 0.875 | 67.9 |
| FX-5-6-m3 | 13.4 | 553.7 | 2.28E+05 | 1.134 | 68.5 |
| FX-6-2-m3 | 11.5 | 556.1 | 3.01E+05 | 0.798 | 68.8 |
| FX-7-6-m3 | 13.2 | 551.5 | 1.89E+05 | 0.703 | 68.8 |
| FX-8-3-m3 | NA | 555.2 | 1.93E+05 | 0.343 | 68.5 |
| FX-8-6-m3 | NA | 553.8 | 1.79E+05 | 1.007 | 68.5 |
| FX-8-7-m3 | NA | 554.9 | 1.58E+05 | 0.795 | 68.5 |
| FX-9-4-m3 | NA | 541.0 | 1.71E+05 | 0.345 | 68.5 |
| FX-10-2-m3 | NA | 554.5 | 2.56E+05 | 0.681 | 68.5 |
| FX-10-4-m3 | NA | 551.8 | 1.67E+05 | 0.585 | 68.5 |
| FX-10-6-m3 | NA | 552.7 | 1.50E+05 | 0.862 | 68.2 |
| FX-11-4-m3 | NA | 545.9 | 1.46E+05 | 0.430 | 70.3 |
| FX-12-2-m3 | NA | 547.9 | 1.79E+05 | 0.391 | 68.8 |

### Engineering of CDRs

Spatial aggregation propensity (SAP) site calculation was performed using Discovery Studio and Aggrescan3D software. A static model of the exemplary anti-HA antibody molecule FX-0-1-m3 was used to identify four hydrophobic sites/patches (I to IV) involving CDR residues (FIG. 5A).

Site I was the largest hydrophobic cluster found on the surface of FX-0-1-m3, composed of five amino acids in the HCDR3. This site was important for HA binding. Single or double mutations of site I residues to less hydrophobic or polar amino acids was carried out (L100bQ/N, Y100cF/M/R, F100dQ, E100eD/R, W100fH/R, L100gM/N, S100hN). Many of these variants decreased the SEC elution time to less than 9 minutes but had little or no impact in the AC-SINS assay (Table 8). Additionally, many of the mutations at this site reduced or abolished binding to H3 HA (Table 8). None of the variants improved the biophysical characteristics while maintaining H3 HA binding.

TABLE 8

Engineering of SAP site I

| | | | | | | |
|---|---|---|---|---|---|---|
| FX-14-1-m3 | 3-30-3*02 | Fl00dC, L100gC | 548.56 | 0.599 | 96.6 | >20000 |
| FX-17-9-m3 | 3-30-3*02 | Wl00fC | 557.67 | 0.619 | 994.8 | >20000 |
| FX-19-1-m3 | 3-30-3*02 | Ll00gM, Sl00hN | 557.78 | 1.127 | 8.7 | 371.9 |
| FX-20-10-m3 | 3-30-3*02 | F100dQ | 537.50 | 0.481 | 23.7 | >20000 |
| FX-26-10-m3 | 3-30-3*02 | L100gN | 545.98 | 0.572 | 10.4 | >20000 |
| FX-37-10-m3 | 3-30-3*02 | L100aN, Fl00dY | 555.96 | 0.749 | 54.9 | >20000 |
| FX-38-10-m3 | 3-30-3*02 | L100aQ, F100dY | 551.76 | 0.635 | 5.5 | >20000 |
| FX-39-10-m3 | 3-30-3*02 | W100fH | 554.31 | 0.510 | 9.0 | >20000 |
| FX-40-10-m3 | 3-30-3*02 | W100fR | 554.23 | 0.549 | 12.6 | >20000 |
| FX-51-1-m3 | 3-30-3*02 | Y100cM | 550.64 | 0.594 | 6.0 | >20000 |
| FX-62-1-m3 | 3-30*18 | Y100cM | 550.41 | 0.534 | 17.1 | >20000 |

SAP Site II was located at the HCDR3 torso domain formed by the L98 residue of HCDR3 and Y32 residue of HCDR1. Mutation of Leu to Asn at position VH 98 (L98N) dramatically improved biophysical properties with low polyreactivity and AC-SINS and typical SEC antibody elution time when compared to the control mAb (Table 9). However, introduction of this single mutation completely abolished group 2 HA binding. Other mutations to L98 such as L98D and L98Q showed moderate improvement in biophysical properties but again with the loss of group 2 HA binding. The hydrophobic side chain of L98 interacted with the conserved N53 residue of HA2. Although the N53 residue was conserved across group 1 and group 2 HA, the lack of binding of the L98N variant to group 2 HA specifically highlighted the importance of this interaction for group 2 HAs such as H3 HA. Additional engineering efforts to restore H3 binding with L98 variants were not successful. The other residue in SAP site II was Y32. The side chain aromatic ring of Y32 supported the HCDR3 conformation. Mutations to this site, such as Y32R, retained HA binding, with some improvement in biophysical properties.

TABLE 9

Engineering of SAP site II

| | | | | | | |
|---|---|---|---|---|---|---|
| FX-65-1-m3 | 3-30-3*02 | L98A | 555.31 | 0.662 | 2.7 | >20000 |
| FX-71-1-m3 | 3-30-3*02 | L98D | 552.47 | 0.387 | 9.1 | >20000 |
| FX-56-1-m3 | 3-30-3*02 | L98I | 552.53 | 0.773 | 8.9 | >20000 |
| FX-72- 1-m3 | 3-30-3*02 | L98K | 553.66 | 0.734 | 3.1 | >20000 |
| FX-69-1-m3 | 3-30-3*02 | L98M | 559.92 | 0.872 | 2.0 | 10367.0 |
| FX-24-1-m3 | 3-30-3*02 | L98N, Y100cM | 535.07 | 0.112 | 16.4 | >20000 |
| FX-27-10-m3 | 3-30-3*02 | L98N, Y100cM, L100gN, S(100H)N | 533.46 | 0.120 | 3005.8 | >20000 |
| FX-52-1-m3 | 3-30-3*02 | L98N | 531.58 | 0.149 | 18.5 | >20000 |
| FX-52-10-m3 | 3-30-3*02 | L98N | 532.98 | 0.126 | 17.6 | >20000 |
| FX-61-1-m3 | 3-30*18 | L98N | 532.38 | 0.123 | 41.9 | 4406.4 |
| FX-61-10-m3 | 3-30*18 | L98N | 531.76 | 0.139 | 19.1 | >200000 |
| FX-64-1-m3 | 3-30-3*02 | T30S, L98N | 534.74 | 0.215 | NA | NA |
| FX-64-21-m3 | 3-30-3*02 | T30S, L98N | 533.67 | 0.206 | NA | NA |
| FX-64-22-m3 | 3-30-3*02 | T30S, L98N | 533.39 | 0.211 | NA | NA |
| FX-64-23-m3 | 3-30-3*02 | T30S, L98N | 536.67 | 0.285 | NA | NA |
| FX-25-1-m3 | 3-30-3*02 | L98Q, Y100cM | 544.18 | 0.391 | 8.5 | >20000 |
| FX-53-1-m3 | 3-30-3*02 | L98Q | 552.28 | 0.581 | 14.5 | 2019.9 |
| FX-70-1-m3 | 3-30-3*02 | L98R | 557.89 | 0.948 | 3.3 | >20000 |
| FX-54-1-m3 | 3-30-3*02 | L98T | 551.45 | 0.600 | 16.0 | >20000 |
| FX-55-1-m3 | 3-30-3*02 | L98V | 552.07 | 0.773 | 17.1 | >20000 |
| FX-76-1-m3 | 3-30-3*02 | Y32F, L98R | 554.26 | 0.778 | 3.8 | |
| FX-113-3-m3 | 3-30-3*02 | T30S, Y32R, G54A, N101D, P102Y | 558.56 | 0.373 | 3.6 | 51.2 |
| FX-127-4-m3 | 1-69*06 | T30S, S31T, Y32R, G54A, R97Q, N101D, P102Y | 553.17 | 0.372 | 10.5 | 2303.9 |

Site III is a hydrophobic cluster located on the light chain composed of three residues, F27d, Y29 and Y92. Since only the side chain hydroxyl group of the Y92 residue was solvent accessible, it was not considered for mutation. It was determined that F27d played an important role in group 2 HA binding. The aromatic side chains of F27d and Y29 were exposed to solvent and sequence-based alignment to germline revealed that polar residues are typically found at positions 27d and 29. Mutation of F27d and Y29 to polar or acidic residues, such as F27dS/Y29N or F27dE/Y29E, showed improved biophysical properties with reduction in H3 HA binding (Table 10).

TABLE 10

Engineering of SAP site III

| | | | | | |
|---|---|---|---|---|---|
| FX-0-14-m3 | F27dS, Y29N, K30Q | 555.70 | 0.351 | 8.6 | 5822.2 |
| FX-0-24-m3 | F27dS, Y29N | 553.86 | 0.386 | 6.0 | 669.0 |
| FX-0-25-m3 | F27dS, Y29D | 543.11 | 0.357 | 6.8 | 4859.3 |
| FX-0-26-m3 | F27dS, Y29E | 545.03 | 0.336 | 2.3 | 4606.3 |
| FX-0-27 -m3 | F27dD, Y29N | 542.63 | 0.365 | 5.9 | 2276.6 |
| FX-0-28-m3 | F27dD, Y29D | 542.34 | 0.283 | 6.4 | >20000 |
| FX-0-29-m3 | F27dD, Y29E | 539.43 | 0.389 | 7.0 | >20000 |
| FX-0-30-m3 | F27dE, Y29N | 544.30 | 0.281 | 8.0 | >20000 |
| FX-0-31-m3 | F27dE, Y29D | 535.78 | 0.288 | 9.1 | >20000 |
| FX-0-32-m3 | F27dE, Y29E | 544.91 | 0.261 | 8.6 | >20000 |
| FX-0-33-m3 | F27dQ, Y29N | 553.99 | 0.365 | 9.1 | 1028.6 |
| FX-0-34-m3 | F27dQ, Y29D | 548.17 | 0.293 | 4.2 | 2824.9 |
| FX-0-35-m3 | F27dQ, Y29E | 544.79 | 0.307 | 8.4 | 10194.0 |
| FX-0-36-m3 | F27dH, Y29N | 550.69 | 0.382 | 9.4 | 670.2 |
| FX-0-37-m3 | F27dH, Y29D | 551.73 | 0.336 | 8.1 | >20000 |
| FX-0-38-m3 | F27dH, Y29E | 546.79 | 0.334 | 7.1 | 4877.0 |

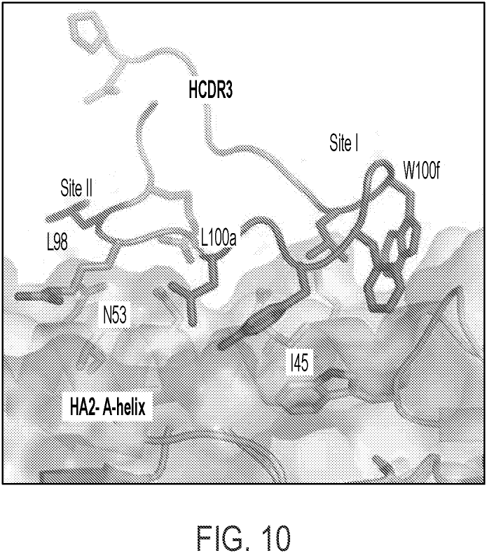

SAP site IV was comprised of a single amino acid, Y53 on the LCDR2. This site was previously identified as a minor site, given it comprised only a single amino acid; however, given the results from mutation of the other sites, the impact of modifying this site on the biophysical properties was evaluated. This site was mutated in the light chain of VK design 3 and VK design 4 to a serine and threonine, respectively. These two light chains were found in antibodies FX-8-3-m3 and FX-9-4-m3, both of which exhibited improved developability profiles and maintained H3 binding. The parental germline, 1-39*01, contained a serine at this location. Removal of the hydrophobic tyrosine from this site had no impact on functionality of the antibody (Table 11). Spatially, Y53 was close to VH residues N101 and P102 of the heavy chain (FIG. 10). Indeed, interrogation of the changes made at positions 101 and 102 in the HCDR3 torso region encoded by the J gene (as part of the rescaffolding exercise) showed modest improvements in biophysical properties (e.g., FX-8-3-m3 containing N101D/P102Y).

TABLE 11

Engineering of SAP Site IV

| | | | | | | |
|---|---|---|---|---|---|---|
| FX-9-4-215 | 4-1*01 | R24K, I27bV, G51A, Y53T, L54R | 541 | 0.345 | 16.0 | 9.6 |
| FX-8-3-215 | 1-39*01 | W50A, G51A, Y53S, E55Q | 555 | 0.334 | 11.7 | 233.0 |
| FX-0-92-m3 | 1-39*01 | Y53D | 559 | 0.491 | NA | 52.6 |
| FX-0-93-m3 | 1-39*01 | Y53E | 554 | 0.772 | NA | 33.7 |

Taken together, the data indicate that engineering of each of the four surface hydrophobic sites improved the biophysical properties of the anti-HA antibody molecule. However, site I and II mutations resulted in reduction in H3 HA binding. Mutations in site III, on the other hand, had lower loss of affinity and conferred modest improvement in biophysical properties. As such, site III engineered mutations were chosen for further evaluation.

### Affinity Enhancement

In parallel to improving the biophysical properties, the exemplary anti-HA antibody molecule FX-0-1-m3 was further engineered for enhanced binding and activity. Within the epitope, the HA1-H38 residue of group 1 was present in group 2 as an N38 which was modified with an N-glycan. Without wishing to be bound by theory, it is believed that in some embodiments, the presence of this bulky N-glycan may cause steric hindrance to antibody binding and be responsible for the reduced affinity of broadly reactive mAbs. In some instances, other significant sequence differences between group 1 and group 2 HAs may also have contributed to the reduction in affinity.

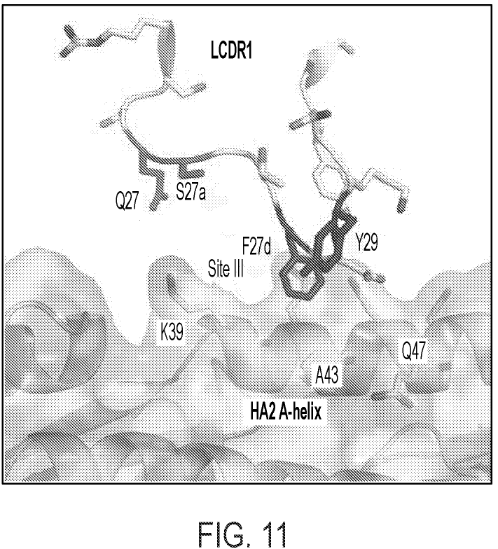

To enhance electrostatic interactions, the charged HA residues in and around the antibody interface were identified and two deemed to be unpaired basic charged HA residues (HA1:R315 and HA2: K39) near the mAb-HA interface. Sulfate ions can interact with these basic residues. Without wising to be bound by theory, it is believed that in some embodiments, if these sulfate ions could be replaced by acidic antibody residues, an enhancement in the antibody affinity may be expected, most likely driven by an improvement in koff rate. To enhance mAb interactions with HA1:R315, the HCDR2 residues G54 and N55 were mutated (G54A/S, N55D/E) and tested for binding; no significant enhancement in affinity was observed. The G54A mutation was considered to reduce HCDR2 conformational flexibility and a less flexible HCDR2 was considered to be beneficial due to its proximity to the highly flexible N38 glycan of group 2 HA. Additionally, introduction of acidic residues in LCDR1 and LCDR3 was considered to enhance electrostatic interactions with the HA2:K39 residue (FIG. 11). Mutation of LCDR1 residues Q27 and/or S27a to acidic amino acids and LCDR3 residue R93 to E were carried out. The Q27E/S27aD double mutant showed modest improvement in affinity for H3 HA (Table 12).

TABLE 12

LCDR1 engineering for affinity enhancement

| Antibody | LCDR1 mutations | AC-SINS | Octet PR | EC₅₀ H3 Babol (pM) | EC₅₀ H3 Perth (pM) |
|---|---|---|---|---|---|
| FX-0-1-m3 | | 554.6 | 0.795 | 37.0 | 101.1 |
| FX-0-14-m3 | F27dS, Y29N, K30Q | 541.5 | 0.346 | 349.4 | 5822.2 |
| FX-0-24-m3 | F27dS, Y29N | 535.9 | 0.395 | 247.8 | 281.3 |
| FX-0-65-m3 | Q27E,S27aD | 542.8 | 0.66 | 62.6 | 47.9 |
| FX-0-77-m3 | F27dW | 536.9 | 0.78 | 34.2 | 29.3 |

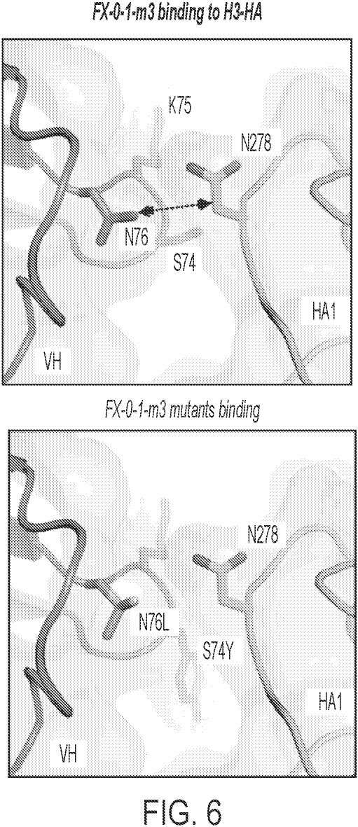

HCDR1 residues T28-S31 and HFWR3 residues S74-N76 make contacts with the lower head domain HA1 residues 277-280 and N278 is part of the predicted FX-0-1-m3 epitope (FIG. 6). It was observed that in all available H3 structures the C beta atom at position 278 of HA faces towards the polar side of VH N76 of FX-0-1-m3. It was considered that insertion of an aliphatic or aromatic amino acid as VH 76 would provide better hydrophobic complementarity with HA N278. To enhance the shape complementarity and increase the interface area these FX-0-1-m3 residues were mutated to bulky polar and hydrophobic residues. Mutation of HCDR1 residues (T28N/Q/R, S31T/N/L/D/Q/H/Y) did not show significant improvement in affinity. Although mutation of HCDR1 residues S30T/T31S did not improve affinity, their intra-molecular hydrogen bonding interactions are thought to stabilize HCDR1 conformation and were therefore retained. However, the HFWR3 residues S74, K75 and N76 mutation (S74Y/W, K75Y/W, N76S/L/F/W) significantly improved the affinity (FIG. 12).

Based on the enhanced affinity of the heavy chain design VH123, mutation of FWR3 residues S74, K75 and N76 for affinity enhancement have been proposed. Binding studies showed that mutation of these residues to hydrophobic or aromatic residues enhanced affinity by 2 to 4-fold (FIG. 12). Mutation of K75 and N76 did not have any significant impact (negative or positive) on the biophysical properties of the antibody. N76L and K75W mutations were selected as affinity enhancing mutations and combined to create VH148 (VH123 + S76L) and VH175 (VH123 + K75W/S76L).

### Pharmacokinetic Properties of Select Antibody Designs

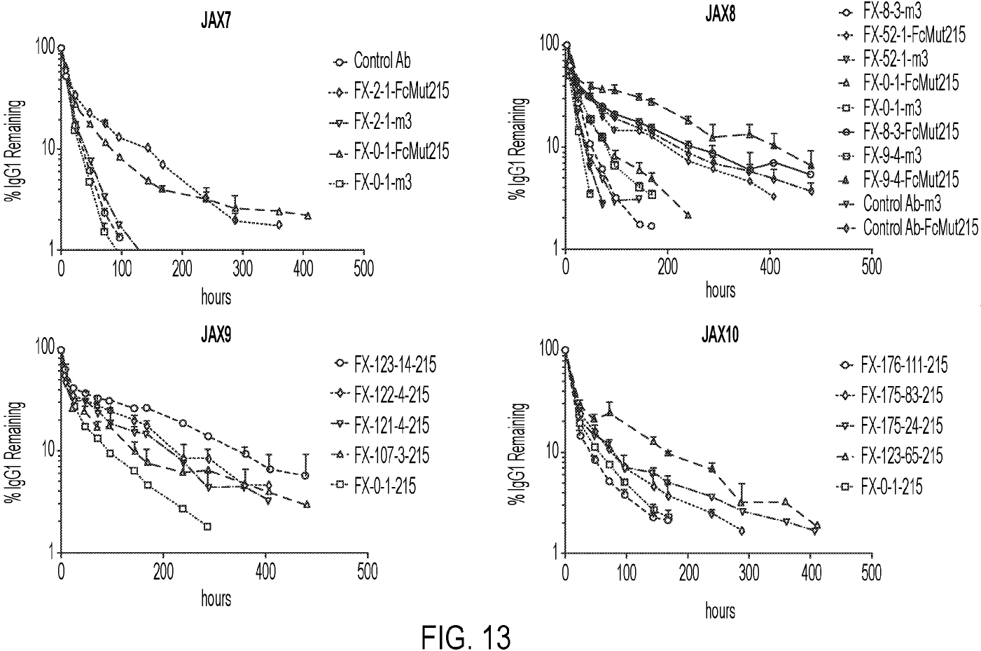

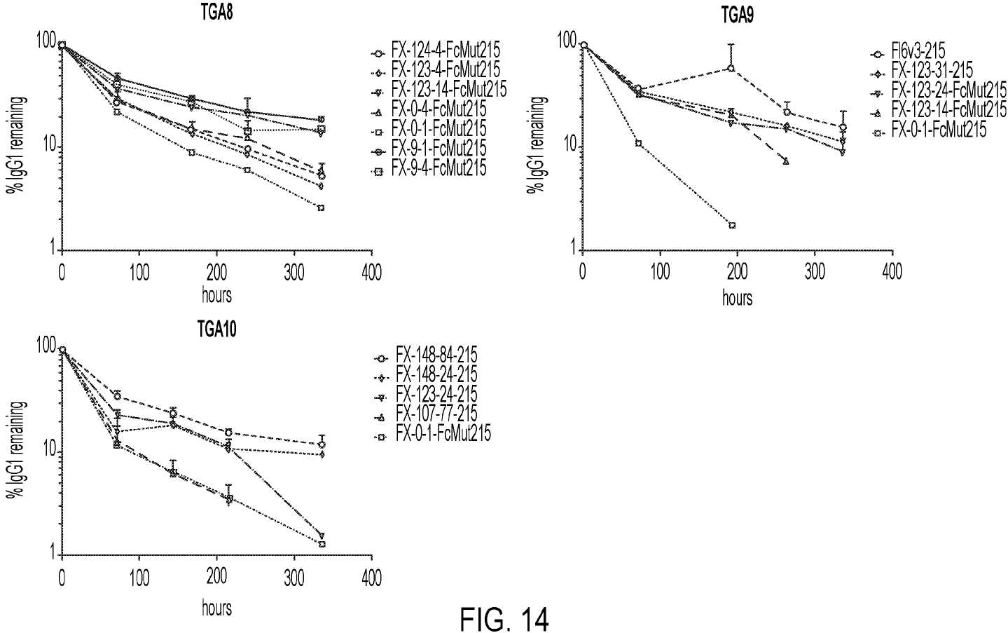

One of the main goals for engineering the Fab and Fc region of FX-0-1-m3 was to improve in vivo half-life. Fc domain variant DF215, when incorporated with the Fab of FX-0-1-m3, extended serum persistence. In addition, the Fab region was re-engineered by rescaffolding the CDRs on alternate germlines or by making specific mutations in identified hydrophobic patches. The engineered constructs were initially evaluated for their biophysical properties and binding to HA. Select constructs that were identified to have improvement in biophysical properties were assessed for their pharmacokinetics in Tg276 transgenic mice that contains human FcRn. The antibodies evaluated in Tg276 mice are listed in Tables 13-14 and the data from the different transgenic mice studies are shown in FIGS. 13-14.

TABLE 13

Antibody constructs studied in Tg276 transgenic mice

| | | |
|---|---|---|
| Antibody | Experiment | Rationale |
| FX-0-1-m3 | JAX7 | WT Control |
| FX-0-1-215 | JAX7 | Impact of incorporating modified Fc domain |
| FX-2-1-m3 | JAX7 | Germline revertant |
| FX-2-1-215 | JAX7 | Germline revertant + modified Fc domain |
| Control Ab-m3 | JAX7 | Control Ab |
| FX-0-1-m3 | JAX8 | WT Control |
| FX-0-1-215 | JAX8 | WT + modified Fc domain |
| FX-52-1-m3 | JAX8 | Site II engineered variant. Dramatic improvement in biophysical properties. No H3 binding. |
| FX-52-1-215 | JAX8 | Site II engineered variant incorporating modified Fc domain. Dramatic improvement in biophysical properties. No H3 binding. |
| FX-8-3-m3 | JAX8 | Rescaffolded variant with improved biophysical properties |
| FX-8-3-215 | JAX8 | Rescaffolded variant with improved biophysical properties incorporating modified Fc domain |
| FX-9-4-m3 | JAX8 | Rescaffolded variant (VH1-69) with improved biophysical properties |
| FX-9-4-215 | JAX8 | Rescaffolded variant (VH1-69) with improved biophysical properties incorporating modified Fc domain |
| Control Ab-m3 | JAX8 | Control antibody |
| Control Ab-215 | JAX8 | Control antibody incorporating modified Fc domain |
| FX-0-1-215 | JAX9 | WT + modified Fc domain |
| FX-107-3-215 | JAX9 | FX-8-3-215 derivative with FX-0-1-m3 HCDR3 |
| FX-121-4-215 | JAX9 | FX-9-4-215 derivative with HCDR3 mutations |
| FX-122-4-215 | JAX9 | FX-9-4-215 derivative with HCDR3 mutations |
| FX-123-14-215 | JAX9 | FX-9-4-215 derivative with FX-0-1-m3 HCDR3 + Site III mutations |
| FX-0-1-215 | JAX10 | WT + modified Fc domain |
| FX-123-65-215 | JAX10 | VH123 (VH1-69 germlined FX-0-1-m3) + LCDR1 mutation |
| FX-175-24-215 | JAX10 | Affinity enhanced variant of FX-123-24-215 |
| FX-175-83-215 | JAX10 | Variant of FX-175-24-215 with mutation in LCDR1 |
| FX-176-111-215 | JAX10 | Variant of FX-107-24-215 with mutation in LCDR1 |

TABLE 14

Antibody constructs studied in abbreviated PK study Tg276 transgenic mice

| | | |
|---|---|---|
| Antibody | Experiment | Rationale |
| FX-0-1-215 | TGA8 | WT + modified Fc domain |
| FX-0-4-215 | TGA8 | Role of VL in conferring PK improvement of FX-9-4-215 |
| FX-123-14-215 | TGA8 | FX-9-4-215 derivative with FX-0-1-m3 HCDR3 + Site III mutations |
| FX-123-4-215 | TGA8 | FX-9-4-215 derivative with FX-0-1-m3 HCDR3 |
| FX-124-4-215 | TGA8 | FX-9-4-215 derivative with alternative HCDR3 torso residues |
| FX-9-1-215 | TGA8 | Role of VH in conferring PK improvement of FX-9-4-215 |
| FX-9-4-215 | TGA8 | Rescaffolded variant (VH1-69) with improved biophysical properties incorporating modified Fc domain |
| FX-0-1-215 | TGA9 | WT + modified Fc domain |
| FX-123-14-215 | TGA9 | FX-9-4-215 derivative with FX-0-1-m3 HCDR3 + Site III mutations |
| FX-123-24-215 | TGA9 | FX-9-4-215 derivative with FX-0-1-m3 HCDR3 + Site III mutations |
| FX-123-31-215 | TGA9 | FX-9-4-215 derivative with FX-0-1-m3 HCDR3 + Site III mutations |
| Control Ab-215 | TGA9 | Control anti-influenza antibody with modified Fc domain |
| FX-0-1-215 | TGA10 | WT + modified Fc domain |
| FX-107-77-215 | TGA10 | FX-8-3-215 derivative with FX-0-1-m3 HCDR3 + Site III mutations |
| FX-123-24-215 | TGA10 | FX-9-4-215 derivative with FX-0-1-m3 HCDR3 + Site III mutations |
| FX-148-24-215 | TGA10 | Affinity enhanced variant of FX-123-24-215 |
| FX-148-84-215 | TGA10 | Affinity enhanced variant of FX-123-24-215 with LCDR1 variation |

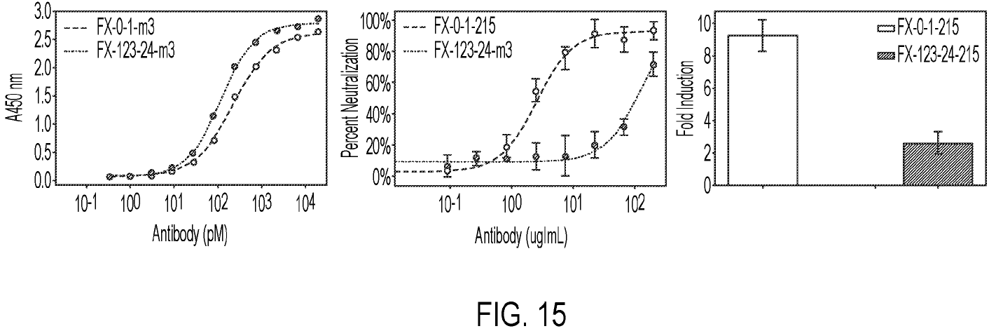

The incorporation of the DF215 Fc domain in each case enhanced persistence of all antibodies in Tg276 mice. Rescaffolding of FX-0-1-m3 CDRs onto VH1-69 germline enhanced half-life (FX-9-4-215). In the case of FX-9-4-215, some of the enhancement was attributed to incorporation of HCDR3 changes (N101D/P102Y encoded by the J-gene). FX-123-4-215, which retained the original HCDR3, also displayed enhanced persistence as compared to FX-0-1-215, however its half-life was lower than FX-9-4-215. Additionally, FX-123-14-215 and FX-123-24-215 which incorporated site III mutations in the light chain had a half-life comparable to FX-9-4-215. Of these, FX-123-24-215 had binding to H1 and H3 HA that was comparable to FX-0-1-215. Surprisingly, in vitro neutralization studies found FX-123-24-215 to be significantly less potent as compared to FX-0-1-215 (FIG. 15). An affinity-enhanced version of FX-123-24-215, FX-174-24-215 retained the potency of FX-0-1-215 while still conferring enhanced persistence in serum. Further, FX-123-65-215 containing Q27D/S27aE affinity-enhancing mutations in LCDR1 also enhanced half-life.

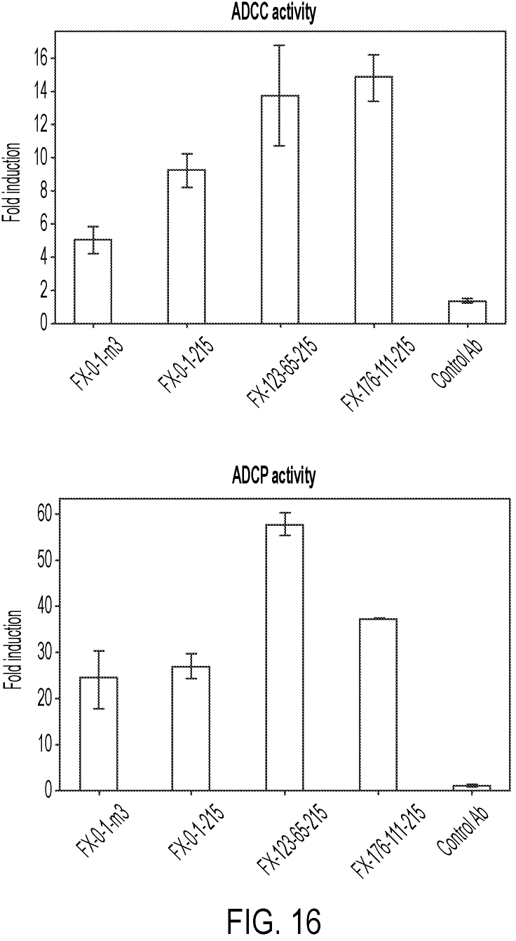

In summary, multiple paths towards improving the PK of FX-0-1-m3 were identified: incorporation of the modified Fc domains; redesign of the HCDR3 torso, redesign of HCDR3 SAP site II, redesign of LCDR1 SAP site I and redesign of LCDR1 at positions 27 and 27a. To maintain functionality of FX-0-1-m3, the optimal strategy was the addition of the modified Fc domain in combination with redesign of LCDR1. Due to a reduction in H3 neutralization for redesigns at LCDR1 SAP site I, the previously described affinity enhancing mutations (VH W75, L76) were combined to retain functionality. FX-175-24-215, which combines affinity enhancing heavy chain with engineered mutations on LCDR1 (S27, N29d) showed improved half-life in Tg276 mice while maintaining the preferred physicochemical and biological properties. Additionally, FX-123-65-215 contains acidic residues on LCDR1 (E27, D27a) that improve half-life and retains potent neutralization with the addition of the affinity enhancing mutations. Further, FX-123-65-215 shows enhanced ADCC activity and ADCP activity (FIG. 16).

### Selection of Anti-HA Antibodies

The VH107 (VH3-30-3*02), VH123 (1-69*06), VH148 (1-69*06), VH175 (1-69*06), VH176 (VH3-30-3*02) and VL24 (VK1-39*01), VL65 (VK1-39*01), VL83 (VK1-39*01), VL107 (VK1-39*01), VL110 (VK1-39*01) and VL111 (VK1-39*01) antibodies were selected for further development. Antibody combination FX-123-65-215 had binding affinity and in vitro neutralization activity (FIG. 15) comparable to the original anti-HA antibody molecule, but significantly improved effector functions (ADCC and ADCP activity; FIG. 16) as well as significantly enhanced half-life (FIG. 13). VH148 and VH175 are affinity-enhanced versions of VH123, and when combined with VL65, exhibited more potent in vitro neutralization activity for both H1 and H3 viruses. Based on the available PK data of other combinations of VH148 and VH175, FX-148-65-215 and FX-175-65-215 are expected to retain the improved half-life exhibited by VH123 while enhancing potency.

### Incorporation by Reference

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An anti-hemagglutinin (HA) antibody molecule comprising (a) a heavy chain variable region segment comprising the CDR1, CDR2, and CDR3 of the heavy chain variable region VH123; and (b) a light chain variable region segment comprising the CDR1, CDR2, or CDR3 of the light chain variable region VK-65.

2. The antibody molecule of claim 1, further comprising an Fc region.

3. The antibody molecule of claim 2, wherein the Fc region comprises a mutation.

4. The antibody molecule of any of claims 1-3, comprising an Fc region comprising the mutations of FcMut215.

5. The antibody molecule of any of claims 1-4, wherein the heavy chain variable region segment comprises one, two, three, or all of the FR1, FR2, FR3, or FR4 of the heavy chain variable region VH123.

6. The antibody molecule of any of claims 1-4, wherein the heavy chain variable region segment comprises one, two, three, or all of the FR1, FR2, FR3, or FR4 of the heavy chain variable region VH148.

7. The antibody molecule of any of claims 1-4, wherein the heavy chain variable region segment comprises one, two, three, or all of the FR1, FR2, FR3, or FR4 of the heavy chain variable region VH175.

8. The antibody molecule of any of claims 1-7, comprising the amino acid sequence of heavy chain variable region VH123, VH148, or VH175, or an amino acid sequence at least 85%, 90%, or 95% identical thereto.

9. The antibody molecule of claim 8, comprising the amino acid sequence of heavy chain variable region VH123, or an amino acid sequence at least 85%, 90%, or 95% identical thereto.

10. The antibody molecule of claim 8, comprising the amino acid sequence of heavy chain variable region VH148, or an amino acid sequence at least 85%, 90%, or 95% identical thereto.

11. The antibody molecule of claim 8, comprising the amino acid sequence of heavy chain variable region VH175, or an amino acid sequence at least 85%, 90%, or 95% identical thereto.

12. The antibody molecule of any of claims 1-11, wherein the light chain variable region segment comprises one, two, three, or all of the FR1, FR2, FR3, or FR4 of the light chain variable region VK-65.

13. The antibody molecule of any of claims 1-12, comprising the amino acid sequence of light chain variable region VK-65, or an amino acid sequence at least 85%, 90%, or 95% identical thereto.

14. An anti-hemagglutinin (HA) antibody molecule comprising (a) the heavy chain variable region VH123; (b) the light chain variable region VK-65; and (c) an Fc region comprising FcMut215.

15. An anti-hemagglutinin (HA) antibody molecule comprising (a) the heavy chain variable region VH148; (b) the light chain variable region VK-65; and (c) an Fc region comprising FcMut215.

16. An anti-hemagglutinin (HA) antibody molecule comprising (a) the heavy chain variable region VH175; (b) the light chain variable region VK-65; and (c) an Fc region comprising FcMut215.

17. A pharmaceutical composition comprising the antibody molecule of any of claims 1-16 and a pharmaceutically acceptable carrier.

18. A nucleic acid encoding a heavy chain variable region segment, a light chain variable region segment, or both, of the antibody molecule of any of claims 1-16.

19. A vector comprising the nucleic acid of claim 18.

20. A cell comprising the nucleic acid of claim 18 or the vector of claim 19.

21. A method of producing an antibody molecule, comprising culturing the cell of claim 20 under conditions that allow production of the antibody molecule, thereby producing the antibody molecule.

22. A kit comprising the antibody molecule of any of claims 1-16 and instructions for use.

23. A method of treating or preventing an influenza virus infection, or a symptom thereof, in a subject, comprising administering to the subject an effective amount of the antibody molecule of any of claims 1-16.

24. The method of claim 23, which prevents an influenza virus infection, optionally wherein the method prevents an influenza virus infection for at least 5, 10, 15, 20, 25, 30, 40, 45, 50, 60, 70, 80, 90, 100, 150, 200 days or more.

25. The method of claim 23 or 24, wherein the subject is at risk of having an influenza infection.

26. The method of any of claims 23-25, wherein the antibody molecule is administered before the subject is exposed to an influenza virus.

27. The method of any of claim 23-26, wherein the antibody molecule is administered subcutaneously or intramuscularly.

28. The method of any of claims 23-27, wherein the antibody molecule is administered once during an influenza season.

29. The method of any of claims 23-28, wherein the influenza virus is an influenza A virus.

30. The method of claim 29, wherein the influenza virus is an H1 or H3 influenza virus.


SEQUENCE LISTING (525 sequences)

SEQ ID NO: 1 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSLLYFEWLSQGYFNPWGQGTTLTVSS

SEQ ID NO: 2 - protein, 129 aa, Artificial Sequence
  source: /note="Description of Artificial Sequence: Synthetic polypeptide"
QVQLVESGGGVVQPGRSLRLSCAASGFTFSSYAMHWVRQAPGKGLEWVAVVSYDGNYKYYADSVQGRFTISRDNSKNTLYLQMNSLRAEDTAVYYCAKDSRLRSLLYFEWLSQGYFNPWGQGTTVTVSS

SEQ ID NO: 3 - protein, 129 aa, Artificial Sequence
  source: /note="Description of Artificial Sequence: Synthetic polypeptide"
QVQLVESGGGVVQPGRSLRLSCAASGFTFSSYAMHWVRQAPGKGLEWVAVVSYDGNYKYYADSVKGRFTISRDNSKNTLYLQMNSLRAEDTAVYYCAKDSRLRSLLYFEWLSQGYFNPWGQGTTVTVSS

SEQ ID NO: 4 - protein, 129 aa, Artificial Sequence
  source: /note="Description of Artificial Sequence: Synthetic polypeptide"
QVQLVESGGGVVQPGRSLRLSCAASGFTFSTYAMHWVRQAPGKGLEWVAVISYDANYKYYADSVKGRFTISRDNSKNTLYLQMNSLRAEDTAVYYCAKDSQLRSLLYFEWLSQGYFDYWGQGTLVTVSS

SEQ ID NO: 5 - protein, 129 aa, Artificial Sequence
  source: /note="Description of Artificial Sequence: Synthetic polypeptide"
QVQLVESGGGVVQPGRSLRLSCAASGFDFSTYAMHWVRQAPGKGLEWVAVISYDANYKYYADSVKGRFTISRDNSKNTLYLQMNSLRAEDTAVYYCARDSRLRSLLYFEWLSQGYFDYWGQGTLVTVSS

SEQ ID NO: 6 - protein, 129 aa, Artificial Sequence
  source: /note="Description of Artificial Sequence: Synthetic polypeptide"
QVQLVESGGGVVQPGRSLRLSCAASGFTFSTYAMHWVRQAPGKGLEWVCVISYDANYKYYADSVKGRFTCSRDNSKNTLYLQMNSLRAEDTAVYYCARDSRLRSLLYFEWLSQGYFDYWGQGTLVTVSS

SEQ ID NO: 7 - protein, 129 aa, Artificial Sequence
  source: /note="Description of Artificial Sequence: Synthetic polypeptide"
QVQLVESGGGVVQPGRSLRLSCAASGFTFDTYAMHWVRQAPGKGLEWVAVISYDANYKYYADSVKGRFTISRDNSKNTLYLQMNSLRAEDTAVYYCARDSRLRSLLYFEWLSQGYFDYWGQGTLVTVSS

SEQ ID NO: 8 - protein, 129 aa, Artificial Sequence
  source: /note="Description of Artificial Sequence: Synthetic polypeptide"
EVQLLESGGGLVQPGGSLRLSCAASGFTFSSYAMHWVRQAPGKGLEWVSVISYDANYKYYADSVKGRFTISRDNSKNTLYLQMNSLRAEDTAVYYCAKDSQLRSLLYFEWLSQGYFDYWGQGTTLTVSS

SEQ ID NO: 9 - protein, 129 aa, Artificial Sequence
  source: /note="Description of Artificial Sequence: Synthetic polypeptide"
EVQLLESGGGLVQPGGSLRLSCAASGFTFSSYAMHWVRQAPGKGLEWVAVISYDANYKYYADSVKGRFTISRDNSKNTLYLQMNSLRAEDTAVYYCAKDSRLRSLLYFEWLSQGYFDYWGQGTLVTVSS

SEQ ID NO: 10 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSQLRSLLYFEWLSQGYFDYWGQGTTLTVSS

SEQ ID NO: 11 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCARDSRLRSLLYFEWLSQGYFDYWGQGTTLTVSS

SEQ ID NO: 12 - protein, 129 aa, Artificial Sequence
  source: /note="Description of Artificial Sequence: Synthetic polypeptide"
QVQLVQSGAEVKKPGASVKVSCKASGFTFSTYAINWVRQATGQGLEWMGWISYDANYKYYAQKFQGRVTMTRDTSISTAYMELSSLRSEDTAVYYCAKDSQLRSLLYFEWLSQGYFDYWGQGTTLTVSS

SEQ ID NO: 13 - protein, 129 aa, Artificial Sequence
  source: /note="Description of Artificial Sequence: Synthetic polypeptide"
QVQLVQSGAEVKKPGASVKVSCKASGFTFSTYAINWVRQATGQGLEWMGVISYDANYKYYAQKFQGRVTMTRDTSISTAYMELSSLRSEDTAVYYCARDSQLRSLLYFEWLSQGYFDYWGQGTTLTVSS

SEQ ID NO: 14 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVCYDGNYKYYADSVQGRFTISRDNSKNTLYLQMNSLRAEDTAVYYCAKDSRLRSLCYFEWLSQGYFNPWGQGTTLTVSS

SEQ ID NO: 15 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSLLYCEWCSQGYFNPWGQGTTLTVSS

SEQ ID NO: 16 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSLLYFEWLSCGYFNPWGQGTTLTVSS

SEQ ID NO: 17 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNCKYYADSVQGRFTISRDNSKNTLYLQMNSLRAEDTAVYYCAKDSRLRSLCYFEWLSQGYFNPWGQGTTLTVSS

SEQ ID NO: 18 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSLLYFECLSQGYFNPWGQGTTLTVSS

SEQ ID NO: 19 - protein, 129 aa, Artificial Sequence
  source: /note="Description of Artificial Sequence: Synthetic polypeptide"
QVQLLETGGGLVKPGQSLKLSCAASGFTFTSYSMHWVRQPPGKGLEWVAVVSYDGNYKYYADSVQGRFTISRDNSKNTLYLQMNSLRAEDTAVYYCAKDSRLRSLHYFEWLSQGYFNPWGQGTTLTVSS

SEQ ID NO: 20 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSLLYFEWMNQGYFNPWGQGTTLTVSS

SEQ ID NO: 21 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSLLYQEWLSQGYFNPWGQGTTLTVSS

SEQ ID NO: 22 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSLLYFDWLSQGYFNPWGQGTTLTVSS

SEQ ID NO: 23 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSLQYFEWLSQGYFNPWGQGTTLTVSS

SEQ ID NO: 24 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSLNYFEWLSQGYFNPWGQGTTLTVSS

SEQ ID NO: 25 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRNRSLLMFEWLSQGYFNPWGQGTTLTVSS

SEQ ID NO: 26 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRQRSLLMFEWLSQGYFNPWGQGTTLTVSS

SEQ ID NO: 27 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSLLYFEWNSQGYFNPWGQGTTLTVSS

SEQ ID NO: 28 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRNRSLLMFEWNNQGYFNPWGQGTTLTVSS

SEQ ID NO: 29 - protein, 129 aa, Artificial Sequence
  source: /note="Description of Artificial Sequence: Synthetic polypeptide"
QVQLVESGGGVVQPGRSLRLSCAASGFTFSSYAMHWVRQAPGKGLEWVAIVSYDGNYKYYADSVKGRFTISRDNSKNTLYLQMNSLRAEDTAVYYCAKDSRLRSLLYFEWLSQGYFNPWGQGTTVTVSS

SEQ ID NO: 30 - protein, 129 aa, Artificial Sequence
  source: /note="Description of Artificial Sequence: Synthetic polypeptide"
QVQLVESGGGVVQPGRSLRLSCAASGFTFSSYAMHWVRQAPGKGLEWVAVVSYDGNYKYYADSVKGRFTISRDNSKNTLYLQMNSLRAEDTAVYYCAKDSRNRVLLYFEWLSQGYFNPWGQGTTVTVSS

SEQ ID NO: 31 - protein, 129 aa, Artificial Sequence
  source: /note="Description of Artificial Sequence: Synthetic polypeptide"
QVQLVESGGGVVQPGRSLRLSCAASGFTFSSYAMHWVRQAPGKGLEWVAVVSYDGNYKYYADSVKGRFTISRDNSKNTLYLQMNSLRAEDTAVYYCAKDSRLRSLLYFEWLSLGYFNPWGQGTTVTVSS

SEQ ID NO: 32 - protein, 129 aa, Artificial Sequence
  source: /note="Description of Artificial Sequence: Synthetic polypeptide"
QVQLVESGGGVVQPGRSLRLSCAASGFTFSSYAMHWVRQAPGKGLEWVAVVSYDGNYKYYADSVKGRFTISRDNSKNTLYLQMNSLRAEDTAVYYCAKDSRLRSLQYFEWLSQGYFNPWGQGTTVTVSS

SEQ ID NO: 33 - protein, 129 aa, Artificial Sequence
  source: /note="Description of Artificial Sequence: Synthetic polypeptide"
QVQLVESGGGVVQPGRSLRLSCAASGFTFSSYAMHWVRQAPGKGLEWVAVVSYDGNYKYYADSVKGRFTISRDNSKNTLYLQMNSLRAEDTAVYYCAKDSRLRLLLYFEWLSQGYFNPWGQGTTVTVSS

SEQ ID NO: 34 - protein, 129 aa, Artificial Sequence
  source: /note="Description of Artificial Sequence: Synthetic polypeptide"
QVQLVESGGGVVQPGRSLRLSCAASGFTFSSYAMHWVRQAPGKGLEWVAVVSYDGNYKYYADSVKGRFTISRDNSKNTLYLQMNSLRAEDTAVYYCAKDSRLRSLLYFEWLSQGRFNPWGQGTTVTVSS

SEQ ID NO: 35 - protein, 129 aa, Artificial Sequence
  source: /note="Description of Artificial Sequence: Synthetic polypeptide"
QVQLVESGGGVVQPGRSLRLSCAASGFTFSSYAMHWVRQAPGKGLEWVAVVSYDGNYKYYADSVRGRFTISRDNSKNTLYLQMNSLRAEDTAVYYCAKDSRLRSLLYFEWLSQGYFNPWGQGTTVTVSS

SEQ ID NO: 36 - protein, 129 aa, Artificial Sequence
  source: /note="Description of Artificial Sequence: Synthetic polypeptide"
QVQLVESGGGVVQPGRSLRLSCAASGFTFSSYAMHWVRQAPGKGLEWVAVVSYDGNYKYYADSVKGRFTISRDNSKNTLYLQMNSLRAEDTAVYYCAKDSELRSLLYFEWLSQGYFNPWGQGTTVTVSS

SEQ ID NO: 37 - protein, 129 aa, Artificial Sequence
  source: /note="Description of Artificial Sequence: Synthetic polypeptide"
QVQLEESGGGVVQPGRSLRLSCAASGFTFSSYAMHWVRQAPGKGLEWVAVVSYDSNYKYYADSVKGRFTISRDNSKNTLYLQMNSLRAEDTAVYYCAKDSRLRSLLYFEWLSQGYFNPWGQGTTVTVSS

SEQ ID NO: 38 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSNLYYEWLSQGYFNPWGQGTTLTVSS

SEQ ID NO: 39 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSQLYYEWLSQGYFNPWGQGTTLTVSS

SEQ ID NO: 40 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSLLYFEHLSQGYFNPWGQGTTLTVSS

SEQ ID NO: 42 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSLLYFERLSQGYFNPWGQGTTLTVSS

SEQ ID NO: 43 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNHKYYADSVQGRFTISRDNSKNTLYLQMNSLRAEDTAVYYCAKDSRLRSLLYFDWLSQGYFNPWGQGTTLTVSS

SEQ ID NO: 44 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNNKYYADSVQGRFTISRDNSKNTLYLQMNSLRAEDTAVYYCAKDSRLRSLLYFDWLSQGYFNPWGQGTTLTVSS

SEQ ID NO: 46 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKESRLRSLLYFEWLSQGYFNPWGQGTTLTVSS

SEQ ID NO: 47 - protein, 129 aa, Artificial Sequence
  source: /note="Description of Artificial Sequence: Synthetic polypeptide"
QVQLLETGGGLVKPGQSLKLSCAASGFTFTSYGMHWVRQPPGKGLEWVAVVSYDGNYKYYADSVQGRFTISRDNSKNTLYLQMNSLRAEDTAVYYCAKDSRLRLLLYFEWLSQGYFNPWGQGTTLTVSS

SEQ ID NO: 48 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSLRYFEWLSGGYFNPWGQGTTLTVSS

SEQ ID NO: 49 - protein, 129 aa, Artificial Sequence
  source: /note="Description of Artificial Sequence: Synthetic polypeptide"
EVQLVESGGGAVQPGESLKLSCAASGFTFSNYGMHWVRQAPGKGLEWVAVISYDGSNKYYADSVKGRFTISRDNSKDTLYLQMNSLRAEDTALFYCAKERPLRLLRYFDWLSGGANDYWGQGTLVTVSS

SEQ ID NO: 50 - protein, 129 aa, Artificial Sequence
  source: /note="Description of Artificial Sequence: Synthetic polypeptide"
QVQLLETGGGLVKPGQSLKLSCAASGFTFTNYGMHWVRQPPGKGLEWVAVVSYDGSYKYYADSVQGRFTISRDNSKNTLYLQMNSLRAEDTAVYYCAKERPLRLLRYFDWLSGGANDYWGQGTTLTVSS

SEQ ID NO: 51 - protein, 129 aa, Artificial Sequence
  source: /note="Description of Artificial Sequence: Synthetic polypeptide"
QVQLLETGGGLVKPGQSLKLSCAASGFTFTNYGMHWVRQPPGKGLEWVAVVSYDGNYKYYADSVQGRFTISRDNSKNTLYLQMNSLRAEDTAVYYCAKERPLRLLRYFDWLSGGANDYWGQGTTLTVSS

SEQ ID NO: 52 - protein, 129 aa, Artificial Sequence
  source: /note="Description of Artificial Sequence: Synthetic polypeptide"
QVQLLETGGGLVKPGQSLKLSCAASGFTFTNYGMHWVRQPPGKGLEWVAVVSYDGNNKYYADSVQGRFTISRDNSKNTLYLQMNSLRAEDTAVYYCAKERPLRLLRYFDWLSGGANDYWGQGTTLTVSS

SEQ ID NO: 53 - protein, 129 aa, Artificial Sequence
  source: /note="Description of Artificial Sequence: Synthetic polypeptide"
EVQLVESGGGAVQPGESLKLSCAASGFTFSNYGMHWVRQAPGKGLEWVAVISYDGSYKYYADSVKGRFTISRDNSKDTLYLQMNSLRAEDTALFYCAKERPLRLLRYFDWLSGGANDYWGQGTLVTVSS

SEQ ID NO: 54 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSLLMFEWLSQGYFNPWGQGTTLTVSS

SEQ ID NO: 55 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRNRSLLYFEWLSQGYFNPWGQGTTLTVSS

SEQ ID NO: 56 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRQRSLLYFEWLSQGYFNPWGQGTTLTVSS

SEQ ID NO: 57 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRTRSLLYFEWLSQGYFNPWGQGTTLTVSS

SEQ ID NO: 58 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRVRSLLYFEWLSQGYFNPWGQGTTLTVSS

SEQ ID NO: 59 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRIRSLLYFEWLSQGYFNPWGQGTTLTVSS

SEQ ID NO: 60 - protein, 129 aa, Artificial Sequence
  source: /note="Description of Artificial Sequence: Synthetic polypeptide"
QVQLLETGGGLVKPGQSLKLSCAASGFTFTSYSMHWVRQPPGKGLEWVAVVSYDGNYKYYADSVQGRFTISRDNSKNTLYLQMNSLRAEDTAVYYCAKDSRLRSLLYFEWLSQGYFNPWGQGTTLTVSS

SEQ ID NO: 61 - protein, 129 aa, Artificial Sequence
  source: /note="Description of Artificial Sequence: Synthetic polypeptide"
QVQLVESGGGVVQPGRSLRLSCAASGFTFSTYAMHWVRQAPGKGLEWVAVISYDANYKYYADSVKGRFTISRDNSKNTLYLQMNSLRAEDTAVYYCAKDSQNRSLLYFEWLSQGYFDYWGQGTLVTVSS

SEQ ID NO: 62 - protein, 129 aa, Artificial Sequence
  source: /note="Description of Artificial Sequence: Synthetic polypeptide"
QVQLVESGGGVVQPGRSLRLSCAASGFTFSTYAMHWVRQAPGKGLEWVAVISYDANYKYYADSVKGRFTISRDNSKNTLYLQMNSLRAEDTAVYYCAKDSQLRSLLMFEWLSQGYFDYWGQGTLVTVSS

SEQ ID NO: 63 - protein, 129 aa, Artificial Sequence
  source: /note="Description of Artificial Sequence: Synthetic polypeptide"
QVQLVESGGGVVQPGRSLRLSCAASGFTFSTYAMHWVRQAPGKGLEWVAVISYDANYKYYADSVKGRFTISRDNSKNTLYLQMNSLRAEDTAVYYCAKDSQNRSLLMFEWLSQGYFDYWGQGTLVTVSS

SEQ ID NO: 64 - protein, 129 aa, Artificial Sequence
  source: /note="Description of Artificial Sequence: Synthetic polypeptide"
QVQLLETGGGLVKPGQSLKLSCAASGFTFTSYSMHWVRQPPGKGLEWVAVVSYDGNYKYYADSVQGRFTISRDNSKNTLYLQMNSLRAEDTAVYYCAKDSRNRSLLYFEWLSQGYFNPWGQGTTLTVSS

SEQ ID NO: 65 - protein, 129 aa, Artificial Sequence
  source: /note="Description of Artificial Sequence: Synthetic polypeptide"
QVQLLETGGGLVKPGQSLKLSCAASGFTFTSYSMHWVRQPPGKGLEWVAVVSYDGNYKYYADSVQGRFTISRDNSKNTLYLQMNSLRAEDTAVYYCAKDSRLRSLLMFEWLSQGYFNPWGQGTTLTVSS

SEQ ID NO: 66 - protein, 129 aa, Artificial Sequence
  source: /note="Description of Artificial Sequence: Synthetic polypeptide"
QVQLVESGGGVVQPGRSLRLSCAASGFTFSSYAMHWVRQAPGKGLEWVAVVSYDGNYKYYADSVKGRFTISRDNSKNTLYLQMNSLRAEDTAVYYCAKDSQLRSLLYFEWLSQGYFNPWGQGTTVTVSS

SEQ ID NO: 67 - protein, 129 aa, Artificial Sequence
  source: /note="Description of Artificial Sequence: Synthetic polypeptide"
QVQLVESGGGVVQPGRSLRLSCAASGFTFSSYAMHWVRQAPGKGLEWVAVVSYDGNYKYYADSVKGRFTISRDNSKNTLYLQMNSLRAEDTAVYYCAKDSRNRSLLYFEWLSQGYFNPWGQGTTVTVSS

SEQ ID NO: 68 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRARSLLYFEWLSQGYFNPWGQGTTLTVSS

SEQ ID NO: 69 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRSRSLLYFEWLSQGYFNPWGQGTTLTVSS

SEQ ID NO: 70 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRHRSLLYFEWLSQGYFNPWGQGTTLTVSS

SEQ ID NO: 71 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRYRSLLYFEWLSQGYFNPWGQGTTLTVSS

SEQ ID NO: 72 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRMRSLLYFEWLSQGYFNPWGQGTTLTVSS

SEQ ID NO: 73 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRRRSLLYFEWLSQGYFNPWGQGTTLTVSS

SEQ ID NO: 74 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRDRSLLYFEWLSQGYFNPWGQGTTLTVSS

SEQ ID NO: 75 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRKRSLLYFEWLSQGYFNPWGQGTTLTVSS

SEQ ID NO: 76 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRPRSLLYFEWLSQGYFNPWGQGTTLTVSS

SEQ ID NO: 77 - protein, 129 aa, Artificial Sequence
  source: /note="Description of Artificial Sequence: Synthetic polypeptide"
QVQLLETGGGLVKPGQSLKLSCAASGFTFTSFAMHWVRQPPGKGLEWVAVVSYDGNYKYYADSVQGRFTISRDNSKNTLYLQMNSLRAEDTAVYYCAKDSRNRSLLYFEWLSQGYFNPWGQGTTLTVSS

SEQ ID NO: 78 - protein, 129 aa, Artificial Sequence
  source: /note="Description of Artificial Sequence: Synthetic polypeptide"
QVQLLETGGGLVKPGQSLKLSCAASGFTFTSFAMHWVRQPPGKGLEWVAVVSYDGNYKYYADSVQGRFTISRDNSKNTLYLQMNSLRAEDTAVYYCAKDSRQRSLLYFEWLSQGYFNPWGQGTTLTVSS

SEQ ID NO: 79 - protein, 129 aa, Artificial Sequence
  source: /note="Description of Artificial Sequence: Synthetic polypeptide"
QVQLLETGGGLVKPGQSLKLSCAASGFTFTSFAMHWVRQPPGKGLEWVAVVSYDGNYKYYADSVQGRFTISRDNSKNTLYLQMNSLRAEDTAVYYCAKDSRRRSLLYFEWLSQGYFNPWGQGTTLTVSS

SEQ ID NO: 80 - protein, 129 aa, Artificial Sequence
  source: /note="Description of Artificial Sequence: Synthetic polypeptide"
QVQLLETGGGLVKPGQSLKLSCAASGFTFTSHAMHWVRQPPGKGLEWVAVVSYDGNYKYYADSVQGRFTISRDNSKNTLYLQMNSLRAEDTAVYYCAKDSRNRSLLYFEWLSQGYFNPWGQGTTLTVSS

SEQ ID NO: 81 - protein, 129 aa, Artificial Sequence
  source: /note="Description of Artificial Sequence: Synthetic polypeptide"
QVQLLETGGGLVKPGQSLKLSCAASGFTFTSHAMHWVRQPPGKGLEWVAVVSYDGNYKYYADSVQGRFTISRDNSKNTLYLQMNSLRAEDTAVYYCAKDSRQRSLLYFEWLSQGYFNPWGQGTTLTVSS

SEQ ID NO: 82 - protein, 129 aa, Artificial Sequence
  source: /note="Description of Artificial Sequence: Synthetic polypeptide"
QVQLLETGGGLVKPGQSLKLSCAASGFTFTSHAMHWVRQPPGKGLEWVAVVSYDGNYKYYADSVQGRFTISRDNSKNTLYLQMNSLRAEDTAVYYCAKDSRRRSLLYFEWLSQGYFNPWGQGTTLTVSS

SEQ ID NO: 83 - protein, 129 aa, Artificial Sequence
  source: /note="Description of Artificial Sequence: Synthetic polypeptide"
QVQLLETGGGLVKPGQSLKLSCAASGFTFTNYAMHWVRQPPGKGLEWVAVVSYDGNYKYYADSVQGRFTISRDNSKNTLYLQMNSLRAEDTAVYYCAKDSRLRSLLYFEWLSQGYFNPWGQGTTLTVSS

SEQ ID NO: 84 - protein, 129 aa, Artificial Sequence
  source: /note="Description of Artificial Sequence: Synthetic polypeptide"
QVQLLETGGGLVKPGQSLKLSCAASGFTFTLYAMHWVRQPPGKGLEWVAVVSYDGNYKYYADSVQGRFTISRDNSKNTLYLQMNSLRAEDTAVYYCAKDSRLRSLLYFEWLSQGYFNPWGQGTTLTVSS

SEQ ID NO: 85 - protein, 129 aa, Artificial Sequence
  source: /note="Description of Artificial Sequence: Synthetic polypeptide"
QVQLLETGGGLVKPGQSLKLSCAASGFTFTDYAMHWVRQPPGKGLEWVAVVSYDGNYKYYADSVQGRFTISRDNSKNTLYLQMNSLRAEDTAVYYCAKDSRLRSLLYFEWLSQGYFNPWGQGTTLTVSS

SEQ ID NO: 86 - protein, 129 aa, Artificial Sequence
  source: /note="Description of Artificial Sequence: Synthetic polypeptide"
QVQLLETGGGLVKPGQSLKLSCAASGFTFTQYAMHWVRQPPGKGLEWVAVVSYDGNYKYYADSVQGRFTISRDNSKNTLYLQMNSLRAEDTAVYYCAKDSRLRSLLYFEWLSQGYFNPWGQGTTLTVSS

SEQ ID NO: 87 - protein, 129 aa, Artificial Sequence
  source: /note="Description of Artificial Sequence: Synthetic polypeptide"
QVQLLETGGGLVKPGQSLKLSCAASGFTFTHYAMHWVRQPPGKGLEWVAVVSYDGNYKYYADSVQGRFTISRDNSKNTLYLQMNSLRAEDTAVYYCAKDSRLRSLLYFEWLSQGYFNPWGQGTTLTVSS

SEQ ID NO: 88 - protein, 129 aa, Artificial Sequence
  source: /note="Description of Artificial Sequence: Synthetic polypeptide"
QVQLLETGGGLVKPGQSLKLSCAASGFTFTYYAMHWVRQPPGKGLEWVAVVSYDGNYKYYADSVQGRFTISRDNSKNTLYLQMNSLRAEDTAVYYCAKDSRLRSLLYFEWLSQGYFNPWGQGTTLTVSS

SEQ ID NO: 89 - protein, 129 aa, Artificial Sequence
  source: /note="Description of Artificial Sequence: Synthetic polypeptide"
QVQLLETGGGLVKPGQSLKLSCAASGFTFTNYAMHWVRQPPGKGLEWVAVVSYDGNYKYYADSVQGRFTISRDNSKNTLYLQMNSLRAEDTAVYYCAKDSRNRSLLYFEWLSQGYFNPWGQGTTLTVSS

SEQ ID NO: 90 - protein, 129 aa, Artificial Sequence
  source: /note="Description of Artificial Sequence: Synthetic polypeptide"
QVQLLETGGGLVKPGQSLKLSCAASGFTFTLYAMHWVRQPPGKGLEWVAVVSYDGNYKYYADSVQGRFTISRDNSKNTLYLQMNSLRAEDTAVYYCAKDSRNRSLLYFEWLSQGYFNPWGQGTTLTVSS

SEQ ID NO: 91 - protein, 129 aa, Artificial Sequence
  source: /note="Description of Artificial Sequence: Synthetic polypeptide"
QVQLLETGGGLVKPGQSLKLSCAASGFTFTDYAMHWVRQPPGKGLEWVAVVSYDGNYKYYADSVQGRFTISRDNSKNTLYLQMNSLRAEDTAVYYCAKDSRNRSLLYFEWLSQGYFNPWGQGTTLTVSS

SEQ ID NO: 92 - protein, 129 aa, Artificial Sequence
  source: /note="Description of Artificial Sequence: Synthetic polypeptide"
QVQLLETGGGLVKPGQSLKLSCAASGFTFTQYAMHWVRQPPGKGLEWVAVVSYDGNYKYYADSVQGRFTISRDNSKNTLYLQMNSLRAEDTAVYYCAKDSRNRSLLYFEWLSQGYFNPWGQGTTLTVSS

SEQ ID NO: 93 - protein, 129 aa, Artificial Sequence
  source: /note="Description of Artificial Sequence: Synthetic polypeptide"
QVQLLETGGGLVKPGQSLKLSCAASGFTFTHYAMHWVRQPPGKGLEWVAVVSYDGNYKYYADSVQGRFTISRDNSKNTLYLQMNSLRAEDTAVYYCAKDSRNRSLLYFEWLSQGYFNPWGQGTTLTVSS

SEQ ID NO: 94 - protein, 129 aa, Artificial Sequence
  source: /note="Description of Artificial Sequence: Synthetic polypeptide"
QVQLLETGGGLVKPGQSLKLSCAASGFTFTYYAMHWVRQPPGKGLEWVAVVSYDGNYKYYADSVQGRFTISRDNSKNTLYLQMNSLRAEDTAVYYCAKDSRNRSLLYFEWLSQGYFNPWGQGTTLTVSS

SEQ ID NO: 95 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLHSLLYFEWLSQGYFNPWGQGTTLTVSS

SEQ ID NO: 96 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLYSLLYFEWLSQGYFNPWGQGTTLTVSS

SEQ ID NO: 97 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRNHSLLYFEWLSQGYFNPWGQGTTLTVSS

SEQ ID NO: 98 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRNYSLLYFEWLSQGYFNPWGQGTTLTVSS

SEQ ID NO: 99 - protein, 129 aa, Artificial Sequence
  source: /note="Description of Artificial Sequence: Synthetic polypeptide"
QVQLLETGGGLVKPGQSLKLSCAASGFQFTSFAMHWVRQPPGKGLEWVAVVSYDGNYKYYADSVQGRFTISRDNSKNTLYLQMNSLRAEDTAVYYCAKDSRNRSLLYFEWLSQGYFNPWGQGTTLTVSS

SEQ ID NO: 100 - protein, 129 aa, Artificial Sequence
  source: /note="Description of Artificial Sequence: Synthetic polypeptide"
QVQLLETGGGLVKPGQSLKLSCAASGFNFTSFAMHWVRQPPGKGLEWVAVVSYDGNYKYYADSVQGRFTISRDNSKNTLYLQMNSLRAEDTAVYYCAKDSRNRSLLYFEWLSQGYFNPWGQGTTLTVSS

SEQ ID NO: 101 - protein, 129 aa, Artificial Sequence
  source: /note="Description of Artificial Sequence: Synthetic polypeptide"
QVQLLETGGGLVKPGQSLKLSCAASGFRFTSFAMHWVRQPPGKGLEWVAVVSYDGNYKYYADSVQGRFTISRDNSKNTLYLQMNSLRAEDTAVYYCAKDSRNRSLLYFEWLSQGYFNPWGQGTTLTVSS

SEQ ID NO: 102 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDANYKYYADSVQGRFTISRDNSKNTLYLQMNSLRAEDTAVYYCAKDSRLRSQLYFEWLSQGYFNPWGQGTTLTVSS

SEQ ID NO: 103 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDANYKYYADSVQGRFTISRDNSKNTLYLQMNSLRAEDTAVYYCAKDSRLRSHLYFEWLSQGYFNPWGQGTTLTVSS

SEQ ID NO: 104 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDANYKYYADSVQGRFTISRDNSKNTLYLQMNSLRAEDTAVYYCAKDSRLRSKLYFEWLSQGYFNPWGQGTTLTVSS

SEQ ID NO: 105 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDANYKYYADSVQGRFTISRDNSKNTLYLQMNSLRAEDTAVYYCAKDSRLRSRLYFEWLSQGYFNPWGQGTTLTVSS

SEQ ID NO: 106 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDANYKYYADSVQGRFTISRDNSKNTLYLQMNSLRAEDTAVYYCAKDSRLRSFLYFEWLSQGYFNPWGQGTTLTVSS

SEQ ID NO: 107 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDANYKYYADSVQGRFTISRDNSKNTLYLQMNSLRAEDTAVYYCAKDSRLRSLLKFEWLSQGYFNPWGQGTTLTVSS

SEQ ID NO: 108 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDANYKYYADSVQGRFTISRDNSKNTLYLQMNSLRAEDTAVYYCAKDSRLRSLLRFEWLSQGYFNPWGQGTTLTVSS

SEQ ID NO: 109 - protein, 129 aa, Artificial Sequence
  source: /note="Description of Artificial Sequence: Synthetic polypeptide"
EVQLLESGGGLVQPGGSLRLSCAASGFTFSSYAMHWVRQAPGKGLEWVAVISYDANYKYYADSVKGRFTISRDNSKNTLYLQMNSLRAEDTAVYYCAKDSQLRSLLYFEWLSQGYFDYWGQGTLVTVSS

SEQ ID NO: 110 - protein, 129 aa, Artificial Sequence
  source: /note="Description of Artificial Sequence: Synthetic polypeptide"
EVQLLESGGGLVQPGGSLRLSCAASGFTFSSYAMHWVRQAPGKGLEWVAVISYDANYKYYADSVKGRFTISRDNSKNTLYLQMNSLRAEDTAVYYCAKDSRLRSLLYFEWLSQGYFNPWGQGTLVTVSS

SEQ ID NO: 111 - protein, 129 aa, Artificial Sequence
  source: /note="Description of Artificial Sequence: Synthetic polypeptide"
EVQLLESGGGLVQPGGSLRLSCAASGFTFSSYAMHWVRQAPGKGLEWVAVISYDANYKYYADSVKGRFTISRDNSKNTLYLQMNSLRAEDTAVYYCAKDSRLRSLLYFEWLSQGYFGVWGQGTLVTVSS

SEQ ID NO: 112 - protein, 129 aa, Artificial Sequence
  source: /note="Description of Artificial Sequence: Synthetic polypeptide"
EVQLLESGGGLVQPGGSLRLSCAASGFTFSSYAMHWVRQAPGKGLEWVAVISYDANYKYYADSVKGRFTISRDNSKNTLYLQMNSLRAEDTAVYYCAKDSRLRSLLYFEWLSQGYFDYWGQGTTLTVSS

SEQ ID NO: 113 - protein, 129 aa, Artificial Sequence
  source: /note="Description of Artificial Sequence: Synthetic polypeptide"
EVQLLESGGGLVQPGGSLRLSCAASGFTFSSYAMHWVRQAPGKGLEWVAVVSYDGNYKYYADSVKGRFTISRDNSKNTLYLQMNSLRAEDTAVYYCAKDSRLRSLLYFEWLSQGYFDYWGQGTLVTVSS

SEQ ID NO: 114 - protein, 129 aa, Artificial Sequence
  source: /note="Description of Artificial Sequence: Synthetic polypeptide"
EVQLLESGGGLVQPGGSLRLSCAASGFTFTSYAMHWVRQAPGKGLEWVAVISYDANYKYYADSVKGRFTISRDNSKNTLYLQMNSLRAEDTAVYYCAKDSRLRSLLYFEWLSQGYFDYWGQGTLVTVSS

SEQ ID NO: 115 - protein, 129 aa, Artificial Sequence
  source: /note="Description of Artificial Sequence: Synthetic polypeptide"
EVQLLESGGGLVQPGGSLRLSCAASGFTFSDYAMHWVRQAPGKGLEWVAVISYDANYKYYADSVKGRFTISRDNSKNTLYLQMNSLRAEDTAVYYCAKDSRLRSLLYFEWLSQGYFDYWGQGTLVTVSS

SEQ ID NO: 116 - protein, 129 aa, Artificial Sequence
  source: /note="Description of Artificial Sequence: Synthetic polypeptide"
EVQLLESGGGLVQPGGSLRLSCAASGFTFSSRAMHWVRQAPGKGLEWVAVISYDANYKYYADSVKGRFTISRDNSKNTLYLQMNSLRAEDTAVYYCAKDSRLRSLLYFEWLSQGYFDYWGQGTLVTVSS

SEQ ID NO: 117 - protein, 129 aa, Artificial Sequence
  source: /note="Description of Artificial Sequence: Synthetic polypeptide"
EVQLLESGGGLVQPGGSLRLSCAASGFTFSSHAMHWVRQAPGKGLEWVAVISYDANYKYYADSVKGRFTISRDNSKNTLYLQMNSLRAEDTAVYYCAKDSRLRSLLYFEWLSQGYFDYWGQGTLVTVSS

SEQ ID NO: 118 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSQLRSLLYFEWLSQGYFDYWGQGTTVTVSS

SEQ ID NO: 119 - protein, 129 aa, Artificial Sequence
  source: /note="Description of Artificial Sequence: Synthetic polypeptide"
QVQLVQSGAEVKKPGSSVKVSCKASGFTFSTYAMHWVRQAPGQGLEWMGVISYDANYKYYAQKFQGRVTITRDNSKSTLYMELSSLRSEDTAVYYCAKDSQLRSLLYFEWLSQGYFDYWGQGTTLTVSS

SEQ ID NO: 120 - protein, 129 aa, Artificial Sequence
  source: /note="Description of Artificial Sequence: Synthetic polypeptide"
QVQLVQSGAEVKKPGSSVKVSCKASGFTFSTYAMHWVRQAPGQGLEWMGVISYDANYKYYAQKFQGRVTITRDNSKNTLYMELSSLRSEDTAVYYCAKDSQLRSLLYFEWLSQGYFDYWGQGTTLTVSS

SEQ ID NO: 121 - protein, 129 aa, Artificial Sequence
  source: /note="Description of Artificial Sequence: Synthetic polypeptide"
QVQLVQSGAEVKKPGSSVKVSCAASGFTFSTYAIHWVRQAPGQGLEWMGVISYDANYKYYAQKFQGRVTITRDNSKSTAYMELSSLRSEDTAVYYCAKDSQLRSLLYFEWLSQGYFDYWGQGTTLTVSS

SEQ ID NO: 122 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMAVISYDANYKYYAQKFQGRVTITRDNSKSTAYMELSSLRSEDTAVYYCAKDSQLRSLLYFEWLSQGYFDYWGQGTTLTVSS

SEQ ID NO: 123 - protein, 129 aa, Artificial Sequence
  source: /note="Description of Artificial Sequence: Synthetic polypeptide"
QVQLVQSGAEVKKPGSSLKLSCKASGFTFSTYAIHWVRQAPGQGLEWVAVISYDANYKYYAQKVQGRFTITRDNSKSTAYLEMSSLRSEDTAVYYCAKDSQLRSLLYFEWLSQGYFDYWGQGTTVTVSS

SEQ ID NO: 124 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSQLRSLLYFEWLSQGYFNPWGQGTTLTVSS

SEQ ID NO: 125 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYFDYWGQGTTLTVSS

SEQ ID NO: 126 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYFNPWGQGTTLTVSS

SEQ ID NO: 127 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYFGVWGQGTTLTVSS

SEQ ID NO: 128 - protein, 129 aa, Artificial Sequence
  source: /note="Description of Artificial Sequence: Synthetic polypeptide"
QVQLVQSGAEVKKPGSSVKVSCKASGFTFTSYAIHWVRQAPGQGLEWMGVISYDANYKYYAQKFQGRVTITRDNSKSTAYMELSSLRSEDTAVYYCAKDSQLRSLLYFEWLSQGYFDYWGQGTTLTVSS

SEQ ID NO: 129 - protein, 129 aa, Artificial Sequence
  source: /note="Description of Artificial Sequence: Synthetic polypeptide"
QVQLVQSGAEVKKPGSSVKVSCKASGFTFSDYAIHWVRQAPGQGLEWMGVISYDANYKYYAQKFQGRVTITRDNSKSTAYMELSSLRSEDTAVYYCAKDSQLRSLLYFEWLSQGYFDYWGQGTTLTVSS

SEQ ID NO: 130 - protein, 129 aa, Artificial Sequence
  source: /note="Description of Artificial Sequence: Synthetic polypeptide"
QVQLVQSGAEVKKPGSSVKVSCKASGFTFSTRAIHWVRQAPGQGLEWMGVISYDANYKYYAQKFQGRVTITRDNSKSTAYMELSSLRSEDTAVYYCAKDSQLRSLLYFEWLSQGYFDYWGQGTTLTVSS

SEQ ID NO: 131 - protein, 129 aa, Artificial Sequence
  source: /note="Description of Artificial Sequence: Synthetic polypeptide"
QVQLVQSGAEVKKPGSSVKVSCKASGFTFSTHAIHWVRQAPGQGLEWMGVISYDANYKYYAQKFQGRVTITRDNSKSTAYMELSSLRSEDTAVYYCAKDSQLRSLLYFEWLSQGYFDYWGQGTTLTVSS

SEQ ID NO: 132 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDANYKYYADSVQGRFTISRDNSKNTLYLQMNSLRAEDTAVYYCAKDSRLRSLLYFEWLSQGYFNPWGQGTTLTVSS

SEQ ID NO: 133 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDADYKYYADSVQGRFTISRDNSKNTLYLQMNSLRAEDTAVYYCAKDSRLRSLLYFEWLSQGYFNPWGQGTTLTVSS

SEQ ID NO: 134 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDAEYKYYADSVQGRFTISRDNSKNTLYLQMNSLRAEDTAVYYCAKDSRLRSLLYFEWLSQGYFNPWGQGTTLTVSS

SEQ ID NO: 135 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDAHYKYYADSVQGRFTISRDNSKNTLYLQMNSLRAEDTAVYYCAKDSRLRSLLYFEWLSQGYFNPWGQGTTLTVSS

SEQ ID NO: 136 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGHYKYYADSVQGRFTISRDNSKNTLYLQMNSLRAEDTAVYYCAKDSRLRSLLYFEWLSQGYFNPWGQGTTLTVSS

SEQ ID NO: 137 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGFYKYYADSVQGRFTISRDNSKNTLYLQMNSLRAEDTAVYYCAKDSRLRSLLYFEWLSQGYFNPWGQGTTLTVSS

SEQ ID NO: 138 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGYYKYYADSVQGRFTISRDNSKNTLYLQMNSLRAEDTAVYYCAKDSRLRSLLYFEWLSQGYFNPWGQGTTLTVSS

SEQ ID NO: 139 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGSYKYYADSVQGRFTISRDNSKNTLYLQMNSLRAEDTAVYYCAKDSRLRSLLYFEWLSQGYFNPWGQGTTLTVSS

SEQ ID NO: 140 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSFDGNYKYYADSVQGRFTISRDNSKNTLYLQMNSLRAEDTAVYYCAKDSRLRSLLYFEWLSQGYFNPWGQGTTLTVSS

SEQ ID NO: 141 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSWDGNYKYYADSVQGRFTISRDNSKNTLYLQMNSLRAEDTAVYYCAKDSRLRSLLYFEWLSQGYFNPWGQGTTLTVSS

SEQ ID NO: 142 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYNGNYKYYADSVQGRFTISRDNSKNTLYLQMNSLRAEDTAVYYCAKDSRLRSLLYFEWLSQGYFNPWGQGTTLTVSS

SEQ ID NO: 143 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYSGNYKYYADSVQGRFTISRDNSKNTLYLQMNSLRAEDTAVYYCAKDSRLRSLLYFEWLSQGYFNPWGQGTTLTVSS

SEQ ID NO: 144 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVISYDGNYKYYADSVQGRFTISRDNSKNTLYLQMNSLRAEDTAVYYCAKDSRLRSLLYFEWLSQGYFNPWGQGTTLTVSS

SEQ ID NO: 145 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISVDNSKNTLYLQMNSLRAEDTAVYYCAKDSRLRSLLYFEWLSQGYFNPWGQGTTLTVSS

SEQ ID NO: 146 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDSSKNTLYLQMNSLRAEDTAVYYCAKDSRLRSLLYFEWLSQGYFNPWGQGTTLTVSS

SEQ ID NO: 147 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSFLYFEWLSQGYFNPWGQGTTLTVSS

SEQ ID NO: 148 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSLLFFEWLSQGYFNPWGQGTTLTVSS

SEQ ID NO: 149 - protein, 129 aa, Artificial Sequence
  source: /note="Description of Artificial Sequence: Synthetic polypeptide"
QVQLLETGGGLVKPGQSLKLSCAASGFTFTSYAMHWVRQPPGKGLEWVAVVSYDGNYKYYADSVQGRFTISRDNSKNTLYLQMNSLRAEDTAVYYCAKDSRLRSFLFFEWLSQGYFNPWGQGTTLTVSS

SEQ ID NO: 150 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKNTAYMELSSLRSEDTAVYYCAKDSRLRSLLYFEWLSQGYFNPWGQGTTLTVSS

SEQ ID NO: 151 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKLTAYMELSSLRSEDTAVYYCAKDSRLRSLLYFEWLSQGYFNPWGQGTTLTVSS

SEQ ID NO: 152 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKWTAYMELSSLRSEDTAVYYCAKDSRLRSLLYFEWLSQGYFNPWGQGTTLTVSS

SEQ ID NO: 153 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKFTAYMELSSLRSEDTAVYYCAKDSRLRSLLYFEWLSQGYFNPWGQGTTLTVSS

SEQ ID NO: 154 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNYKSTAYMELSSLRSEDTAVYYCAKDSRLRSLLYFEWLSQGYFNPWGQGTTLTVSS

SEQ ID NO: 155 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNWKSTAYMELSSLRSEDTAVYYCAKDSRLRSLLYFEWLSQGYFNPWGQGTTLTVSS

SEQ ID NO: 156 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSYSTAYMELSSLRSEDTAVYYCAKDSRLRSLLYFEWLSQGYFNPWGQGTTLTVSS

SEQ ID NO: 157 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSWSTAYMELSSLRSEDTAVYYCAKDSRLRSLLYFEWLSQGYFNPWGQGTTLTVSS

SEQ ID NO: 158 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCARDSRLRSLLYFEWLSQGYFNPWGQGTTLTVSS

SEQ ID NO: 159 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCARDSRLRSLLYFEWLSQGYFGVWGQGTTLTVSS

SEQ ID NO: 160 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYFDPWGQGTTLTVSS

SEQ ID NO: 161 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCARDSRLRSLLYFEWLSQGYFDPWGQGTTLTVSS

SEQ ID NO: 162 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYFQPWGQGTTLTVSS

SEQ ID NO: 163 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYFNDWGQGTTLTVSS

SEQ ID NO: 164 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYFDDWGQGTTLTVSS

SEQ ID NO: 165 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYFQHWGQGTLVTVSS

SEQ ID NO: 166 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYFDLWGRGTLVTVSS

SEQ ID NO: 167 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYFDIWGQGTMVTVSS

SEQ ID NO: 168 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYFDYWGQGTLVTVSS

SEQ ID NO: 169 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYFDPWGQGTLVTVSS

SEQ ID NO: 170 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYMDVWGQGTTVTVSS

SEQ ID NO: 171 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYFDFWGQGTTLTVSS

SEQ ID NO: 172 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYFDVWGQGTTLTVSS

SEQ ID NO: 173 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCARDSRLRSLLYFEWLSQGYFEIWGQGTTLTVSS

SEQ ID NO: 174 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCARDSRLRSLLYFEWLSQGYFEYWGQGTTLTVSS

SEQ ID NO: 175 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYFSYWGQGTTLTVSS

SEQ ID NO: 176 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRLRSLLYFEWLSQGYMGVWGQGTTLTVSS

SEQ ID NO: 177 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSKSTAYMELSSLRSEDTAVYYCAKDSRNRSLLYFEWLSQGYFNPWGQGTTLTVSS

SEQ ID NO: 178 - protein, 129 aa, Artificial Sequence
  source: /note="Description of Artificial Sequence: Synthetic polypeptide"
QVQLVQSGAEVKKPGSSVKVSCKASGFTFSTYAIHWVRQAPGQGLEWMGVISYDANYKYYAQKFQGRVTITRDNSWLTAYMELSSLRSEDTAVYYCAKDSRLRSLLYFEWLSQGYFNPWGQGTTLTVSS

SEQ ID NO: 179 - protein, 129 aa, Artificial Sequence
  source: /note="Description of Artificial Sequence: Synthetic polypeptide"
EVQLLESGGGLVQPGGSLRLSCAASGFTFSSYAMHWVRQAPGKGLEWVAVISYDANYKYYADSVKGRFTISRDNSWLTLYLQMNSLRAEDTAVYYCAKDSRLRSLLYFEWLSQGYFNPWGQGTLVTVSS

SEQ ID NO: 180 - protein, 129 aa, Artificial Sequence
  source: /note="Description of Artificial Sequence: Synthetic polypeptide"
EVQLLESGGGLVQPGGSLRLSCAASGFTFSSYAMHWVRQAPGKGLEWVAVISYDANYKYYADSVKGRFTISRDNSKSTLYLQMNSLRAEDTAVYYCAKDSRLRSLLYFEWLSQGYFNPWGQGTLVTVSS

SEQ ID NO: 181 - protein, 129 aa, Artificial Sequence
  source: /note="Description of Artificial Sequence: Synthetic polypeptide"
EVQLLESGGGLVQPGGSLRLSCAASGFTFSSYAMHWVRQAPGKGLEWVAVISYDANYKYYADSVKGRFTISRDNSKLTLYLQMNSLRAEDTAVYYCAKDSRLRSLLYFEWLSQGYFNPWGQGTLVTVSS

SEQ ID NO: 182 - protein, 129 aa, Artificial Sequence
  source: /note="Description of Artificial Sequence: Synthetic polypeptide"
EVQLLESGGGLVQPGGSLRLSCAASGFTFSSYAMHWVRQAPGKGLEWVAVISYDANYKYYADSVKGRFTISRDNSWNTLYLQMNSLRAEDTAVYYCAKDSRLRSLLYFEWLSQGYFNPWGQGTLVTVSS

SEQ ID NO: 183 - protein, 129 aa, Artificial Sequence
  source: /note="Description of Artificial Sequence: Synthetic polypeptide"
QVQLVQSGAEVKKPGSSVKVSCKASGFTFTSYAIHWVRQAPGQGLEWMGVISYDGNYKYYAQKFQGRVTITRDNSKLTAYMELSSLRSEDTAVYYCAKDSRLRSLLYFEWLSQGYFNPWGQGTTLTVSS

SEQ ID NO: 184 - protein, 129 aa, Artificial Sequence
  source: /note="Description of Artificial Sequence: Synthetic polypeptide"
QVQLVQSGAEVKKPGSSVKVSCKASGFTFTSYAIHWVRQAPGQGLEWMGVISYDGNYKYYAQKFQGRVTITRDNSWLTAYMELSSLRSEDTAVYYCAKDSRLRSLLYFEWLSQGYFNPWGQGTTLTVSS

SEQ ID NO: 185 - protein, 129 aa, Artificial Sequence
  source: /note="Description of Artificial Sequence: Synthetic polypeptide"
QVQLVQSGAEVKKPGSSVKVSCKASGFTFTSYAIHWVRQAPGQGLEWMGVISYDGNYKYYAQKFQGRVTITRDNSKSTAYMELSSLRSEDTAVYYCAKDSRLRSLLYFEWLSQGYFNPWGQGTTLTVSS

SEQ ID NO: 186 - protein, 129 aa, Artificial Sequence
  source: /note="Description of Artificial Sequence: Synthetic polypeptide"
EVQLLESGGGLVQPGGSLRLSCAASGFTFTSYAMHWVRQAPGKGLEWVAVISYDGNYKYYADSVKGRFTISRDNSKNTLYLQMNSLRAEDTAVYYCAKDSRLRSLLYFEWLSQGYFNPWGQGTLVTVSS

SEQ ID NO: 187 - protein, 129 aa, Artificial Sequence
  source: /note="Description of Artificial Sequence: Synthetic polypeptide"
EVQLLESGGGLVQPGGSLRLSCAASGFTFTSYAMHWVRQAPGKGLEWVAVISYDGNYKYYADSVKGRFTISRDNSWLTLYLQMNSLRAEDTAVYYCAKDSRLRSLLYFEWLSQGYFNPWGQGTLVTVSS

SEQ ID NO: 188 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYWGSYLESGVPSRFSGSGSGTDFTLTISSLQPEDFATYYCQQHYRTPPSFGQGTKVEIK

SEQ ID NO: 189 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYWGSSLQSGVPSRFSGSGSGTDFTLTISSLQPEDFATYYCQQHYRTPPSFGQGTKVEIK

SEQ ID NO: 190 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYAASSLQSGVPSRFSGSGSGTDFTLTISSLQPEDFATYYCQQHYRTPPSFGQGTKVEIK

SEQ ID NO: 191 - protein, 111 aa, Artificial Sequence
  source: /note="Description of Artificial Sequence: Synthetic polypeptide"
DIVMTQSPDSLAVSLGERATINCKSSQSVTFDYKNYLAWYQQKPGQPPKLLIYWASTRESGVPDRFSGSGSGTDFTLTISSLQAEDVAVYYCQQHYRTPPSFGQGTKVEIK

SEQ ID NO: 192 - protein, 111 aa, Artificial Sequence
  source: /note="Description of Artificial Sequence: Synthetic polypeptide"
DIVMTQSPDSLAVSLGERATINCKSSQSVTFDYKNYLAWYQQKPGQPPKLLIYEASTRESGVPDRFSGSGSGTDFTLTISSLQAEDVAVYYCQQHYRTPPSFGQGTKVEIK

SEQ ID NO: 193 - protein, 111 aa, Artificial Sequence
  source: /note="Description of Artificial Sequence: Synthetic polypeptide"
EIVMTQSPATLSVSPGERATLSCRSSQSITFDYKNYLAWYQQKPGQAPRLLIYWGSTRATGIPARFSGSGSGTEFTLTISSLQSEDFAVYYCQQHYRTPPSFGQGTKVEIK

SEQ ID NO: 194 - protein, 111 aa, Artificial Sequence
  source: /note="Description of Artificial Sequence: Synthetic polypeptide"
EIVMTQSPATLSVSPGERATLSCRSSQSITFDYKNYLAWYQQKPGQAPRLLIYSASTRATGIPARFSGSGSGTEFTLTISSLQSEDFAVYYCQQHYRTPPSFGQGTKVEIK

SEQ ID NO: 195 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYWGSYLESGVPSRFSGSGSGTDFTLTISSLQPEDFATYYCQQCYRTPPSFGQGTKVEIK

SEQ ID NO: 196 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYWGSYLESGVPSRFSGSGSGTDFTLTISSLQPEDFATYYCQQHYCTPPSFGQGTKVEIK

SEQ ID NO: 197 - protein, 111 aa, Artificial Sequence
  source: /note="Description of Artificial Sequence: Synthetic polypeptide"
DIQMTQSPSSLSASVGDRVTITCRSSQSITFDYQNYLAWYQQKPGKAPKLLIYWGSYLESGVPSRFSGSGSGTDFTLTISSLQPEDFATYYCQQHYRTPPSFGQGTKVEIK

SEQ ID NO: 198 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYWGSYLESGVPSRFSGSGSGTDFTLTISSLQPEDFATYYCQQFYRTPPSFGQGTKVEIK

SEQ ID NO: 199 - protein, 111 aa, Artificial Sequence
  source: /note="Description of Artificial Sequence: Synthetic polypeptide"
QIQMTQSPSSLSASVGDRVTITCRSSQSITFDYKNYLAWYQQKPGKAPKLLIYWGSYLESGVPSRFSGSGSGTDFTLTISSLQPEDFATYYCQQHYRTPPSFGQGTKVEIK

SEQ ID NO: 200 - protein, 111 aa, Artificial Sequence
  source: /note="Description of Artificial Sequence: Synthetic polypeptide"
DIQMTQSPSSLSASVGDRVTITCRSSQSITFDYQNYLAWYQQKPGKAPKLLIYWGSTLESGVPSRFSGSGSGTDFTLTISSLQPEDFATYYCQQHYRTPPSFGQGTKVEIK

SEQ ID NO: 201 - protein, 111 aa, Artificial Sequence
  source: /note="Description of Artificial Sequence: Synthetic polypeptide"
DIQMTQSPSSLSASVGDRVTITCRSSQSITSDNQNYLAWYQQKPGKAPKLLIYWGSYLESGVPSRFSGSGSGTDFTLTISSLQPEDFATYYCQQHYRTPPSFGQGTKVEIK

SEQ ID NO: 202 - protein, 112 aa, Artificial Sequence
  source: /note="Description of Artificial Sequence: Synthetic polypeptide"
DIVMTQSPDSLAVSLGERATINCKSSQSVLYSSNNKNYLAWYQQKPGQPPKLLIDWASTRESGVPDRFSGSGSGTDFTLTISNLQVEDVAVYYCQQYYRSPSFGQGTKLEIK

SEQ ID NO: 203 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIDWGSYLESGVPSRFSGSGSGTDFTLTISSLQPEDFATYYCQQHYRTPPSFGQGTKVEIK

SEQ ID NO: 204 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIDWGSYLESGVPSRFSGSGSGTDFTLTISSLQPEDFATYYCQQYYRTPPSFGQGTKVEIK

SEQ ID NO: 205 - protein, 111 aa, Artificial Sequence
  source: /note="Description of Artificial Sequence: Synthetic polypeptide"
DIVMTQSPDSLAVSLGERATINCKSSQSVSFNYKNYLAWYQQKPGQPPKLLIDWASTRESGVPDRFSGSGSGTDFTLTISNLQVEDVAVYYCQQYYRSPPSFGQGTKLEIK

SEQ ID NO: 206 - protein, 111 aa, Artificial Sequence
  source: /note="Description of Artificial Sequence: Synthetic polypeptide"
DIQMTQSPDSLAVSLGARATINCKSSQSVTFNYKNYLAWYQQKPGQPPKVLIDWASARESGVPDRFSGSGSGTDFTLTISSLQAEDVAVYYCQQHYRTPPTFGQGTKVEIK

SEQ ID NO: 207 - protein, 111 aa, Artificial Sequence
  source: /note="Description of Artificial Sequence: Synthetic polypeptide"
DIVMTQSPDSLAVSLGERATINCKSSQSVTFNYKNYLAWYQQKPGQPPKLLIYWASTRESGVPDRFSGSGSGTDFTLTISSLQAEDVAVYYCQQHYRTPPTFGQGTKVEIK

SEQ ID NO: 208 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYEGSYLESGVPSRFSGSGSGTDFTLTISSLQPEDFATYYCQQHYRTPPSFGQGTKVEIK

SEQ ID NO: 209 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYSGSYLESGVPSRFSGSGSGTDFTLTISSLQPEDFATYYCQQHYRTPPSFGQGTKVEIK

SEQ ID NO: 210 - protein, 111 aa, Artificial Sequence
  source: /note="Description of Artificial Sequence: Synthetic polypeptide"
DIQMTQSPSSLSASVGDRVTITCRSSQSITFDYKNRLAWYQQKPGKAPKLLIYWGSYLESGVPSRFSGSGSGTDFTLTISSLQPEDFATYYCQQHYRTPPSFGQGTKVEIK

SEQ ID NO: 211 - protein, 111 aa, Artificial Sequence
  source: /note="Description of Artificial Sequence: Synthetic polypeptide"
DIQMTQSPSSLSASVGDRVTITCRSSQSITSDNKNYLAWYQQKPGKAPKLLIYWGSYLESGVPSRFSGSGSGTDFTLTISSLQPEDFATYYCQQHYRTPPSFGQGTKVEIK

SEQ ID NO: 212 - protein, 111 aa, Artificial Sequence
  source: /note="Description of Artificial Sequence: Synthetic polypeptide"
DIQMTQSPSSLSASVGDRVTITCRSSQSITSDDKNYLAWYQQKPGKAPKLLIYWGSYLESGVPSRFSGSGSGTDFTLTISSLQPEDFATYYCQQHYRTPPSFGQGTKVEIK

SEQ ID NO: 213 - protein, 111 aa, Artificial Sequence
  source: /note="Description of Artificial Sequence: Synthetic polypeptide"
DIQMTQSPSSLSASVGDRVTITCRSSQSITSDEKNYLAWYQQKPGKAPKLLIYWGSYLESGVPSRFSGSGSGTDFTLTISSLQPEDFATYYCQQHYRTPPSFGQGTKVEIK

SEQ ID NO: 214 - protein, 111 aa, Artificial Sequence
  source: /note="Description of Artificial Sequence: Synthetic polypeptide"
DIQMTQSPSSLSASVGDRVTITCRSSQSITDDNKNYLAWYQQKPGKAPKLLIYWGSYLESGVPSRFSGSGSGTDFTLTISSLQPEDFATYYCQQHYRTPPSFGQGTKVEIK

SEQ ID NO: 215 - protein, 111 aa, Artificial Sequence
  source: /note="Description of Artificial Sequence: Synthetic polypeptide"
DIQMTQSPSSLSASVGDRVTITCRSSQSITDDDKNYLAWYQQKPGKAPKLLIYWGSYLESGVPSRFSGSGSGTDFTLTISSLQPEDFATYYCQQHYRTPPSFGQGTKVEIK

SEQ ID NO: 216 - protein, 111 aa, Artificial Sequence
  source: /note="Description of Artificial Sequence: Synthetic polypeptide"
DIQMTQSPSSLSASVGDRVTITCRSSQSITDDEKNYLAWYQQKPGKAPKLLIYWGSYLESGVPSRFSGSGSGTDFTLTISSLQPEDFATYYCQQHYRTPPSFGQGTKVEIK

SEQ ID NO: 217 - protein, 111 aa, Artificial Sequence
  source: /note="Description of Artificial Sequence: Synthetic polypeptide"
DIQMTQSPSSLSASVGDRVTITCRSSQSITEDNKNYLAWYQQKPGKAPKLLIYWGSYLESGVPSRFSGSGSGTDFTLTISSLQPEDFATYYCQQHYRTPPSFGQGTKVEIK

SEQ ID NO: 218 - protein, 111 aa, Artificial Sequence
  source: /note="Description of Artificial Sequence: Synthetic polypeptide"
DIQMTQSPSSLSASVGDRVTITCRSSQSITEDDKNYLAWYQQKPGKAPKLLIYWGSYLESGVPSRFSGSGSGTDFTLTISSLQPEDFATYYCQQHYRTPPSFGQGTKVEIK

SEQ ID NO: 219 - protein, 111 aa, Artificial Sequence
  source: /note="Description of Artificial Sequence: Synthetic polypeptide"
DIQMTQSPSSLSASVGDRVTITCRSSQSITEDEKNYLAWYQQKPGKAPKLLIYWGSYLESGVPSRFSGSGSGTDFTLTISSLQPEDFATYYCQQHYRTPPSFGQGTKVEIK

SEQ ID NO: 220 - protein, 111 aa, Artificial Sequence
  source: /note="Description of Artificial Sequence: Synthetic polypeptide"
DIQMTQSPSSLSASVGDRVTITCRSSQSITQDNKNYLAWYQQKPGKAPKLLIYWGSYLESGVPSRFSGSGSGTDFTLTISSLQPEDFATYYCQQHYRTPPSFGQGTKVEIK

SEQ ID NO: 221 - protein, 111 aa, Artificial Sequence
  source: /note="Description of Artificial Sequence: Synthetic polypeptide"
DIQMTQSPSSLSASVGDRVTITCRSSQSITQDDKNYLAWYQQKPGKAPKLLIYWGSYLESGVPSRFSGSGSGTDFTLTISSLQPEDFATYYCQQHYRTPPSFGQGTKVEIK

SEQ ID NO: 222 - protein, 111 aa, Artificial Sequence
  source: /note="Description of Artificial Sequence: Synthetic polypeptide"
DIQMTQSPSSLSASVGDRVTITCRSSQSITQDEKNYLAWYQQKPGKAPKLLIYWGSYLESGVPSRFSGSGSGTDFTLTISSLQPEDFATYYCQQHYRTPPSFGQGTKVEIK

SEQ ID NO: 223 - protein, 111 aa, Artificial Sequence
  source: /note="Description of Artificial Sequence: Synthetic polypeptide"
DIQMTQSPSSLSASVGDRVTITCRSSQSITHDNKNYLAWYQQKPGKAPKLLIYWGSYLESGVPSRFSGSGSGTDFTLTISSLQPEDFATYYCQQHYRTPPSFGQGTKVEIK

SEQ ID NO: 224 - protein, 111 aa, Artificial Sequence
  source: /note="Description of Artificial Sequence: Synthetic polypeptide"
DIQMTQSPSSLSASVGDRVTITCRSSQSITHDDKNYLAWYQQKPGKAPKLLIYWGSYLESGVPSRFSGSGSGTDFTLTISSLQPEDFATYYCQQHYRTPPSFGQGTKVEIK

SEQ ID NO: 225 - protein, 111 aa, Artificial Sequence
  source: /note="Description of Artificial Sequence: Synthetic polypeptide"
DIQMTQSPSSLSASVGDRVTITCRSSQSITHDEKNYLAWYQQKPGKAPKLLIYWGSYLESGVPSRFSGSGSGTDFTLTISSLQPEDFATYYCQQHYRTPPSFGQGTKVEIK

SEQ ID NO: 226 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYAASSRQSGVPDRFSGSGSGTDFTLTISSLQPEDFATYYCQQHYRTPPSFGQGTKVEIK

SEQ ID NO: 227 - protein, 111 aa, Artificial Sequence
  source: /note="Description of Artificial Sequence: Synthetic polypeptide"
DIQMTQSPSSLSASVGDRVTITCRSSQSITFNYKNYLAWYQQKPGKAPKLLIYAASSLQSGVPSRFSGSGSGTDFTLTISSLQPEDFATYYCQQHYRTPPSFGQGTKVEIK

SEQ ID NO: 228 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYAASSLESGVPSRFSGSGSGTDFTLTISSLQPEDFATYYCQQHYRTPPSFGQGTKVEIK

SEQ ID NO: 229 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYSASSLQSGVPSRFSGSGSGTDFTLTISSLQPEDFATYYCQQHYRTPPSFGQGTKVEIK

SEQ ID NO: 230 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYSGSSLQSGVPSRFSGSGSGTDFTLTISSLQPEDFATYYCQQHYRTPPSFGQGTKVEIK

SEQ ID NO: 231 - protein, 111 aa, Artificial Sequence
  source: /note="Description of Artificial Sequence: Synthetic polypeptide"
DIQMTQSPSSLSASVGDRVTITCRSSQSVTFDYKNYLAWYQQKPGKAPKLLIYAASSLQSGVPSRFSGSGSGTDFTLTISSLQPEDFATYYCQQHYRTPPSFGQGTKVEIK

SEQ ID NO: 232 - protein, 111 aa, Artificial Sequence
  source: /note="Description of Artificial Sequence: Synthetic polypeptide"
DIQMTQSPSSLSASVGDRVTITCRSSQSITDDNKNYLAWYQQKPGKAPKLLIYAASSLQSGVPSRFSGSGSGTDFTLTISSLQPEDFATYYCQQHYRTPPSFGQGTKVEIK

SEQ ID NO: 233 - protein, 111 aa, Artificial Sequence
  source: /note="Description of Artificial Sequence: Synthetic polypeptide"
DIQMTQSPSSLSASVGDRVTITCRSSQSITSDYKNYLAWYQQKPGKAPKLLIYAASSLQSGVPSRFSGSGSGTDFTLTISSLQPEDFATYYCQQHYRTPPSFGQGTKVEIK

SEQ ID NO: 234 - protein, 111 aa, Artificial Sequence
  source: /note="Description of Artificial Sequence: Synthetic polypeptide"
DIQMTQSPSSLSASVGDRVTITCRSSQSITSDNKNYLAWYQQKPGKAPKLLIYAASSLQSGVPSRFSGSGSGTDFTLTISSLQPEDFATYYCQQHYRTPPSFGQGTKVEIK

SEQ ID NO: 235 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYAASSLQSGVPSRFSGSGSGTDFTLTISSLQPEDFATYYCQQHYSTPPSFGQGTKVEIK

SEQ ID NO: 236 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYAASSLQSGVPSRFSGSGSGTDFTLTISSLQPEDFATYYCQQHYQTPPSFGQGTKVEIK

SEQ ID NO: 237 - protein, 111 aa, Artificial Sequence
  source: /note="Description of Artificial Sequence: Synthetic polypeptide"
DIQMTQSPDSLSASVGDRVTITCRSSQSITFDYKNYLAWYQQKPGKAPKLLIYAASSLQSGVPSRFSGSGSGTDFTLTISSLQPEDFATYYCQQHYRTPPTFGQGTKVEIK

SEQ ID NO: 238 - protein, 111 aa, Artificial Sequence
  source: /note="Description of Artificial Sequence: Synthetic polypeptide"
DIVMTQSPDSLAVSLGERATINCKSSQSVTFDYKNYLAWYQQKPGQPPKLLIYWASTRESGVPDRFSGSGSGTDFTLTISSLQAEDVAVYYCQQHYRTPPTFGQGTKVEIK

SEQ ID NO: 239 - protein, 111 aa, Artificial Sequence
  source: /note="Description of Artificial Sequence: Synthetic polypeptide"
DIVMTQSPDSLAVSLGERATINCKSSQSVTFNYKNYLAWYQQKPGQPPKLLIYWASTRESGVPDRFSGSGSGTDFTLTISSLQAEDVAVYYCQQHYRTPPSFGQGTKVEIK

SEQ ID NO: 240 - protein, 111 aa, Artificial Sequence
  source: /note="Description of Artificial Sequence: Synthetic polypeptide"
DIVMTQSPDSLAVSLGERATINCKSSQSVTFDYKNYLAWYQQKPGQPPKLLIYSASTRESGVPDRFSGSGSGTDFTLTISSLQAEDVAVYYCQQHYRTPPSFGQGTKVEIK

SEQ ID NO: 241 - protein, 111 aa, Artificial Sequence
  source: /note="Description of Artificial Sequence: Synthetic polypeptide"
DIVMTQSPDSLAVSLGERATINCKSSQSVTFDYKNYLAWYQQKPGQPPKLLIYWASTRQSGVPDRFSGSGSGTDFTLTISSLQAEDVAVYYCQQHYRTPPSFGQGTKVEIK

SEQ ID NO: 242 - protein, 111 aa, Artificial Sequence
  source: /note="Description of Artificial Sequence: Synthetic polypeptide"
DIVMTQSPDSLAVSLGERATINCKSSQSITFDYKNYLAWYQQKPGQPPKLLIYWASTRESGVPDRFSGSGSGTDFTLTISSLQAEDVAVYYCQQHYRTPPSFGQGTKVEIK

SEQ ID NO: 243 - protein, 111 aa, Artificial Sequence
  source: /note="Description of Artificial Sequence: Synthetic polypeptide"
DIVMTQSPDSLAVSLGERATINCKSSQSVTFDYKNYLAWYQQKPGQPPKLLIYSGSTRESGVPDRFSGSGSGTDFTLTISSLQAEDVAVYYCQQHYRTPPSFGQGTKVEIK

SEQ ID NO: 244 - protein, 111 aa, Artificial Sequence
  source: /note="Description of Artificial Sequence: Synthetic polypeptide"
DIQMTQSPDSLSVSLGERATINCKSSQSVTFDYKNYLAWYQQKPGQPPKLLIYWASTRESGVPDRFSGSGSGTDFTLTISSLQAEDVAVYYCQQHYRTPPSFGQGTKVEIK

SEQ ID NO: 245 - protein, 111 aa, Artificial Sequence
  source: /note="Description of Artificial Sequence: Synthetic polypeptide"
DIVMTQSPDSLAVSLGERATINCKSSQSVTFDYKNYLAWYQQKPGQPPKLLIYWASTRESGVPDRFSGSGSGTDFTLTISSLQAEDVATYYCQQHYRTPPSFGQGTKVEIK

SEQ ID NO: 246 - protein, 111 aa, Artificial Sequence
  source: /note="Description of Artificial Sequence: Synthetic polypeptide"
DIVMTQSPDSLAVSLGERATINCKSSQSVTDDNKNYLAWYQQKPGQPPKLLIYWASTRESGVPDRFSGSGSGTDFTLTISSLQAEDVAVYYCQQHYRTPPSFGQGTKVEIK

SEQ ID NO: 247 - protein, 111 aa, Artificial Sequence
  source: /note="Description of Artificial Sequence: Synthetic polypeptide"
DIVMTQSPDSLAVSLGERATINCKSSQSVTSDYKNYLAWYQQKPGQPPKLLIYWASTRESGVPDRFSGSGSGTDFTLTISSLQAEDVAVYYCQQHYRTPPSFGQGTKVEIK

SEQ ID NO: 248 - protein, 111 aa, Artificial Sequence
  source: /note="Description of Artificial Sequence: Synthetic polypeptide"
DIVMTQSPDSLAVSLGERATINCKSSQSVTSDNKNYLAWYQQKPGQPPKLLIYWASTRESGVPDRFSGSGSGTDFTLTISSLQAEDVAVYYCQQHYRTPPSFGQGTKVEIK

SEQ ID NO: 249 - protein, 111 aa, Artificial Sequence
  source: /note="Description of Artificial Sequence: Synthetic polypeptide"
DIVMTQSPDSLAVSLGERATINCKSSQSVTFDYKNYLAWYQQKPGQPPKLLIYWASTRESGVPDRFSGSGSGTDFTLTISSLQAEDVAVYYCQQHYSTPPSFGQGTKVEIK

SEQ ID NO: 250 - protein, 111 aa, Artificial Sequence
  source: /note="Description of Artificial Sequence: Synthetic polypeptide"
DIVMTQSPDSLAVSLGERATINCKSSQSVTFDYKNYLAWYQQKPGQPPKLLIYWASTRESGVPDRFSGSGSGTDFTLTISSLQAEDVAVYYCQQHYQTPPSFGQGTKVEIK

SEQ ID NO: 251 - protein, 111 aa, Artificial Sequence
  source: /note="Description of Artificial Sequence: Synthetic polypeptide"
DIQMTQSPSSLSASVGDRVTITCRSSESITFDYKNYLAWYQQKPGKAPKLLIYWGSYLESGVPSRFSGSGSGTDFTLTISSLQPEDFATYYCQQHYRTPPSFGQGTKVEIK

SEQ ID NO: 252 - protein, 111 aa, Artificial Sequence
  source: /note="Description of Artificial Sequence: Synthetic polypeptide"
DIQMTQSPSSLSASVGDRVTITCRSSEDITFDYKNYLAWYQQKPGKAPKLLIYWGSYLESGVPSRFSGSGSGTDFTLTISSLQPEDFATYYCQQHYRTPPSFGQGTKVEIK

SEQ ID NO: 253 - protein, 111 aa, Artificial Sequence
  source: /note="Description of Artificial Sequence: Synthetic polypeptide"
DIQMTQSPSSLSASVGDRVTITCRSSESITFDYKNYLAWYQQKPGKAPKLLIYWGSYLESGVPSRFSGSGSGTDFTLTISSLQPEDFATYYCQQHYQTPPSFGQGTKVEIK

SEQ ID NO: 254 - protein, 111 aa, Artificial Sequence
  source: /note="Description of Artificial Sequence: Synthetic polypeptide"
DIQMTQSPSSLSASVGDRVTITCRSSEDITFDYKNYLAWYQQKPGKAPKLLIYWGSYLESGVPSRFSGSGSGTDFTLTISSLQPEDFATYYCQQHYQTPPSFGQGTKVEIK

SEQ ID NO: 255 - protein, 113 aa, Artificial Sequence
  source: /note="Description of Artificial Sequence: Synthetic polypeptide"
DIQMTQSPSSLSASVGDRVTITCRSSQSITFSSDYKNYLAWYQQKPGKAPKLLIYWGSYLESGVPSRFSGSGSGTDFTLTISSLQPEDFATYYCQQHYRTPPSFGQGTKVEIK

SEQ ID NO: 256 - protein, 113 aa, Artificial Sequence
  source: /note="Description of Artificial Sequence: Synthetic polypeptide"
DIQMTQSPSSLSASVGDRVTITCRSSQSITFSPDYKNYLAWYQQKPGKAPKLLIYWGSYLESGVPSRFSGSGSGTDFTLTISSLQPEDFATYYCQQHYRTPPSFGQGTKVEIK

SEQ ID NO: 257 - protein, 113 aa, Artificial Sequence
  source: /note="Description of Artificial Sequence: Synthetic polypeptide"
DIQMTQSPSSLSASVGDRVTITCRSSQSITLSPDYKNYLAWYQQKPGKAPKLLIYWGSYLESGVPSRFSGSGSGTDFTLTISSLQPEDFATYYCQQHYRTPPSFGQGTKVEIK

SEQ ID NO: 258 - protein, 113 aa, Artificial Sequence
  source: /note="Description of Artificial Sequence: Synthetic polypeptide"
DIQMTQSPSSLSASVGDRVTITCRSSQSITISPDYKNYLAWYQQKPGKAPKLLIYWGSYLESGVPSRFSGSGSGTDFTLTISSLQPEDFATYYCQQHYRTPPSFGQGTKVEIK

SEQ ID NO: 259 - protein, 113 aa, Artificial Sequence
  source: /note="Description of Artificial Sequence: Synthetic polypeptide"
DIQMTQSPSSLSASVGDRVTITCRSSQSITFGGDYKNYLAWYQQKPGKAPKLLIYWGSYLESGVPSRFSGSGSGTDFTLTISSLQPEDFATYYCQQHYRTPPSFGQGTKVEIK

SEQ ID NO: 260 - protein, 112 aa, Artificial Sequence
  source: /note="Description of Artificial Sequence: Synthetic polypeptide"
DIQMTQSPSSLSASVGDRVTITCRSSQSITFSDYKNYLAWYQQKPGKAPKLLIYWGSYLESGVPSRFSGSGSGTDFTLTISSLQPEDFATYYCQQHYRTPPSFGQGTKVEIK

SEQ ID NO: 261 - protein, 112 aa, Artificial Sequence
  source: /note="Description of Artificial Sequence: Synthetic polypeptide"
DIQMTQSPSSLSASVGDRVTITCRSSQSITFGDYKNYLAWYQQKPGKAPKLLIYWGSYLESGVPSRFSGSGSGTDFTLTISSLQPEDFATYYCQQHYRTPPSFGQGTKVEIK

SEQ ID NO: 262 - protein, 113 aa, Artificial Sequence
  source: /note="Description of Artificial Sequence: Synthetic polypeptide"
DIQMTQSPSSLSASVGDRVTITCRSSQSITFGPDYKNYLAWYQQKPGKAPKLLIYWGSYLESGVPSRFSGSGSGTDFTLTISSLQPEDFATYYCQQHYRTPPSFGQGTKVEIK

SEQ ID NO: 263 - protein, 111 aa, Artificial Sequence
  source: /note="Description of Artificial Sequence: Synthetic polypeptide"
DIQMTQSPSSLSASVGDRVTITCRSSQSITYDYKNYLAWYQQKPGKAPKLLIYWGSYLESGVPSRFSGSGSGTDFTLTISSLQPEDFATYYCQQHYRTPPSFGQGTKVEIK

SEQ ID NO: 264 - protein, 111 aa, Artificial Sequence
  source: /note="Description of Artificial Sequence: Synthetic polypeptide"
DIQMTQSPSSLSASVGDRVTITCRSSQSITWDYKNYLAWYQQKPGKAPKLLIYWGSYLESGVPSRFSGSGSGTDFTLTISSLQPEDFATYYCQQHYRTPPSFGQGTKVEIK

SEQ ID NO: 265 - protein, 111 aa, Artificial Sequence
  source: /note="Description of Artificial Sequence: Synthetic polypeptide"
DIQMTQSPSSLSASVGDRVTITCRSSQSITFWYKNYLAWYQQKPGKAPKLLIYWGSYLESGVPSRFSGSGSGTDFTLTISSLQPEDFATYYCQQHYRTPPSFGQGTKVEIK

SEQ ID NO: 266 - protein, 112 aa, Artificial Sequence
  source: /note="Description of Artificial Sequence: Synthetic polypeptide"
DIQMTQSPSSLSASVGDRVTITCRSSQSITFLDYKNYLAWYQQKPGKAPKLLIYWGSYLESGVPSRFSGSGSGTDFTLTISSLQPEDFATYYCQQHYRTPPSFGQGTKVEIK

SEQ ID NO: 267 - protein, 113 aa, Artificial Sequence
  source: /note="Description of Artificial Sequence: Synthetic polypeptide"
DIQMTQSPSSLSASVGDRVTITCRSSQSITLSPWYKNYLAWYQQKPGKAPKLLIYWGSYLESGVPSRFSGSGSGTDFTLTISSLQPEDFATYYCQQHYRTPPSFGQGTKVEIK

SEQ ID NO: 268 - protein, 113 aa, Artificial Sequence
  source: /note="Description of Artificial Sequence: Synthetic polypeptide"
DIQMTQSPSSLSASVGDRVTITCRSSQSITLSPYYKNYLAWYQQKPGKAPKLLIYWGSYLESGVPSRFSGSGSGTDFTLTISSLQPEDFATYYCQQHYRTPPSFGQGTKVEIK

SEQ ID NO: 269 - protein, 113 aa, Artificial Sequence
  source: /note="Description of Artificial Sequence: Synthetic polypeptide"
DIQMTQSPSSLSASVGDRVTITCRSSQSITLSPYDKNYLAWYQQKPGKAPKLLIYWGSYLESGVPSRFSGSGSGTDFTLTISSLQPEDFATYYCQQHYRTPPSFGQGTKVEIK

SEQ ID NO: 270 - protein, 111 aa, Artificial Sequence
  source: /note="Description of Artificial Sequence: Synthetic polypeptide"
DIQMTQSPSSLSASVGDRVTITCRSSQSITFDNKNYLAWYQQKPGKAPKLLIYWGSYLESGVPSRFSGSGSGTDFTLTISSLQPEDFATYYCQQHYRTPPSFGQGTKVEIK

SEQ ID NO: 271 - protein, 111 aa, Artificial Sequence
  source: /note="Description of Artificial Sequence: Synthetic polypeptide"
DIQMTQSPSSLSASVGDRVTITCRSSQSITSDNKNYLAWYQQKPGKAPKLLIYWGSELESGVPSRFSGSGSGTDFTLTISSLQPEDFATYYCQQHYRTPPSFGQGTKVEIK

SEQ ID NO: 272 - protein, 111 aa, Artificial Sequence
  source: /note="Description of Artificial Sequence: Synthetic polypeptide"
DIQMTQSPSSLSASVGDRVTITCRSSQSITADNKNYLAWYQQKPGKAPKLLIYWGSYLESGVPSRFSGSGSGTDFTLTISSLQPEDFATYYCQQHYRTPPSFGQGTKVEIK

SEQ ID NO: 273 - protein, 111 aa, Artificial Sequence
  source: /note="Description of Artificial Sequence: Synthetic polypeptide"
DIQMTQSPSSLSASVGDRVTITCRSSQSITNDNKNYLAWYQQKPGKAPKLLIYWGSYLESGVPSRFSGSGSGTDFTLTISSLQPEDFATYYCQQHYRTPPSFGQGTKVEIK

SEQ ID NO: 274 - protein, 111 aa, Artificial Sequence
  source: /note="Description of Artificial Sequence: Synthetic polypeptide"
DIQMTQSPSSLSASVGDRVTITCRSSQSITMDNKNYLAWYQQKPGKAPKLLIYWGSYLESGVPSRFSGSGSGTDFTLTISSLQPEDFATYYCQQHYRTPPSFGQGTKVEIK

SEQ ID NO: 275 - protein, 111 aa, Artificial Sequence
  source: /note="Description of Artificial Sequence: Synthetic polypeptide"
DIQMTQSPSSLSASVGDRVTITCRSSQSITRDNKNYLAWYQQKPGKAPKLLIYWGSYLESGVPSRFSGSGSGTDFTLTISSLQPEDFATYYCQQHYRTPPSFGQGTKVEIK

SEQ ID NO: 276 - protein, 111 aa, Artificial Sequence
  source: /note="Description of Artificial Sequence: Synthetic polypeptide"
DIQMTQSPSSLSASVGDRVTITCRSSQSITSDSKNYLAWYQQKPGKAPKLLIYWGSYLESGVPSRFSGSGSGTDFTLTISSLQPEDFATYYCQQHYRTPPSFGQGTKVEIK

SEQ ID NO: 277 - protein, 111 aa, Artificial Sequence
  source: /note="Description of Artificial Sequence: Synthetic polypeptide"
DIQMTQSPSSLSASVGDRVTITCRSSQSITSDQKNYLAWYQQKPGKAPKLLIYWGSYLESGVPSRFSGSGSGTDFTLTISSLQPEDFATYYCQQHYRTPPSFGQGTKVEIK

SEQ ID NO: 278 - protein, 111 aa, Artificial Sequence
  source: /note="Description of Artificial Sequence: Synthetic polypeptide"
DIQMTQSPSSLSASVGDRVTITCRSSQSITSDRKNYLAWYQQKPGKAPKLLIYWGSYLESGVPSRFSGSGSGTDFTLTISSLQPEDFATYYCQQHYRTPPSFGQGTKVEIK

SEQ ID NO: 279 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYWGSDLESGVPSRFSGSGSGTDFTLTISSLQPEDFATYYCQQHYRTPPSFGQGTKVEIK

SEQ ID NO: 280 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYWGSELESGVPSRFSGSGSGTDFTLTISSLQPEDFATYYCQQHYRTPPSFGQGTKVEIK

SEQ ID NO: 281 - protein, 111 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYWGEYLESGVPSRFSGSGSGTDFTLTISSLQPEDFATYYCQQHYRTPPSFGQGTKVEIK

SEQ ID NO: 282 - protein, 110 aa, Artificial Sequence
  source: /note="Description of Artificial Sequence: Synthetic polypeptide"
DIQMTQSPSSLSASVGDRVTITCRSSQSITFDYKNYLAWYQQKPGKAPKLLIYWGSYLESGVPSRFSGSGSGTDFTLTISSLQPEDFATYYCQQHYRTPSFGQGTKVEIK

SEQ ID NO: 283 - protein, 111 aa, Artificial Sequence
  source: /note="Description of Artificial Sequence: Synthetic polypeptide"
DIVMTQSPDSLAVSLGERATINCKSSQSVTFDYKNYLAWYQQKPGQPPKLLIYAASTRESGVPDRFSGSGSGTDFTLTISSLQAEDVAVYYCQQHYRTPPSFGQGTKVEIK

SEQ ID NO: 284 - protein, 111 aa, Artificial Sequence
  source: /note="Description of Artificial Sequence: Synthetic polypeptide"
DIQMTQSPSSLSASVGDRVTITCRSSEDITFWYKNYLAWYQQKPGKAPKLLIYWGSYLESGVPSRFSGSGSGTDFTLTISSLQPEDFATYYCQQHYRTPPSFGQGTKVEIK

SEQ ID NO: 285 - protein, 111 aa, Artificial Sequence
  source: /note="Description of Artificial Sequence: Synthetic polypeptide"
DIQMTQSPSSLSASVGDRVTITCRSSQSITFWEKNYLAWYQQKPGKAPKLLIYWGSYLESGVPSRFSGSGSGTDFTLTISSLQPEDFATYYCQQHYRTPPSFGQGTKVEIK

SEQ ID NO: 286 - protein, 111 aa, Artificial Sequence
  source: /note="Description of Artificial Sequence: Synthetic polypeptide"
DIQMTQSPSSLSASVGDRVTITCRSSQSITSWNKNYLAWYQQKPGKAPKLLIYWGSYLESGVPSRFSGSGSGTDFTLTISSLQPEDFATYYCQQHYRTPPSFGQGTKVEIK

SEQ ID NO: 287 - protein, 111 aa, Artificial Sequence
  source: /note="Description of Artificial Sequence: Synthetic polypeptide"
DIQMTQSPSSLSASVGDRVTITCRSSEDITSWNKNYLAWYQQKPGKAPKLLIYWGSYLESGVPSRFSGSGSGTDFTLTISSLQPEDFATYYCQQHYRTPPSFGQGTKVEIK

SEQ ID NO: 288 - protein, 111 aa, Artificial Sequence
  source: /note="Description of Artificial Sequence: Synthetic polypeptide"
DIQMTQSPSSLSASVGDRVTITCRSSEDITSWDKNYLAWYQQKPGKAPKLLIYWGSYLESGVPSRFSGSGSGTDFTLTISSLQPEDFATYYCQQHYRTPPSFGQGTKVEIK

SEQ ID NO: 289 - protein, 111 aa, Artificial Sequence
  source: /note="Description of Artificial Sequence: Synthetic polypeptide"
DIQMTQSPSSLSASVGDRVTITCRSSQSITFYYKNYLAWYQQKPGKAPKLLIYWGSYLESGVPSRFSGSGSGTDFTLTISSLQPEDFATYYCQQHYRTPPSFGQGTKVEIK

SEQ ID NO: 290 - protein, 111 aa, Artificial Sequence
  source: /note="Description of Artificial Sequence: Synthetic polypeptide"
DIQMTQSPSSLSASVGDRVTITCRSSQSITFRYKNYLAWYQQKPGKAPKLLIYWGSYLESGVPSRFSGSGSGTDFTLTISSLQPEDFATYYCQQHYRTPPSFGQGTKVEIK

SEQ ID NO: 291 - protein, 111 aa, Artificial Sequence
  source: /note="Description of Artificial Sequence: Synthetic polypeptide"
DIQMTQSPSSLSASVGDRVTITCRSSQSITFRDKNYLAWYQQKPGKAPKLLIYWGSYLESGVPSRFSGSGSGTDFTLTISSLQPEDFATYYCQQHYRTPPSFGQGTKVEIK

SEQ ID NO: 292 - protein, 111 aa, Artificial Sequence
  source: /note="Description of Artificial Sequence: Synthetic polypeptide"
DIQMTQSPSSLSASVGDRVTITCRSSQSITDRYKNYLAWYQQKPGKAPKLLIYWGSYLESGVPSRFSGSGSGTDFTLTISSLQPEDFATYYCQQHYRTPPSFGQGTKVEIK

SEQ ID NO: 293 - protein, 111 aa, Artificial Sequence
  source: /note="Description of Artificial Sequence: Synthetic polypeptide"
DIQMTQSPSSLSASVGDRVTITCRSSESITFYYKNYLAWYQQKPGKAPKLLIYWGSYLESGVPSRFSGSGSGTDFTLTISSLQPEDFATYYCQQHYRTPPSFGQGTKVEIK

SEQ ID NO: 294 - protein, 111 aa, Artificial Sequence
  source: /note="Description of Artificial Sequence: Synthetic polypeptide"
DIQMTQSPSSLSASVGDRVTITCRSSQSITSDYKNYLAWYQQKPGKAPKLLIYWGSYLESGVPSRFSGSGSGTDFTLTISSLQPEDFATYYCQQHYRTPPSFGQGTKVEIK

SEQ ID NO: 295 - protein, 111 aa, Artificial Sequence
  source: /note="Description of Artificial Sequence: Synthetic polypeptide"
DIVMTQSPDSLAVSLGERATINCKSSQSVTFWYKNYLAWYQQKPGQPPKLLIYWASTRESGVPDRFSGSGSGTDFTLTISSLQAEDVAVYYCQQHYRTPPTFGQGTKVEIK

SEQ ID NO: 296 - protein, 111 aa, Artificial Sequence
  source: /note="Description of Artificial Sequence: Synthetic polypeptide"
DIQMTQSPSSLSASVGDRVTITCRSSEDITSDNQNYLAWYQQKPGKAPKLLIYWGSYLESGVPSRFSGSGSGTDFTLTISSLQPEDFATYYCQQHYRTPPSFGQGTKVEIK

SEQ ID NO: 297 - protein, 111 aa, Artificial Sequence
  source: /note="Description of Artificial Sequence: Synthetic polypeptide"
DIQMTQSPSSLSASVGDRVTITCRSSEDITSDNKNYLAWYQQKPGKAPKLLIYWGSYLESGVPSRFSGSGSGTDFTLTISSLQPEDFATYYCQQHYRTPPSFGQGTKVEIK

SEQ ID NO: 298 - protein, 111 aa, Artificial Sequence
  source: /note="Description of Artificial Sequence: Synthetic polypeptide"
DIQMTQSPSSLSASVGDRVTITCRSSQSITWDNKNYLAWYQQKPGKAPKLLIYWGSYLESGVPSRFSGSGSGTDFTLTISSLQPEDFATYYCQQHYRTPPSFGQGTKVEIK

SEQ ID NO: 299 - protein, 5 aa, Artificial Sequence
  source: /note="Description of Artificial Sequence: Synthetic peptide"
DYAIH

SEQ ID NO: 300 - protein, 5 aa, Artificial Sequence
  source: /note="Description of Artificial Sequence: Synthetic peptide"
DYAMH

SEQ ID NO: 301 - protein, 5 aa, Artificial Sequence
  source: /note="Description of Artificial Sequence: Synthetic peptide"
HYAMH

SEQ ID NO: 302 - protein, 5 aa, Artificial Sequence
  source: /note="Description of Artificial Sequence: Synthetic peptide"
LYAMH

SEQ ID NO: 303 - protein, 5 aa, Artificial Sequence
  source: /note="Description of Artificial Sequence: Synthetic peptide"
NYAMH

SEQ ID NO: 304 - protein, 5 aa, Artificial Sequence
  source: /note="Description of Artificial Sequence: Synthetic peptide"
NYGMH

SEQ ID NO: 305 - protein, 5 aa, Artificial Sequence
  source: /note="Description of Artificial Sequence: Synthetic peptide"
QYAMH

SEQ ID NO: 306 - protein, 5 aa, Artificial Sequence
  source: /note="Description of Artificial Sequence: Synthetic peptide"
SFAMH

SEQ ID NO: 307 - protein, 5 aa, Artificial Sequence
  source: /note="Description of Artificial Sequence: Synthetic peptide"
SHAMH

SEQ ID NO: 308 - protein, 5 aa, Artificial Sequence
  source: /note="Description of Artificial Sequence: Synthetic peptide"
SRAMH

SEQ ID NO: 309 - protein, 5 aa, Artificial Sequence
  source: /note="Description of Artificial Sequence: Synthetic peptide"
SYAIH

SEQ ID NO: 310 - protein, 5 aa, Artificial Sequence
  source: /note="Description of Artificial Sequence: Synthetic peptide"
SYAMH

SEQ ID NO: 311 - protein, 5 aa, Artificial Sequence
  source: /note="Description of Artificial Sequence: Synthetic peptide"
SYGMH

SEQ ID NO: 312 - protein, 5 aa, Artificial Sequence
  source: /note="Description of Artificial Sequence: Synthetic peptide"
SYSMH

SEQ ID NO: 313 - protein, 5 aa, Artificial Sequence
  source: /note="Description of Artificial Sequence: Synthetic peptide"
THAIH

SEQ ID NO: 314 - protein, 5 aa, Artificial Sequence
  source: /note="Description of Artificial Sequence: Synthetic peptide"
TRAIH

SEQ ID NO: 315 - protein, 5 aa, Artificial Sequence
  source: /note="Description of Artificial Sequence: Synthetic peptide"
TYAIH

SEQ ID NO: 316 - protein, 5 aa, Artificial Sequence
  source: /note="Description of Artificial Sequence: Synthetic peptide"
TYAIN

SEQ ID NO: 317 - protein, 5 aa, Artificial Sequence
  source: /note="Description of Artificial Sequence: Synthetic peptide"
TYAMH

SEQ ID NO: 318 - protein, 5 aa, Artificial Sequence
  source: /note="Description of Artificial Sequence: Synthetic peptide"
YYAMH

SEQ ID NO: 319 - protein, 17 aa, Artificial Sequence
  source: /note="Description of Artificial Sequence: Synthetic peptide"
IVSYDGNYKYYADSVKG

SEQ ID NO: 320 - protein, 17 aa, Artificial Sequence
  source: /note="Description of Artificial Sequence: Synthetic peptide"
VISYDANYKYYADSVKG

SEQ ID NO: 321 - protein, 17 aa, Artificial Sequence
  source: /note="Description of Artificial Sequence: Synthetic peptide"
VISYDANYKYYAQKFQG

SEQ ID NO: 322 - protein, 17 aa, Artificial Sequence
  source: /note="Description of Artificial Sequence: Synthetic peptide"
VISYDANYKYYAQKVQG

SEQ ID NO: 323 - protein, 17 aa, Artificial Sequence
  source: /note="Description of Artificial Sequence: Synthetic peptide"
VISYDGNYKYYADSVKG

SEQ ID NO: 324 - protein, 17 aa, Artificial Sequence
  source: /note="Description of Artificial Sequence: Synthetic peptide"
VISYDGNYKYYADSVQG

SEQ ID NO: 325 - protein, 17 aa, Artificial Sequence
  source: /note="Description of Artificial Sequence: Synthetic peptide"
VISYDGNYKYYAQKFQG

SEQ ID NO: 326 - protein, 17 aa, Artificial Sequence
  source: /note="Description of Artificial Sequence: Synthetic peptide"
VISYDGSNKYYADSVKG

SEQ ID NO: 327 - protein, 17 aa, Artificial Sequence
  source: /note="Description of Artificial Sequence: Synthetic peptide"
VISYDGSYKYYADSVKG

SEQ ID NO: 328 - protein, 17 aa, Artificial Sequence
  source: /note="Description of Artificial Sequence: Synthetic peptide"
VVCYDGNYKYYADSVQG

SEQ ID NO: 329 - protein, 17 aa, Artificial Sequence
  source: /note="Description of Artificial Sequence: Synthetic peptide"
VVSFDGNYKYYADSVQG

SEQ ID NO: 330 - protein, 17 aa, Artificial Sequence
  source: /note="Description of Artificial Sequence: Synthetic peptide"
VVSWDGNYKYYADSVQG

SEQ ID NO: 331 - protein, 17 aa, Artificial Sequence
  source: /note="Description of Artificial Sequence: Synthetic peptide"
VVSYDADYKYYADSVQG

SEQ ID NO: 332 - protein, 17 aa, Artificial Sequence
  source: /note="Description of Artificial Sequence: Synthetic peptide"
VVSYDAEYKYYADSVQG

SEQ ID NO: 333 - protein, 17 aa, Artificial Sequence
  source: /note="Description of Artificial Sequence: Synthetic peptide"
VVSYDAHYKYYADSVQG

SEQ ID NO: 334 - protein, 17 aa, Artificial Sequence
  source: /note="Description of Artificial Sequence: Synthetic peptide"
VVSYDANYKYYADSVQG

SEQ ID NO: 335 - protein, 17 aa, Artificial Sequence
  source: /note="Description of Artificial Sequence: Synthetic peptide"
VVSYDGFYKYYADSVQG

SEQ ID NO: 336 - protein, 17 aa, Artificial Sequence
  source: /note="Description of Artificial Sequence: Synthetic peptide"
VVSYDGHYKYYADSVQG

SEQ ID NO: 337 - protein, 17 aa, Artificial Sequence
  source: /note="Description of Artificial Sequence: Synthetic peptide"
VVSYDGNCKYYADSVQG

SEQ ID NO: 338 - protein, 17 aa, Artificial Sequence
  source: /note="Description of Artificial Sequence: Synthetic peptide"
VVSYDGNHKYYADSVQG

SEQ ID NO: 339 - protein, 17 aa, Artificial Sequence
  source: /note="Description of Artificial Sequence: Synthetic peptide"
VVSYDGNNKYYADSVQG

SEQ ID NO: 340 - protein, 17 aa, Artificial Sequence
  source: /note="Description of Artificial Sequence: Synthetic peptide"
VVSYDGNYKYYADSVKG

SEQ ID NO: 341 - protein, 17 aa, Artificial Sequence
  source: /note="Description of Artificial Sequence: Synthetic peptide"
VVSYDGNYKYYADSVQG

SEQ ID NO: 342 - protein, 17 aa, Artificial Sequence
  source: /note="Description of Artificial Sequence: Synthetic peptide"
VVSYDGNYKYYADSVRG

SEQ ID NO: 343 - protein, 17 aa, Artificial Sequence
  source: /note="Description of Artificial Sequence: Synthetic peptide"
VVSYDGSYKYYADSVQG

SEQ ID NO: 344 - protein, 17 aa, Artificial Sequence
  source: /note="Description of Artificial Sequence: Synthetic peptide"
VVSYDGYYKYYADSVQG

SEQ ID NO: 345 - protein, 17 aa, Artificial Sequence
  source: /note="Description of Artificial Sequence: Synthetic peptide"
VVSYDSNYKYYADSVKG

SEQ ID NO: 346 - protein, 17 aa, Artificial Sequence
  source: /note="Description of Artificial Sequence: Synthetic peptide"
VVSYNGNYKYYADSVQG

SEQ ID NO: 347 - protein, 17 aa, Artificial Sequence
  source: /note="Description of Artificial Sequence: Synthetic peptide"
VVSYSGNYKYYADSVQG

SEQ ID NO: 348 - protein, 17 aa, Artificial Sequence
  source: /note="Description of Artificial Sequence: Synthetic peptide"
WISYDANYKYYAQKFQG

SEQ ID NO: 349 - protein, 20 aa, Artificial Sequence
  source: /note="Description of Artificial Sequence: Synthetic peptide"
DSELRSLLYFEWLSQGYFNP

SEQ ID NO: 350 - protein, 20 aa, Artificial Sequence
  source: /note="Description of Artificial Sequence: Synthetic peptide"
DSQLRSLLMFEWLSQGYFDY

SEQ ID NO: 351 - protein, 20 aa, Artificial Sequence
  source: /note="Description of Artificial Sequence: Synthetic peptide"
DSQLRSLLYFEWLSQGYFDY

SEQ ID NO: 352 - protein, 20 aa, Artificial Sequence
  source: /note="Description of Artificial Sequence: Synthetic peptide"
DSQLRSLLYFEWLSQGYFNP

SEQ ID NO: 353 - protein, 20 aa, Artificial Sequence
  source: /note="Description of Artificial Sequence: Synthetic peptide"
DSQNRSLLMFEWLSQGYFDY

SEQ ID NO: 354 - protein, 20 aa, Artificial Sequence
  source: /note="Description of Artificial Sequence: Synthetic peptide"
DSQNRSLLYFEWLSQGYFDY

SEQ ID NO: 355 - protein, 20 aa, Artificial Sequence
  source: /note="Description of Artificial Sequence: Synthetic peptide"
DSRARSLLYFEWLSQGYFNP

SEQ ID NO: 356 - protein, 20 aa, Artificial Sequence
  source: /note="Description of Artificial Sequence: Synthetic peptide"
DSRDRSLLYFEWLSQGYFNP

SEQ ID NO: 357 - protein, 20 aa, Artificial Sequence
  source: /note="Description of Artificial Sequence: Synthetic peptide"
DSRHRSLLYFEWLSQGYFNP

SEQ ID NO: 358 - protein, 20 aa, Artificial Sequence
  source: /note="Description of Artificial Sequence: Synthetic peptide"
DSRIRSLLYFEWLSQGYFNP

SEQ ID NO: 359 - protein, 20 aa, Artificial Sequence
  source: /note="Description of Artificial Sequence: Synthetic peptide"
DSRKRSLLYFEWLSQGYFNP

SEQ ID NO: 360 - protein, 20 aa, Artificial Sequence
  source: /note="Description of Artificial Sequence: Synthetic peptide"
DSRLHSLLYFEWLSQGYFNP

SEQ ID NO: 361 - protein, 20 aa, Artificial Sequence
  source: /note="Description of Artificial Sequence: Synthetic peptide"
DSRLRLLLYFEWLSQGYFNP

SEQ ID NO: 362 - protein, 20 aa, Artificial Sequence
  source: /note="Description of Artificial Sequence: Synthetic peptide"
DSRLRSFLFFEWLSQGYFNP

SEQ ID NO: 363 - protein, 20 aa, Artificial Sequence
  source: /note="Description of Artificial Sequence: Synthetic peptide"
DSRLRSFLYFEWLSQGYFNP

SEQ ID NO: 364 - protein, 20 aa, Artificial Sequence
  source: /note="Description of Artificial Sequence: Synthetic peptide"
DSRLRSHLYFEWLSQGYFNP

SEQ ID NO: 365 - protein, 20 aa, Artificial Sequence
  source: /note="Description of Artificial Sequence: Synthetic peptide"
DSRLRSKLYFEWLSQGYFNP

SEQ ID NO: 366 - protein, 20 aa, Artificial Sequence
  source: /note="Description of Artificial Sequence: Synthetic peptide"
DSRLRSLCYFEWLSQGYFNP

SEQ ID NO: 367 - protein, 20 aa, Artificial Sequence
  source: /note="Description of Artificial Sequence: Synthetic peptide"
DSRLRSLHYFEWLSQGYFNP

SEQ ID NO: 368 - protein, 20 aa, Artificial Sequence
  source: /note="Description of Artificial Sequence: Synthetic peptide"
DSRLRSLLFFEWLSQGYFNP

SEQ ID NO: 369 - protein, 20 aa, Artificial Sequence
  source: /note="Description of Artificial Sequence: Synthetic peptide"
DSRLRSLLKFEWLSQGYFNP

SEQ ID NO: 370 - protein, 20 aa, Artificial Sequence
  source: /note="Description of Artificial Sequence: Synthetic peptide"
DSRLRSLLMFEWLSQGYFNP

SEQ ID NO: 371 - protein, 20 aa, Artificial Sequence
  source: /note="Description of Artificial Sequence: Synthetic peptide"
DSRLRSLLRFEWLSQGYFNP

SEQ ID NO: 372 - protein, 20 aa, Artificial Sequence
  source: /note="Description of Artificial Sequence: Synthetic peptide"
DSRLRSLLYCEWCSQGYFNP

SEQ ID NO: 373 - protein, 20 aa, Artificial Sequence
  source: /note="Description of Artificial Sequence: Synthetic peptide"
DSRLRSLLYFDWLSQGYFNP

SEQ ID NO: 374 - protein, 20 aa, Artificial Sequence
  source: /note="Description of Artificial Sequence: Synthetic peptide"
DSRLRSLLYFECLSQGYFNP

SEQ ID NO: 375 - protein, 20 aa, Artificial Sequence
  source: /note="Description of Artificial Sequence: Synthetic peptide"
DSRLRSLLYFEHLSQGYFNP

SEQ ID NO: 376 - protein, 20 aa, Artificial Sequence
  source: /note="Description of Artificial Sequence: Synthetic peptide"
DSRLRSLLYFERLSQGYFNP

SEQ ID NO: 377 - protein, 20 aa, Artificial Sequence
  source: /note="Description of Artificial Sequence: Synthetic peptide"
DSRLRSLLYFEWLSCGYFNP

SEQ ID NO: 378 - protein, 20 aa, Artificial Sequence
  source: /note="Description of Artificial Sequence: Synthetic peptide"
DSRLRSLLYFEWLSLGYFNP

SEQ ID NO: 379 - protein, 20 aa, Artificial Sequence
  source: /note="Description of Artificial Sequence: Synthetic peptide"
DSRLRSLLYFEWLSQGRFNP

SEQ ID NO: 380 - protein, 20 aa, Artificial Sequence
  source: /note="Description of Artificial Sequence: Synthetic peptide"
DSRLRSLLYFEWLSQGYFDD

SEQ ID NO: 381 - protein, 20 aa, Artificial Sequence
  source: /note="Description of Artificial Sequence: Synthetic peptide"
DSRLRSLLYFEWLSQGYFDF

SEQ ID NO: 382 - protein, 20 aa, Artificial Sequence
  source: /note="Description of Artificial Sequence: Synthetic peptide"
DSRLRSLLYFEWLSQGYFDI

SEQ ID NO: 383 - protein, 20 aa, Artificial Sequence
  source: /note="Description of Artificial Sequence: Synthetic peptide"
DSRLRSLLYFEWLSQGYFDL

SEQ ID NO: 384 - protein, 20 aa, Artificial Sequence
  source: /note="Description of Artificial Sequence: Synthetic peptide"
DSRLRSLLYFEWLSQGYFDP

SEQ ID NO: 385 - protein, 20 aa, Artificial Sequence
  source: /note="Description of Artificial Sequence: Synthetic peptide"
DSRLRSLLYFEWLSQGYFDV

SEQ ID NO: 386 - protein, 20 aa, Artificial Sequence
  source: /note="Description of Artificial Sequence: Synthetic peptide"
DSRLRSLLYFEWLSQGYFDY

SEQ ID NO: 387 - protein, 20 aa, Artificial Sequence
  source: /note="Description of Artificial Sequence: Synthetic peptide"
DSRLRSLLYFEWLSQGYFEY

SEQ ID NO: 388 - protein, 20 aa, Artificial Sequence
  source: /note="Description of Artificial Sequence: Synthetic peptide"
DSRLRSLLYFEWLSQGYFGV

SEQ ID NO: 389 - protein, 20 aa, Artificial Sequence
  source: /note="Description of Artificial Sequence: Synthetic peptide"
DSRLRSLLYFEWLSQGYFND

SEQ ID NO: 390 - protein, 20 aa, Artificial Sequence
  source: /note="Description of Artificial Sequence: Synthetic peptide"
DSRLRSLLYFEWLSQGYFNP

SEQ ID NO: 391 - protein, 20 aa, Artificial Sequence
  source: /note="Description of Artificial Sequence: Synthetic peptide"
DSRLRSLLYFEWLSQGYFQH

SEQ ID NO: 392 - protein, 20 aa, Artificial Sequence
  source: /note="Description of Artificial Sequence: Synthetic peptide"
DSRLRSLLYFEWLSQGYFQP

SEQ ID NO: 393 - protein, 20 aa, Artificial Sequence
  source: /note="Description of Artificial Sequence: Synthetic peptide"
DSRLRSLLYFEWLSQGYFSY

SEQ ID NO: 394 - protein, 20 aa, Artificial Sequence
  source: /note="Description of Artificial Sequence: Synthetic peptide"
DSRLRSLLYFEWLSQGYMDV

SEQ ID NO: 395 - protein, 20 aa, Artificial Sequence
  source: /note="Description of Artificial Sequence: Synthetic peptide"
DSRLRSLLYFEWLSQGYMGV

SEQ ID NO: 396 - protein, 20 aa, Artificial Sequence
  source: /note="Description of Artificial Sequence: Synthetic peptide"
DSRLRSLLYFEWMNQGYFNP

SEQ ID NO: 397 - protein, 20 aa, Artificial Sequence
  source: /note="Description of Artificial Sequence: Synthetic peptide"
DSRLRSLLYFEWNSQGYFNP

SEQ ID NO: 398 - protein, 20 aa, Artificial Sequence
  source: /note="Description of Artificial Sequence: Synthetic peptide"
DSRLRSLLYQEWLSQGYFNP

SEQ ID NO: 399 - protein, 20 aa, Artificial Sequence
  source: /note="Description of Artificial Sequence: Synthetic peptide"
DSRLRSLNYFEWLSQGYFNP

SEQ ID NO: 400 - protein, 20 aa, Artificial Sequence
  source: /note="Description of Artificial Sequence: Synthetic peptide"
DSRLRSLQYFEWLSQGYFNP

SEQ ID NO: 401 - protein, 20 aa, Artificial Sequence
  source: /note="Description of Artificial Sequence: Synthetic peptide"
DSRLRSLRYFEWLSGGYFNP

SEQ ID NO: 402 - protein, 20 aa, Artificial Sequence
  source: /note="Description of Artificial Sequence: Synthetic peptide"
DSRLRSNLYYEWLSQGYFNP

SEQ ID NO: 403 - protein, 20 aa, Artificial Sequence
  source: /note="Description of Artificial Sequence: Synthetic peptide"
DSRLRSQLYFEWLSQGYFNP

SEQ ID NO: 404 - protein, 20 aa, Artificial Sequence
  source: /note="Description of Artificial Sequence: Synthetic peptide"
DSRLRSQLYYEWLSQGYFNP

SEQ ID NO: 405 - protein, 20 aa, Artificial Sequence
  source: /note="Description of Artificial Sequence: Synthetic peptide"
DSRLRSRLYFEWLSQGYFNP

SEQ ID NO: 406 - protein, 20 aa, Artificial Sequence
  source: /note="Description of Artificial Sequence: Synthetic peptide"
DSRLYSLLYFEWLSQGYFNP

SEQ ID NO: 407 - protein, 20 aa, Artificial Sequence
  source: /note="Description of Artificial Sequence: Synthetic peptide"
DSRMRSLLYFEWLSQGYFNP

SEQ ID NO: 408 - protein, 20 aa, Artificial Sequence
  source: /note="Description of Artificial Sequence: Synthetic peptide"
DSRNHSLLYFEWLSQGYFNP

SEQ ID NO: 409 - protein, 20 aa, Artificial Sequence
  source: /note="Description of Artificial Sequence: Synthetic peptide"
DSRNRSLLMFEWLSQGYFNP

SEQ ID NO: 410 - protein, 20 aa, Artificial Sequence
  source: /note="Description of Artificial Sequence: Synthetic peptide"
DSRNRSLLMFEWNNQGYFNP

SEQ ID NO: 411 - protein, 20 aa, Artificial Sequence
  source: /note="Description of Artificial Sequence: Synthetic peptide"
DSRNRSLLYFEWLSQGYFNP

SEQ ID NO: 412 - protein, 20 aa, Artificial Sequence
  source: /note="Description of Artificial Sequence: Synthetic peptide"
DSRNRVLLYFEWLSQGYFNP

SEQ ID NO: 413 - protein, 20 aa, Artificial Sequence
  source: /note="Description of Artificial Sequence: Synthetic peptide"
DSRNYSLLYFEWLSQGYFNP

SEQ ID NO: 414 - protein, 20 aa, Artificial Sequence
  source: /note="Description of Artificial Sequence: Synthetic peptide"
DSRPRSLLYFEWLSQGYFNP

SEQ ID NO: 415 - protein, 20 aa, Artificial Sequence
  source: /note="Description of Artificial Sequence: Synthetic peptide"
DSRQRSLLMFEWLSQGYFNP

SEQ ID NO: 416 - protein, 20 aa, Artificial Sequence
  source: /note="Description of Artificial Sequence: Synthetic peptide"
DSRQRSLLYFEWLSQGYFNP

SEQ ID NO: 417 - protein, 20 aa, Artificial Sequence
  source: /note="Description of Artificial Sequence: Synthetic peptide"
DSRRRSLLYFEWLSQGYFNP

SEQ ID NO: 418 - protein, 20 aa, Artificial Sequence
  source: /note="Description of Artificial Sequence: Synthetic peptide"
DSRSRSLLYFEWLSQGYFNP

SEQ ID NO: 419 - protein, 20 aa, Artificial Sequence
  source: /note="Description of Artificial Sequence: Synthetic peptide"
DSRTRSLLYFEWLSQGYFNP

SEQ ID NO: 420 - protein, 20 aa, Artificial Sequence
  source: /note="Description of Artificial Sequence: Synthetic peptide"
DSRVRSLLYFEWLSQGYFNP

SEQ ID NO: 421 - protein, 20 aa, Artificial Sequence
  source: /note="Description of Artificial Sequence: Synthetic peptide"
DSRYRSLLYFEWLSQGYFNP

SEQ ID NO: 422 - protein, 20 aa, Artificial Sequence
  source: /note="Description of Artificial Sequence: Synthetic peptide"
ERPLRLLRYFDWLSGGANDY

SEQ ID NO: 423 - protein, 20 aa, Artificial Sequence
  source: /note="Description of Artificial Sequence: Synthetic peptide"
ESRLRSLLYFEWLSQGYFNP

SEQ ID NO: 424 - protein, 15 aa, Artificial Sequence
  source: /note="Description of Artificial Sequence: Synthetic peptide"
RSSQSITFDYKNYLA

SEQ ID NO: 425 - protein, 15 aa, Artificial Sequence
  source: /note="Description of Artificial Sequence: Synthetic peptide"
KSSQSVTFDYKNYLA

SEQ ID NO: 426 - protein, 15 aa, Artificial Sequence
  source: /note="Description of Artificial Sequence: Synthetic peptide"
RSSQSITFDYQNYLA

SEQ ID NO: 427 - protein, 15 aa, Artificial Sequence
  source: /note="Description of Artificial Sequence: Synthetic peptide"
RSSQSITSDNQNYLA

SEQ ID NO: 428 - protein, 17 aa, Artificial Sequence
  source: /note="Description of Artificial Sequence: Synthetic peptide"
KSSQSVLYSSNNKNYLA

SEQ ID NO: 429 - protein, 15 aa, Artificial Sequence
  source: /note="Description of Artificial Sequence: Synthetic peptide"
KSSQSVSFNYKNYLA

SEQ ID NO: 430 - protein, 15 aa, Artificial Sequence
  source: /note="Description of Artificial Sequence: Synthetic peptide"
KSSQSVTFNYKNYLA

SEQ ID NO: 431 - protein, 15 aa, Artificial Sequence
  source: /note="Description of Artificial Sequence: Synthetic peptide"
RSSQSITFDYKNRLA

SEQ ID NO: 432 - protein, 15 aa, Artificial Sequence
  source: /note="Description of Artificial Sequence: Synthetic peptide"
RSSQSITSDNKNYLA

SEQ ID NO: 433 - protein, 15 aa, Artificial Sequence
  source: /note="Description of Artificial Sequence: Synthetic peptide"
RSSQSITSDDKNYLA

SEQ ID NO: 434 - protein, 15 aa, Artificial Sequence
  source: /note="Description of Artificial Sequence: Synthetic peptide"
RSSQSITSDEKNYLA

SEQ ID NO: 435 - protein, 15 aa, Artificial Sequence
  source: /note="Description of Artificial Sequence: Synthetic peptide"
RSSQSITDDNKNYLA

SEQ ID NO: 436 - protein, 15 aa, Artificial Sequence
  source: /note="Description of Artificial Sequence: Synthetic peptide"
RSSQSITDDDKNYLA

SEQ ID NO: 437 - protein, 15 aa, Artificial Sequence
  source: /note="Description of Artificial Sequence: Synthetic peptide"
RSSQSITDDEKNYLA

SEQ ID NO: 438 - protein, 15 aa, Artificial Sequence
  source: /note="Description of Artificial Sequence: Synthetic peptide"
RSSQSITEDNKNYLA

SEQ ID NO: 439 - protein, 15 aa, Artificial Sequence
  source: /note="Description of Artificial Sequence: Synthetic peptide"
RSSQSITEDDKNYLA

SEQ ID NO: 440 - protein, 15 aa, Artificial Sequence
  source: /note="Description of Artificial Sequence: Synthetic peptide"
RSSQSITEDEKNYLA

SEQ ID NO: 441 - protein, 15 aa, Artificial Sequence
  source: /note="Description of Artificial Sequence: Synthetic peptide"
RSSQSITQDNKNYLA

SEQ ID NO: 442 - protein, 15 aa, Artificial Sequence
  source: /note="Description of Artificial Sequence: Synthetic peptide"
RSSQSITQDDKNYLA

SEQ ID NO: 443 - protein, 15 aa, Artificial Sequence
  source: /note="Description of Artificial Sequence: Synthetic peptide"
RSSQSITQDEKNYLA

SEQ ID NO: 444 - protein, 15 aa, Artificial Sequence
  source: /note="Description of Artificial Sequence: Synthetic peptide"
RSSQSITHDNKNYLA

SEQ ID NO: 445 - protein, 15 aa, Artificial Sequence
  source: /note="Description of Artificial Sequence: Synthetic peptide"
RSSQSITHDDKNYLA

SEQ ID NO: 446 - protein, 15 aa, Artificial Sequence
  source: /note="Description of Artificial Sequence: Synthetic peptide"
RSSQSITHDEKNYLA

SEQ ID NO: 447 - protein, 15 aa, Artificial Sequence
  source: /note="Description of Artificial Sequence: Synthetic peptide"
RSSQSITFNYKNYLA

SEQ ID NO: 448 - protein, 15 aa, Artificial Sequence
  source: /note="Description of Artificial Sequence: Synthetic peptide"
RSSQSVTFDYKNYLA

SEQ ID NO: 449 - protein, 15 aa, Artificial Sequence
  source: /note="Description of Artificial Sequence: Synthetic peptide"
RSSQSITSDYKNYLA

SEQ ID NO: 450 - protein, 15 aa, Artificial Sequence
  source: /note="Description of Artificial Sequence: Synthetic peptide"
KSSQSITFDYKNYLA

SEQ ID NO: 451 - protein, 15 aa, Artificial Sequence
  source: /note="Description of Artificial Sequence: Synthetic peptide"
KSSQSVTDDNKNYLA

SEQ ID NO: 452 - protein, 15 aa, Artificial Sequence
  source: /note="Description of Artificial Sequence: Synthetic peptide"
KSSQSVTSDYKNYLA

SEQ ID NO: 453 - protein, 15 aa, Artificial Sequence
  source: /note="Description of Artificial Sequence: Synthetic peptide"
KSSQSVTSDNKNYLA

SEQ ID NO: 454 - protein, 15 aa, Artificial Sequence
  source: /note="Description of Artificial Sequence: Synthetic peptide"
RSSESITFDYKNYLA

SEQ ID NO: 455 - protein, 15 aa, Artificial Sequence
  source: /note="Description of Artificial Sequence: Synthetic peptide"
RSSEDITFDYKNYLA

SEQ ID NO: 456 - protein, 17 aa, Artificial Sequence
  source: /note="Description of Artificial Sequence: Synthetic peptide"
RSSQSITFSSDYKNYLA

SEQ ID NO: 457 - protein, 17 aa, Artificial Sequence
  source: /note="Description of Artificial Sequence: Synthetic peptide"
RSSQSITFSPDYKNYLA

SEQ ID NO: 458 - protein, 17 aa, Artificial Sequence
  source: /note="Description of Artificial Sequence: Synthetic peptide"
RSSQSITLSPDYKNYLA

SEQ ID NO: 459 - protein, 17 aa, Artificial Sequence
  source: /note="Description of Artificial Sequence: Synthetic peptide"
RSSQSITISPDYKNYLA

SEQ ID NO: 460 - protein, 17 aa, Artificial Sequence
  source: /note="Description of Artificial Sequence: Synthetic peptide"
RSSQSITFGGDYKNYLA

SEQ ID NO: 461 - protein, 16 aa, Artificial Sequence
  source: /note="Description of Artificial Sequence: Synthetic peptide"
RSSQSITFSDYKNYLA

SEQ ID NO: 462 - protein, 16 aa, Artificial Sequence
  source: /note="Description of Artificial Sequence: Synthetic peptide"
RSSQSITFGDYKNYLA

SEQ ID NO: 463 - protein, 17 aa, Artificial Sequence
  source: /note="Description of Artificial Sequence: Synthetic peptide"
RSSQSITFGPDYKNYLA

SEQ ID NO: 464 - protein, 15 aa, Artificial Sequence
  source: /note="Description of Artificial Sequence: Synthetic peptide"
RSSQSITYDYKNYLA

SEQ ID NO: 465 - protein, 15 aa, Artificial Sequence
  source: /note="Description of Artificial Sequence: Synthetic peptide"
RSSQSITWDYKNYLA

SEQ ID NO: 466 - protein, 15 aa, Artificial Sequence
  source: /note="Description of Artificial Sequence: Synthetic peptide"
RSSQSITFWYKNYLA

SEQ ID NO: 467 - protein, 16 aa, Artificial Sequence
  source: /note="Description of Artificial Sequence: Synthetic peptide"
RSSQSITFLDYKNYLA

SEQ ID NO: 468 - protein, 17 aa, Artificial Sequence
  source: /note="Description of Artificial Sequence: Synthetic peptide"
RSSQSITLSPWYKNYLA

SEQ ID NO: 469 - protein, 17 aa, Artificial Sequence
  source: /note="Description of Artificial Sequence: Synthetic peptide"
RSSQSITLSPYYKNYLA

SEQ ID NO: 470 - protein, 17 aa, Artificial Sequence
  source: /note="Description of Artificial Sequence: Synthetic peptide"
RSSQSITLSPYDKNYLA

SEQ ID NO: 471 - protein, 15 aa, Artificial Sequence
  source: /note="Description of Artificial Sequence: Synthetic peptide"
RSSQSITFDNKNYLA

SEQ ID NO: 472 - protein, 15 aa, Artificial Sequence
  source: /note="Description of Artificial Sequence: Synthetic peptide"
RSSQSITADNKNYLA

SEQ ID NO: 473 - protein, 15 aa, Artificial Sequence
  source: /note="Description of Artificial Sequence: Synthetic peptide"
RSSQSITNDNKNYLA

SEQ ID NO: 474 - protein, 15 aa, Artificial Sequence
  source: /note="Description of Artificial Sequence: Synthetic peptide"
RSSQSITMDNKNYLA

SEQ ID NO: 475 - protein, 15 aa, Artificial Sequence
  source: /note="Description of Artificial Sequence: Synthetic peptide"
RSSQSITRDNKNYLA

SEQ ID NO: 476 - protein, 15 aa, Artificial Sequence
  source: /note="Description of Artificial Sequence: Synthetic peptide"
RSSQSITSDSKNYLA

SEQ ID NO: 477 - protein, 15 aa, Artificial Sequence
  source: /note="Description of Artificial Sequence: Synthetic peptide"
RSSQSITSDQKNYLA

SEQ ID NO: 478 - protein, 15 aa, Artificial Sequence
  source: /note="Description of Artificial Sequence: Synthetic peptide"
RSSQSITSDRKNYLA

SEQ ID NO: 479 - protein, 15 aa, Artificial Sequence
  source: /note="Description of Artificial Sequence: Synthetic peptide"
RSSEDITFWYKNYLA

SEQ ID NO: 480 - protein, 15 aa, Artificial Sequence
  source: /note="Description of Artificial Sequence: Synthetic peptide"
RSSQSITFWEKNYLA

SEQ ID NO: 481 - protein, 15 aa, Artificial Sequence
  source: /note="Description of Artificial Sequence: Synthetic peptide"
RSSQSITSWNKNYLA

SEQ ID NO: 482 - protein, 15 aa, Artificial Sequence
  source: /note="Description of Artificial Sequence: Synthetic peptide"
RSSEDITSWNKNYLA

SEQ ID NO: 483 - protein, 15 aa, Artificial Sequence
  source: /note="Description of Artificial Sequence: Synthetic peptide"
RSSEDITSWDKNYLA

SEQ ID NO: 484 - protein, 15 aa, Artificial Sequence
  source: /note="Description of Artificial Sequence: Synthetic peptide"
RSSQSITFYYKNYLA

SEQ ID NO: 485 - protein, 15 aa, Artificial Sequence
  source: /note="Description of Artificial Sequence: Synthetic peptide"
RSSQSITFRYKNYLA

SEQ ID NO: 486 - protein, 15 aa, Artificial Sequence
  source: /note="Description of Artificial Sequence: Synthetic peptide"
RSSQSITFRDKNYLA

SEQ ID NO: 487 - protein, 15 aa, Artificial Sequence
  source: /note="Description of Artificial Sequence: Synthetic peptide"
RSSQSITDRYKNYLA

SEQ ID NO: 488 - protein, 15 aa, Artificial Sequence
  source: /note="Description of Artificial Sequence: Synthetic peptide"
RSSESITFYYKNYLA

SEQ ID NO: 489 - protein, 15 aa, Artificial Sequence
  source: /note="Description of Artificial Sequence: Synthetic peptide"
KSSQSVTFWYKNYLA

SEQ ID NO: 490 - protein, 15 aa, Artificial Sequence
  source: /note="Description of Artificial Sequence: Synthetic peptide"
RSSEDITSDNQNYLA

SEQ ID NO: 491 - protein, 15 aa, Artificial Sequence
  source: /note="Description of Artificial Sequence: Synthetic peptide"
RSSEDITSDNKNYLA

SEQ ID NO: 492 - protein, 15 aa, Artificial Sequence
  source: /note="Description of Artificial Sequence: Synthetic peptide"
RSSQSITWDNKNYLA

SEQ ID NO: 493 - protein, 7 aa, Artificial Sequence
  source: /note="Description of Artificial Sequence: Synthetic peptide"
WGSYLES

SEQ ID NO: 494 - protein, 7 aa, Artificial Sequence
  source: /note="Description of Artificial Sequence: Synthetic peptide"
WGSSLQS

SEQ ID NO: 495 - protein, 7 aa, Artificial Sequence
  source: /note="Description of Artificial Sequence: Synthetic peptide"
AASSLQS

SEQ ID NO: 496 - protein, 7 aa, Artificial Sequence
  source: /note="Description of Artificial Sequence: Synthetic peptide"
WASTRES

SEQ ID NO: 497 - protein, 7 aa, Artificial Sequence
  source: /note="Description of Artificial Sequence: Synthetic peptide"
EASTRES

SEQ ID NO: 498 - protein, 7 aa, Artificial Sequence
  source: /note="Description of Artificial Sequence: Synthetic peptide"
WGSTRAT

SEQ ID NO: 499 - protein, 7 aa, Artificial Sequence
  source: /note="Description of Artificial Sequence: Synthetic peptide"
SASTRAT

SEQ ID NO: 500 - protein, 7 aa, Artificial Sequence
  source: /note="Description of Artificial Sequence: Synthetic peptide"
WGSTLES

SEQ ID NO: 501 - protein, 7 aa, Artificial Sequence
  source: /note="Description of Artificial Sequence: Synthetic peptide"
WASARES

SEQ ID NO: 502 - protein, 7 aa, Artificial Sequence
  source: /note="Description of Artificial Sequence: Synthetic peptide"
EGSYLES

SEQ ID NO: 503 - protein, 7 aa, Artificial Sequence
  source: /note="Description of Artificial Sequence: Synthetic peptide"
SGSYLES

SEQ ID NO: 504 - protein, 7 aa, Artificial Sequence
  source: /note="Description of Artificial Sequence: Synthetic peptide"
AASSRQS

SEQ ID NO: 505 - protein, 7 aa, Artificial Sequence
  source: /note="Description of Artificial Sequence: Synthetic peptide"
AASSLES

SEQ ID NO: 506 - protein, 7 aa, Artificial Sequence
  source: /note="Description of Artificial Sequence: Synthetic peptide"
SASSLQS

SEQ ID NO: 507 - protein, 7 aa, Artificial Sequence
  source: /note="Description of Artificial Sequence: Synthetic peptide"
SGSSLQS

SEQ ID NO: 508 - protein, 7 aa, Artificial Sequence
  source: /note="Description of Artificial Sequence: Synthetic peptide"
SASTRES

SEQ ID NO: 509 - protein, 7 aa, Artificial Sequence
  source: /note="Description of Artificial Sequence: Synthetic peptide"
WASTRQS

SEQ ID NO: 510 - protein, 7 aa, Artificial Sequence
  source: /note="Description of Artificial Sequence: Synthetic peptide"
SGSTRES

SEQ ID NO: 511 - protein, 7 aa, Artificial Sequence
  source: /note="Description of Artificial Sequence: Synthetic peptide"
WGSELES

SEQ ID NO: 512 - protein, 7 aa, Artificial Sequence
  source: /note="Description of Artificial Sequence: Synthetic peptide"
WGSDLES

SEQ ID NO: 513 - protein, 7 aa, Artificial Sequence
  source: /note="Description of Artificial Sequence: Synthetic peptide"
WGEYLES

SEQ ID NO: 514 - protein, 7 aa, Artificial Sequence
  source: /note="Description of Artificial Sequence: Synthetic peptide"
AASTRES

SEQ ID NO: 515 - protein, 9 aa, Artificial Sequence
  source: /note="Description of Artificial Sequence: Synthetic peptide"
QQHYRTPPS

SEQ ID NO: 516 - protein, 9 aa, Artificial Sequence
  source: /note="Description of Artificial Sequence: Synthetic peptide"
QQCYRTPPS

SEQ ID NO: 517 - protein, 9 aa, Artificial Sequence
  source: /note="Description of Artificial Sequence: Synthetic peptide"
QQHYCTPPS

SEQ ID NO: 518 - protein, 9 aa, Artificial Sequence
  source: /note="Description of Artificial Sequence: Synthetic peptide"
QQFYRTPPS

SEQ ID NO: 519 - protein, 8 aa, Artificial Sequence
  source: /note="Description of Artificial Sequence: Synthetic peptide"
QQYYRSPS

SEQ ID NO: 520 - protein, 9 aa, Artificial Sequence
  source: /note="Description of Artificial Sequence: Synthetic peptide"
QQYYRTPPS

SEQ ID NO: 521 - protein, 9 aa, Artificial Sequence
  source: /note="Description of Artificial Sequence: Synthetic peptide"
QQYYRSPPS

SEQ ID NO: 522 - protein, 9 aa, Artificial Sequence
  source: /note="Description of Artificial Sequence: Synthetic peptide"
QQHYRTPPT

SEQ ID NO: 523 - protein, 9 aa, Artificial Sequence
  source: /note="Description of Artificial Sequence: Synthetic peptide"
QQHYSTPPS

SEQ ID NO: 524 - protein, 9 aa, Artificial Sequence
  source: /note="Description of Artificial Sequence: Synthetic peptide"
QQHYQTPPS

SEQ ID NO: 525 - protein, 8 aa, Artificial Sequence
  source: /note="Description of Artificial Sequence: Synthetic peptide"
QQHYRTPS